SEQUENCE LISTING (609 sequences)

SEQ ID NO: 1 - protein, 12 aa, Herpes simplex virus type 1
VPPGPLGYVYAR

SEQ ID NO: 2 - protein, 11 aa, Herpes simplex virus type 1
VIAEPFNANHR

SEQ ID NO: 3 - protein, 11 aa, Herpes simplex virus type 1
EALHTVVNNVR

SEQ ID NO: 4 - protein, 15 aa, Herpes simplex virus type 1
VLFAGASANASEAAK

SEQ ID NO: 5 - protein, 10 aa, Herpes simplex virus type 1
LQFTYNHIQR

SEQ ID NO: 6 - protein, 12 aa, Herpes simplex virus type 1
DVTVSQVWFGHR

SEQ ID NO: 7 - protein, 10 aa, Herpes simplex virus type 1
FTVAWDWVPK

SEQ ID NO: 8 - protein, 10 aa, Herpes simplex virus type 1
NNLETTAFHR

SEQ ID NO: 9 - protein, 8 aa, Herpes simplex virus type 1
SHPSTPPK

SEQ ID NO: 10 - protein, 26 aa, Herpes simplex virus type 1
FVGDQVPHTTYYDGGVELWHYPMGHK

SEQ ID NO: 11 - protein, 24 aa, Herpes simplex virus type 1
ATDSTHSPAYPTLELNLAQQPLLR

SEQ ID NO: 12 - protein, 12 aa, Herpes simplex virus type 1
VVHVVTVTACPR

SEQ ID NO: 13 - protein, 15 aa, Herpes simplex virus type 1
GSGGPGVEGGHGPSR

SEQ ID NO: 14 - protein, 12 aa, Herpes simplex virus type 1
HPWSGEQGASTR

SEQ ID NO: 15 - protein, 17 aa, Herpes simplex virus type 1
DLPDSSDSEAETEVGGR

SEQ ID NO: 16 - protein, 14 aa, Herpes simplex virus type 1
AGTAVDFIWTGNQR

SEQ ID NO: 17 - protein, 19 aa, Herpes simplex virus type 1
YLPISGVSSVVALSPYVNK

SEQ ID NO: 18 - protein, 14 aa, Herpes simplex virus type 1
LLQVSGGTWGMHLR

SEQ ID NO: 19 - protein, 9 aa, Herpes simplex virus type 1
WTPDLGYMR

SEQ ID NO: 20 - protein, 14 aa, Herpes simplex virus type 1
APTPSAPSPNAMLR

SEQ ID NO: 21 - protein, 7 aa, Herpes simplex virus type 1
DGVIFPK

SEQ ID NO: 22 - protein, 11 aa, Herpes simplex virus type 1
SGADAPGSDAR

SEQ ID NO: 23 - protein, 13 aa, Herpes simplex virus type 1
VAGGSEAAVAAVR

SEQ ID NO: 24 - protein, 15 aa, Herpes simplex virus type 1
ALLEPDDDAPPLVLR

SEQ ID NO: 25 - protein, 19 aa, Herpes simplex virus type 1
APAAGTDAGEDAGDAVSPR

SEQ ID NO: 26 - protein, 12 aa, Herpes simplex virus type 1
GPSSAAPAAPGR

SEQ ID NO: 27 - protein, 19 aa, Herpes simplex virus type 1
DGYVSGEPWPGAGPPPPGR

SEQ ID NO: 28 - protein, 17 aa, Herpes simplex virus type 1
VLGASETIANIYTTQHR

SEQ ID NO: 29 - protein, 10 aa, Herpes simplex virus type 1
VYGLLANTVR

SEQ ID NO: 30 - protein, 14 aa, Herpes simplex virus type 1
DDIVYVPEPMTYWR

SEQ ID NO: 31 - protein, 11 aa, Herpes simplex virus type 1
VYIDGPHGMGK

SEQ ID NO: 32 - protein, 11 aa, Herpes simplex virus type 1
DIHGYLAPVPK

SEQ ID NO: 33 - protein, 15 aa, Herpes simplex virus type 1
HTISPVSWSSGDLVR

SEQ ID NO: 34 - protein, 12 aa, Herpes simplex virus type 1
ADDGGEAGADTR

SEQ ID NO: 35 - protein, 11 aa, Herpes simplex virus type 1
APLAIGPLWAR

SEQ ID NO: 36 - protein, 7 aa, Herpes simplex virus type 1
GPDSPPK

SEQ ID NO: 37 - protein, 15 aa, Herpes simplex virus type 1
LEDGAGALGAAGPSK

SEQ ID NO: 38 - protein, 7 aa, Herpes simplex virus type 1
WSLVAER

SEQ ID NO: 39 - protein, 14 aa, Herpes simplex virus type 1
QAGGQPAAGDPGVR

SEQ ID NO: 40 - protein, 13 aa, Herpes simplex virus type 1
AADLFVSQMMGAR

SEQ ID NO: 41 - protein, 19 aa, Herpes simplex virus type 1
LGTIVTYDTGLDAAIAPFR

SEQ ID NO: 42 - protein, 12 aa, Herpes simplex virus type 1
LAAEAELALSGR

SEQ ID NO: 43 - protein, 14 aa, Herpes simplex virus type 1
IPQSSPEEAVLLDK

SEQ ID NO: 44 - protein, 9 aa, Herpes simplex virus type 1
ESLLSILLR

SEQ ID NO: 45 - protein, 10 aa, Herpes simplex virus type 1
LNAVAEAVLK

SEQ ID NO: 46 - protein, 14 aa, Herpes simplex virus type 1
GISADHFEAEVVER

SEQ ID NO: 47 - protein, 16 aa, Herpes simplex virus type 1
DLPTGEASPGAFSAFR

SEQ ID NO: 48 - protein, 21 aa, Herpes simplex virus type 1
EEGVSSSTSTQVQILSNALTK

SEQ ID NO: 49 - protein, 12 aa, Herpes simplex virus type 1
VELAITGQAPFR

SEQ ID NO: 50 - protein, 13 aa, Herpes simplex virus type 1
TSLLDSLLVMGDR

SEQ ID NO: 51 - protein, 9 aa, Herpes simplex virus type 1
EHLNLPLVR

SEQ ID NO: 52 - protein, 10 aa, Herpes simplex virus type 1
LSFLPAGHTR

SEQ ID NO: 53 - protein, 12 aa, Herpes simplex virus type 1
DGLGLYYEALSR

SEQ ID NO: 54 - protein, 20 aa, Herpes simplex virus type 1
FLSTLPSDVVEWGDAYVPER

SEQ ID NO: 55 - protein, 13 aa, Herpes simplex virus type 1
APPPPAGSGGAGR

SEQ ID NO: 56 - protein, 17 aa, Herpes simplex virus type 1
LHFSTAPPNPDAPWTPR

SEQ ID NO: 57 - protein, 15 aa, Herpes simplex virus type 1
TDEDLNELLGITTIR

SEQ ID NO: 58 - protein, 27 aa, Herpes simplex virus type 1
FVQYDESDYALYGGSSSEDDEHPEVPR

SEQ ID NO: 59 - protein, 12 aa, Herpes simplex virus type 1
GSEQPDPPGGQR

SEQ ID NO: 60 - protein, 12 aa, Herpes simplex virus type 1
APAADTIDATTR

SEQ ID NO: 61 - protein, 15 aa, Herpes simplex virus type 1
VSWETLVAHGPSLYR

SEQ ID NO: 62 - protein, 17 aa, Herpes simplex virus type 1
GVAEIDYATLGVGVGEK

SEQ ID NO: 63 - protein, 12 aa, Herpes simplex virus type 1
GPAYTTAAGVVR

SEQ ID NO: 64 - protein, 12 aa, Herpes simplex virus type 1
GGALLDAEHYWR

SEQ ID NO: 65 - protein, 21 aa, Herpes simplex virus type 1
AQLLAALADLGGSGLADPHGR

SEQ ID NO: 66 - protein, 10 aa, Herpes simplex virus type 1
ASWGPPAAPR

SEQ ID NO: 67 - protein, 11 aa, Herpes simplex virus type 1
YQADLYTYLSR

SEQ ID NO: 68 - protein, 12 aa, Herpes simplex virus type 1
AGWYTSTSHEAR

SEQ ID NO: 69 - protein, 11 aa, Herpes simplex virus type 1
QLLSAVDYIHR

SEQ ID NO: 70 - protein, 18 aa, Herpes simplex virus type 1
DEIGATGFTAEELDAMDR

SEQ ID NO: 71 - protein, 9 aa, Herpes simplex virus type 1
NFITPEFPR

SEQ ID NO: 72 - protein, 12 aa, Herpes simplex virus type 1
DFWMSPVFNLPR

SEQ ID NO: 73 - protein, 11 aa, Herpes simplex virus type 1
GASGSDSGAIK

SEQ ID NO: 74 - protein, 14 aa, Herpes simplex virus type 1
GHNLFLWEDQTLLR

SEQ ID NO: 75 - protein, 9 aa, Herpes simplex virus type 1
LTALELINR

SEQ ID NO: 76 - protein, 11 aa, Herpes simplex virus type 1
APVTFGDLLGR

SEQ ID NO: 77 - protein, 8 aa, Herpes simplex virus type 1
AALQGGPR

SEQ ID NO: 78 - protein, 8 aa, Herpes simplex virus type 1
AWLGEVTR

SEQ ID NO: 79 - protein, 11 aa, Herpes simplex virus type 1
VVPLGDDLPAR

SEQ ID NO: 80 - protein, 8 aa, Herpes simplex virus type 1
GLLVVPTR

SEQ ID NO: 81 - protein, 8 aa, Herpes simplex virus type 1
EAIAFLPK

SEQ ID NO: 82 - protein, 11 aa, Herpes simplex virus type 1
VSGDSGWAVGR

SEQ ID NO: 83 - protein, 12 aa, Herpes simplex virus type 1
GGGVVQLNLVNR

SEQ ID NO: 84 - protein, 20 aa, Herpes simplex virus type 1
TTPTEPASPPTTPKPTSTPK

SEQ ID NO: 85 - protein, 7 aa, Herpes simplex virus type 1
DSVTFSR

SEQ ID NO: 86 - protein, 12 aa, Herpes simplex virus type 1
SPPTSTPDPKPK

SEQ ID NO: 87 - protein, 11 aa, Herpes simplex virus type 1
ATCTAAAYYPR

SEQ ID NO: 88 - protein, 6 aa, Herpes simplex virus type 1
YALPLR

SEQ ID NO: 89 - protein, 8 aa, Herpes simplex virus type 1
SLGACPIR

SEQ ID NO: 90 - protein, 15 aa, Herpes simplex virus type 1
SVLLNAPSEAPQIVR

SEQ ID NO: 91 - protein, 15 aa, Herpes simplex virus type 1
DLPVLDQLTDPPGVR

SEQ ID NO: 92 - protein, 9 aa, Herpes simplex virus type 1
YALVDASLK

SEQ ID NO: 93 - protein, 8 aa, Herpes simplex virus type 1
TDFVWQER

SEQ ID NO: 94 - protein, 11 aa, Herpes simplex virus type 1
NAVVEQPLPQR

SEQ ID NO: 95 - protein, 18 aa, Herpes simplex virus type 1
VSVGEDVSLLPAPGPTGR

SEQ ID NO: 96 - protein, 8 aa, Herpes simplex virus type 1
SLVIHGVR

SEQ ID NO: 97 - protein, 19 aa, Herpes simplex virus type 1
DTLPQSPGPAFPLAEDVEK

SEQ ID NO: 98 - protein, 9 aa, Herpes simplex virus type 1
VFVLGSLTR

SEQ ID NO: 99 - protein, 21 aa, Herpes simplex virus type 1
VGDPADENPPGALPGPPGGPR

SEQ ID NO: 100 - protein, 13 aa, Herpes simplex virus type 1
QPFLAGVPSAVQR

SEQ ID NO: 101 - protein, 10 aa, Herpes simplex virus type 1
ALFYASAVLR

SEQ ID NO: 102 - protein, 13 aa, Herpes simplex virus type 1
IVSSVFLQYPYTK

SEQ ID NO: 103 - protein, 14 aa, Herpes simplex virus type 1
MVAPATYLLNYAGR

SEQ ID NO: 104 - protein, 29 aa, Herpes simplex virus type 1
HRPAPGSPPGIPEYAEDPYAISYGGQLDR

SEQ ID NO: 105 - protein, 9 aa, Herpes simplex virus type 1
GVLSGTYLR

SEQ ID NO: 106 - protein, 8 aa, Herpes simplex virus type 1
GGVAVPLR

SEQ ID NO: 107 - protein, 10 aa, Herpes simplex virus type 1
GSPDSAPPTK

SEQ ID NO: 108 - protein, 10 aa, Herpes simplex virus type 1
LNSAYVAMSR

SEQ ID NO: 109 - protein, 8 aa, Herpes simplex virus type 1
TTSSEFLR

SEQ ID NO: 110 - protein, 10 aa, Herpes simplex virus type 1
AAGGVYAGDK

SEQ ID NO: 111 - protein, 6 aa, Herpes simplex virus type 1
YLDNGR

SEQ ID NO: 112 - protein, 8 aa, Herpes simplex virus type 1
ATLVAELK

SEQ ID NO: 113 - protein, 12 aa, Herpes simplex virus type 1
APPLALLLPMQR

SEQ ID NO: 114 - protein, 7 aa, Herpes simplex virus type 1
LFDFFSR

SEQ ID NO: 115 - protein, 7 aa, Herpes simplex virus type 1
LLQSFLK

SEQ ID NO: 116 - protein, 12 aa, Herpes simplex virus type 1
SSAAGGTLGVVR

SEQ ID NO: 117 - protein, 8 aa, Herpes simplex virus type 1
SLSVPLVK

SEQ ID NO: 118 - protein, 14 aa, Herpes simplex virus type 1
DISFDGGLMLEYQR

SEQ ID NO: 119 - protein, 17 aa, Herpes simplex virus type 1
EAYGAEAGLGVAGTGFR

SEQ ID NO: 120 - protein, 9 aa, Herpes simplex virus type 1
FMGPEDAGR

SEQ ID NO: 121 - protein, 17 aa, Herpes simplex virus type 1
GGDGEAGPYSPSSLPSR

SEQ ID NO: 122 - protein, 6 aa, Herpes simplex virus type 1
VQLAFR

SEQ ID NO: 123 - protein, 8 aa, Herpes simplex virus type 1
LIVPSTLR

SEQ ID NO: 124 - protein, 8 aa, Herpes simplex virus type 1
FLGDAVNR

SEQ ID NO: 125 - protein, 13 aa, Herpes simplex virus type 1
DGETLRPNTLLLK

SEQ ID NO: 126 - protein, 12 aa, Herpes simplex virus type 1
ADGTLPASTLVR

SEQ ID NO: 127 - protein, 10 aa, Herpes simplex virus type 1
LSEGELSFYR

SEQ ID NO: 128 - protein, 7 aa, Herpes simplex virus type 1
VDWLEAR

SEQ ID NO: 129 - protein, 11 aa, Herpes simplex virus type 1
VTCQSNDLISR

SEQ ID NO: 130 - protein, 6 aa, Herpes simplex virus type 1
VYGLFR

SEQ ID NO: 131 - protein, 8 aa, Herpes simplex virus type 1
TYSPFVVR

SEQ ID NO: 132 - protein, 17 aa, Herpes simplex virus type 1
NPDFPLAGLAANPQTPR

SEQ ID NO: 133 - protein, 9 aa, Herpes simplex virus type 1
AGTLLLALR

SEQ ID NO: 134 - protein, 10 aa, Herpes simplex virus type 1
GGAALIGSPR

SEQ ID NO: 135 - protein, 9 aa, Herpes simplex virus type 1
DTAGQGLLR

SEQ ID NO: 136 - protein, 8 aa, Herpes simplex virus type 1
GLEPALER

SEQ ID NO: 137 - protein, 8 aa, Herpes simplex virus type 1
VVFLPTIR

SEQ ID NO: 138 - protein, 7 aa, Herpes simplex virus type 1
FPAVITR

SEQ ID NO: 139 - protein, 9 aa, Herpes simplex virus type 1
LDSLDLTLR

SEQ ID NO: 140 - protein, 11 aa, Herpes simplex virus type 1
FPVPLPSPLAR

SEQ ID NO: 141 - protein, 11 aa, Herpes simplex virus type 1
VCTFDGAAVVR

SEQ ID NO: 142 - protein, 9 aa, Herpes simplex virus type 1
EGVSTQDPR

SEQ ID NO: 143 - protein, 9 aa, Herpes simplex virus type 1
IVNSVFVWR

SEQ ID NO: 144 - protein, 34 aa, Herpes simplex virus type 1
AATPPEETPDPTTEQLAEEPVVGELDGAYLVPAK

SEQ ID NO: 145 - protein, 8 aa, Herpes simplex virus type 1
LQEVVGGR

SEQ ID NO: 146 - protein, 13 aa, Herpes simplex virus type 1
DGVLLLNTTLTVK

SEQ ID NO: 147 - protein, 9 aa, Herpes simplex virus type 1
FSHPSPLSK

SEQ ID NO: 148 - protein, 10 aa, Herpes simplex virus type 1
ANVPPPPSLR

SEQ ID NO: 149 - protein, 8 aa, Herpes simplex virus type 1
EDVFSWTR

SEQ ID NO: 150 - protein, 7 aa, Herpes simplex virus type 1
NVLAAVK

SEQ ID NO: 151 - protein, 11 aa, Herpes simplex virus type 1
TSPGVLISGLR

SEQ ID NO: 152 - protein, 8 aa, Herpes simplex virus type 1
TDVVITGR

SEQ ID NO: 153 - protein, 9 aa, Herpes simplex virus type 1
LFGSAGAPR

SEQ ID NO: 154 - protein, 9 aa, Herpes simplex virus type 1
LSPFPALVR

SEQ ID NO: 155 - protein, 12 aa, Herpes simplex virus type 1
AAGYLDVLLTVR

SEQ ID NO: 156 - protein, 18 aa, Herpes simplex virus type 1
APRPIAGSGAGSGGAGAK

SEQ ID NO: 157 - protein, 10 aa, Herpes simplex virus type 1
VSGNITAVVR

SEQ ID NO: 158 - protein, 17 aa, Herpes simplex virus type 1
GAAVASPDQPLHGGPER

SEQ ID NO: 159 - protein, 9 aa, Herpes simplex virus type 1
ATLVGSFAR

SEQ ID NO: 160 - protein, 8 aa, Herpes simplex virus type 1
TDGGVPGR

SEQ ID NO: 161 - protein, 8 aa, Herpes simplex virus type 1
SALAVLIR

SEQ ID NO: 162 - protein, 13 aa, Herpes simplex virus type 1
VLPAFSAGGPPTR

SEQ ID NO: 163 - protein, 11 aa, Herpes simplex virus type 1
VSGGPGPLVLR

SEQ ID NO: 164 - protein, 8 aa, Herpes simplex virus type 1
LAGGLLER

SEQ ID NO: 165 - protein, 10 aa, Herpes simplex virus type 1
AGAVEELGGR

SEQ ID NO: 166 - protein, 9 aa, Herpes simplex virus type 1
LLASLAGLR

SEQ ID NO: 167 - protein, 10 aa, Herpes simplex virus type 1
FSSGQPSLLR

SEQ ID NO: 168 - protein, 13 aa, Herpes simplex virus type 1
LGALITLLEPAAR

SEQ ID NO: 169 - protein, 9 aa, Herpes simplex virus type 1
GVLQDLAER

SEQ ID NO: 170 - protein, 10 aa, Herpes simplex virus type 1
QLEDAIVLLR

SEQ ID NO: 171 - protein, 10 aa, Herpes simplex virus type 1
ALVASLASLR

SEQ ID NO: 172 - protein, 22 aa, Herpes simplex virus type 1
ASLTSALPDAAQVVHVFEYGTR

SEQ ID NO: 173 - protein, 6 aa, Herpes simplex virus type 1
FLEQLR

SEQ ID NO: 174 - protein, 10 aa, Herpes simplex virus type 1
SLFPIFVTDR

SEQ ID NO: 175 - protein, 9 aa, Herpes simplex virus type 1
GTSLASGTR

SEQ ID NO: 176 - protein, 18 aa, Herpes simplex virus type 1
STETRPPLPPAAGGTETR

SEQ ID NO: 177 - protein, 7 aa, Herpes simplex virus type 1
GPWTLLK

SEQ ID NO: 178 - protein, 10 aa, Herpes simplex virus type 1
TQAPPPAFYK

SEQ ID NO: 179 - protein, 13 aa, Herpes simplex virus type 1
SSGYWWVSGDGIR

SEQ ID NO: 180 - protein, 17 aa, Herpes simplex virus type 1
FLPLGGSPEAPAETFAR

SEQ ID NO: 181 - protein, 9 aa, Herpes simplex virus type 1
LANLWQTGK

SEQ ID NO: 182 - protein, 13 aa, Herpes simplex virus type 1
AWFGAALAADLLR

SEQ ID NO: 183 - protein, 9 aa, Herpes simplex virus type 1
VYEEIPWMR

SEQ ID NO: 184 - protein, 16 aa, Herpes simplex virus type 1
TGPPPPPLSPSPVLAR

SEQ ID NO: 185 - protein, 11 aa, Herpes simplex virus type 1
QGGYLGPVDAR

SEQ ID NO: 186 - protein, 7 aa, Herpes simplex virus type 1
VEVLEGR

SEQ ID NO: 187 - protein, 11 aa, Herpes simplex virus type 1
AILAAGLVLQR

SEQ ID NO: 188 - protein, 12 aa, Herpes simplex virus type 1
LLMGDEGAAALR

SEQ ID NO: 189 - protein, 10 aa, Herpes simplex virus type 1
ATGLGGPPRP

SEQ ID NO: 190 - protein, 11 aa, Herpes simplex virus type 1
AAVPPSEAEPR

SEQ ID NO: 191 - protein, 11 aa, Herpes simplex virus type 1
SADVLVSQAIR

SEQ ID NO: 192 - protein, 13 aa, Herpes simplex virus type 1
GAAAAGFPLYVER

SEQ ID NO: 193 - protein, 9 aa, Herpes simplex virus type 1
ETLTEVLGR

SEQ ID NO: 194 - protein, 9 aa, Herpes simplex virus type 1
LVAPPDITR

SEQ ID NO: 195 - protein, 18 aa, Herpes simplex virus type 1
SFGPGGLLATPLFLPETR

SEQ ID NO: 196 - protein, 10 aa, Herpes simplex virus type 1
EADIAGVAER

SEQ ID NO: 197 - protein, 6 aa, Herpes simplex virus type 1
VFDTWR

SEQ ID NO: 198 - protein, 6 aa, Herpes simplex virus type 1
LIINMK

SEQ ID NO: 199 - protein, 16 aa, Herpes simplex virus type 1
SLVEAAEAISQQTLLR

SEQ ID NO: 200 - protein, 8 aa, Herpes simplex virus type 1
FTATSIAR

SEQ ID NO: 201 - protein, 7 aa, Herpes simplex virus type 1
FSLFNPR

SEQ ID NO: 202 - protein, 7 aa, Herpes simplex virus type 1
LPGPPPR

SEQ ID NO: 203 - protein, 13 aa, Herpes simplex virus type 1
APLVYWWLSETPK

SEQ ID NO: 204 - protein, 13 aa, Herpes simplex virus type 1
IIWGELFGVQMAK

SEQ ID NO: 205 - protein, 12 aa, Herpes simplex virus type 1
DHPENLGNPEYR

SEQ ID NO: 206 - protein, 19 aa, Herpes simplex virus type 1
YAPLASPDPFSPQHGAYAR

SEQ ID NO: 207 - protein, 8 aa, Herpes simplex virus type 1
LVHTQWLR

SEQ ID NO: 208 - protein, 21 aa, Herpes simplex virus type 1
VGADTTISKPSEAVRPPTIPR

SEQ ID NO: 209 - protein, 17 aa, Herpes simplex virus type 1
GDNDQAAGQCGDSGLLR

SEQ ID NO: 210 - protein, 8 aa, Herpes simplex virus type 1
MISGPPQR

SEQ ID NO: 211 - protein, 10 aa, Herpes simplex virus type 1
GVPSAGMHPR

SEQ ID NO: 212 - protein, 10 aa, Herpes simplex virus type 1
TGGTVTDSPR

SEQ ID NO: 213 - protein, 15 aa, Herpes simplex virus type 1
TSVDASPALWSFLLR

SEQ ID NO: 214 - protein, 7 aa, Herpes simplex virus type 1
DQPSPPR

SEQ ID NO: 215 - protein, 15 aa, Herpes simplex virus type 1
TPDAWVGEPWAVPTR

SEQ ID NO: 216 - protein, 15 aa, Herpes simplex virus type 1
YEYQYGVVLPGTNGR

SEQ ID NO: 217 - protein, 12 aa, Herpes simplex virus type 1
GPQETDPLIAVR

SEQ ID NO: 218 - protein, 9 aa, Herpes simplex virus type 1
LSDPNSSAR

SEQ ID NO: 219 - protein, 34 aa, Herpes simplex virus type 1
SDMSVPLYPTASPVSVEAYYSESEDEAANDFLVR

SEQ ID NO: 220 - protein, 11 aa, Human cytomegalovirus
NAHTQSWYWLR

SEQ ID NO: 221 - protein, 16 aa, Human cytomegalovirus
YHMLQDTVSESEFIVR

SEQ ID NO: 222 - protein, 10 aa, Human cytomegalovirus
YNTDIENEDR

SEQ ID NO: 223 - protein, 11 aa, Human cytomegalovirus
NAHTQSWYWLR

SEQ ID NO: 224 - protein, 16 aa, Human cytomegalovirus
YHMLQDTVSESEFIVR

SEQ ID NO: 225 - protein, 10 aa, Human cytomegalovirus
YNTDIENEDR

SEQ ID NO: 226 - protein, 11 aa, Human cytomegalovirus
VVVTDSQEADR

SEQ ID NO: 227 - protein, 12 aa, Human cytomegalovirus
MPPLCIITDAYK

SEQ ID NO: 228 - protein, 12 aa, Human cytomegalovirus
MPPLCIITDAYK

SEQ ID NO: 229 - protein, 11 aa, Human cytomegalovirus
VVVTDSQEADR

SEQ ID NO: 230 - protein, 17 aa, Human cytomegalovirus
TDLDTEPLLLTVDGDLQ

SEQ ID NO: 231 - protein, 17 aa, Human cytomegalovirus
TDLDTEPLLLTVDGDLQ

SEQ ID NO: 232 - protein, 14 aa, Human cytomegalovirus
TAGVPAPMAATVAR

SEQ ID NO: 233 - protein, 9 aa, Human cytomegalovirus
LVYLQSSAR

SEQ ID NO: 234 - protein, 13 aa, Human cytomegalovirus
FLNDVDILGSFGR

SEQ ID NO: 235 - protein, 12 aa, Human cytomegalovirus
SSNVFDLEEIMR

SEQ ID NO: 236 - protein, 16 aa, Human cytomegalovirus
TMQLIPDDYSNTHSTR

SEQ ID NO: 237 - protein, 19 aa, Human cytomegalovirus
DTSLQAPPSYEESVYNSGR

SEQ ID NO: 238 - protein, 12 aa, Human cytomegalovirus
QEEAGTEISIPR

SEQ ID NO: 239 - protein, 14 aa, Human cytomegalovirus
TTLLLYPTAVDLAK

SEQ ID NO: 240 - protein, 24 aa, Human cytomegalovirus
YQALATASGEEVAVLSHHDSLESR

SEQ ID NO: 241 - protein, 9 aa, Human cytomegalovirus
GMSLNQSTR

SEQ ID NO: 242 - protein, 7 aa, Human cytomegalovirus
SLFFFSR

SEQ ID NO: 243 - protein, 8 aa, Human cytomegalovirus
HANDDFYK

SEQ ID NO: 244 - protein, 12 aa, Human cytomegalovirus
LSSTTHDPNVMR

SEQ ID NO: 245 - protein, 11 aa, Human cytomegalovirus
EPHGQWEFMFR

SEQ ID NO: 246 - protein, 13 aa, Human cytomegalovirus
TLGLDLTTVMTER

SEQ ID NO: 247 - protein, 11 aa, Human cytomegalovirus
TGPGPPPLPPK

SEQ ID NO: 248 - protein, 12 aa, Human cytomegalovirus
TSAGEEMFEALR

SEQ ID NO: 249 - protein, 11 aa, Human cytomegalovirus
VASGAGLPTSR

SEQ ID NO: 250 - protein, 11 aa, Human cytomegalovirus
NMEGVQVVADR

SEQ ID NO: 251 - protein, 11 aa, Human cytomegalovirus
LTLHDLHDIFR

SEQ ID NO: 252 - protein, 17 aa, Human cytomegalovirus
QHTCLDISPYGNEQVSR

SEQ ID NO: 253 - protein, 9 aa, Human cytomegalovirus
AASTSLAVR

SEQ ID NO: 254 - protein, 11 aa, Human cytomegalovirus
VLHQDLVQATR

SEQ ID NO: 255 - protein, 11 aa, Human cytomegalovirus
FEGEVLESVLK

SEQ ID NO: 256 - protein, 13 aa, Human cytomegalovirus
ELVTEAVVWGNAR

SEQ ID NO: 257 - protein, 15 aa, Human cytomegalovirus
VSSGLSTFNPAGATR

SEQ ID NO: 258 - protein, 10 aa, Human cytomegalovirus
IYPEVVAQNR

SEQ ID NO: 259 - protein, 11 aa, Human cytomegalovirus
EYNLFTDIPER

SEQ ID NO: 260 - protein, 8 aa, Human cytomegalovirus
LMNGFLYR

SEQ ID NO: 261 - protein, 12 aa, Human cytomegalovirus
VYDLLLPSLDAR

SEQ ID NO: 262 - protein, 10 aa, Human cytomegalovirus
TQLLTAIVSK

SEQ ID NO: 263 - protein, 11 aa, Human cytomegalovirus
LMDINGILEGK

SEQ ID NO: 264 - protein, 15 aa, Human cytomegalovirus
DPQFQQINNFMTDFK

SEQ ID NO: 265 - protein, 16 aa, Human cytomegalovirus
EAFNTILGFLAQNTTK

SEQ ID NO: 266 - protein, 14 aa, Human cytomegalovirus
YSTLNTNAYDYFGK

SEQ ID NO: 267 - protein, 9 aa, Human cytomegalovirus
DDEDWKPSR

SEQ ID NO: 268 - protein, 15 aa, Human cytomegalovirus
VTFSNIATHYHYNAQ

SEQ ID NO: 269 - protein, 14 aa, Human cytomegalovirus
DPADEDNELVTALK

SEQ ID NO: 270 - protein, 15 aa, Human cytomegalovirus
AALQWLDLGPHLLHR

SEQ ID NO: 271 - protein, 12 aa, Human cytomegalovirus
LDNFSVELGDFR

SEQ ID NO: 272 - protein, 12 aa, Human cytomegalovirus
AAVQQLQTITFR

SEQ ID NO: 273 - protein, 11 aa, Human cytomegalovirus
LVAAVVPIPQR

SEQ ID NO: 274 - protein, 12 aa, Human cytomegalovirus
LALEDSSMLLVK

SEQ ID NO: 275 - protein, 14 aa, Human cytomegalovirus
DYLGPDLFETGAAR

SEQ ID NO: 276 - protein, 11 aa, Human cytomegalovirus
VSVSELEAVYR

SEQ ID NO: 277 - protein, 11 aa, Human cytomegalovirus
SLAVDAQHAAK

SEQ ID NO: 278 - protein, 13 aa, Human cytomegalovirus
LTGQDEAHSFSLK

SEQ ID NO: 279 - protein, 16 aa, Human cytomegalovirus
QNVMIMEPQVLDFTVR

SEQ ID NO: 280 - protein, 11 aa, Human cytomegalovirus
GVLEVHTDFTR

SEQ ID NO: 281 - protein, 15 aa, Human cytomegalovirus
VPPNSQESAAPQPPR

SEQ ID NO: 282 - protein, 13 aa, Human cytomegalovirus
ILHQSVNQTFDVR

SEQ ID NO: 283 - protein, 19 aa, Human cytomegalovirus
LLPETSGGTVVVNHSSVAR

SEQ ID NO: 284 - protein, 8 aa, Human cytomegalovirus
YVFEEVSR

SEQ ID NO: 285 - protein, 8 aa, Human cytomegalovirus
FFHQDPNR

SEQ ID NO: 286 - protein, 8 aa, Human cytomegalovirus
YAPLFASK

SEQ ID NO: 287 - protein, 10 aa, Human cytomegalovirus
SFTTTYVFIK

SEQ ID NO: 288 - protein, 19 aa, Human cytomegalovirus
GPFTQVGYLSAFPPDNEGK

SEQ ID NO: 289 - protein, 8 aa, Human cytomegalovirus
LEEPVEEK

SEQ ID NO: 290 - protein, 8 aa, Human cytomegalovirus
GIQIIYTR

SEQ ID NO: 291 - protein, 19 aa, Human cytomegalovirus
IFSTNQGGFMLPIYETAAK

SEQ ID NO: 292 - protein, 9 aa, Human cytomegalovirus
VHEPFEEMK

SEQ ID NO: 293 - protein, 8 aa, Human cytomegalovirus
LGGALQAK

SEQ ID NO: 294 - protein, 13 aa, Human cytomegalovirus
VFAQYILGADPLR

SEQ ID NO: 295 - protein, 9 aa, Human cytomegalovirus
QQLTGEETR

SEQ ID NO: 296 - protein, 21 aa, Human cytomegalovirus
QNPAAEAQELAVIPPAPTVLR

SEQ ID NO: 297 - protein, 9 aa, Human cytomegalovirus
HDASENAVR

SEQ ID NO: 298 - protein, 12 aa, Human cytomegalovirus
DWDEEEEASAAR

SEQ ID NO: 299 - protein, 15 aa, Human cytomegalovirus
DGNLDTSFVNPNYGR

SEQ ID NO: 300 - protein, 19 aa, Human cytomegalovirus
GSPLTIESHLSDNEEDPIR

SEQ ID NO: 301 - protein, 20 aa, Human cytomegalovirus
SVTVEQPSTSADGSNTTPSK

SEQ ID NO: 302 - protein, 11 aa, Human cytomegalovirus
AWSLGLDTMAR

SEQ ID NO: 303 - protein, 10 aa, Human cytomegalovirus
FSGHNGIYDR

SEQ ID NO: 304 - protein, 14 aa, Human cytomegalovirus
DYNVLFYTAHYTSR

SEQ ID NO: 305 - protein, 8 aa, Human cytomegalovirus
SATEDLDR

SEQ ID NO: 306 - protein, 12 aa, Human cytomegalovirus
FVLQDFDVQHLR

SEQ ID NO: 307 - protein, 10 aa, Human cytomegalovirus
LLVVTQGQLR

SEQ ID NO: 308 - protein, 12 aa, Human cytomegalovirus
GLLHSYFEDVER

SEQ ID NO: 309 - protein, 13 aa, Human cytomegalovirus
APDGGLNLDDFMR

SEQ ID NO: 310 - protein, 10 aa, Human cytomegalovirus
DLGLSASMLR

SEQ ID NO: 311 - protein, 11 aa, Human cytomegalovirus
VGLALLIDDFR

SEQ ID NO: 312 - protein, 11 aa, Human cytomegalovirus
SSLYEANPELR

SEQ ID NO: 313 - protein, 17 aa, Human cytomegalovirus
TAEQEVAAADPETGNER

SEQ ID NO: 314 - protein, 9 aa, Human cytomegalovirus
HMDPEQDYR

SEQ ID NO: 315 - protein, 10 aa, Human cytomegalovirus
DAADEVWALR

SEQ ID NO: 316 - protein, 10 aa, Human cytomegalovirus
DVVALVNFLR

SEQ ID NO: 317 - protein, 16 aa, Human cytomegalovirus
SGTGPQPGSAGMGGAK

SEQ ID NO: 318 - protein, 15 aa, Human cytomegalovirus
AEPSVSNDDGNGGER

SEQ ID NO: 319 - protein, 9 aa, Human cytomegalovirus
DNVAGSISK

SEQ ID NO: 320 - protein, 15 aa, Human cytomegalovirus
EPPTPADELQTAVSR

SEQ ID NO: 321 - protein, 9 aa, Human cytomegalovirus
EGELFFFSK

SEQ ID NO: 322 - protein, 12 aa, Human cytomegalovirus
TQLDVLYSDPLK

SEQ ID NO: 323 - protein, 10 aa, Human cytomegalovirus
ITTETYHLQR

SEQ ID NO: 324 - protein, 12 aa, Human cytomegalovirus
GDVESLSAEVTK

SEQ ID NO: 325 - protein, 9 aa, Human cytomegalovirus
SALGPFVGK

SEQ ID NO: 326 - protein, 11 aa, Human cytomegalovirus
NVMTHEEAESR

SEQ ID NO: 327 - protein, 10 aa, Human cytomegalovirus
LEDPLPPWLR

SEQ ID NO: 328 - protein, 14 aa, Human cytomegalovirus
QHGVIDGENSETER

SEQ ID NO: 329 - protein, 17 aa, Human cytomegalovirus
TPEVNPIDAEGLSGLAR

SEQ ID NO: 330 - protein, 8 aa, Human cytomegalovirus
IPMTFVDR

SEQ ID NO: 331 - protein, 9 aa, Human cytomegalovirus
QLVLFMTPK

SEQ ID NO: 332 - protein, 6 aa, Human cytomegalovirus
ISWLER

SEQ ID NO: 333 - protein, 16 aa, Human cytomegalovirus
VYVYSPVVESLYLVSR

SEQ ID NO: 334 - protein, 19 aa, Human cytomegalovirus
SASLSFLDWPDGSVTEGVR

SEQ ID NO: 335 - protein, 10 aa, Human cytomegalovirus
FLLPVGTVSR

SEQ ID NO: 336 - protein, 10 aa, Human cytomegalovirus
LSEPPTLALR

SEQ ID NO: 337 - protein, 14 aa, Human cytomegalovirus
IVTEHDTLLYVASR

SEQ ID NO: 338 - protein, 17 aa, Human cytomegalovirus
NGLFAVENFLTEEPFQR

SEQ ID NO: 339 - protein, 10 aa, Human cytomegalovirus
VTLALEQAQR

SEQ ID NO: 340 - protein, 15 aa, Human cytomegalovirus
EFLEAPWESAPQPPR

SEQ ID NO: 341 - protein, 13 aa, Human cytomegalovirus
DASGLMFPIISTR

SEQ ID NO: 342 - protein, 13 aa, Human cytomegalovirus
YQQLVDEVEQELK

SEQ ID NO: 343 - protein, 14 aa, Human cytomegalovirus
FNQDLLSALQQLSK

SEQ ID NO: 344 - protein, 12 aa, Human cytomegalovirus
IDTQQELTAADR

SEQ ID NO: 345 - protein, 10 aa, Human cytomegalovirus
LDALSTASER

SEQ ID NO: 346 - protein, 10 aa, Human cytomegalovirus
LVDPTYVIDK

SEQ ID NO: 347 - protein, 10 aa, Human cytomegalovirus
VTNEAVLFGR

SEQ ID NO: 348 - protein, 10 aa, Human cytomegalovirus
GLTPQALVAR

SEQ ID NO: 349 - protein, 14 aa, Human cytomegalovirus
EVIASVGELVPEPR

SEQ ID NO: 350 - protein, 16 aa, Human cytomegalovirus
IGEYLLEQGFPVYEVR

SEQ ID NO: 351 - protein, 15 aa, Human cytomegalovirus
VAASASVPLNPHYGK

SEQ ID NO: 352 - protein, 24 aa, Human cytomegalovirus
ALQVTGTPQFFDQFDTNNAMGTYR

SEQ ID NO: 353 - protein, 11 aa, Human cytomegalovirus
TDPATLTAYDK

SEQ ID NO: 354 - protein, 11 aa, Human cytomegalovirus
ELQAFLDENFK

SEQ ID NO: 355 - protein, 11 aa, Human cytomegalovirus
ILEALDILILK

SEQ ID NO: 356 - protein, 14 aa, Human cytomegalovirus
ESAPQETLPTNHER

SEQ ID NO: 357 - protein, 15 aa, Human cytomegalovirus
DSPGGMDEPPSGWER

SEQ ID NO: 358 - protein, 17 aa, Human cytomegalovirus
SDSYGLLGNSVDALYIR

SEQ ID NO: 359 - protein, 15 aa, Human cytomegalovirus
HFADYVDPHYPGWGR

SEQ ID NO: 360 - protein, 14 aa, Human cytomegalovirus
LITNVEGGSLEAGR

SEQ ID NO: 361 - protein, 11 aa, Human cytomegalovirus
LLVDLNNFGPR

SEQ ID NO: 362 - protein, 10 aa, Human cytomegalovirus
TAADISSTLR

SEQ ID NO: 363 - protein, 11 aa, Human cytomegalovirus
GDTPVLPHETR

SEQ ID NO: 364 - protein, 15 aa, Human cytomegalovirus
GPQYSEHPTFTSQYR

SEQ ID NO: 365 - protein, 16 aa, Human cytomegalovirus
IFAELEGVWQPAAQPK

SEQ ID NO: 366 - protein, 14 aa, Human cytomegalovirus
YSGSPQTILTLTDK

SEQ ID NO: 367 - protein, 11 aa, Human cytomegalovirus
SGAADEGLEVR

SEQ ID NO: 368 - protein, 9 aa, Human cytomegalovirus
SVIDQQLTR

SEQ ID NO: 369 - protein, 18 aa, Human cytomegalovirus
ALLADYAETFSPLGSFTR

SEQ ID NO: 370 - protein, 12 aa, Human cytomegalovirus
VEENDLVNVVLR

SEQ ID NO: 371 - protein, 22 aa, Human cytomegalovirus
HVIWPGTSVLWAPDVVITTVQR

SEQ ID NO: 372 - protein, 8 aa, Human cytomegalovirus
TDVWDLVK

SEQ ID NO: 373 - protein, 7 aa, Human cytomegalovirus
VEEPVSR

SEQ ID NO: 374 - protein, 18 aa, Human cytomegalovirus
TAAQVTLGDGLDYHIGVK

SEQ ID NO: 375 - protein, 18 aa, Human cytomegalovirus
AAGEQQQPPSLVGTGVHR

SEQ ID NO: 376 - protein, 16 aa, Human cytomegalovirus
VGGVDAVLEENDVELR

SEQ ID NO: 377 - protein, 14 aa, Human cytomegalovirus
LGQGSFGEVWPLDR

SEQ ID NO: 378 - protein, 9 aa, Human cytomegalovirus
HAVSTVLDR

SEQ ID NO: 379 - protein, 11 aa, Human cytomegalovirus
EYLADLLYLNK

SEQ ID NO: 380 - protein, 12 aa, Human cytomegalovirus
HGGSDDYVWLSR

SEQ ID NO: 381 - protein, 7 aa, Human cytomegalovirus
VTAPYLR

SEQ ID NO: 382 - protein, 8 aa, Human cytomegalovirus
TAHWLFHK

SEQ ID NO: 383 - protein, 10 aa, Human cytomegalovirus
SVQHFLWMVR

SEQ ID NO: 384 - protein, 11 aa, Human cytomegalovirus
TLSMLQGLYLR

SEQ ID NO: 385 - protein, 16 aa, Human cytomegalovirus
TEVEPLLMLGSIEGLR

SEQ ID NO: 386 - protein, 10 aa, Human cytomegalovirus
WHGALGTITR

SEQ ID NO: 387 - protein, 10 aa, Human cytomegalovirus
WEDGAPTFTR

SEQ ID NO: 388 - protein, 13 aa, Human cytomegalovirus
TEAEVSEAEVEAR

SEQ ID NO: 389 - protein, 13 aa, Human cytomegalovirus
INLNTDLSPEWVK

SEQ ID NO: 390 - protein, 9 aa, Human cytomegalovirus
ILPVGSMYR

SEQ ID NO: 391 - protein, 11 aa, Human cytomegalovirus
YETELPQVDAR

SEQ ID NO: 392 - protein, 8 aa, Human cytomegalovirus
VTPSDLER

SEQ ID NO: 393 - protein, 15 aa, Human cytomegalovirus
LSWSSDESSASSSSR

SEQ ID NO: 394 - protein, 14 aa, Human cytomegalovirus
NNTGTEVDQCLAYR

SEQ ID NO: 395 - protein, 8 aa, Human cytomegalovirus
VATNCLVK

SEQ ID NO: 396 - protein, 8 aa, Human cytomegalovirus
VATNCLVK

SEQ ID NO: 397 - protein, 14 aa, Human cytomegalovirus
NNTGTEVDQCLAYR

SEQ ID NO: 398 - protein, 10 aa, Human cytomegalovirus
QPVVTLLLAR

SEQ ID NO: 399 - protein, 10 aa, Human cytomegalovirus
QPVVTLLLAR

SEQ ID NO: 400 - protein, 10 aa, Human cytomegalovirus
DGLADWNVVR

SEQ ID NO: 401 - protein, 9 aa, Human cytomegalovirus
VTLHGLAQR

SEQ ID NO: 402 - protein, 13 aa, Human cytomegalovirus
SAPKPQELLFGPR

SEQ ID NO: 403 - protein, 21 aa, Human cytomegalovirus
FTLADLLGSDAVAGGGLPGGR

SEQ ID NO: 404 - protein, 10 aa, Human cytomegalovirus
LLEDAAVTMR

SEQ ID NO: 405 - protein, 20 aa, Human cytomegalovirus
GVIQHLPGYGTITEELVQER

SEQ ID NO: 406 - protein, 10 aa, Human cytomegalovirus
FWETPTLIMK

SEQ ID NO: 407 - protein, 13 aa, Human cytomegalovirus
ALLTLLSSDTAPR

SEQ ID NO: 408 - protein, 8 aa, Human cytomegalovirus
VNLPAHSR

SEQ ID NO: 409 - protein, 9 aa, Human cytomegalovirus
HQLDPPLLR

SEQ ID NO: 410 - protein, 8 aa, Human cytomegalovirus
SIMATQLR

SEQ ID NO: 411 - protein, 12 aa, Human cytomegalovirus
TTPTSPSMGFQR

SEQ ID NO: 412 - protein, 9 aa, Human cytomegalovirus
QLETTISTK

SEQ ID NO: 413 - protein, 16 aa, Human cytomegalovirus
HVPLADSAVSHETLER

SEQ ID NO: 414 - protein, 18 aa, Human cytomegalovirus
IINYSQFVDHNLSSEITK

SEQ ID NO: 415 - protein, 8 aa, Human cytomegalovirus
MSSLFNDK

SEQ ID NO: 416 - protein, 9 aa, Human cytomegalovirus
TLFDTITVR

SEQ ID NO: 417 - protein, 8 aa, Human cytomegalovirus
VANAPEVR

SEQ ID NO: 418 - protein, 20 aa, Human cytomegalovirus
LVTHQGAEEAIVYSNYTVER

SEQ ID NO: 419 - protein, 11 aa, Human cytomegalovirus
GSLLTPDEQAR

SEQ ID NO: 420 - protein, 13 aa, Human cytomegalovirus
FLSLPDHDTVLLR

SEQ ID NO: 421 - protein, 14 aa, Human cytomegalovirus
AQYLLGAAGSVPYR

SEQ ID NO: 422 - protein, 16 aa, Human cytomegalovirus
LTSCNYNPLYLEADGR

SEQ ID NO: 423 - protein, 12 aa, Human cytomegalovirus
GIVTTMTHSLTR

SEQ ID NO: 424 - protein, 17 aa, Human cytomegalovirus
VLTDPEPIQSEAEGENK

SEQ ID NO: 425 - protein, 14 aa, Human cytomegalovirus
TEDFLHWLLGWGHK

SEQ ID NO: 426 - protein, 8 aa, Human cytomegalovirus
SICSFFPK

SEQ ID NO: 427 - protein, 11 aa, Human cytomegalovirus
LQGNYNEQHYR

SEQ ID NO: 428 - protein, 14 aa, Human cytomegalovirus
SLGVLPNDHHYALK

SEQ ID NO: 429 - protein, 21 aa, Human cytomegalovirus
GGGSTAAAAAVGHAGAGQQAR

SEQ ID NO: 430 - protein, 12 aa, Human cytomegalovirus
TAQTYTTGTLTR

SEQ ID NO: 431 - protein, 12 aa, Human cytomegalovirus
LLPLFIVPDAYR

SEQ ID NO: 432 - protein, 13 aa, Human cytomegalovirus
TPVLSPEHGGEVR

SEQ ID NO: 433 - protein, 12 aa, Human cytomegalovirus
TTYSDADDQSVR

SEQ ID NO: 434 - protein, 18 aa, Human cytomegalovirus
GGGQVWSVVPSLVFFQQK

SEQ ID NO: 435 - protein, 10 aa, Human cytomegalovirus
GGGAVEPAVR

SEQ ID NO: 436 - protein, 12 aa, Human cytomegalovirus
DLDDSFDYLVER

SEQ ID NO: 437 - protein, 16 aa, Human cytomegalovirus
ETMNNLGVSDHAVLSR

SEQ ID NO: 438 - protein, 8 aa, Human cytomegalovirus
GLETLLLR

SEQ ID NO: 439 - protein, 9 aa, Human cytomegalovirus
DFFLLELQK

SEQ ID NO: 440 - protein, 16 aa, Human cytomegalovirus
TTEDPPENHVVADVAR

SEQ ID NO: 441 - protein, 18 aa, Human cytomegalovirus
LIETPDENFLLVTNVIPR

SEQ ID NO: 442 - protein, 9 aa, Human cytomegalovirus
AAVQQELQR

SEQ ID NO: 443 - protein, 18 aa, Human cytomegalovirus
QLQSGNVDDALDSLTELK

SEQ ID NO: 444 - protein, 17 aa, Human cytomegalovirus
LCGSGCGGNDSSSGSHR

SEQ ID NO: 445 - protein, 11 aa, Human cytomegalovirus
ISQRPPTPGTK

SEQ ID NO: 446 - protein, 11 aa, Human cytomegalovirus
LPWTTVFAAFR

SEQ ID NO: 447 - protein, 11 aa, Human cytomegalovirus
SLEEGSTISSR

SEQ ID NO: 448 - protein, 21 aa, Human cytomegalovirus
GFQVPTDGTVIYVPPGIQETR

SEQ ID NO: 449 - protein, 15 aa, Human cytomegalovirus
SHALVAEQQLFQWLK

SEQ ID NO: 450 - protein, 10 aa, Human cytomegalovirus
VANTITEFFR

SEQ ID NO: 451 - protein, 10 aa, Human cytomegalovirus
FDPHEGAWER

SEQ ID NO: 452 - protein, 17 aa, Human cytomegalovirus
EPLTPLGYAVILLPEPR

SEQ ID NO: 453 - protein, 9 aa, Human cytomegalovirus
DGVLDAVWR

SEQ ID NO: 454 - protein, 9 aa, Kaposi?s Sarcoma herpesvirus
DHFPLLTTK

SEQ ID NO: 455 - protein, 13 aa, Kaposi?s Sarcoma herpesvirus
EGDLGPVYGFQWR

SEQ ID NO: 456 - protein, 14 aa, Kaposi?s Sarcoma herpesvirus
SVSSMEEFTPDDFR

SEQ ID NO: 457 - protein, 18 aa, Kaposi?s Sarcoma herpesvirus
EAIVFHNTHLFQPIFQGK

SEQ ID NO: 458 - protein, 14 aa, Kaposi?s Sarcoma herpesvirus
LASMGISEGGDALR

SEQ ID NO: 459 - protein, 10 aa, Kaposi?s Sarcoma herpesvirus
HVIMTPLVDR

SEQ ID NO: 460 - protein, 12 aa, Kaposi?s Sarcoma herpesvirus
GIVIDVSEWGPR

SEQ ID NO: 461 - protein, 9 aa, Kaposi?s Sarcoma herpesvirus
ICQIVFVER

SEQ ID NO: 462 - protein, 10 aa, Kaposi?s Sarcoma herpesvirus
GEIQVILLNK

SEQ ID NO: 463 - protein, 13 aa, Kaposi?s Sarcoma herpesvirus
LASSVFDLETMFR

SEQ ID NO: 464 - protein, 14 aa, Kaposi?s Sarcoma herpesvirus
NILLGMHQLQQEER

SEQ ID NO: 465 - protein, 9 aa, Kaposi?s Sarcoma herpesvirus
LPDAPEFEK

SEQ ID NO: 466 - protein, 12 aa, Kaposi?s Sarcoma herpesvirus
TLGWDIQEELNK

SEQ ID NO: 467 - protein, 13 aa, Kaposi?s Sarcoma herpesvirus
HFASFYVLSAMEK

SEQ ID NO: 468 - protein, 26 aa, Kaposi?s Sarcoma herpesvirus
AGPSSLVDILPQGLPGGGYGSMGVIR

SEQ ID NO: 469 - protein, 8 aa, Kaposi?s Sarcoma herpesvirus
AYAAVNTR

SEQ ID NO: 470 - protein, 13 aa, Kaposi?s Sarcoma herpesvirus
DSEESVDEAAGYK

SEQ ID NO: 471 - protein, 7 aa, Kaposi?s Sarcoma herpesvirus
VTVLPYR

SEQ ID NO: 472 - protein, 15 aa, Kaposi?s Sarcoma herpesvirus
DGSSGDEPDGGPNDR

SEQ ID NO: 473 - protein, 9 aa, Kaposi?s Sarcoma herpesvirus
ALYAANNTR

SEQ ID NO: 474 - protein, 11 aa, Kaposi?s Sarcoma herpesvirus
GPHISQQLPTR

SEQ ID NO: 475 - protein, 9 aa, Kaposi?s Sarcoma herpesvirus
QLQQALEEK

SEQ ID NO: 476 - protein, 7 aa, Kaposi?s Sarcoma herpesvirus
EQIIFLR

SEQ ID NO: 477 - protein, 9 aa, Kaposi?s Sarcoma herpesvirus
VSPVAENGR

SEQ ID NO: 478 - protein, 11 aa, Kaposi?s Sarcoma herpesvirus
VPFSATTTTTR

SEQ ID NO: 479 - protein, 9 aa, Kaposi?s Sarcoma herpesvirus
DAHYNAEIR

SEQ ID NO: 480 - protein, 17 aa, Kaposi?s Sarcoma herpesvirus
GVESPAIQSTETWVVNK

SEQ ID NO: 481 - protein, 9 aa, Kaposi?s Sarcoma herpesvirus
HAPPVFILK

SEQ ID NO: 482 - protein, 10 aa, Kaposi?s Sarcoma herpesvirus
GLVDTVLAVK

SEQ ID NO: 483 - protein, 15 aa, Kaposi?s Sarcoma herpesvirus
TVMLPLDLSTVAPGR

SEQ ID NO: 484 - protein, 16 aa, Kaposi?s Sarcoma herpesvirus
QSFLQPGLLYANLVLK

SEQ ID NO: 485 - protein, 10 aa, Kaposi?s Sarcoma herpesvirus
ALHLGETALR

SEQ ID NO: 486 - protein, 17 aa, Kaposi?s Sarcoma herpesvirus
VVSSYLGQSGQSLDLER

SEQ ID NO: 487 - protein, 11 aa, Kaposi?s Sarcoma herpesvirus
QVLQFLVVTPK

SEQ ID NO: 488 - protein, 17 aa, Kaposi?s Sarcoma herpesvirus
ACTSDVTAVYWAGQGGR

SEQ ID NO: 489 - protein, 10 aa, Kaposi?s Sarcoma herpesvirus
YLTFTQTGER

SEQ ID NO: 490 - protein, 12 aa, Kaposi?s Sarcoma herpesvirus
FVSFPAVLLPGK

SEQ ID NO: 491 - protein, 19 aa, Kaposi?s Sarcoma herpesvirus
IVAPGVINNFSEPIGIWVR

SEQ ID NO: 492 - protein, 16 aa, Kaposi?s Sarcoma herpesvirus
ILPTVVESSSSVLIFR

SEQ ID NO: 493 - protein, 19 aa, Kaposi?s Sarcoma herpesvirus
GEPVWDSVIHPSHIVISNR

SEQ ID NO: 494 - protein, 12 aa, Kaposi?s Sarcoma herpesvirus
AYPNFTFDNTHR

SEQ ID NO: 495 - protein, 8 aa, Kaposi?s Sarcoma herpesvirus
AIDASFIR

SEQ ID NO: 496 - protein, 16 aa, Kaposi?s Sarcoma herpesvirus
NPYDPWYYSPQLPGYR

SEQ ID NO: 497 - protein, 19 aa, Kaposi?s Sarcoma herpesvirus
GAEDDEGHLFPGEEPAYHK

SEQ ID NO: 498 - protein, 15 aa, Kaposi?s Sarcoma herpesvirus
GMLLEQSQSPYPALK

SEQ ID NO: 499 - protein, 11 aa, Kaposi?s Sarcoma herpesvirus
VIFHGGQDALK

SEQ ID NO: 500 - protein, 18 aa, Kaposi?s Sarcoma herpesvirus
FPSVTHGYPIQGFSLTYK

SEQ ID NO: 501 - protein, 16 aa, Kaposi?s Sarcoma herpesvirus
CVLEDIDDPNNSNPGR

SEQ ID NO: 502 - protein, 13 aa, Kaposi?s Sarcoma herpesvirus
QSTASSGSEDVVR

SEQ ID NO: 503 - protein, 13 aa, Kaposi?s Sarcoma herpesvirus
AHTPKPVAVVAGR

SEQ ID NO: 504 - protein, 9 aa, Kaposi?s Sarcoma herpesvirus
MLPIEGAPR

SEQ ID NO: 505 - protein, 10 aa, Kaposi?s Sarcoma herpesvirus
IMAPLEIMTK

SEQ ID NO: 506 - protein, 17 aa, Kaposi?s Sarcoma herpesvirus
GQLAPENFYSITGSAEK

SEQ ID NO: 507 - protein, 15 aa, Kaposi?s Sarcoma herpesvirus
DSTAAATAAEATTPK

SEQ ID NO: 508 - protein, 9 aa, Kaposi?s Sarcoma herpesvirus
ISQLTVENR

SEQ ID NO: 509 - protein, 17 aa, Kaposi?s Sarcoma herpesvirus
EAQLTATVGALSAAAAK

SEQ ID NO: 510 - protein, 7 aa, Kaposi?s Sarcoma herpesvirus
QVDDALK

SEQ ID NO: 511 - protein, 8 aa, Kaposi?s Sarcoma herpesvirus
FDDSIIPR

SEQ ID NO: 512 - protein, 13 aa, Kaposi?s Sarcoma herpesvirus
LPASMIIDGESPR

SEQ ID NO: 513 - protein, 14 aa, Kaposi?s Sarcoma herpesvirus
GTQSAQGIPPPLGR

SEQ ID NO: 514 - protein, 15 aa, Kaposi?s Sarcoma herpesvirus
TTISYGDNLTSTVHK

SEQ ID NO: 515 - protein, 14 aa, Kaposi?s Sarcoma herpesvirus
SVDVGVVHTDALSR

SEQ ID NO: 516 - protein, 9 aa, Kaposi?s Sarcoma herpesvirus
VPDSIPDSR

SEQ ID NO: 517 - protein, 9 aa, Kaposi?s Sarcoma herpesvirus
ELGLQEWAR

SEQ ID NO: 518 - protein, 13 aa, Kaposi?s Sarcoma herpesvirus
ALPQELLPVPAWR

SEQ ID NO: 519 - protein, 11 aa, Kaposi?s Sarcoma herpesvirus
WAVNLETNVSK

SEQ ID NO: 520 - protein, 11 aa, Kaposi?s Sarcoma herpesvirus
AYDTQQYAVQK

SEQ ID NO: 521 - protein, 8 aa, Kaposi?s Sarcoma herpesvirus
LLTVWLAK

SEQ ID NO: 522 - protein, 10 aa, Kaposi?s Sarcoma herpesvirus
SSQVLDLILR

SEQ ID NO: 523 - protein, 17 aa, Kaposi?s Sarcoma herpesvirus
TMGTEPVYESVAQMFMR

SEQ ID NO: 524 - protein, 15 aa, Kaposi?s Sarcoma herpesvirus
LWYLFDPATAPNLIK

SEQ ID NO: 525 - protein, 15 aa, Kaposi?s Sarcoma herpesvirus
APFVDQSQSMSFFLK

SEQ ID NO: 526 - protein, 13 aa, Kaposi?s Sarcoma herpesvirus
LDHDYAHHPLVAR

SEQ ID NO: 527 - protein, 18 aa, Kaposi?s Sarcoma herpesvirus
MNTLDQGNMSQAEYLVQK

SEQ ID NO: 528 - protein, 8 aa, Kaposi?s Sarcoma herpesvirus
TPVTVDYR

SEQ ID NO: 529 - protein, 10 aa, Kaposi?s Sarcoma herpesvirus
LQFIVADADK

SEQ ID NO: 530 - protein, 16 aa, Kaposi?s Sarcoma herpesvirus
LSATDDDSGDYAPMDR

SEQ ID NO: 531 - protein, 11 aa, Kaposi?s Sarcoma herpesvirus
GLASEASAWIR

SEQ ID NO: 532 - protein, 11 aa, Kaposi?s Sarcoma herpesvirus
GPLQVLTGLLR

SEQ ID NO: 533 - protein, 15 aa, Kaposi?s Sarcoma herpesvirus
LSFNPVNADVPATWR

SEQ ID NO: 534 - protein, 9 aa, Kaposi?s Sarcoma herpesvirus
LDLLITQLK

SEQ ID NO: 535 - protein, 10 aa, Kaposi?s Sarcoma herpesvirus
LNLVSLLGPK

SEQ ID NO: 536 - protein, 15 aa, Kaposi?s Sarcoma herpesvirus
FQPGALPDPNAPMLK

SEQ ID NO: 537 - protein, 9 aa, Kaposi?s Sarcoma herpesvirus
SATQLINGR

SEQ ID NO: 538 - protein, 10 aa, Kaposi?s Sarcoma herpesvirus
LVLGDIFASK

SEQ ID NO: 539 - protein, 15 aa, Kaposi?s Sarcoma herpesvirus
SSQETVLAMVQLGAR

SEQ ID NO: 540 - protein, 11 aa, Kaposi?s Sarcoma herpesvirus
LGSWASQENLR

SEQ ID NO: 541 - protein, 7 aa, Kaposi?s Sarcoma herpesvirus
LLWYLQR

SEQ ID NO: 542 - protein, 16 aa, Kaposi?s Sarcoma herpesvirus
TGDITVETCVNGFNLR

SEQ ID NO: 543 - protein, 8 aa, Kaposi?s Sarcoma herpesvirus
FLMSSWVK

SEQ ID NO: 544 - protein, 14 aa, Kaposi?s Sarcoma herpesvirus
TPTVHQKPPPLPAK

SEQ ID NO: 545 - protein, 8 aa, Kaposi?s Sarcoma herpesvirus
DISTPAPR

SEQ ID NO: 546 - protein, 11 aa, Kaposi?s Sarcoma herpesvirus
VTQSSIEQLQR

SEQ ID NO: 547 - protein, 12 aa, Kaposi?s Sarcoma herpesvirus
GQYSSLSQSAFR

SEQ ID NO: 548 - protein, 18 aa, Kaposi?s Sarcoma herpesvirus
TLEYNLDISPELMDYIDR

SEQ ID NO: 549 - protein, 10 aa, Kaposi?s Sarcoma herpesvirus
ADIVSVTWQK

SEQ ID NO: 550 - protein, 24 aa, Kaposi?s Sarcoma herpesvirus
QLPGPVNVATYSHSYGVVVQTQYR

SEQ ID NO: 551 - protein, 10 aa, Kaposi?s Sarcoma herpesvirus
LMQQLPVTAR

SEQ ID NO: 552 - protein, 27 aa, Kaposi?s Sarcoma herpesvirus
FHSTTGINQLGLSMLQVSGDGNMNWGR

SEQ ID NO: 553 - protein, 8 aa, Kaposi?s Sarcoma herpesvirus
LYITGLMR

SEQ ID NO: 554 - protein, 24 aa, Kaposi?s Sarcoma herpesvirus
TLETPPPGAHFLASSLDAALGLAR

SEQ ID NO: 555 - protein, 10 aa, Kaposi?s Sarcoma herpesvirus
EALTGPWPVR

SEQ ID NO: 556 - protein, 9 aa, Kaposi?s Sarcoma herpesvirus
YSTLPDLWR

SEQ ID NO: 557 - protein, 15 aa, Kaposi?s Sarcoma herpesvirus
FGTPDSSTLPLYAAR

SEQ ID NO: 558 - protein, 13 aa, Kaposi?s Sarcoma herpesvirus
LAITSDQSYTNFK

SEQ ID NO: 559 - protein, 15 aa, Kaposi?s Sarcoma herpesvirus
DLVGFQLALNQLVSR

SEQ ID NO: 560 - protein, 15 aa, Kaposi?s Sarcoma herpesvirus
TLGSSTVSDMLEPTK

SEQ ID NO: 561 - protein, 14 aa, Kaposi?s Sarcoma herpesvirus
NANFISFVATTGHR

SEQ ID NO: 562 - protein, 10 aa, Kaposi?s Sarcoma herpesvirus
DYGLFISQPR

SEQ ID NO: 563 - protein, 13 aa, Kaposi?s Sarcoma herpesvirus
DAFLSDGIVDMAR

SEQ ID NO: 564 - protein, 14 aa, Kaposi?s Sarcoma herpesvirus
LPYTVPIINTTFGR

SEQ ID NO: 565 - protein, 23 aa, Kaposi?s Sarcoma herpesvirus
TLGPQAGSHAPPTVGIATQEPYR

SEQ ID NO: 566 - protein, 10 aa, Kaposi?s Sarcoma herpesvirus
FPYIAPPPSR

SEQ ID NO: 567 - protein, 14 aa, Kaposi?s Sarcoma herpesvirus
NFLGLLFDPIVQSR

SEQ ID NO: 568 - protein, 7 aa, Kaposi?s Sarcoma herpesvirus
APWVLLR

SEQ ID NO: 569 - protein, 13 aa, Kaposi?s Sarcoma herpesvirus
GTQYITGNVQTQR

SEQ ID NO: 570 - protein, 11 aa, Kaposi?s Sarcoma herpesvirus
DLLSFVNHALK

SEQ ID NO: 571 - protein, 9 aa, Kaposi?s Sarcoma herpesvirus
EAELGTLVR

SEQ ID NO: 572 - protein, 10 aa, Kaposi?s Sarcoma herpesvirus
FGGSSAVFEK

SEQ ID NO: 573 - protein, 13 aa, Kaposi?s Sarcoma herpesvirus
WTNLVVTSNLGFK

SEQ ID NO: 574 - protein, 15 aa, Kaposi?s Sarcoma herpesvirus
ATSSQSLNPVWDALR

SEQ ID NO: 575 - protein, 22 aa, Kaposi?s Sarcoma herpesvirus
IFSPWPITNNHFVAGPLAFGLR

SEQ ID NO: 576 - protein, 8 aa, Kaposi?s Sarcoma herpesvirus
ANLFVNVR

SEQ ID NO: 577 - protein, 10 aa, Kaposi?s Sarcoma herpesvirus
DLAIWELALR

SEQ ID NO: 578 - protein, 14 aa, Kaposi?s Sarcoma herpesvirus
TASLPVVLIMTVGR

SEQ ID NO: 579 - protein, 11 aa, Kaposi?s Sarcoma herpesvirus
NNALVGSNTPK

SEQ ID NO: 580 - protein, 8 aa, Kaposi?s Sarcoma herpesvirus
ATPLVMFK

SEQ ID NO: 581 - protein, 7 aa, Kaposi?s Sarcoma herpesvirus
NPGVFFR

SEQ ID NO: 582 - protein, 8 aa, Kaposi?s Sarcoma herpesvirus
ELLNLLMK

SEQ ID NO: 583 - protein, 18 aa, Kaposi?s Sarcoma herpesvirus
GPGSVNLLTANTFQSLGR

SEQ ID NO: 584 - protein, 8 aa, Kaposi?s Sarcoma herpesvirus
TPLINAQK

SEQ ID NO: 585 - protein, 18 aa, Kaposi?s Sarcoma herpesvirus
HLVLTAQHPSPLASLGGR

SEQ ID NO: 586 - protein, 14 aa, Kaposi?s Sarcoma herpesvirus
AQIVAFGTTSGFVK

SEQ ID NO: 587 - protein, 12 aa, Kaposi?s Sarcoma herpesvirus
ANLAYSLQQLYK

SEQ ID NO: 588 - protein, 36 aa, Kaposi?s Sarcoma herpesvirus
LATAMASDDSVIWASEISHSLSEPTSVLPLTPAVTR

SEQ ID NO: 589 - protein, 10 aa, Kaposi?s Sarcoma herpesvirus
IPAGCQETVK

SEQ ID NO: 590 - protein, 11 aa, Kaposi?s Sarcoma herpesvirus
LGDPLEGCPER

SEQ ID NO: 591 - protein, 17 aa, Kaposi?s Sarcoma herpesvirus
DTASLLATISQQVPHLR

SEQ ID NO: 592 - protein, 14 aa, Kaposi?s Sarcoma herpesvirus
IFYHDLSLSLPTLK

SEQ ID NO: 593 - protein, 13 aa, Kaposi?s Sarcoma herpesvirus
SIEDWLHSAVWDK

SEQ ID NO: 594 - protein, 16 aa, Kaposi?s Sarcoma herpesvirus
ALAFFVPPAPINTLQR

SEQ ID NO: 595 - protein, 12 aa, Kaposi?s Sarcoma herpesvirus
SLIQAFNAEGIR

SEQ ID NO: 596 - protein, 12 aa, Kaposi?s Sarcoma herpesvirus
LALSVAGPVPGR

SEQ ID NO: 597 - protein, 16 aa, Kaposi?s Sarcoma herpesvirus
LPPTAPTPATAALETK

SEQ ID NO: 598 - protein, 12 aa, Kaposi?s Sarcoma herpesvirus
AVLANNAALAIR

SEQ ID NO: 599 - protein, 11 aa, Kaposi?s Sarcoma herpesvirus
GFQGNMPSWAR

SEQ ID NO: 600 - protein, 11 aa, Kaposi?s Sarcoma herpesvirus
ILGISNLQFLK

SEQ ID NO: 601 - protein, 10 aa, Kaposi?s Sarcoma herpesvirus
LIASDMSELK

SEQ ID NO: 602 - protein, 9 aa, Kaposi?s Sarcoma herpesvirus
VVSEDVLFR

SEQ ID NO: 603 - protein, 10 aa, Kaposi?s Sarcoma herpesvirus
GFAEVVAMIK

SEQ ID NO: 604 - protein, 12 aa, Kaposi?s Sarcoma herpesvirus
LFADELAALQSK

SEQ ID NO: 605 - protein, 15 aa, Kaposi?s Sarcoma herpesvirus
ETSPDYIQIMQYLSK

SEQ ID NO: 606 - protein, 22 aa, Kaposi?s Sarcoma herpesvirus
SESPGEGPSGTGGSAAAGDITR

SEQ ID NO: 607 - protein, 16 aa, Kaposi?s Sarcoma herpesvirus
IHFPLATYAPVISAEK

SEQ ID NO: 608 - protein, 20 aa, Kaposi?s Sarcoma herpesvirus
TIGGGDDSFNTFFSETGAGK

SEQ ID NO: 609 - protein, 8 aa, Kaposi?s Sarcoma herpesvirus
EDMAALEK

(19) United States
(12) Patent Application Publication
(10) Pub. No.: US 2024/0027453 A1
(43) Pub. Date: Jan. 25, 2024

(54) METHOD FOR DETECTION AND QUANTITATIVE MONITORING OF INFECTIONS WITH HERPESVIRUSES

(71) Applicant: The Trustees of Princeton University, Princeton, NJ (US)

(72) Inventors: Ileana M. Cristea, Princeton, NJ (US); Joel Federspiel, North Reading, MA (US); Michelle A. Kennedy, West Windsor, NJ (US)

(73) Assignee: The Trustees of Princeton University, Princeton, NJ (US)

(21) Appl. No.: 18/006,035

(22) PCT Filed: Jul. 28, 2021
(86) PCT No.: PCT/US2021/043436

§ 371 Date: Jan. 19, 2023

Related U.S. Application Data
(60) Provisional application No. 63/057,853, filed on Jul. 28, 2020.

(51) Int. Cl. G01N 33/569

(52) U.S. Cl. CPC G01N 33/56994; G01N 2800/26; G01N 2800/52

(57) ABSTRACT

Described are systems and assays that monitor presence and/or quantity of herpesviruses viral proteins. Embodiments offer accurate detection and quantification of viral proteins from all temporal classes of viral replication. Three exemplary assays provide specific detection of: herpes simplex vims type 1 (HSV1), human cytomegalovirus (HCMV), and Kaposi's sarcoma-associated herpesvirus (KSHV). These assays can be utilized in combination with drug treatments, genetic modifications, or other perturbations to assess the impact of the intervention on viral protein production. Also provided are kits for use with such assays, peptides useful in the describes assays (including labeled peptides and collections of a plurality of different peptides), nucleic acids and other genetic constructs encoding such peptides, systems for carrying out the described assays (including computer-based or computer-assisted systems), and methods for using the assays for instance in drug development and analysis, vaccine development and analysis, genetic analysis, environmental analysis, etc.

### STATEMENT REGARDING FEDERALLY SPONSORED RESEARCH OR DEVELOPMENT

This invention was made with government support under Grant No. GM114141 awarded by the National Institutes of Health. The government has certain rights in the invention.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD OF THE DISCLOSURE

The present disclosure relates generally to compositions, devices, systems, and methods for detection and quantification of herpesvirus infection. Embodiments of the disclosure describe methods of identifying and using protein signatures of herpesvirus infection, as well as exemplary protein signatures for use in such methods.

### BACKGROUND OF THE DISCLOSURE

Herpesviruses infect up to 90% of the population and are dangerous in immunocompromised individuals and pregnant women. However, there are currently no effective non-toxic antiviral treatments or vaccines for these viruses. The replication of herpesviruses in host cells and the spread of infection to neighboring cells relies on a finely controlled virus replication cycle with a temporally tuned cascade of viral gene expression.

Despite the importance of herpesvirus infection, there exists an on-going need for methods to detect viral proteins or quantitatively track herpesvirus infections. Few antibodies specific for herpesvirus proteins are available, which inhibits accurate detection and tracking of herpesvirus infections.

### SUMMARY OF THE DISCLOSURE

In order to effectively identify potential antiviral compounds, as well as gain an understanding of their impact on specific stages of a viral infection, described herein is development of a novel assay to monitor viral proteins from herpesviruses, such as the important human pathogens HSV-1 (an alpha herpesvirus), HMCV (a beta herpesvirus), and KSHV (a gamma herpesvirus). The described assays offer accurate detection and quantification of viral proteins from all distinct temporal classes (also referred to as kinetic classes) of viral replication (immediate-early (alpha), early (beta), and late (gamma)). These assays can be used to effectively screen and characterize potential antiviral compounds and any other infection modulators, as well as to gain mechanistic insights for instance by identifying the stage of infection and specific viral proteins affected by a compound. This is highly relevant for pharmaceutical companies and in clinical and biological research settings.

This disclosure describes the development of a method to assess the effects of small molecule treatment (or other perturbations) on herpesvirus infections by directly monitoring the temporal production and abundance levels of viral proteins. Assay embodiments described herein focus on herpesviruses due to the clear unmet medical need that they represent. This method is demonstrated herein for the three groups of herpesviruses (alpha, beta and gamma), including herpes simplex virus type 1 (HSV-1), human cytomegalovirus (HCMV) and Kaposi's sarcoma-associated herpesvirus (KSHV). The methods describe herein address at least three aims: (1) provide assays that allow accurate monitoring of the different temporal stages of viral infections, (2) enable use of these assays to screen for potential drugs that directly inhibit viral replication, determining the precise infection time point when these small molecules act, and (3) provide kits useful with the described assays.

One embodiment is an assay, including: obtaining a sample including: a cell or tissue infected with a herpesvirus, an extract from a cell or tissue infected with a herpesvirus, or a protein preparation from a cell or tissue infected with a herpesvirus; determining the abundance level of a plurality of herpesvirus proteins in the sample using parallel reaction monitoring (PRM) to quantify signature peptide(s) corresponding to the herpesvirus proteins; wherein the herpesvirus is HSV-1 and the signature peptides are selected from peptides in Table 1; or the herpesvirus is HCMV and the signature peptides are selected from peptides in Table 2; or the herpesvirus is KSHV and the signature peptides are selected from peptides in Table 3.

In examples of the assay embodiments, for at least the one herpesvirus protein for which the abundance level is determined, at least two signature peptides are quantified.

In examples of the assay embodiments, determining the abundance level of the plurality of herpesvirus proteins using PRM includes subjecting the sample to liquid chromatography coupled to tandem mass spectrometry (LC-MS/MS).

In examples of the assay embodiments, the plurality of herpesvirus proteins includes at least one herpesvirus protein from each temporal class of viral replication for that herpesvirus.

In examples of the assay embodiments, the cell or tissue infected with the herpesvirus is a human cell or human tissue.

In examples of the assay embodiments, the plurality of herpesvirus proteins constitutes approximately 30-70%, or 50-80%, of the predicted viral proteome.

Also provided are time course assay embodiments, which assays involve repeating a herpesvirus protein assay as describe herein a plurality of times, where for each repetition the sample is obtained at a different timepoint in a time course. By way of example, the different timepoints in some instances are different times post infection of the cell or tissue with the herpesvirus. For instance, the different times after infection of the cell or tissue with the herpesvirus include at least one time from each state of a replication cycle of the herpesvirus. In yet other examples, the different timepoints are different times post exposure of the cell or tissue to a compound or a genetic or environmental variable.

Another provided embodiment is an exposure or dosage course assay (that is, an assay that is sampled across multiple exposures or dosages), the assay including: repeating a herpesvirus protein assay as described herein a plurality of times, where for each repetition the sample is obtained from a cell or tissue that has been exposed to a different compound or condition or a different dosage of a compound or a condition. By way of example, the different compounds include one or more of known antiviral compounds, proposed antiviral compounds, test compounds, small molecule drugs or drug candidates, or siRNAs or other biologically active non-coding RNAs. For instance, the known antiviral compounds may include one or more of acyclovir, ganciclovir, another nucleoside, penciclovir, famciclovir, valacyclovir, valganciclovir, cidofovir, another nucleotide phosphonate, fomivirsen, or foscarnet. In additional examples of the exposure or dosage course, the different compounds can include honokiol.

In additional embodiments of the exposure or dosage course, the different exposures include one or more of genetic modification of the cell or tissue, genetic modification of the herpesvirus, environmental conditions, or cell or tissue growth or harvesting conditions. For instance, the genetic modification of the cell or tissue includes knock out or up-regulation of one or more host factors.

Yet another embodiment is a method for quantification of herpesvirus proteins from multiple temporal classes of viral replication, which method includes: subjecting a cell sample or cell extract to parallel reaction monitoring (PRM) to generate abundance data; analyzing the abundance data to quantify signature peptide(s) corresponding to at least one herpesvirus protein from each of at least two temporal classes of viral replication; and providing the quantified peptide(s) results from the analyzing to a database, a computer memory, a display, a printer, or another output device; wherein the herpesvirus is HSV-1 and the signature peptides are selected from peptides in Table 1; or the herpesvirus is HCMV and the signature peptides are selected from peptides in Table 2; or the herpesvirus is KSHV and the signature peptides are selected from peptides in Table 3.

Also described is use of any of the assays of the disclosure to: screen drug candidates as modulators of viral infection; analyze the stage of infection at which a test compound acts; determine what functional family(s) of viral proteins are affected by a drug or drug candidate; characterize viral and/or host responses to viral infection; characterize viral and/or host responses to drug treatment; or characterize viral and/or host responses to genetic manipulation of either the viral genome or the host genome.

Another embodiment is a kit for use with an assay or use embodiment, which kit includes: parameters for performing the assay for a target herpesvirus, a set of heavy isotope labeled peptides for use as controls, and a USB drive or other non-transitory computer readable medium containing software for assay analysis and/or standardized report generation. In examples of this kit embodiment, the target herpesvirus is HSV-1 and the set of heavy isotope labeled peptides includes: at least two signature peptides in Table 1; at least one signature peptide for each protein in Table 1; or at least one signature peptide from Table 1 for at least one protein from each temporal stage of HSV-1 viral replication. In further examples of the kit embodiment, the target herpesvirus is HCMV and the set of heavy isotope labeled peptides includes: at least two signature peptides in Table 2; at least one signature peptide for each protein in Table 2; or at least one signature peptide from Table 2 for at least one protein from each temporal stage of HCMV viral replication. In yet further examples, the target herpesvirus is KSHV and the set of heavy isotope labeled peptides includes: at least two signature peptides in Table 3; at least one signature peptide for each protein in Table 3; or at least one signature peptide from Table 3 for at least one protein from each temporal stage of KSHV viral replication.

Another embodiment is a service, the service including: performing an assay or a use as described herein on one or more biological samples provided by another/a third party (such as a researcher, a medical practitioner, and so forth). By way of example, such a service may be carried out for a fee. Optionally, results of the assay analysis may be provided to the third party by way of internet or other computerized correspondence.

This disclosure also provides assays, such as quantitative assays, for herpesviral proteins, substantially as described herein.

Yet another embodiment is a non-naturally occurring, labeled peptide having the amino acid sequence of a peptide in Table 1, Table 2, or Table 3. In examples of this non-naturally occurring, labeled peptide embodiment, the label enables the peptide to be distinguished from an unlabeled peptide with the same amino acid sequence in liquid chromatography coupled to tandem mass spectrometry (LC-MS/MS) analysis.

Also described is a collection of non-naturally occurring, labeled signature peptides specific for HSV-1, the collection including: at least one peptide from Table 1 for each of the 60 proteins listed in Table 1; at least two peptides from Table 1 for each of the 60 proteins listed in Table 1; at least three peptides from Table 1 for each of the 60 proteins listed in Table 1; at least one peptide from Table 1 for at least one protein listed in Table 1 from each temporal stage of HSV-viral replication; at least 60 of the peptides listed in Table 1; more than 60 of the peptides listed in Table 1; at least 30 of the peptides listed in Table 1; at least 50 of the peptides listed in Table 1; at least 60 of the peptides listed in Table 1; substantially all of the peptides listed in Table 1; or all of the peptides listed in Table 1; wherein each peptide in the collection includes a label that enables the labeled peptide to be distinguished from an unlabeled peptide with the same amino acid sequence in liquid chromatography coupled to tandem mass spectrometry (LC-MS/MS) analysis.

Also described is a collection of non-naturally occurring, labeled signature peptides specific for HCMV, the collection including: at least one peptide from Table 2 for each of the 90 proteins listed in Table 2; at least two peptides from Table 2 for a plurality of the 90 proteins listed in Table 2; at least three peptides from Table 2 for a plurality of the 90 proteins listed in Table 2; at least one peptide from Table 2 for at least one protein listed in Table 2 from each temporal stage of HCMV-viral replication; at least 90 of the peptides listed in Table 2; more than 90 of the peptides listed in Table 2; at least 30 of the peptides listed in Table 2; at least 50 of the peptides listed in Table 2; at least 100 of the peptides listed in Table 2; at least 150 of the peptides listed in Table 2; at least 200 of the peptides listed in Table 2; substantially all of the peptides listed in Table 2; or all of the peptides listed in Table 2; wherein each peptide in the collection includes a label that enables the labeled peptide to be distinguished from an unlabeled peptide with the same amino acid sequence in liquid chromatography coupled to tandem mass spectrometry (LC-MS/MS) analysis.

Also described is a collection of non-naturally occurring, labeled signature peptides specific for KSHV, the collection including: at least one peptide from Table 3 for each of the 62 proteins listed in Table 3; at least two peptides from Table 3 for a plurality of the 62 proteins listed in Table 3; at least three peptides from Table 3 for a plurality of the 62 proteins listed in Table 3; at least one peptide from Table 3 for at least one protein listed in Table 3 from each temporal stage of KSHV-viral replication; at least 62 of the peptides listed in Table 3; more than 62 of the peptides listed in Table 3; at least 30 of the peptides listed in Table 3; at least 50 of the peptides listed in Table 3; at least 75 of the peptides listed in Table 3; at least 100 of the peptides listed in Table 3; at least 150 of the peptides listed in Table 3; substantially all of the peptides listed in Table 3; or all of the peptides listed in Table 3; wherein each peptide in the collection includes a label that enables the labeled peptide to be distinguished from an unlabeled peptide with the same amino acid sequence in liquid chromatography coupled to tandem mass spectrometry (LC-MS/MS) analysis.

In any of the embodiments of non-naturally occurring, labeled signature peptides, the label on at least one peptide in the collection may include a heavy isotope. In some examples, all of the peptides in the collection include a heavy isotope.

### DESCRIPTION OF THE DRAWINGS

One or more of the drawings submitted herewith are better understood in color, which is not available in patent application publications at the time of filing. Applicant considers the color versions of the drawings as part of the original submission and reserve the right to present color images of the drawings in later proceedings.

FIG. 1. Representative workflow for signature detection of viral proteins by targeted mass spectrometry. First, a library of peptides unique to the proteins of interest and signature information derived from their mass spectrometry (MS) analysis is generated and experimentally validated. Next, this signature information is used for targeted MS analyses by parallel reaction monitoring (PRM). This offers accurate detection and quantification of the proteins of interest during the progression of infection, and can be implemented in any cell or tissue sample. This system can also be employed across different analysis labs, as the peptides provide self-balancing internal controls useful no matter who carries out the analysis.

FIG. 2. is a computer architecture diagram showing one illustrative computer hardware architecture for implementing a computing device that might be utilized to implement aspects of the various embodiments presented herein. For instance, a computing device may be useful in recording, processing, analyzing, and/or presenting information including the quantification of peptide(s) indicative of the presence and/or quantity of a virus such as a herpesvirus. A computing device may be useful in analysis of raw information provided by a mass spectrophotometer, for instance in order to calculate protein level (in absolute or relative numbers) based on the quantification of one or more signature peptide(s) corresponding to that protein.

FIGS. 3A-3F: Developing and validating TRUSTED, a PRM-based method for monitoring HSV-1, HCMV, and KSHV viral proteins (FIG. 3A) Schematic representation of the herpesvirus replication cycle consisting of stages of entry, viral gene expression, genome replication, and the assembly and egress of newly formed virus particles. Timeline below the schematic depicts the relative time scale of replication in hours post-infection (HPI) for the alpha, beta, and gamma-herpesviruses HSV-1, HCMV, and KSHV, respectively. (FIG. 3B) Overview of the PRM assay development process and its subsequent applications. (FIG. 3C) Table of PRM assay specifications and protein targets. (FIG. 3D) Traces of maximum concurrent precursors vs. retention time (RT) for different RT windows. Dashed grey line denotes 30 concurrent precursors, which is the maximum number of precursors that can be monitored at a given RT in a single injection to achieve reliable quantitation with the instrument settings utilized in this study. (FIG. 3E) Normalized abundances across infection time for selected host proteins used for data normalization. (FIG. 3F) Coefficient of variation (CV) between normalized abundance values. (Left) CV across different peptides from a given protein within the same biological replicate. (Middle) CV across biological replicates for a given peptide. (Right) Overall CV for a given protein across peptides and biological replicates. Note: Data is derived from experiments conducted under wild type infection conditions (HSV-1 and HCMV: MOI=3; KSHV: 100% reactivation) and error bars represent a 95% confidence interval (CI) across biological replicates (HSV-1: n=2; HCMV: n=7; KSHV: n=2).

FIGS. 4A-4D: Herpesvirus PRM assay captures the signature temporal cascade of viral gene expression Abundance plots of: HSV-1 viral proteins (FIG. 4A), HSV-1 host proteins (FIG. 4B), HCMV viral proteins (FIG. 4C), and KSHV viral proteins (FIG. 4D). Plots of proteins are stratified by temporal expression class (IE=immediate early; DE=delayed early; E=early; LL=leaky late; L=late). Protein abundance levels are represented as fold-change (log-2) relative to the first time point at which peptides were detected. Data is derived from experiments conducted under wild type infection conditions (HSV-1 and HCMV: MOI=3; KSHV: 100% reactivation) and error bars represent a 95% CI across biological replicates (HSV-1: n=2; HCMV: n=7; KSHV: n=2). Significance was determined by two-tailed Student's t-test; *p<0.05, ** p<0.01, *** p<0.001, and **** p<0.0001.

FIGS. 5A-5E: Differing levels of infection (MOI) are robustly detected via PRM (FIG. 5A) Percent of HCMV peptides detected at different time points across increasing amounts of input virus (multiplicities of infection; MOI); in each set (MOI level), the bars represent 24, 72, and 102 hours post infection (HPI) from left to right. A peptide was considered to be “detected” if it was observed in at least one biological replicate (n=3). (FIG. 5B) Number of viral proteins detected at increasing MOIs. All reported values are inclusive; i.e. all proteins detected at the previous MOI were also detected at the next MOI. (FIG. 5C) Time point of first detection for HCMV proteins at increasing MOIs. The symbol preceding protein gene names corresponds to the part of the virion they are reported to associate with. (FIG. 5D) Average HCMV protein abundance across infection time for increasing amounts of input virus (MOI), stratified by temporal class. Error bars represent a 95% CI across biological replicates (n=3). (FIG. 5E) Protein abundance plots of HCMV proteins US12 and US15 at increasing MOIs. Error bars represent a 95% CI across biological replicates (n=3). Key: IE=Immediate Early, DE=Delayed Early, LL=Leaky Late, and L=Late Early.

FIGS. 6A-6E: PRM application to investigations of clinically employed herpesvirus antiviral drugs (FIGS. 6A-6B) Normalized protein abundance plots of HSV-1 protein levels during treatment with 1 μM acyclovir or DMSO (control) averaged across protein expression temporality classes (FIG. 6A) or individual proteins (FIG. 6B). (FIG. 6C) Heatmap of HCMV protein levels after treatment with 1 μM cidofovir or PBS (control); the corresponding numerical values are provided in Table FIG. 6C. (FIG. 6D) Average HCMV protein abundance following 1 μM cidofovir or PBS (control) treatment stratified by protein expression temporality. (FIG. 6E) Selected individual HCMV protein plots after 1 μM cidofovir or PBS (control) treatment. Error bars represent a 95% CI across biological replicates (HSV-1: n=2; HCMV: n=2). Significance was determined by two-tailed Student's t-test; *p<0.05, ** p<0.01, *** p<0.001, and **** p<0.0001.

FIGS. 7A-7C: Modulation of sirtuin enzymatic activity regulates HCMV viral protein levels (FIG. 7A) Heatmap of average HCMV protein levels after treatment with 10 μM EX-527, 12.5 μM CAY10602, 50 μM trans-Resveratrol (trans-Res.), or DMSO (control); N.D.=not detected; the corresponding numerical values are provided in Table FIG. 7A. (FIG. 7B) Average mean normalized (left) or log-2-fold-change (right; treatment/control) HCMV protein abundances following 10 μM EX-527, 12.5 μM CAY10602, 50 μM trans-Resveratrol, or DMSO (control) treatment stratified by protein expression temporality. (FIG. 7C) Selected individual HCMV protein plots after 10 μM EX-527, 12.5 μM CAY10602, 50 μM trans-Resveratrol, or DMSO (control) treatment. Error bars represent a 95% CI across biological replicates (n=3). Significance was determined by two-tailed Student's t-test; *p<0.05, ** p<0.01, *** p<0.001, and **** p<0.0001.

FIGS. 8A-8D: Modulation of sirtuin enzymatic activity differentially regulates HSV-1 and KSHV viral protein levels throughout infections (FIG. 8A) Heatmap of average HSV-1 protein levels after treatment with 10 μM EX-527, 12.5 μM CAY10602, 50 μM trans-Resveratrol (trans-Res.), or DMSO (control); N.D.=not detected; the corresponding numerical values are provided in Table FIG. 8A. (FIG. 8B) Mean normalized HSV-1 protein abundances following 10 μM EX-527, 12.5 μM CAY10602, 50 μM trans-Resveratrol, or DMSO treatment stratified by protein expression temporality. (FIG. 8C) Heatmap of average KSHV protein levels after treatment with 10 μM EX-527, 12.5 μM CAY10602, or DMSO (control); N.D.=not detected; the corresponding numerical values are provided in Table FIG. 8C. (FIG. 8D) Mean normalized KSHV protein abundances following 10 μM EX-527, 12.5 μM CAY10602, or DMSO (control) treatment stratified by protein expression temporality. Significance was determined by two-tailed Student's t-test; *p<0.05, ** p<0.01, *** p<0.001, and **** p<0.0001. Note: trans-Resveratrol was toxic at 50 μM to the iSLK.219 cell line, so its effect on KSHV protein levels could not be measured.

FIGS. 9A-9E: Conservation of TRUSTED peptides indicates assay utility across several laboratory and clinical virus strains (FIG. 9A) Phylogenetic tree of human herpesviruses from strains annotated in the NCBI taxonomy database. (FIGS. 9B-9E) Predicted conservation of PRM assay peptides and proteins across different species and strains of human herpesviruses as represented by potential peptide sequences reported in complete genome sequences deposited in the NCBI nucleotide database. Peptides were only considered to be conserved if they matched with 100% identity to a consecutive string of amino acids in a given computationally translated nucleotide sequence. (FIG. 9B) Number of proteins in the PRM assays that are conserved across different human herpesvirus strains. Any protein with at least one conserved peptide is depicted and protein representation by temporal class is shown as a stacked bar graph. (FIGS. 9C-9E) Numbers of conserved peptides for all proteins targeted in the PRM assays for HSV-1 (FIG. 9C; corresponding numerical values provided in Table FIG. 9C), HCMV (FIG. 9D; corresponding numerical values provided in Table FIG. 9D), and KSHV (FIG. 9E; corresponding numerical values provided in Table FIG. 9E). The number of conserved peptides is denoted within each box and its color corresponds to the percent of peptides that are conserved for a given protein.

### REFERENCE TO SEQUENCES

The amino acid sequences described herein are shown using standard letter abbreviations, as defined in 37 C.F.R. § 1.822. Only one strand of each nucleic acid sequence is shown, but the complementary strand is understood as included in embodiments where it would be appropriate. A computer readable text file, entitled P172-0004US_SeqList created on or about Jan. 18, 2023, with a file size of 116 KB, contains the sequence listing for this application and is hereby incorporated by reference in its entirety.

Information about sequences in the Sequence Listing is provided in the following three Tables. Temporality abbreviations: IE=Immediate Early, DE=Delayed Early, LL=Leaky Late, and L=Late Early; and Virion component abbreviations: NS=non-structural, E=envelope, T=tegument, C=Capsid.

TABLE 1
List of Signature Peptides for The Identification of HSV-1 Proteins:

| | Protein | Protein | | component | | SEQ ID |
|---|---|---|---|---|---|---|
| # | Accession | Gene | temporality | virion | Protein Description | NO: |
| 1 | P04296 | DBP | E | NS | Major DNA-binding protein OS = Human | 1-4 |
| | | | | | herpesvirus 1 (strain 17) OX = 10299 | |
| | | | | | GN = DBP PE = 1 SV = 1 | |
| 2 | P10211 | gB | L | E | Envelope glycoprotein B OS = Human | 5-8 |
| | | | | | herpesvirus 1 (strain 17) OX = 10299 GN = gB | |
| | | | | | PE = 1 SV = 1 | |
| 3 | P06487 | gI | L | E | Envelope glycoprotein I OS = Human | 9-12 |
| | | | | | herpesvirus 1 (strain 17) OX = 10299 GN = gI | |
| | | | | | PE = 1 SV = 1 | |
| 4 | P08393 | ICPO | IE | T | E3 ubiquitin-protein ligase ICP0 OS = Human | 13-17 |
| | | | | | herpesvirus 1 (strain 17) OX = 10299 | |
| | | | | | GN = ICPO PE = 1 SV = 1 | |
| 5 | P04485 | ICP22 | IE | NS | Transcriptional regulator ICP22 OS = Human | 18-21 |
| | | | | | herpesvirus 1 (strain 17) OX = 10299 | |
| | | | | | GN = ICP22 PE = 1 SV = 1 | |
| 6 | P08392 | ICP4 | IE | T | Major viral transcription factor ICP4 | 22-27 |
| | | | | | OS = Human herpesvirus 1 (strain 17) | |
| | | | | | OX = 10299 GN = ICP4 PE = 1 SV = 1 | |
| 7 | P03176 | TK | E | T | Thymidine kinase OS = Human herpesvirus 1 | 28-31 |
| | | | | | (strain 17) OX = 10299 GN = TK PE = 1 SV = 4 | |
| 8 | P04294 | UL12 | E | NS | Alkaline nuclease OS = Human herpesvirus 1 | 32-37 |
| | | | | | (strain 17) OX = 10299 GN = UL12 PE = 1 | |
| | | | | | SV = 1 | |
| 9 | P10210 | UL26 | L | C | Capsid scaffolding protein OS = Human | 38-42 |
| | | | | | herpesvirus 1 (strain 17) OX = 10299 | |
| | | | | | GN = UL26 PE = 1 SV = 1 | |
| 10 | P04293 | UL30 | E | NS | DNA polymerase catalytic subunit | 43-46 |
| | | | | | OS = Human herpesvirus 1 (strain 17) | |
| | | | | | OX = 10299 GN = UL30 PE = 1 SV = 2 | |
| 11 | P10226 | UL42 | E | NS | DNA polymerase processivity factor | 47-50 |
| | | | | | OS = Human herpesvirus 1 (strain 17) | |
| | | | | | OX = 10299 GN = UL42 PE = 1 SV = 1 | |
| 12 | P06492 | UL48 | L | T | Tegument protein VP16 OS = Human | 51-54 |
| | | | | | herpesvirus 1 (strain 17) OX = 10299 | |
| | | | | | GN = UL48 PE = 1 SV = 1 | |
| 13 | P10233 | UL49 | L | T | Tegument protein VP22 OS = Human | 55-58 |
| | | | | | herpesvirus 1 (strain 17) OX = 10299 | |
| | | | | | GN = UL49 PE = 1 SV = 1 | |
| 14 | P10238 | UL54 | IE | NS | mRNA export factor OS = Human | 59-62 |
| | | | | | herpesvirus 1 (strain 17) OX = 10299 | |
| | | | | | GN = UL54 PE = 1 SV = 1 | |
| 15 | P10192 | UL8 | E | NS | DNA helicase/primase complex-associated | 63-66 |
| | | | | | protein OS = Human herpesvirus 1 (strain 17) | |
| | | | | | OX = 10299 GN = UL8 PE = 1 SV = 1 | |
| 16 | P04413 | US3 | E | T | Serine/threonine-protein kinase US3 | 67-70 |
| | | | | | OS = Human herpesvirus 1 (strain 17) | |
| | | | | | OX = 10299 GN = US3 PE = 1 SV = 1 | |
| 17 | P10209 | CVC2 | L | C | Capsid vertex component 2 OS = Human | 71-76 |
| | | | | | herpesvirus 1 (strain 17) OX = 10299 | |
| | | | | | GN = CVC2 PE = 1 SV = 1 | |
| 18 | P10201 | CVC1 | L | C | Capsid vertex component 1 OS = Human | 77-79 |
| | | | | | herpesvirus 1 (strain 17) OX = 10299 | |
| | | | | | GN = CVC1 PE = 1 SV = 1 | |
| 19 | P10234 | DUT | E | T | Deoxyuridine 5′-triphosphate | 80-83 |
| | | | | | nucleotidohydrolase OS = Human | |
| | | | | | herpesvirus 1 (strain 17) OX = 10299 | |
| | | | | | GN = DUT PE = 3 SV = 1 | |
| 20 | P10228 | gC | L | E | Envelope glycoprotein C OS = Human | 84-87 |
| | | | | | herpesvirus 1 (strain 17) OX = 10299 GN = gC | |
| | | | | | PE = 1 SV = 1 | |
| 21 | Q69091 | gD | L | E | Envelope glycoprotein D OS = Human | 88-92 |
| | | | | | herpesvirus 1 (strain 17) OX = 10299 GN = gD | |
| | | | | | PE = 1 SV = 1 | |
| 22 | P04488 | gE | L | E | Envelope glycoprotein E OS = Human | 93-96 |
| | | | | | herpesvirus 1 (strain 17) OX = 10299 GN = gE | |
| | | | | | PE = 1 SV = 1 | |
| 23 | P06484 | gG | L | E | Envelope glycoprotein G OS = Human | 97 |
| | | | | | herpesvirus 1 (strain 17) OX = 10299 GN = gG | |
| | | | | | PE = 3 SV = 1 | |
| 24 | P06477 | gH | L | E | Envelope glycoprotein H OS = Human | 98-101 |
| | | | | | herpesvirus 1 (strain 17) OX = 10299 GN = gH | |
| | | | | | PE = 1 SV = 1 | |
| 25 | P68331 | gk | L | E | Envelope glycoprotein K OS = Human | 102-103 |
| | | | | | herpesvirus 1 (strain 17) OX = 10299 GN = gK | |
| | | | | | PE = 1 SV = 1 | |
| 26 | P04288 | gM | L | E | Envelope glycoprotein M OS = Human | 104-107 |
| | | | | | herpesvirus 1 (strain 17) OX = 10299 GN = gM | |
| | | | | | PE = 1 SV = 1 | |
| 27 | P10189 | HELI | E | NS | DNA replication helicase OS = Human | 108-109 |
| | | | | | herpesvirus 1 (strain 17) OX = 10299 | |
| | | | | | GN = HELI PE = 3 SV = 3 | |
| 28 | P06491 | MCP | L | C | Major capsid protein OS = Human | 110-115 |
| | | | | | herpesvirus 1 (strain 17) OX = 10299 | |
| | | | | | GN = MCP PE = 1 SV = 1 | |
| 29 | P10215 | NEC1 | L | NS | Nuclear egress protein 1 OS = Human | 116-118 |
| | | | | | herpesvirus 1 (strain 17) OX = 10299 | |
| | | | | | GN = NEC1 PE = 1 SV = 1 | |
| 30 | P10218 | NEC2 | L | NS | Nuclear egress protein 2 OS = Human | 119-123 |
| | | | | | herpesvirus 1 (strain 17) OX = 10299 | |
| | | | | | GN = NEC2 PE = 1 SV = 1 | |
| 31 | P08543 | RIR1 | E | NS | Ribonucleoside-diphosphate reductase | 124-126 |
| | | | | | large subunit OS = Human herpesvirus 1 | |
| | | | | | (strain 17) OX = 10299 GN = RIR1 PE = 1 | |
| | | | | | SV = 2 | |
| 32 | P10224 | RIR2 | E | NS | Ribonucleoside-diphosphate reductase | 127-130 |
| | | | | | small subunit OS = Human herpesvirus 1 | |
| | | | | | (strain 17) OX = 10299 GN = RIR2 PE = 3 | |
| | | | | | SV = 1 | |
| 33 | P10219 | SCP | L | C | Small capsomere-interacting protein | 131 |
| | | | | | OS = Human herpesvirus 1 (strain 17) | |
| | | | | | OX = 10299 GN = SCP PE = 1 SV = 1 | |
| 34 | P32888 | TRX1 | L | C | Triplex capsid protein 1 OS = Human | 132-136 |
| | | | | | herpesvirus 1 (strain 17) OX = 10299 | |
| | | | | | GN = TRX1 PE = 1 SV = 1 | |
| 35 | P10202 | TRX2 | L | C | Triplex capsid protein 2 OS = Human | 137-141 |
| | | | | | herpesvirus 1 (strain 17) OX = 10299 | |
| | | | | | GN = TRX2 PE = 1 SV = 1 | |
| 36 | P04291 | UL14 | L | T | Tegument protein UL14 OS = Human | 142 |
| | | | | | herpesvirus 1 (strain 17) OX = 10299 | |
| | | | | | GN = UL14 PE = 1 SV = 2 | |
| 37 | P10200 | UL16 | L | T | Cytoplasmic envelopment protein 2 | 143-145 |
| | | | | | OS = Human herpesvirus 1 (strain 17) | |
| | | | | | OX = 10299 GN = UL16 PE = 1 SV = 1 | |
| 38 | P10186 | UL2 | E | NS | Uracil-DNA glycosylase OS = Human | 146-150 |
| | | | | | herpesvirus 1 (strain 17) OX = 10299 | |
| | | | | | GN = UL2 PE = 1 SV = 1 | |
| 39 | P10205 | UL21 | L | T | Tegument protein UL21 OS = Human | 151-156 |
| | | | | | herpesvirus 1 (strain 17) OX = 10299 | |
| | | | | | GN = UL21 PE = 1 SV = 1 | |
| 40 | P10208 | UL24 | L | NS | Protein UL24 OS = Human herpesvirus 1 | 157 |
| | | | | | (strain 17) OX = 10299 GN = UL24 PE = 1 | |
| | | | | | SV = 1 | |
| 41 | P10187 | UL3 | L | NS | Nuclear phosphoprotein UL3 OS = Human | 158 |
| | | | | | herpesvirus 1 (strain 17) OX = 10299 | |
| | | | | | GN = UL3 PE = 3 SV = 1 | |
| 42 | P10216 | UL32 | L | NS | Packaging protein UL32 OS = Human | 159-160 |
| | | | | | herpesvirus 1 (strain 17) OX = 10299 | |
| | | | | | GN = UL32 PE = 1 SV = 1 | |
| 43 | P10220 | UL36 | L | T | Large tegument protein deneddylase | 161-165 |
| | | | | | OS = Human herpesvirus 1 (strain 17) | |
| | | | | | OX = 10299 GN = UL36 PE = 1 SV = 1 | |
| 44 | P10221 | UL37 | L | T | Inner tegument protein OS = Human | 166-171 |
| | | | | | herpesvirus 1 (strain 17) OX = 10299 | |
| | | | | | GN = UL37 PE = 1 SV = 1 | |
| 45 | P10188 | UL4 | L | T | Nuclear protein UL4 OS = Human | 172 |
| | | | | | herpesvirus 1 (strain 17) OX = 10299 | |
| | | | | | GN = UL4 PE = 3 SV = 1 | |
| 46 | P10225 | UL41 | L | T | Virion host shutoff protein OS = Human | 173-178 |
| | | | | | herpesvirus 1 (strain 17) OX = 10299 | |
| | | | | | GN = UL41 PE = 1 SV = 1 | |
| 47 | P10229 | UL45 | L | E | Envelope protein UL45 OS = Human | 179 |
| | | | | | herpesvirus 1 (strain 17) OX = 10299 | |
| | | | | | GN = UL45 PE = 3 SV = 1 | |
| 48 | P10230 | UL46 | L | T | Tegument protein UL46 OS = Human | 180-184 |
| | | | | | herpesvirus 1 (strain 17) OX = 10299 | |
| | | | | | GN = UL46 PE = 1 SV = 2 | |
| 49 | P10231 | UL47 | L | T | Tegument protein UL47 OS = Human | 185-189 |
| | | | | | herpesvirus 1 (strain 17) OX = 10299 | |
| | | | | | GN = UL47 PE = 1 SV = 1 | |
| 50 | P10235 | UL51 | L | T | Tegument protein UL51 OS = Human | 190-191 |
| | | | | | herpesvirus 1 (strain 17) OX = 10299 | |
| | | | | | GN = UL51 PE = 1 SV = 1 | |
| 51 | P10236 | UL52 | E | NS | DNA primase OS = Human herpesvirus 1 | 192-93 |
| | | | | | (strain 17) OX = 10299 GN = UL52 PE = 1 | |
| | | | | | SV = 1 | |
| 52 | P10240 | UL56 | E | E | Protein UL56 OS = Human herpesvirus 1 | 194-195 |
| | | | | | (strain 17) OX = 10299 GN = UL56 PE = 1 | |
| | | | | | SV = 2 | |
| 53 | P10190 | UL6 | E | C | Portal protein OS = Human herpesvirus 1 | 196-198 |
| | | | | | (strain 17) OX = 10299 GN = UL6 PE = 1 SV = 1 | |
| 54 | P10191 | UL7 | L | T | Cytoplasmic envelopment protein 1 | 199-203 |
| | | | | | OS = Human herpesvirus 1 (strain 17) | |
| | | | | | OX = 10299 GN = UL7 PE = 1 SV = 1 | |
| 55 | P10193 | UL9 | L | NS | Replication origin-binding protein | 204 |
| | | | | | OS = Human herpesvirus 1 (strain 17) | |
| | | | | | OX = 10299 GN = UL9 PE = 1 SV = 1 | |
| 56 | P06486 | US10 | E | T | Virion protein US10 OS = Human herpesvirus | 205-207 |
| | | | | | 1 (strain 17) OX = 10299 GN = US10 PE = 1 | |
| | | | | | SV = 1 | |
| 57 | P04487 | US11 | L | T | Accessory factor US11 OS = Human | 208-211 |
| | | | | | herpesvirus 1 (strain 17) OX = 10299 | |
| | | | | | GN = US11 PE = 1 SV = 1 | |
| 58 | P03170 | US12 | IE | NS | ICP47 protein OS = Human herpesvirus 1 | 212 |
| | | | | | (strain 17) OX = 10299 GN = US12 PE = 1 | |
| | | | | | SV = 2 | |
| 59 | P06485 | US2 | L | T | Protein US2 OS = Human herpesvirus 1 | 213-217 |
| | | | | | (strain 17) OX = 10299 GN = US2 PE = 1 SV = 3 | |
| 60 | P06481 | US9 | E | E | Envelope protein US9 OS = Human | 218-219 |
| | | | | | herpesvirus 1 (strain 17) OX = 10299 | |
| | | | | | GN = US9 PE = 1 SV = 1 | |

TABLE 2
List of Signature Peptides for The Identification of HCMV Proteins:

| | Protein | Protein | | component | | SEQ ID |
|---|---|---|---|---|---|---|
| # | Accession | Gene | temporality | virion | Protein Description | NO(s): |
| 1 | P16810 | IRL12 | LL | NS | Uncharacterized protein IRL12 OS = Human | 220-225 |
| | | | | | cytomegalovirus (strain AD169) OX = 10360 | |
| | | | | | PE = 4 SV = 1 | |
| 2 | P16809 | IR11 | L | E | Viral Fc-gamma receptor-like protein IR11 | 226-231 |
| | | | | | OS = Human cytomegalovirus (strain AD169) | |
| | | | | | OX = 10360 PE = 3 SV = 1 | |
| 3 | P17147 | DBP | DE | NS | Major DNA-binding protein OS = Human | 232-234 |
| | | | | | cytomegalovirus (strain AD169) OX = 10360 | |
| | | | | | GN = DBP PE = 1 SV = 1 | |
| 4 | P06473 | gB | DE | E | Envelope glycoprotein B OS = Human | 235-237 |
| | | | | | cytomegalovirus (strain AD169) OX = 10360 | |
| | | | | | GN = gB PE = 1 SV = 1 | |
| 5 | P12824 | gH | L | E | Envelope glycoprotein H OS = Human | 238-239 |
| | | | | | cytomegalovirus (strain AD169) OX = 10360 | |
| | | | | | GN = gH PE = 1 SV = 1 | |
| 6 | P16733 | gM | L | E | Envelope glycoprotein M OS = Human | 240-242 |
| | | | | | cytomegalovirus (strain AD169) OX = 10360 | |
| | | | | | GN = gM PE = 1 SV = 1 | |
| 7 | P16795 | gN | L | E | Envelope glycoprotein N OS = Human | 243-244 |
| | | | | | cytomegalovirus (strain AD169) OX = 10360 | |
| | | | | | GN = gN PE = 1 SV = 1 | |
| 8 | P09715 | IRS1 | IE | T | Protein IRS1 OS = Human cytomegalovirus | 245-247 |
| | | | | | (strain AD169) OX = 10360 GN = IRS1 PE = 1 | |
| | | | | | SV = 1 | |
| 9 | P16729 | MCP | L | C | Major capsid protein OS = Human | 248-250 |
| | | | | | cytomegalovirus (strain AD169) OX = 10360 | |
| | | | | | GN = MCP PE = 1 SV = 1 | |
| 10 | P16794 | NEC1 | DE | T | Nuclear egress protein 1 OS = Human | 251-252 |
| | | | | | cytomegalovirus (strain AD169) OX = 10360 | |
| | | | | | GN = NEC1 PE = 1 SV = 1 | |
| 11 | P16791 | NEC2 | DE | T | Nuclear egress protein 2 OS = Human | 253-254 |
| | | | | | cytomegalovirus (strain AD169) OX = 10360 | |
| | | | | | GN = NEC2 PE = 1 SV = 1 | |
| 12 | P16782 | RIR1 | DE | T | Ribonucleoside-diphosphate reductase | 255-257 |
| | | | | | large subunit-like protein OS = Human | |
| | | | | | cytomegalovirus (strain AD169) OX = 10360 | |
| | | | | | GN = RIR1 PE = 3 SV = 1 | |
| 13 | P16724 | TRM1 | DE | NS | Tripartite terminase subunit 1 OS = Human | 258-260 |
| | | | | | cytomegalovirus (strain AD169) OX = 10360 | |
| | | | | | GN = TRM1 PE = 3 SV = 1 | |
| 14 | P16792 | TRM2 | L | NS | Tripartite terminase subunit 2 OS = Human | 261-263 |
| | | | | | cytomegalovirus (strain AD169) OX = 10360 | |
| | | | | | GN = TRM2 PE = 3 SV = 1 | |
| 15 | P16732 | TRM3 | LL | NS | Tripartite terminase subunit 3 OS = Human | 264-266 |
| | | | | | cytomegalovirus (strain AD169) OX = 10360 | |
| | | | | | GN = TRM3 PE = 1 SV = 1 | |
| 16 | P09695 | TRS1 | IE | T | Protein HHLF1 OS = Human | 267-268 |
| | | | | | cytomegalovirus (strain AD169) OX = 10360 | |
| | | | | | GN = TRS1 PE = 1 SV = 1 | |
| 17 | P16783 | TRX1 | LL | C | Triplex capsid protein 1 OS = Human | 269-271 |
| | | | | | cytomegalovirus (strain AD169) OX = 10360 | |
| | | | | | GN = TRX1 PE = 1 SV = 1 | |
| 18 | P16728 | TRX2 | LL | C | Triplex capsid protein 2 OS = Human | 272-274 |
| | | | | | cytomegalovirus (strain AD169) OX = 10360 | |
| | | | | | GN = TRX2 PE = 1 SV = 1 | |
| 19 | P16827 | UL102 | DE | NS | DNA helicase/primase complex-associated | 275-277 |
| | | | | | protein OS = Human cytomegalovirus (strain | |
| | | | | | AD169) OX = 10360 GN = UL102 PE = 2 SV = 2 | |
| 20 | P16734 | UL103 | L | T | Cytoplasmic envelopment protein 1 | 278-280 |
| | | | | | OS = Human cytomegalovirus (strain AD169) | |
| | | | | | OX = 10360 GN = UL103 PE = 3 SV = 1 | |
| 21 | P17151 | UL112/ | DE | NS | Early phosphoprotein p84 OS = Human | 281-283 |
| | | UL113 | | | cytomegalovirus (strain AD169) OX = 10360 | |
| | | | | | GN = UL112/UL113 PE = 1 SV = 2 | |
| 22 | P16770 | UL117 | L | NS | Protein UL117 OS = Human cytomegalovirus | 284-286 |
| | | | | | (strain AD169) OX = 10360 GN = UL117 PE = 3 | |
| | | | | | SV = 1 | |
| 23 | P16739 | UL119/ | DE | NS | Viral Fc-gamma receptor-like protein UL119 | 287-289 |
| | | UL118 | | | OS = Human cytomegalovirus (strain AD169) | |
| | | | | | OX = 10360 GN = UL119/UL118 PE = 2 SV = 2 | |
| 24 | P19893 | UL122 | IE | NS | Viral transcription factor IE2 OS = Human | 290-291 |
| | | | | | cytomegalovirus (strain AD169) OX = 10360 | |
| | | | | | GN = UL122 PE = 1 SV = 2 | |
| 25 | P13202 | UL123 | IE | NS | 55 kDa immediate-early protein 1 | 292-294 |
| | | | | | OS = Human cytomegalovirus (strain AD169) | |
| | | | | | OX = 10360 GN = UL123 PE = 1 SV = 1 | |
| 26 | P16755 | UL13 | IE | T | Uncharacterized protein UL13 OS = Human | 295-297 |
| | | | | | cytomegalovirus (strain AD169) OX = 10360 | |
| | | | | | GN = UL13 PE = 3 SV = 1 | |
| 27 | P69338 | UL132 | LL | E | Envelope glycoprotein UL132 OS = Human | 298-300 |
| | | | | | cytomegalovirus (strain AD169) OX = 10360 | |
| | | | | | GN = UL132 PE = 3 SV = 1 | |
| 28 | P16845 | UL22A | L | E | Glycoprotein UL22A OS = Human | 301 |
| | | | | | cytomegalovirus (strain AD169) OX = 10360 | |
| | | | | | GN = UL22A PE = 3 SV = 2 | |
| 29 | P16760 | UL24 | LL | T | Protein UL24 OS = Human cytomegalovirus | 302-303 |
| | | | | | (strain AD169) OX = 10360 GN = UL24 PE = 3 | |
| | | | | | SV = 3 | |
| 30 | P16761 | UL25 | L | T | Phosphoprotein 85 OS = Human | 304-306 |
| | | | | | cytomegalovirus (strain AD169) OX = 10360 | |
| | | | | | GN = UL25 PE = 3 SV = 1 | |
| 31 | P16762 | UL26 | DE | T | Tegument protein UL26 OS = Human | 307-309 |
| | | | | | cytomegalovirus (strain AD169) OX = 10360 | |
| | | | | | GN = UL26 PE = 3 SV = 2 | |
| 32 | P16764 | UL29 | L | T | Uncharacterized protein UL29 OS = Human | 310-312 |
| | | | | | cytomegalovirus (strain AD169) OX = 10360 | |
| | | | | | GN = UL29 PE = 3 SV = 1 | |
| 33 | P16848 | UL31 | L | NS | Uncharacterized protein UL31 OS = Human | 313-314 |
| | | | | | cytomegalovirus (strain AD169) OX = 10360 | |
| | | | | | GN = UL31 PE = 3 SV = 2 | |
| 34 | P08318 | UL32 | DE | C | Large structural phosphoprotein | 315-317 |
| | | | | | OS = Human cytomegalovirus (strain AD169) | |
| | | | | | OX = 10360 GN = UL32 PE = 1 SV = 1 | |
| 35 | P16812 | UL34 | DE | NS | Transcriptional regulator UL34 OS = Human | 318-320 |
| | | | | | cytomegalovirus (strain AD169) OX = 10360 | |
| | | | | | GN = UL34 PE = 3 SV = 2 | |
| 36 | P16766 | UL35 | DE | T | Protein UL35 OS = Human cytomegalovirus | 321-323 |
| | | | | | (strain AD169) OX = 10360 GN = UL35 PE = 1 | |
| | | | | | SV = 1 | |
| 37 | P16767 | UL36 | IE | T | Uncharacterized protein UL36 OS = Human | 324-326 |
| | | | | | cytomegalovirus (strain AD169) OX = 10360 | |
| | | | | | GN = UL36 PE = 3 SV = 1 | |
| 38 | P16778 | UL37 | IE | NS | UL37 immediate early glycoprotein | 327-329 |
| | | | | | OS = Human cytomegalovirus (strain AD169) | |
| | | | | | OX = 10360 GN = UL37 PE = 1 SV = 2 | |
| 39 | P16779 | UL38 | DE | T | Apoptosis inhibitor UL38 OS = Human | 330-332 |
| | | | | | cytomegalovirus (strain AD169) OX = 10360 | |
| | | | | | GN = UL38 PE = 3 SV = 1 | |
| 40 | P16781 | UL43 | L | T | Tegument protein UL43 OS = Human | 333-335 |
| | | | | | cytomegalovirus (strain AD169) OX = 10360 | |
| | | | | | GN = UL43 PE = 3 SV = 2 | |
| 41 | P16790 | UL44 | DE | NS | DNA polymerase processivity factor | 336-338 |
| | | | | | OS = Human cytomegalovirus (strain AD169) | |
| | | | | | OX = 10360 GN = UL44 PE = 1 SV = 1 | |
| 42 | P16784 | UL47 | LL | T | Inner tegument protein OS = Human | 339-341 |
| | | | | | cytomegalovirus (strain AD169) OX = 10360 | |
| | | | | | GN = UL47 PE = 3 SV = 2 | |
| 43 | P16785 | UL48 | DE | T | Large tegument protein deneddylase | 342-344 |
| | | | | | OS = Human cytomegalovirus (strain AD169) | |
| | | | | | OX = 10360 GN = UL48 PE = 3 SV = 1 | |
| 44 | P16793 | UL52 | L | NS | Packaging protein UL32 homolog | 345-347 |
| | | | | | OS = Human cytomegalovirus (strain AD169) | |
| | | | | | OX = 10360 GN = UL52 PE = 3 SV = 1 | |
| 45 | P08546 | UL54 | DE | T | DNA polymerase catalytic subunit | 348-350 |
| | | | | | OS = Human cytomegalovirus (strain AD169) | |
| | | | | | OX = 10360 GN = UL54 PE = 1 SV = 2 | |
| 46 | P16749 | UL69 | LL | T | mRNA export factor ICP27 homolog | 351-353 |
| | | | | | OS = Human cytomegalovirus (strain AD169) | |
| | | | | | OX = 10360 GN = UL69 PE = 1 SV = 1 | |
| 47 | P16823 | UL71 | DE | T | Tegument protein UL51 homolog | 354-356 |
| | | | | | OS = Human cytomegalovirus (strain AD169) | |
| | | | | | OX = 10360 GN = UL71 PE = 1 SV = 2 | |
| 48 | P16753 | UL80 | L | NS | Capsid scaffolding protein OS = Human | 357-359 |
| | | | | | cytomegalovirus (strain AD169) OX = 10360 | |
| | | | | | GN = UL80 PE = 1 SV = 1 | |
| 49 | P06726 | UL82 | L | T | Protein pp71 OS = Human cytomegalovirus | 360-362 |
| | | | | | (strain AD169) OX = 10360 GN = UL82 PE = 1 | |
| | | | | | SV = 2 | |
| 50 | P06725 | UL83 | LL | T | 65 kDa phosphoprotein OS = Human | 363-365 |
| | | | | | cytomegalovirus (strain AD169) OX = 10360 | |
| | | | | | GN = UL83 PE = 1 SV = 2 | |
| 51 | P16727 | UL84 | DE | T | Protein UL84 OS = Human cytomegalovirus | 366-368 |
| | | | | | (strain AD169) OX = 10360 GN = UL84 PE = 1 | |
| | | | | | SV = 1 | |
| 52 | P16731 | UL88 | L | T | Protein UL88 OS = Human cytomegalovirus | 369-371 |
| | | | | | (strain AD169) OX = 10360 GN = UL88 PE = 3 | |
| | | | | | SV = 1 | |
| 53 | P16800 | UL94 | L | T | Cytoplasmic envelopment protein 2 | 372-374 |
| | | | | | OS = Human cytomegalovirus (strain AD169) | |
| | | | | | OX = 10360 GN = UL94 PE = 1 SV = 1 | |
| 54 | P16788 | UL97 | DE | T | Serine/threonine protein kinase UL97 | 375-377 |
| | | | | | OS = Human cytomegalovirus (strain AD169) | |
| | | | | | OX = 10360 GN = UL97 PE = 1 SV = 1 | |
| 55 | P16789 | UL98 | DE | NS | Alkaline nuclease OS = Human | 378-380 |
| | | | | | cytomegalovirus (strain AD169) OX = 10360 | |
| | | | | | GN = UL98 PE = 3 SV = 2 | |
| 56 | P09721 | US12 | DE | NS | Uncharacterized protein HVLF6 OS = Human | 381-383 |
| | | | | | cytomegalovirus (strain AD169) OX = 10360 | |
| | | | | | GN = US12 PE = 3 SV = 1 | |
| 57 | P09722 | US22 | DE | T | Early nuclear protein HWLF1 OS = Human | 384-386 |
| | | | | | cytomegalovirus (strain AD169) OX = 10360 | |
| | | | | | GN = US22 PE = 3 SV = 2 | |
| 58 | P09701 | US23 | DE | T | Tegument protein US23 OS = Human | 387-389 |
| | | | | | cytomegalovirus (strain AD169) OX = 10360 | |
| | | | | | GN = US23 PE = 3 SV = 2 | |
| 59 | P09709 | US34 | DE | NS | Protein US34 OS = Human cytomegalovirus | 390 |
| | | | | | (strain AD169) OX = 10360 GN = US34 PE = 3 | |
| | | | | | SV = 1 | |
| 60 | P09729 | US9 | DE | NS | Unique short US9 glycoprotein OS = Human | 391-393 |
| | | | | | cytomegalovirus (strain AD169) OX = 10360 | |
| | | | | | GN = US9 PE = 3 SV = 1 | |
| 61 | P16808 | IRL10 | LL | E | Protein IRL10 OS = Human cytomegalovirus | 394-397 |
| | | | | | (strain AD169) OX = 10360 PE = 3 SV = 1 | |
| 62 | P09710 | HKLF1 | DE | NS | Uncharacterized protein HKLF1 OS = Human | 398-400 |
| | | | | | cytomegalovirus (strain AD169) OX = 10360 | |
| | | | | | PE = 3 SV = 1 | |
| 63 | P16799 | CVC1 | L | C | Capsid vertex component 1 OS = Human | 401-402 |
| | | | | | cytomegalovirus (strain AD169) OX = 10360 | |
| | | | | | GN = CVC1 PE = 3 SV = 1 | |
| 64 | P16726 | CVC2 | DE | C | Capsid vertex component 2 OS = Human | 403-405 |
| | | | | | cytomegalovirus (strain AD169) OX = 10360 | |
| | | | | | GN = CVC2 PE = 3 SV = 1 | |
| 65 | P16824 | DUT | LL | T | Deoxyuridine 5′-triphosphate | 406 |
| | | | | | nucleotidohydrolase OS = Human | |
| | | | | | cytomegalovirus (strain AD169) OX = 10360 | |
| | | | | | GN = DUT PE = 3 SV = 1 | |
| 66 | P16832 | gL | L | E | Envelope glycoprotein L OS = Human | 407-409 |
| | | | | | cytomegalovirus (strain AD169) OX = 10360 | |
| | | | | | GN = gL PE = 1 SV = 2 | |
| 67 | P16750 | GO | L | E | Glycoprotein O OS = Human | 410-412 |
| | | | | | cytomegalovirus (strain AD169) OX = 10360 | |
| | | | | | GN = GO PE = 1 SV = 1 | |
| 68 | P16736 | HELI | DE | NS | DNA replication helicase OS = Human | 413-414 |
| | | | | | cytomegalovirus (strain AD169) OX = 10360 | |
| | | | | | GN = HELI PE = 3 SV = 1 | |
| 69 | Q7M6N6 | SCP | L | C | Small capsomere-interacting protein | 415 |
| | | | | | OS = Human cytomegalovirus (strain AD169) | |
| | | | | | OX = 10360 GN = SCP PE = 1 SV = 1 | |
| 70 | P16735 | UL104 | DE | NS | Portal protein OS = Human cytomegalovirus | 416-418 |
| | | | | | (strain AD169) OX = 10360 GN = UL104 PE = 3 | |
| | | | | | SV = 2 | |
| 71 | P16769 | UL114 | DE | NS | Uracil-DNA glycosylase OS = Human | 419-420 |
| | | | | | cytomegalovirus (strain AD169) OX = 10360 | |
| | | | | | GN = UL114 PE = 3 SV = 1 | |
| 72 | P16837 | UL128 | DE | NS | Uncharacterized protein UL 128 OS = Human | 421-423 |
| | | | | | cytomegalovirus (strain AD169) OX = 10360 | |
| | | | | | GN = UL128 PE = 1 SV = 2 | |
| 73 | P16765 | UL30 | L | NS | Uncharacterized protein UL30 OS = Human | 424 |
| | | | | | cytomegalovirus (strain AD169) OX = 10360 | |
| | | | | | GN = UL30 PE = 3 SV = 1 | |
| 74 | P17146 | UL4 | DE | E | Early glycoprotein GP48 OS = Human | 425-427 |
| | | | | | cytomegalovirus (strain AD169) OX = 10360 | |
| | | | | | GN = UL4 PE = 3 SV = 1 | |
| 75 | P16780 | UL40 | LL | NS | Protein UL40 OS = Human cytomegalovirus | 428 |
| | | | | | (strain AD169) OX = 10360 GN = UL40 PE = 1 | |
| | | | | | SV = 1 | |
| 76 | P16786 | UL49 | LL | NS | Uncharacterized protein UL49 OS = Human | 429 |
| | | | | | cytomegalovirus (strain AD169) OX = 10360 | |
| | | | | | GN = UL49 PE = 3 SV = 1 | |
| 77 | P17149 | UL70 | LL | NS | DNA primase OS = Human cytomegalovirus | 430-432 |
| | | | | | (strain AD169) OX = 10360 GN = UL70 PE = 1 | |
| | | | | | SV = 2 | |
| 78 | P16725 | UL76 | L | T | Protein UL76 OS = Human cytomegalovirus | 433-435 |
| | | | | | (strain AD169) OX = 10360 GN = UL76 PE = 2 | |
| | | | | | SV = 1 | |
| 79 | P16751 | UL78 | DE | NS | Uncharacterized protein UL78 OS = Human | 436 |
| | | | | | cytomegalovirus (strain AD169) OX = 10360 | |
| | | | | | GN = UL78 PE = 4 SV = 1 | |
| 80 | P16752 | UL79 | L | T | Protein UL79 OS = Human cytomegalovirus | 437-438 |
| | | | | | (strain AD169) OX = 10360 GN = UL79 PE = 3 | |
| | | | | | SV = 1 | |
| 81 | P16730 | UL87 | L | NS | Protein UL87 OS = Human cytomegalovirus | 439 |
| | | | | | (strain AD169) OX = 10360 GN = UL87 PE = 3 | |
| | | | | | SV = 1 | |
| 82 | P16801 | UL95 | DE | NS | Protein UL95 OS = Human cytomegalovirus | 440-441 |
| | | | | | (strain AD169) OX = 10360 GN = UL95 PE = 3 | |
| | | | | | SV = 1 | |
| 83 | P16787 | UL96 | DE | T | Protein UL96 OS = Human cytomegalovirus | 442-443 |
| | | | | | (strain AD169) OX = 10360 GN = UL96 PE = 3 | |
| | | | | | SV = 2 | |
| 84 | P13200 | UL99 | L | T | Cytoplasmic envelopment protein 3 | 444-445 |
| | | | | | OS = Human cytomegalovirus (strain AD169) | |
| | | | | | OX = 10360 GN = UL99 PE = 1 SV = 3 | |
| 85 | P09720 | US13 | DE | NS | Uncharacterized protein HVLF5 OS = Human | 446 |
| | | | | | cytomegalovirus (strain AD169) OX = 10360 | |
| | | | | | GN = US13 PE = 3 SV = 1 | |
| 86 | P09719 | US14 | DE | NS | Uncharacterized protein HVLF4 OS = Human | 447 |
| | | | | | cytomegalovirus (strain AD169) OX = 10360 | |
| | | | | | GN = US14 PE = 3 SV = 2 | |
| 87 | P09718 | US15 | LL | NS | Uncharacterized protein HVLF3 OS = Human | 448 |
| | | | | | cytomegalovirus (strain AD169) OX = 10360 | |
| | | | | | GN = US15 PE = 3 SV = 2 | |
| 88 | P69334 | US18 | DE | NS | Transmembrane protein US18 OS = Human | 449 |
| | | | | | cytomegalovirus (strain AD169) OX = 10360 | |
| | | | | | GN = US18 PE = 3 SV = 1 | |
| 89 | P09700 | US24 | DE | T | Tegument protein US24 OS = Human | 450-452 |
| | | | | | cytomegalovirus (strain AD169) OX = 10360 | |
| | | | | | GN = US24 PE = 3 SV = 3 | |
| 90 | P09730 | US8 | DE | NS | Unique short US8 glycoprotein OS = Human | 453 |
| | | | | | cytomegalovirus (strain AD169) OX = 10360 | |
| | | | | | GN = US8 PE = 3 SV = 2 | |

TABLE 3
List of Signature Peptides for The Identification of KSHV Proteins:

| | Protein | Protein | | component | | SEQ ID |
|---|---|---|---|---|---|---|
| # | Accession | Gene | temporality | virion | Protein Description | NO(s) |
| 1 | P90463 | 70 | DE | NS | Thymidylate synthase OS = Human herpesvirus 8 | 454-456 |
| | | | | | type P (isolate GK18) OX = 868565 GN = 70 PE = 1 | |
| | | | | | SV = 1 | |
| 2 | Q2HRD3 | DBP | DE | NS | Major DNA-binding protein OS = Human | 457-459 |
| | | | | | herpesvirus 8 type P (isolate GK18) OX = 868565 | |
| | | | | | GN = DBP PE = 1 SV = 1 | |
| 3 | Q2HR78 | DUT | DE | T | Deoxyuridine 5′-triphosphate | 460-462 |
| | | | | | nucleotidohydrolase OS = Human herpesvirus 8 | |
| | | | | | type P (isolate GK18) OX = 868565 GN = DUT | |
| | | | | | PE = 3 SV = 1 | |
| 4 | F5HB81 | gB | L | E | Envelope glycoprotein B OS = Human herpesvirus | 463-464 |
| | | | | | 8 type P (isolate GK18) OX = 868565 GN = gB | |
| | | | | | PE = 1 SV = 1 | |
| 5 | Q2HRC7 | K2 | DE | T | Viral interleukin-6 homolog OS = Human | 465-467 |
| | | | | | herpesvirus 8 type P (isolate GK18) OX = 868565 | |
| | | | | | GN = K2 PE = 1 SV = 1 | |
| 6 | P90495 | K3 | DE | T | E3 ubiquitin-protein ligase MIR1 OS = Human | 468-470 |
| | | | | | herpesvirus 8 type P (isolate GK18) OX = 868565 | |
| | | | | | GN = K3 PE = 1 SV = 1 | |
| 7 | P90489 | K5 | DE | NS | E3 ubiquitin-protein ligase MIR2 OS = Human | 471-473 |
| | | | | | herpesvirus 8 type P (isolate GK18) OX = 868565 | |
| | | | | | GN = K5 PE = 1 SV = 1 | |
| 8 | Q2HR82 | K8 | IE | NS | E3 SUMO-protein ligase K-bZIP OS = Human | 474-476 |
| | | | | | herpesvirus 8 type P (isolate GK18) OX = 868565 | |
| | | | | | GN = K8 PE = 1 SV = 1 | |
| 9 | F5HB98 | K8.1 | L | E | Protein K8.1 OS = Human herpesvirus 8 type P | 477-479 |
| | | | | | (isolate GK18) OX = 868565 GN = K8.1 PE = 3 | |
| | | | | | SV = 1 | |
| 10 | Q2HRA7 | MCP | L | C | Major capsid protein OS = Human herpesvirus 8 | 480-482 |
| | | | | | type P (isolate GK18) OX = 868565 GN = MCP | |
| | | | | | PE = 3 SV = 1 | |
| 11 | F5H982 | NEC1 | DE | NS | Nuclear egress protein 1 OS = Human | 483-485 |
| | | | | | herpesvirus 8 type P (isolate GK18) OX = 868565 | |
| | | | | | GN = NEC1 PE = 1 SV = 1 | |
| 12 | F5HA27 | NEC2 | DE | NS | Nuclear egress protein 2 OS = Human | 486-488 |
| | | | | | herpesvirus 8 type P (isolate GK18) OX = 868565 | |
| | | | | | GN = NEC2 PE = 1 SV = 1 | |
| 13 | Q2HRC9 | ORF10 | DE | NS | Protein ORF10 OS = Human herpesvirus 8 type P | 489-491 |
| | | | | | (isolate GK18) OX = 868565 GN = ORF10 PE = 4 | |
| | | | | | SV = 1 | |
| 14 | Q2HRC8 | ORF11 | DE | T | Protein ORF11 OS = Human herpesvirus 8 type P | 492-494 |
| | | | | | (isolate GK18) OX = 868565 GN = ORF11 PE = 4 | |
| | | | | | SV = 1 | |
| 15 | Q2HRB6 | ORF17 | DE | T | Capsid scaffolding protein OS = Human | 495-497 |
| | | | | | herpesvirus 8 type P (isolate GK18) OX = 868565 | |
| | | | | | GN = ORF17 PE = 1 SV = 1 | |
| 16 | F5HHY1 | ORF38 | L | T | Cytoplasmic envelopment protein 3 OS = Human | 498 |
| | | | | | herpesvirus 8 type P (isolate GK18) OX = 868565 | |
| | | | | | GN = ORF38 PE = 3 SV = 1 | |
| 17 | Q2HRD4 | ORF4 | L | NS | Complement control protein OS = Human | 499-501 |
| | | | | | herpesvirus 8 type P (isolate GK18) OX = 868565 | |
| | | | | | GN = ORF4 PE = 3 SV = 1 | |
| 18 | F5HDE4 | ORF45 | IE | T | Protein ORF45 OS = Human herpesvirus 8 type P | 502-504 |
| | | | | | (isolate GK18) OX = 868565 GN = ORF45 PE = 1 | |
| | | | | | SV = 1 | |
| 19 | F5HCV3 | ORF50 | IE | NS | Putative transcription activator ORF50 | 505-507 |
| | | | | | OS = Human herpesvirus 8 type P (isolate GK18) | |
| | | | | | OX = 868565 GN = ORF50 PE = 3 SV = 1 | |
| 20 | Q2HR80 | ORF52 | L | T | Tegument protein ORF52 OS = Human | 508-510 |
| | | | | | herpesvirus 8 type P (isolate GK18) OX = 868565 | |
| | | | | | GN = ORF52 PE = 1 SV = 1 | |
| 21 | Q2HR75 | ORF57 | IE | NS | mRNA export factor ICP27 homolog OS = Human | 511-513 |
| | | | | | herpesvirus 8 type P (isolate GK18) OX = 868565 | |
| | | | | | GN = ORF57 PE = 1 SV = 1 | |
| 22 | F5HID2 | ORF59 | DE | NS | DNA polymerase processivity factor OS = Human | 514-516 |
| | | | | | herpesvirus 8 type P (isolate GK18) OX = 868565 | |
| | | | | | GN = ORF59 PE = 3 SV = 1 | |
| 23 | Q9QR70 | ORF75 | L | T | Protein ORF75 OS = Human herpesvirus 8 type P | 517-519 |
| | | | | | (isolate GK18) OX = 868565 GN = ORF75 PE = 4 | |
| | | | | | SV = 1 | |
| 24 | Q2HRD0 | ORF9 | DE | T | DNA polymerase catalytic subunit OS = Human | 520-522 |
| | | | | | herpesvirus 8 type P (isolate GK18) OX = 868565 | |
| | | | | | GN = ORF9 PE = 3 SV = 1 | |
| 25 | Q2HR67 | RIR1 | DE | NS | Ribonucleoside-diphosphate reductase large | 523-525 |
| | | | | | subunit OS = Human herpesvirus 8 type P (isolate | |
| | | | | | GK18) OX = 868565 GN = RIR1 PE = 3 SV = 1 | |
| 26 | Q2HR63 | SCP | L | C | Small capsomere-interacting protein OS = Human | 526-527 |
| | | | | | herpesvirus 8 type P (isolate GK18) OX = 868565 | |
| | | | | | GN = SCP PE = 1 SV = 1 | |
| 27 | F5HB62 | TK | DE | T | Thymidine kinase OS = Human herpesvirus 8 type | 528-530 |
| | | | | | P (isolate GK18) OX = 868565 GN = TK PE = 3 | |
| | | | | | SV = 1 | |
| 28 | F5HB39 | CVC1 | L | C | Capsid vertex component 1 OS = Human | 531-533 |
| | | | | | herpesvirus 8 type P (isolate GK18) OX = 868565 | |
| | | | | | GN = CVC1 PE = 3 SV = 1 | |
| 29 | Q2HRB3 | CVC2 | L | C | Capsid vertex component 2 OS = Human | 534-536 |
| | | | | | herpesvirus 8 type P (isolate GK18) OX = 868565 | |
| | | | | | GN = CVC2 PE = 3 SV = 1 | |
| 30 | F5HAK9 | gH | L | E | Envelope glycoprotein H OS = Human herpesvirus | 537-539 |
| | | | | | 8 type P (isolate GK18) OX = 868565 GN = gH | |
| | | | | | PE = 1 SV = 1 | |
| 31 | F5HDB7 | gL | L | E | Envelope glycoprotein L OS = Human herpesvirus | 540-542 |
| | | | | | 8 type P (isolate GK18) OX = 868565 GN = gL | |
| | | | | | PE = 1 SV = 1 | |
| 32 | F5HDD0 | gM | L | E | Envelope glycoprotein M OS = Human | 543-545 |
| | | | | | herpesvirus 8 type P (isolate GK18) OX = 868565 | |
| | | | | | GN = gM PE = 1 SV = 1 | |
| 33 | Q2HR89 | HELI | DE | NS | DNA replication helicase OS = Human | 546-548 |
| | | | | | herpesvirus 8 type P (isolate GK18) OX = 868565 | |
| | | | | | GN = HELI PE = 3 SV = 1 | |
| 34 | P0C788 | K14 | DE | NS | OX-2 membrane glycoprotein homolog | 549-550 |
| | | | | | OS = Human herpesvirus 8 type P (isolate GK18) | |
| | | | | | OX = 868565 GN = K14 PE = 1 SV = 1 | |
| 35 | Q98157 | ORF K4 | DE | NS | Viral macrophage inflammatory protein 2 | 551 |
| | | | | | OS = Human herpesvirus 8 type P (isolate GK18) | |
| | | | | | OX = 868565 GN = ORF K4 PE = 1 SV = 1 | |
| 36 | F5HGJ3 | ORF16 | IE | NS | Apoptosis regulator Bcl-2 homolog OS = Human | 552-553 |
| | | | | | herpesvirus 8 type P (isolate GK18) OX = 868565 | |
| | | | | | GN = ORF16 PE = 1 SV = 1 | |
| 37 | Q2HRC6 | ORF2 | DE | NS | Putative Dihydrofolate reductase OS = Human | 554-555 |
| | | | | | herpesvirus 8 type P (isolate GK18) OX = 868565 | |
| | | | | | GN = ORF2 PE = 3 SV = 1 | |
| 38 | Q2HRB2 | ORF20 | L | NS | Protein UL24 homolog OS = Human herpesvirus 8 | 556 |
| | | | | | type P (isolate GK18) OX = 868565 GN = ORF20 | |
| | | | | | PE = 2 SV = 1 | |
| 39 | F5HIM6 | ORF23 | L | T | Protein ORF23 OS = Human herpesvirus 8 type P | 557-558 |
| | | | | | (isolate GK18) OX = 868565 GN = ORF23 PE = 3 | |
| | | | | | SV = 1 | |
| 40 | F5HFD2 | ORF24 | L | T | Protein ORF24 OS = Human herpesvirus 8 type P | 559-561 |
| | | | | | (isolate GK18) OX = 868565 GN = ORF24 PE = 3 | |
| | | | | | SV = 1 | |
| 41 | F5HDY6 | ORF27 | L | E | Protein ORF27 OS = Human herpesvirus 8 type P | 562-564 |
| | | | | | (isolate GK18) OX = 868565 GN = ORF27 PE = 4 | |
| | | | | | SV = 1 | |
| 42 | F5HI25 | ORF28 | L | E | Protein ORF28 OS = Human herpesvirus 8 type P | 565 |
| | | | | | (isolate GK18) OX = 868565 GN = ORF28 PE = 4 | |
| | | | | | SV = 1 | |
| 43 | F5HEF2 | ORF33 | L | T | Cytoplasmic envelopment protein 2 OS = Human | 566-568 |
| | | | | | herpesvirus 8 type P (isolate GK18) OX = 868565 | |
| | | | | | GN = ORF33 PE = 3 SV = 1 | |
| 44 | Q2HR98 | ORF34 | L | NS | Protein UL95 homolog OS = Human herpesvirus 8 | 569-570 |
| | | | | | type P (isolate GK18) OX = 868565 GN = ORF34 | |
| | | | | | PE = 3 SV = 1 | |
| 45 | F5HCD4 | ORF35 | L | T | Protein ORF35 OS = Human herpesvirus 8 type P | 571-572 |
| | | | | | (isolate GK18) OX = 868565 GN = ORF35 PE = 4 | |
| | | | | | SV = 1 | |
| 46 | F5HGH5 | ORF36 | DE | T | Protein ORF36 OS = Human herpesvirus 8 type P | 573 |
| | | | | | (isolate GK18) OX = 868565 GN = ORF36 PE = 1 | |
| | | | | | SV = 1 | |
| 47 | Q2HR95 | ORF37 | DE | NS | Shutoff alkaline exonuclease OS = Human | 574-576 |
| | | | | | herpesvirus 8 type P (isolate GK18) OX = 868565 | |
| | | | | | GN = ORF37 PE = 1 SV = 1 | |
| 48 | Q2HR92 | ORF40 | DE | NS | DNA helicase/primase complex-associated | 577-578 |
| | | | | | protein OS = Human herpesvirus 8 type P (isolate | |
| | | | | | GK18) OX = 868565 GN = ORF40 PE = 3 SV = 1 | |
| 49 | F5HAI6 | ORF42 | L | T | Cytoplasmic envelopment protein 1 OS = Human | 579-580 |
| | | | | | herpesvirus 8 type P (isolate GK18) OX = 868565 | |
| | | | | | GN = ORF42 PE = 3 SV = 1 | |
| 49 | F5HAI6 | ORF42 | L | T | Cytoplasmic envelopment protein 1 OS = Human | 580 |
| | | | | | herpesvirus 8 type P (isolate GK18) OX = 868565 | |
| | | | | | GN = ORF42 PE = 3 SV = 1 | |
| 50 | F5HGK9 | ORF43 | L | C | Portal protein OS = Human herpesvirus 8 type P | 581-583 |
| | | | | | (isolate GK18) OX = 868565 GN = ORF43 PE = 3 | |
| | | | | | SV = 1 | |
| 51 | F5HFA1 | ORF46 | DE | NS | Uracil-DNA glycosylase OS = Human herpesvirus | 584-585 |
| | | | | | 8 type P (isolate GK18) OX = 868565 GN = ORF46 | |
| | | | | | PE = 3 SV = 1 | |
| 52 | Q2HR85 | ORF48 | IE | T | Tegument protein ORF48 OS = Human | 586 |
| | | | | | herpesvirus 8 type P (isolate GK18) OX = 868565 | |
| | | | | | GN = ORF48 PE = 3 SV = 1 | |
| 53 | Q2HR83 | ORF49 | DE | NS | Protein ORF49 OS = Human herpesvirus 8 type P | 587 |
| | | | | | (isolate GK18) OX = 868565 GN = ORF49 PE = 3 | |
| | | | | | SV = 1 | |
| 54 | F5H9W9 | ORF55 | L | T | Tegument protein ORF55 OS = Human | 588-590 |
| | | | | | herpesvirus 8 type P (isolate GK18) OX = 868565 | |
| | | | | | GN = ORF55 PE = 3 SV = 1 | |
| 55 | F5HIN0 | ORF56 | DE | T | DNA primase OS = Human herpesvirus 8 type P | 591-593 |
| | | | | | (isolate GK18) OX = 868565 GN = ORF56 PE = 3 | |
| | | | | | SV = 1 | |
| 56 | F5HEU7 | ORF63 | L | T | Inner tegument protein OS = Human herpesvirus | 594 |
| | | | | | 8 type P (isolate GK18) OX = 868565 GN = ORF63 | |
| | | | | | PE = 3 SV = 1 | |
| 57 | Q2HR64 | ORF64 | L | T | Large tegument protein deneddylase OS = Human | 595-597 |
| | | | | | herpesvirus 8 type P (isolate GK18) OX = 868565 | |
| | | | | | GN = ORF64 PE = 3 SV = 1 | |
| 58 | F5HG20 | ORF66 | DE | NS | Protein ORF66 OS = Human herpesvirus 8 type P | 598-599 |
| | | | | | (isolate GK18) OX = 868565 GN = ORF66 PE = 3 | |
| | | | | | SV = 1 | |
| 59 | F5HF47 | ORF68 | L | E | Packaging protein UL32 homolog OS = Human | 600-602 |
| | | | | | herpesvirus 8 type P (isolate GK18) OX = 868565 | |
| | | | | | GN = ORF68 PE = 3 SV = 1 | |
| 60 | F5H8Y5 | TRX1 | L | C | Triplex capsid protein 1 OS = Human herpesvirus | 603 |
| | | | | | 8 type P (isolate GK18) OX = 868565 GN = TRX1 | |
| | | | | | PE = 3 SV = 1 | |
| 61 | F5HGN8 | TRX2 | L | C | Triplex capsid protein 2 OS = Human herpesvirus | 604-605 |
| | | | | | 8 type P (isolate GK18) OX = 868565 GN = TRX2 | |
| | | | | | PE = 3 SV = 1 | |
| 62 | F5HF68 | VIRF-1 | DE | T | VIRF-1 OS = Human herpesvirus 8 type P (isolate | 606 |
| | | | | | GK18) OX = 868565 GN = VIRF-1 PE = 1 SV = 1 | |

### DETAILED DESCRIPTION

Herpesviruses infect up to 90% of the population and are dangerous in immune-compromised individuals and pregnant women. However, effective non-toxic antiviral treatments or vaccines for these viruses are currently lacking. The replication of a herpesvirus in an infected cell and the spread of infection to neighboring cells rely on a finely controlled lifecycle with a temporally tuned cascade of viral gene expression.

In order to effectively identify potential virus modulatory compounds, as well as gain an understanding of their impact on specific stages of a viral infection, described herein is a novel assay format to monitor viral proteins from herpesviruses. These assays offer the accurate detection and quantification of viral proteins from all distinct temporal classes of viral replication. Three exemplary assays have been designed for the specific detection of three herpesviruses: herpes simplex virus 1 (HSV1), human cytomegalovirus (HCMV), and Kaposi's sarcoma-associated herpesvirus (KSHV). These assays can be utilized in combination with drug treatments, genetic modifications, or other perturbations to assess the impact of the intervention on viral protein production. Given the temporal nature of herpesvirus infection, the acquired protein abundance measurements made available using these assays provide information regarding the stage of infection (e.g. entry, viral genome replication, assembly, egress) that is affected, the specific viral proteins that are impacted, as well as additional mechanistic understanding of how a given compound or other perturbation impacts viral replication. Thus, the provided methods can be used as either primary or secondary screens for the purposes of anti-viral drug discovery, as well as in vaccine development assays.

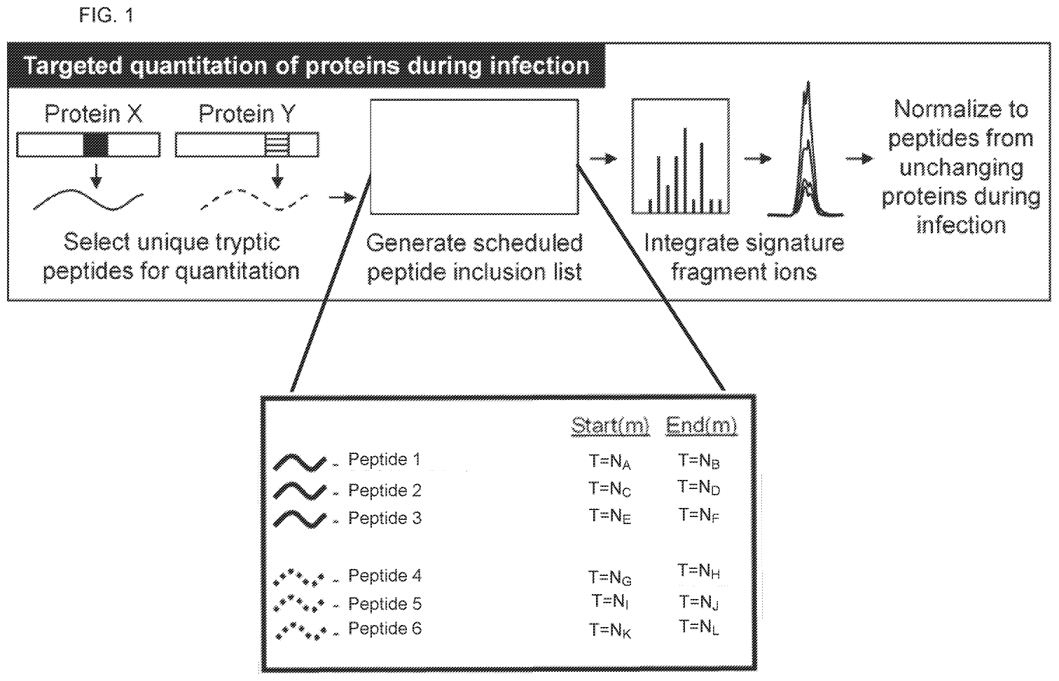

Described herein is the development of a novel series of assays to determine the protein abundance levels of viral proteins during the progression of herpesvirus infections. These assays can be used to support the discovery of antiviral compounds, as well as other purposes. Liquid chromatography coupled to tandem mass spectrometry (LC-MS/MS) is used to perform a targeted mass spectrometry technique called parallel reaction monitoring (PRM) to quantitatively monitor signature peptides from target proteins (FIG. 1). A “signature” peptide in this context refers to a peptide that can be used to distinguish one protein from all others in a sample.

While these assays have been designed on a quadrupole-Orbitrap instrument platform, they can easily be ported to additional instrument platforms (including the triple quadrupole instrumentation favored by industry and clinical facilities) with minimal modification and time investment. Thus, transfer of this technology to interested commercial entities will be readily achieved.

Noteworthy, mass spectrometry instruments are now part of the common infrastructure of academic, industry, and clinical settings. Almost all pharmaceutical and clinical companies currently either have a mass spectrometry group in house or a close relationship with a mass spectrometry contract research organization, and thus will be able to easily make use of this assay.

As exemplified herein, three assays have been developed for monitoring viral proteins in HSV-1, HCMV, and KSHV, monitoring up to 60 (see Table 1), up to 90 (see Table 2), and up to 62 (see Table 3) viral proteins from each virus, respectively. In each case, this constitutes approximately 50-80% of the predicted viral proteome. In Tables 1-3, many of the viral proteins are associated with more than one (that is, two, three, or four) signature peptides. While measurement of more than one signature peptide (including all of the listed signature peptides) for any one protein may provide the most redundant data for detection and/or quantification of the corresponding protein, it is understood that fewer than all of the provided peptides may be used in some embodiments. Thus, specific embodiments include assays in which only one signature peptide is detected for each viral protein being monitored, as well as assays in which two or more signature peptides are detected for one or more viral proteins being monitored.

In example herpesvirus PRM assay methods shown herein, cell pellets were lysed in 2% SDS, 100 mM NaCl, 0.5 mM EDTA, 50 mM Tris, pH 8.2, and 50 μg of protein was reduced and alkylated with 25 mM TCEP and 50 mM CAM respectively for 20 min at 70° C. Proteins were then precipitated via methanol chloroform precipitation (Wessel & Flugg, Anal Biochem. 138(1):141-143, 1984), resuspended in 50 mM HEPES, pH 8.2 and digested overnight with trypsin (50:1 protein:enzyme w/w ratio). Digested peptides were desalted by SDB-RPS StageTip as previously described (Lum et al., Cell Syst., 7(6):627-242, 2018; Greco et al., Methods Mol Biol 1410:39-63, 2016; Federspiel & Cristea, Methods Mol Biol., 1977:115-143, 2019).

Peptides (1.0 μg on column) were analyzed by LC-MS/MS using a Dionex Ultimate 3000 UHPLC coupled online to an EASYSpray ion source and a Q Exactive HF. Peptides were separated on an EASYSpray C18 column (75 μm×25 cm) heated to 50° C. using a linear gradient of 5% B to 32% B over 60 min at a flow rate of 250 nL/min and were ionized at 1.7 kv. Mobile phase A consisted of 0.1% FA in H₂O and mobile phase B consisted of 0.1% FA, 2.9% H₂O in ACN.

The PRM method was controlled by a peptide inclusion list with retention time windows of 6 min for selected precursor ions. The PRM method consisted of MS2 scans that were acquired at a resolution of 30,000 with an AGC setting of 1e5, an MIT of 60 ms, an isolation window of 0.8 m/z, fixed first mass of 125.0 m/z, and normalized collision energy of 27 recorded in profile.

The PRM assay was developed and analyzed using the open-source software Skyline (Maclean et al., Bioinformatics 26(7):966-968, 2010). Summed area under the curve of 3-4 transitions per peptide was used for quantitation. Targeted peptides were normalized to host protein loading control peptides. Peptide values for each sample were scaled to the average of each peptide across all runs. The average of multiple peptides was used as the inferred value for the protein measurement when more than one peptide was quantified (Federspiel et al., PLoS Biol. 17(9):e3000437. Doi: 10.1371/journal.pbio.3000437). PRM quantitation data were graphed using the Python Seaborn and Matplotlib libraries.

The assays provided herein can be expanded to complete coverage of each viral proteome, as well as to incorporate host proteins useful as markers of infection. Importantly, in each assay, viral proteins from every temporal class (e.g., immediate early (IE), early (E), and late (L) genes for HSV-1; IE, delayed early (DE), leaky late (LL), and L genes for HCMV, and IE, DE, and L genes for KSHV) can be monitored based on the systems provided herein. Concurrent with the addition of more protein targets, it is also possible to scale down the number of cells used in the assays, from ˜150,000 to ˜10,000 cells, thereby facilitating automation, as well as reducing cost.

Another important consideration for a screening assay is the speed at which the information can be acquired. The current assays can be completed in one to two hours for each time point, and the expanded assays are designed to stay within this short timeframe.

Also contemplated as a component is the development of an automated pipeline for data analysis that will allow users to analyze the acquired data and generate standardized reports with the click of a button. Using the existing automation capabilities of the open-source data analysis tool Skyline, in conjunction with custom written code, a simple user interface can be provided for each targeted assay. This will allow non-expert users to analyze and interpret their data quickly and easily. The output of this analysis pipeline will be a report with defined structure and components to allow for simple reporting and tracking, as well as for direct comparisons of results run at different times or laboratories and by different users.

It is demonstrated herein that the described assays can be used to effectively screen small molecule modulators of viral infection (Example 1). These screens can readily be expanded to a range of antiviral compounds, which will demonstrate the broad value of this assay and enhance its marketability.

As an initial demonstration of the use of this assay for testing compounds, sirtuin modulators have been assessed. Based on earlier work related to whether a single therapeutic strategy can be used to inhibit the infection with different viruses, in collaboration with others, a class of human enzymes called sirtuins was identified that have broad-spectrum antiviral functions against a range of DNA and RNA viruses, including herpesviruses (Koyunku et al., mBio 5:6):302249-14, 2014). Building on this prior work, described herein is use of the newly developed assay system to investigate an activator (CAY10602) and an inhibitor (EX-527) of sirtuin 1 to better define the precise stage of infection when these molecules impact HCMV replication (Example 1).

Using this assay, it was found that CAY10602 inhibits early stages of infection, as the levels of viral immediate early proteins were reduced (Example 1). Furthermore, this inhibitory effect was maintained throughout infection, as the levels of delayed-early and late viral proteins were also affected. However, the impact on the immediate early viral proteins was more pronounced for a specific subset of viral proteins. Therefore, the herein-described assay has the ability to not only pinpoint the stage of infection when a compound acts, but also the specific functional family of viral proteins that are affected. This is important for understanding the potential downstream impact of a compound on virus-induced alterations on cellular pathways. This assay also showed that EX-527 slightly elevates viral protein production.

The described screen can also readily be expanded to analysis of other compounds, for instance that are either antiviral (i.e., with therapeutic potential) or enhance virus infectivity (i.e., for vaccine development). For instance, other sirtuin activators that inhibit viral infection, and for which the specifics of their impact on virus replication remain unknown, may be tested. A range of other antiviral compounds can also be tested, as well as genetic manipulations (knockouts and over-expressions) known to affect viral infection. Altogether, this will prove the value of these assays as screening tools for compounds that modulate virus infections, determining not only if an intervention will inhibit viral replication, but also when during infection this inhibition takes place and via which specific viral proteins.

Also contemplated as embodiments are ready-to-use kits that will provide some or optionally all the components needed to perform an assay described herein. For instance, three kits can be provided, one each for HSV-1, HCMV, and KSHV. Embodiments of each kit will include the parameters for performing the assay for the target virus, a set of heavy isotope labeled peptides that can be added to every sample run, and a USB drive or other non-transitory computer readable medium containing software develop for assayed analysis and standardized report generation. The inclusion of a heavy labeled peptide corresponding to each of the signature viral and host peptides that have been selected for the kit/assay allows for rapid and easy transfer of the assay across different instrument platforms, and further enhances the accuracy of the quantification. The licensing of the assays (and preparation of the kits) can be performed in a modular fashion based on which virus(es) a prospective client is interested in. It is also contemplated that analysis of samples can be provided using with the described platform, for instance as a service provided through a Mass Spectrometry Facility (e.g., the Princeton Facility) if a client desires.

Current techniques for monitoring herpesvirus lifecycle progression are limited compared to the method described herein. The predominant technologies for monitoring protein levels during viral infection are western blotting and ELISA assays. Both rely on the generation of high-quality antibodies and are relatively expensive, time intensive, and not amenable to multiplex analysis. Antibodies frequently have cross-reactivity with other proteins, thereby impacting the confidence of the measurement. Also, western blotting is inherently more variable, affecting the accuracy of the quantification. Currently, for high-throughput analysis of gene products during viral infection, microarray technology is used, which measures mRNA levels in infected samples. While this does allow for multiplexed analysis of many targets, it does not measure the actual resulting protein level, and thus does not measure the molecule most closely associated with the infection phenotype. The assays described here enable more direct high-throughput measurements of the molecules of interest, with greater precision and accuracy than antibody-based techniques. Importantly, these methods can be easily transferred to interested commercial partners and are not locked into any individual analysis platform. Thus, the described assays will be useful to industry and readily commercialized.

Representative Computer Architecture.

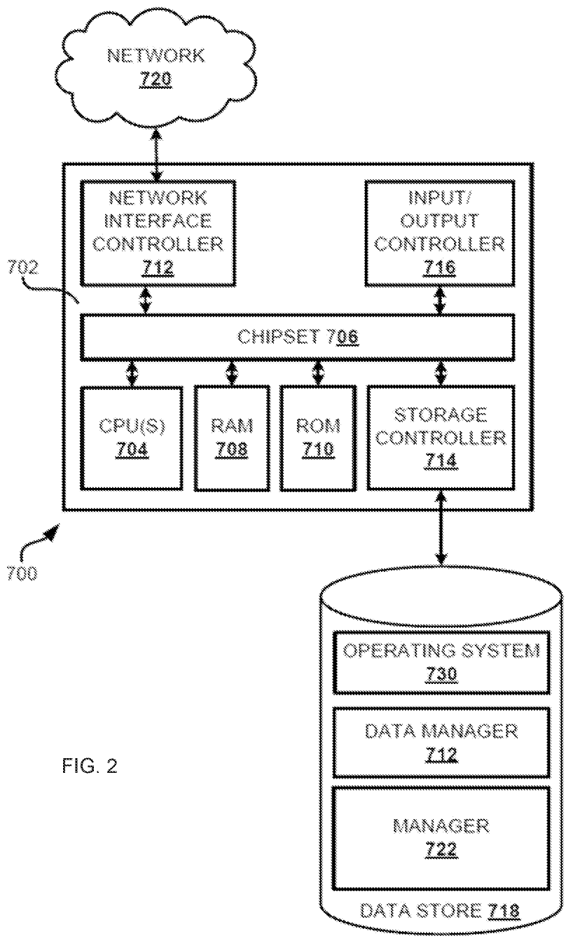

FIG. 2 shows an example computer architecture for a computer 700 capable of executing program components for detecting and measuring peptide level(s) in a herpesvirus assay described herein, and for calculating viral protein quantity in accordance with such assays. The computer architecture shown in FIG. 2 illustrates a conventional server computer, workstation, desktop computer, laptop, tablet, network appliance, digital cellular phone, smart watch, or other computing device, and may be utilized to execute any of the software components presented herein. For example, the computer architecture shown in FIG. 2 may be utilized to execute software components for performing operations as described herein. The computer architecture shown in FIG. 2 might also be utilized to implement a computing device, or any other of the computing systems described herein.

The computer 700 includes a baseboard 702, or “motherboard,” which is a printed circuit board to which a multitude of components or devices may be connected by way of a system bus or other electrical communication paths. In one illustrative example, one or more central processing units (“CPUs”) 704 operate in conjunction with a chipset 706. The CPUs 704 may be standard programmable processors that perform arithmetic and logical operations necessary for the operation of the computer 700.

The CPUs 704 perform operations by transitioning from one discrete, physical state to the next through the manipulation of switching elements that differentiate between and change these states. Switching elements may generally include electronic circuits that maintain one of two binary states, such as flip-flops and electronic circuits that provide an output state based on the logical combination of the states of one or more other switching elements, such as logic gates. These basic switching elements may be combined to create more complex logic circuits, including registers, adders-subtractors, arithmetic logic units, floating-point units and the like.

The chipset 706 provides an interface between the CPUs 704 and the remainder of the components and devices on the baseboard 702. The chipset 706 may provide an interface to a RAM 708, used as the main memory in the computer 700. The chipset 706 may further provide an interface to a computer-readable storage medium such as a read-only memory (“ROM”) 710 or non-volatile RAM (“NVRAM”) for storing basic routines that help to startup the computer 700 and to transfer information between the various components and devices. The ROM 710 or NVRAM may also store other software components necessary for the operation of the computer 700 in accordance with the description herein.

The computer 700 may operate in a networked environment using logical connections to remote computing devices and computer systems through a network, such as the network 720. The chipset 706 may include functionality for providing network connectivity through a network interface controller (“NIC”) 712, such as a mobile cellular network adapter, WiFi network adapter or gigabit Ethernet adapter. The NIC 712 is capable of connecting the computer 700 to other computing devices over the network 720. It should be appreciated that multiple NICs 712 may be present in the computer 700, connecting the computer to other types of networks and remote computer systems.

The computer 700 may be connected to a mass storage device 718 that provides non-volatile storage for the computer. The mass storage device 718 may store system programs, application programs, other program modules and data, which have been described in greater detail herein. The mass storage device 718 may be connected to the computer 700 through a storage controller 714 connected to the chipset 706. The mass storage device 718 may consist of one or more physical storage units. The storage controller 714 may interface with the physical storage units through a serial attached SCSI (“SAS”) interface, a serial advanced technology attachment (“SATA”) interface, a fiber channel (“FC”) interface, or other type of interface for physically connecting and transferring data between computers and physical storage units.

The computer 700 may store data on the mass storage device 718 by transforming the physical state of the physical storage units to reflect the information being stored. The specific transformation of physical state may depend on various factors, in different implementations of this description. Examples of such factors may include, but are not limited to, the technology used to implement the physical storage units, whether the mass storage device 718 is characterized as primary or secondary storage and the like.

For example, the computer 700 may store information to the mass storage device 718 by issuing instructions through the storage controller 714 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computer 700 may further read information from the mass storage device 718 by detecting the physical states or characteristics of one or more particular locations within the physical storage units.

In addition to the mass storage device 718 described above, the computer 700 may have access to other computer-readable storage media to store and retrieve information, such as program modules, data structures, or other data. It will be appreciated by those skilled in the art that computer-readable storage media is any available media that provides for the non-transitory storage of data and that may be accessed by the computer 700.

By way of example, and not limitation, computer-readable storage media may include volatile and non-volatile, removable and non-removable media implemented in any method or technology. Computer-readable storage media includes, but is not limited to, RAM, ROM, erasable programmable ROM (“EPROM”), electrically-erasable programmable ROM (“EEPROM”), flash memory or other solid-state memory technology, compact disc ROM (“CD-ROM”), digital versatile disk (“DVD”), high definition DVD (“HD-DVD”), BLU-RAY, or other optical storage, magnetic cassettes, magnetic tape, magnetic disk storage or other magnetic storage devices, or any other medium that can be used to store the desired information in a non-transitory fashion.

The mass storage device 718 may store an operating system 730 utilized to control the operation of the computer 700. According to one example, the operating system comprises the LINUX operating system. According to another example, the operating system comprises the WINDOWS® SERVER operating system from MICROSOFT Corporation. According to another example, the operating system comprises the iOS operating system from Apple. According to another example, the operating system comprises the Android operating system from Google or its ecosystem partners. According to further examples, the operating system may comprise the UNIX operating system. It should be appreciated that other operating systems may also be utilized. The mass storage device 718 may store other system or application programs and data utilized by the computer 700, such as components that include the data manager 740, the flow manager 750 and/or any of the other software components and data described herein. The mass storage device 718 might also store other programs and data not specifically identified herein.

In one example, the mass storage device 718 or other computer-readable storage media is encoded with computer-executable instructions that, when loaded into the computer 700, create a special-purpose computer capable of implementing one or more of the embodiments or examples described herein. These computer-executable instructions transform the computer 700 by specifying how the CPUs 704 transition between states, as described above. According to one example, the computer 700 has access to computer-readable storage media storing computer-executable instructions which, when executed by the computer 700, perform one or more of the various processes described herein. The computer 700 might also include computer-readable storage media for performing any of the other computer-implemented operations described herein.

The computer 700 may also include one or more input/output controllers 716 for receiving and processing input from a number of input devices, such as a keyboard, a mouse, a touchpad, a touch screen, an electronic stylus, or other type of input device. Similarly, the input/output controller 716 may provide output to a display, such as a computer monitor, a flat-panel display, a digital projector, a printer, a plotter, or other type of output device. It will be appreciated that the computer 700 may not include all of the components shown in FIG. 2, may include other components that are not explicitly shown in FIG. 2, or may utilize an architecture completely different than that shown in FIG. 2.

Further, the processes discussed herein may be implemented in hardware, software, or a combination thereof. In the context of software, the described operations represent computer-executable instructions stored on one or more computer-readable storage media that, when executed by one or more hardware processors, perform the recited operations. Generally, computer-executable instructions include routines, programs, objects, components, data structures, and the like that perform particular functions or implement particular abstract data types. Those having ordinary skill in the art will readily recognize that certain steps or operations illustrated in the figures above may be eliminated, combined, or performed in an alternate order. Any steps or operations may be performed serially or in parallel (unless context requires one or the other). Furthermore, the order in which the operations are described is not intended to be construed as a limitation.

Embodiments may be provided as a software program or computer program product including a non-transitory computer-readable storage medium having stored thereon instructions (in compressed or uncompressed form) that may be used to program a computer (or other electronic device) to perform processes or methods described herein. The computer-readable storage medium may be one or more of an electronic storage medium, a magnetic storage medium, an optical storage medium, a quantum storage medium, and so forth. For example, the computer-readable storage media may include, but is not limited to, hard drives, floppy diskettes, optical disks, read-only memories (ROMs), random access memories (RAMs), erasable programmable ROMs (EPROMs), electrically erasable programmable ROMs (EEPROMs), flash memory, magnetic or optical cards, solid-state memory devices, or other types of physical media suitable for storing electronic instructions. Further, embodiments may also be provided as a computer program product including a transitory machine-readable signal (in compressed or uncompressed form). Examples of machine-readable signals, whether modulated using a carrier or unmodulated, include, but are not limited to, signals that a computer system or machine hosting or running a computer program can be configured to access, including signals transferred by one or more networks. For example, the transitory machine-readable signal may comprise transmission of software by the Internet.

Separate instances of these programs can be executed on or distributed across any number of separate computer systems. Thus, although certain steps have been described as being performed by certain devices, software programs, processes, or entities, this need not be the case, and a variety of alternative implementations will be understood by those having ordinary skill in the art.

Additionally, those having ordinary skill in the art readily recognize that the techniques described above can be utilized in a variety of devices, environments, and situations. Although the subject matter has been described in language specific to structural features or methodological acts, it is to be understood that the subject matter defined in the appended claims is not necessarily limited to the specific features or acts described. Rather, the specific features and acts are disclosed as exemplary forms of implementing the claims.

The Exemplary Embodiments below, and the exemplary methods described herein, are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXEMPLARY EMBODIMENTS

1. An assay, including: obtaining a sample including: a cell or tissue infected with a herpesvirus, an extract from a cell or tissue infected with a herpesvirus, or a protein preparation from a cell or tissue infected with a herpesvirus; determining the abundance level of a plurality of herpesvirus proteins in the sample using parallel reaction monitoring (PRM) to quantify signature peptide(s) corresponding to the herpesvirus proteins; wherein the herpesvirus is HSV-1 and the signature peptides are selected from peptides in Table 1; or the herpesvirus is HCMV and the signature peptides are selected from peptides in Table 2; or the herpesvirus is KSHV and the signature peptides are selected from peptides in Table 3.
2. The assay of embodiment 1, wherein for at least the one herpesvirus protein for which the abundance level is determined, at least two signature peptides are quantified.
3. The assay of embodiment 1, wherein determining the abundance level of the plurality of herpesvirus proteins using PRM includes subjecting the sample to liquid chromatography coupled to tandem mass spectrometry (LC-MS/MS).
4. The assay of embodiment 1, wherein the plurality of herpesvirus proteins includes at least one herpesvirus protein from each temporal class of viral replication for that herpesvirus.
5. The assay of embodiment 1, wherein the cell or tissue infected with the herpesvirus is a human cell or human tissue.
6. The assay of embodiment 1, wherein the plurality of herpesvirus proteins constitutes approximately 30-70%, or 50-80%, of the predicted viral proteome.
7 A time course assay, including: repeating the assay of embodiment 1 a plurality of times, where for each repetition the sample is obtained at a different timepoint in a time course.
8. The time course assay of embodiment 7, where the different timepoints are different times post infection of the cell or tissue with the herpesvirus.
9. The time course assay of embodiment 8, wherein the different times after infection of the cell or tissue with the herpesvirus include at least one time from each state of a replication cycle of the herpesvirus.
10. The time course assay of embodiment 7, where the different timepoints are different times post exposure of the cell or tissue to a compound or environmental variable.
11. An exposure or dosage course assay, including: repeating the assay of embodiment 1 a plurality of times, where for each repetition the sample is obtained from a cell or tissue that has been exposed to a different compound or condition or a different dosage of a compound or a condition.
12. The exposure or dosage course assay of embodiment 11, wherein the different compounds include one or more of known antiviral compounds, proposed antiviral compounds, test compounds, small molecule drugs or drug candidates, or siRNAs or other biologically active non-coding RNAs.
13. The exposure or dosage course assay of embodiment 12, wherein the known antiviral compounds include one or more of acyclovir, ganciclovir, another nucleoside, penciclovir, famciclovir, valacyclovir, valganciclovir, cidofovir, another nucleotide phosphonate, fomivirsen, or foscarnet.
14. The exposure or dosage course assay of embodiment 11, wherein different compounds include honokiol.
15. The exposure or dosage course assay of embodiment 11, wherein the different include one or more of genetic modification of the cell or tissue, genetic modification of the herpesvirus, environmental conditions, or cell or tissue growth or harvesting conditions.
16. The exposure or dosage course assay of embodiment 15, wherein the genetic modification of the cell or tissue includes knock out or up-regulation of one or more host factors.
17. A method for quantification of herpesvirus proteins from multiple temporal classes of viral replication, including: subjecting a cell sample or cell extract to parallel reaction monitoring (PRM) to generate abundance data; analyzing the abundance data to quantify signature peptide(s) corresponding to at least one herpesvirus protein from each of at least two temporal classes of viral replication; and providing the quantified peptide(s) results from the analyzing to a database, a computer memory, a display, a printer, or another output device; wherein the herpesvirus is HSV-1 and the signature peptides are selected from peptides in Table 1; or the herpesvirus is HCMV and the signature peptides are selected from peptides in Table 2; or the herpesvirus is KSHV and the signature peptides are selected from peptides in Table 3.
18. Use of any of the assays of embodiments 1-17, to: screen drug candidates as modulators of viral infection; analyze the stage of infection at which a test compound acts; determine what functional family(s) of viral proteins are affected by a drug or drug candidate; characterize viral and/or host responses to viral infection; characterize viral and/or host responses to drug treatment; or characterize viral and/or host responses to genetic manipulation of either the viral genome or the host genome.
19. A kit for use with an assay of any one of embodiments 1-16 or the use of embodiment 18, including: parameters for performing the assay for a target herpesvirus, a set of heavy isotope labeled peptides for use as controls, and a USB drive or other non-transitory computer readable medium containing software for assay analysis and/or standardized report generation.
20. The kit of embodiment 19, wherein the target herpesvirus is HSV-1 and the set of heavy isotope labeled peptides includes: at least two signature peptides in Table 1; at least one signature peptide for each protein in Table 1; or at least one signature peptide from Table 1 for at least one protein from each temporal stage of HSV-1 viral replication.
21. The kit of embodiment 19, wherein the target herpesvirus is HCMV and the set of heavy isotope labeled peptides includes: at least two signature peptides in Table 2; at least one signature peptide for each protein in Table 2; or at least one signature peptide from Table 2 for at least one protein from each temporal stage of HCMV viral replication.
22. The kit of embodiment 19, wherein the target herpesvirus is KSHV and the set of heavy isotope labeled peptides includes: at least two signature peptides in Table 3; at least one signature peptide for each protein in Table 3; or at least one signature peptide from Table 3 for at least one protein from each temporal stage of KSHV viral replication.
23. A service, including: performing the assay of any one of embodiments 1-17 or the use of embodiment 18 on one or more biological samples provided by another.
24. A quantitative assay for herpesviral proteins, substantially as described herein.
25. A non-naturally occurring, labeled peptide having the amino acid sequence of a peptide in Table 1, Table 2, or Table 3.
26. The non-naturally occurring, labeled peptide of embodiment 25, wherein the label enables the peptide to be distinguished from an unlabeled peptide with the same amino acid sequence in liquid chromatography coupled to tandem mass spectrometry (LC-MS/MS) analysis.
27. A collection of non-naturally occurring, labeled signature peptides specific for HSV-1, including: at least one peptide from Table 1 for each of the 60 proteins listed in Table 1; at least two peptides from Table 1 for each of the 60 proteins listed in Table 1; at least three peptides from Table 1 for each of the 60 proteins listed in Table 1; at least one peptide from Table 1 for at least one protein listed in Table 1 from each temporal stage of HSV-viral replication; at least 60 of the peptides listed in Table 1; more than 17 of the peptides listed in Table 1; at least 30 of the peptides listed in Table 1; at least 50 of the peptides listed in Table 1; at least 60 of the peptides listed in Table 1; substantially all of the peptides listed in Table 1; or all of the peptides listed in Table 1; wherein each peptide in the collection includes a label that enables the labeled peptide to be distinguished from an unlabeled peptide with the same amino acid sequence in liquid chromatography coupled to tandem mass spectrometry (LC-MS/MS) analysis.
28. A collection of non-naturally occurring, labeled signature peptides specific for HCMV, including: at least one peptide from Table 2 for each of the 90 proteins listed in Table 2; at least two peptides from Table 2 for a plurality of the 90 proteins listed in Table 2; at least three peptides from Table 2 for a plurality of the 90 proteins listed in Table 2; at least one peptide from Table 2 for at least one protein listed in Table 2 from each temporal stage of HCMV-viral replication; at least 90 of the peptides listed in Table 2; more than 90 of the peptides listed in Table 2; at least 30 of the peptides listed in Table 2; at least 50 of the peptides listed in Table 2; at least 100 of the peptides listed in Table 2; at least 150 of the peptides listed in Table 2; at least 200 of the peptides listed in Table 2; substantially all of the peptides listed in Table 2; or all of the peptides listed in Table 2; wherein each peptide in the collection includes a label that enables the labeled peptide to be distinguished from an unlabeled peptide with the same amino acid sequence in liquid chromatography coupled to tandem mass spectrometry (LC-MS/MS) analysis.
29. A collection of non-naturally occurring, labeled signature peptides specific for KSHV, including: at least one peptide from Table 3 for each of the 62 proteins listed in Table 3; at least two peptides from Table 3 for a plurality of the 62 proteins listed in Table 3; at least three peptides from Table 3 for a plurality of the 62 proteins listed in Table 3; at least one peptide from Table 3 for at least one protein listed in Table 3 from each temporal stage of KSHV-viral replication; at least 62 of the peptides listed in Table 3; more than 62 of the peptides listed in Table 3; at least 30 of the peptides listed in Table 3; at least 50 of the peptides listed in Table 3; at least 75 of the peptides listed in Table 3; at least 100 of the peptides listed in Table 3; at least 150 of the peptides listed in Table 3; substantially all of the peptides listed in Table 3; or all of the peptides listed in Table 3; wherein each peptide in the collection includes a label that enables the labeled peptide to be distinguished from an unlabeled peptide with the same amino acid sequence in liquid chromatography coupled to tandem mass spectrometry (LC-MS/MS) analysis.
30. The peptide collection of any one of embodiments 27-29, wherein the label on at least one peptide in the collection includes a heavy isotope.

### Example 1: A Trusted Targeted Mass Spectrometry Assay for Pan-Herpesvirus Protein Detection

The presence and abundance of viral proteins within host cells are part of the essential signatures of the cellular stages of viral infections. Viral proteins are either brought into host cells by infectious particles or expressed at specific steps of the replication cycle. However, methods that can comprehensively detect and quantify these proteins are still limited, particularly for viruses with large protein coding capacity. Here, a mass spectrometry-based Targeted herpesviRUS proTEin Detection (TRUSTED) assay was designed and experimentally validated for monitoring human viruses representing the three Herpesviridae subfamilies—herpes simplex virus type 1 (HSV-1), human cytomegalovirus (HCMV), and Kaposi's sarcoma-associated herpesvirus (KSHV). Assay applicability was demonstrated for 1) capturing the temporal cascades of viral replication, 2) detecting proteins throughout a range of virus concentrations, 3) assessing the effects of clinical therapeutic agents, 4) characterizing the impact of sirtuin-modulating compounds, and 5) studies using different laboratory and clinical viral strains.

As evidenced by the global burden of viral infectious disease, there is a need for methods that can quickly and accurately detect viral infections and monitor their progression in both laboratory and clinical settings. An indicator of the presence of a viral infection and the stage of a replication cycle is the expression and abundance of viral proteins (Greco et al., Annu. Rev. Virol. 1, 581-604, 2014; Gruffat et al., Front. Microbiol. 7, 2016). Numerous human viruses proceed through their replication cycle by initiating a temporal cascade of viral gene expression, and the expression of different viral proteins can provide signatures of infection progression. However, the genome size and subsequent number of proteins expressed by different viruses varies widely. For example, viruses range from those expressing a single polyprotein that is cleaved into 10-20 individual proteins (e.g. hepatitis C virus, coronaviruses, poliovirus, etc.) to those with hundreds (e.g. human cytomegalovirus (HCMV)) or thousands (e.g. pandoravirus) of predicted open reading frames (Philippe et al., Science 341, 281-286, 2013; Spall et al., Semin. Virol. 8, 15-23, 1997; Stern-Ginossar et al., Science 338, 1088-1093, 2012). Consequently, it can be challenging to comprehensively monitor viral protein levels for viruses with large protein coding capacity, given that the complexity of such a detection method would scale with the size of the viral proteome. Additionally, the study of viruses with large proteomes has historically suffered from the especially small percentage of viral proteins for which commercially produced antibodies are available.

Among these large viruses are herpesviruses, which first emerged over 200 million years ago, and consequently have coevolved with humans and other hosts into modernity. This long history of virus-host co-evolution has allowed these viruses to acquire relatively large proteomes (70-250 putative proteins) that facilitate their diverse means for co-opting cellular processes and evading host defense mechanisms. The herpesvirus family consists of three subfamilies of alpha-, beta-, and gamma-herpesviruses—each of which encompass prevalent human pathogens that establish latent, life-long infections that can sporadically reactivate to cause acute disease. For example, alpha-herpesviruses, like herpes simplex virus type I (HSV-1) and type II (HSV-2), cause symptoms ranging from skin lesions to deadly encephalitis (Whitley & Roizman, Lancet 357, 1513-1518, 2001) and the beta-herpesvirus HCMV is linked to cardiac disease (Courivaud et al., J. Infect. Dis. 207, 1569-1575, 2013) and is the leading cause of virally induced birth defects (Cheeran et al., Clin. Microbiol. Rev. 22, 99-126, 2009). Furthermore, some herpesviruses can exacerbate infections with other viral agents. For example, HSV-2 increases the likelihood of contraction and spread of human immunodeficiency virus (HIV-1) (Zhu et al., Nat. Med. 15, 886-892, 2009), and the gamma-herpesvirus Kaposi's sarcoma-associated herpesvirus (KSHV) is the leading cause of cancer in untreated HIV-infected individuals (Mesri et al., Nat. Rev. Cancer 10, 707-719, 2010). However, despite their prevalence as human pathogens and the global health burden of herpesvirus-induced diseases, the available antiviral treatments suffer from toxicity issues (Adair et al., South. Med. J. 87, 1227-1231, 1994; Asahi et al., Eur. J. Neurol. 16, 457-460, 2009; Bedard et al., Antimicrob. Agents Chemother. 43, 557-567, 1999) and vaccines for these viruses do not exist.

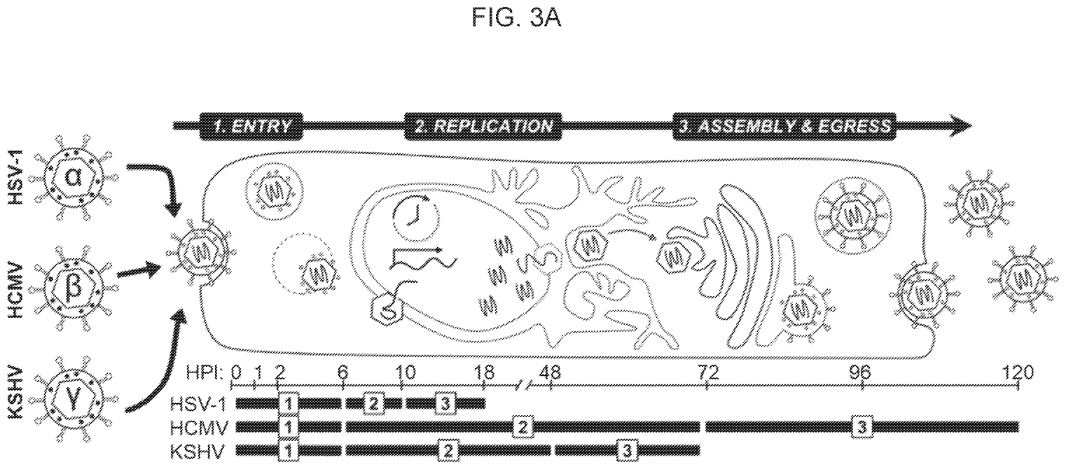

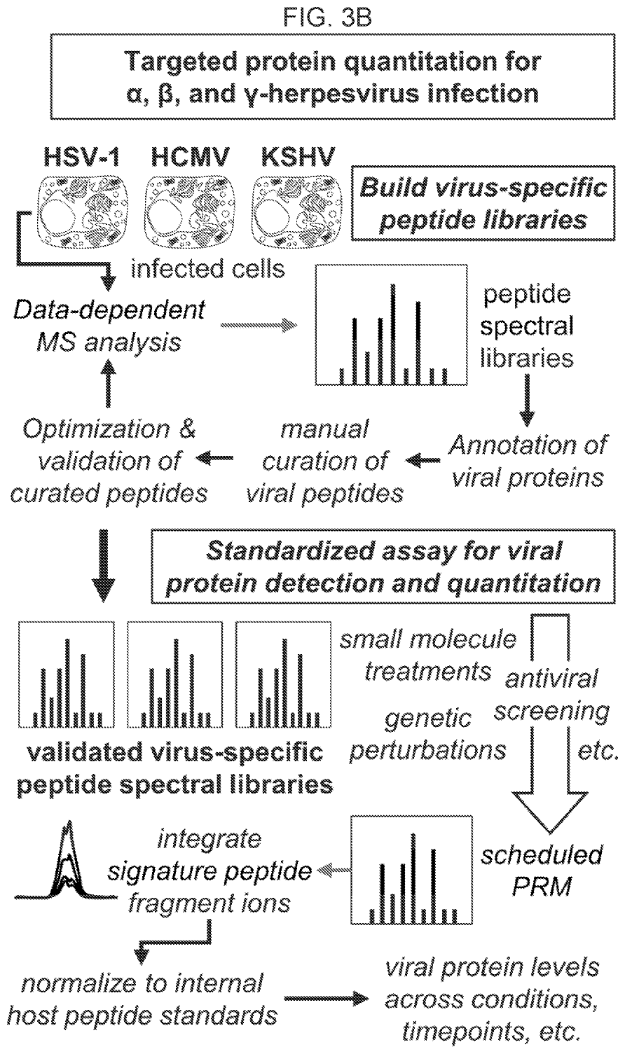

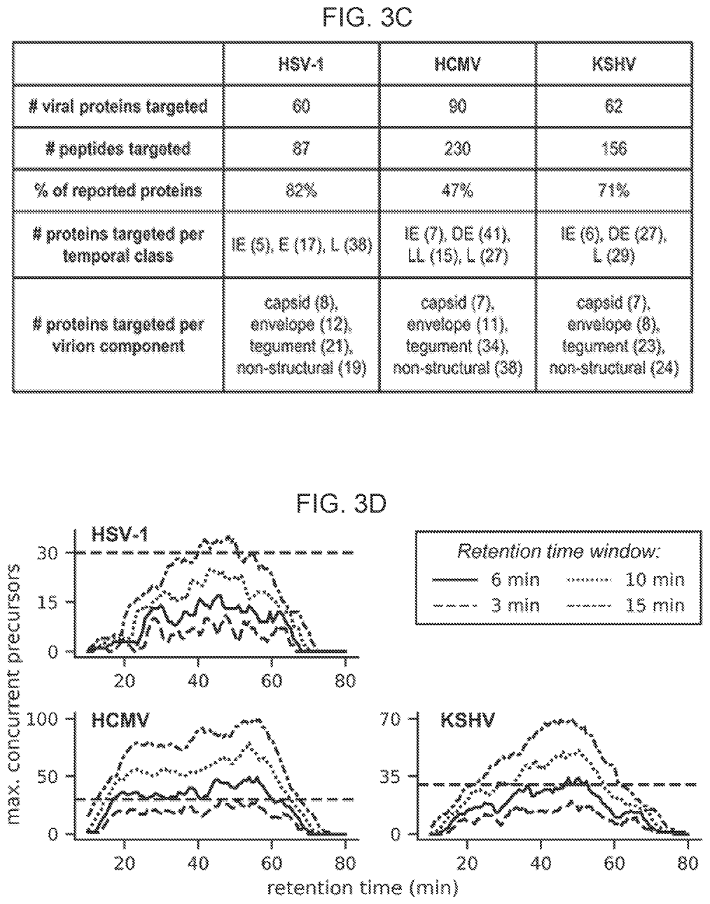

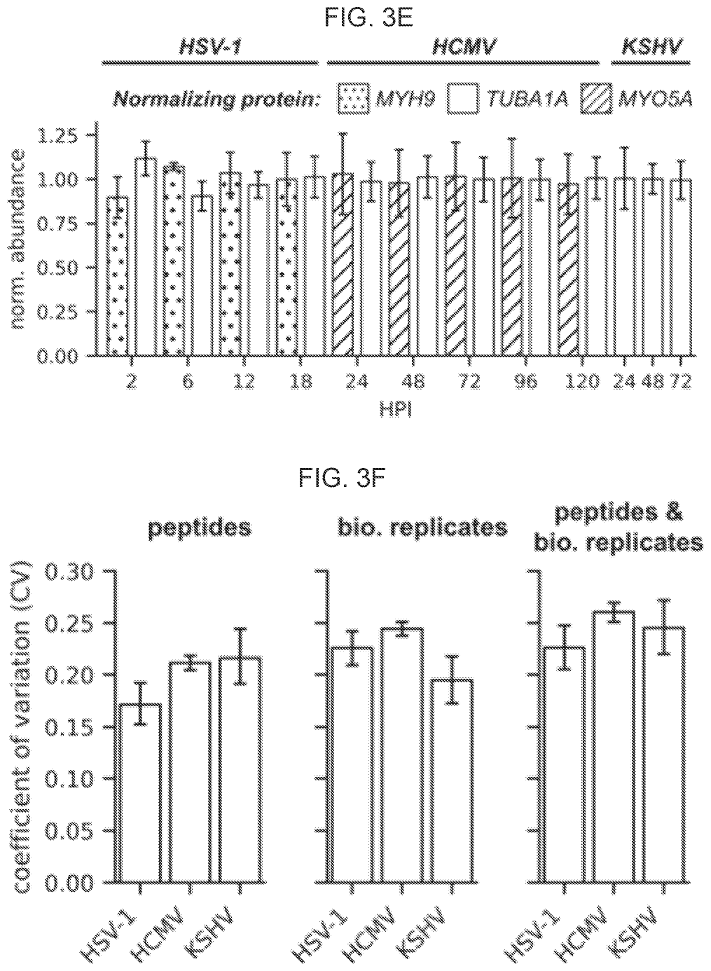

In addition to sharing a proclivity for causing critical diseases, herpesviruses also share a common structure and replication cycle (FIG. 3A). As enveloped, double-stranded DNA viruses, herpesviruses enter the cell, traffic to the nucleus where they replicate their viral genomes, and finally package this newly synthesized viral DNA into progeny virions that can egress from the cell to continue the infection cycle (Adler et al., Trends Microbiol. 25, 229-241, 2017). Although many of these stages are shared between these viruses, they complete their replication cycles over different lengths of time. For example, HSV-1 replicates in under 24 hours, while KSHV takes ˜3 days, and HCMV takes 4-5 days. Despite these differences, a shared characteristic feature of herpesvirus replication is the tightly regulated temporal cascade of viral gene expression that ensues following viral entry into the cell, which can include the expression of immediate early (IE), early (E), delayed early (DE), leaky late (LL), and late (L) classes of viral genes (Honess & Roizman, J. Virol. 14, 8-19, 1974; Schulz & Chang, In Human Herpesviruses: Biology, Therapy, and Immunoprophylaxis, (Cambridge University Press), Chapter 28, 2007; Stinski, J. Virol. 26, 686-701, 1978). Consequently, monitoring the levels of herpesvirus proteins not only allows establishment of the presence of infection, but also the stage at which a particular sample is in the infection cycle. The monitoring of few IE, DE, or L marker proteins is standard for assessing replication progression. Traditionally, common methods for monitoring herpesvirus replication include antibody-based techniques such as Western blot (Omoto & Mocarski, J. Virol. 87, 8651-8664, 2013; Sheng & Cristea, PLOS Pathog. 17, e1009506, 2021) and ELISA (Inoue et al., Clin. Diagn. Lab. Immunol. 7, 427-435, 2000) or nucleic acid-based approaches such as microarrays (Bresnahan & Shenk, Science 288, 2373-2376, 2000 Polson et al., Cancer Res. 62, 4525-4530, 2002) and RNA-seq (Boldogköi et al., Sci. Data 5, 1-14, 2018; Wyler et al., Nat. Commun. 10, 1-14, 2019). However, each of these methods suffers from drawbacks including that RNA-based approaches frequently do not accurately reflect the protein abundances which drive cellular phenotypes (Ruggles et al., Mol. Cell. Proteomics 16, 959-981, 2017; Vogel & Marcotte, Nat. Rev. Genet. 13, 227-232, 2012; Zhang et al., Nature 513, 382-387, 2014) and that antibodies against viral proteins often either do not exist or are insufficiently characterized. Being able to accurately monitor the abundances of most viral proteins would provide the ability to comprehensively characterize specific stages of infection and to identify the temporal regulation of viral effectors that inhibit host defense factors and modulate cellular processes. Such a detection method would also allow for the screening of small molecules for their potential anti- or pro-viral activities and discovering their putative viral targets.

Targeted mass spectrometry (MS) offers a robust method to directly detect and quantify specific proteins of interest with high sensitivity and accuracy. Targeted MS methods, such as parallel reaction monitoring (PRM) and selected reaction monitoring (SRM), rely on the curation of libraries of peptides that fulfill a series of detection requirements, such as being unique to a given protein, well-ionized, and amenable to chromatography separation and MS/MS fragmentation during the nLC-MS/MS analysis. Such libraries provide signature peptides for an array of proteins of interest. With iterative development and validation steps, these methods can be scaled up for high throughput monitoring of hundreds of proteins in a single run (Ebhardt et al., Proteomics 15, 3193-3208, 2015; Lum et al., Cell Syst. 7, 627-642.e6, 2018). Once such a library is developed, these targeted MS approaches can be implemented on several mass spectrometry instrumentation platforms and within different experimental workflows. Ultimately, the established detection parameters for these signature peptides are readily transferrable to other research, clinical, or industry labs.

Here, a PRM detection library was designed and experimentally validated for the broad detection of viral proteins from all three herpesvirus families: the alpha-herpesvirus HSV-1, the beta-herpesvirus HCMV, and the gamma-herpesvirus KSHV. This assay is called TRUSTED (Targeted herpesviRUS proTEin Detection). The breadth of proteins monitored by the method captures the temporal cascades of the replication cycles of these viruses. The targeted MS assay accurately quantified the effects of clinically relevant antiviral agents, further capturing their precise temporal regulation of specific viral proteins. Further establishing the applicability of this method for characterizing small molecule compounds, the effects of drugs that modulate the antiviral activity of sirtuin proteins was investigate. Finally, a computational analysis of peptide conservation was performed, demonstrating the applicability of TRUSTED across different viral strains, including laboratory and clinical isolates. Overall, this method provides a sensitive, reliable, and scalable assay for monitoring herpesvirus protein levels and has been deposited online to the PRIDE repository to be readily implementable by other research groups. These results support the broad applicability of these assays for probing viral protein abundances in a wide variety of model systems and contexts, including antiviral drug screening, detecting infections in clinical settings, and genetic manipulations of virus or host factors.

### Results

### A Targeted Mass Spectrometry Assay for Detecting and Quantifying Signature Alpha-, Beta-, and Gamma-Herpesvirus Proteins

Considering the biological and clinical relevance of herpesviruses and the lack of methods to comprehensively monitor herpesvirus protein expression in laboratory and clinical settings, a targeted PRM-based assay was developed that offers the ability to systematically quantify viral protein abundances during HSV-1, HCMV, and KSHV infections. To accomplish this, infections were performed in human fibroblast cells for HSV-1 and HCMV, and used a latently-infected cell model (iSLK.219) that can be reactivated to study lytic KSHV infection (Myoung & Ganem, J. Virol. Methods 174, 12-21, 2011). Although both of these cell types represent standard model systems for the study of each aforementioned infection, the assay was designed to be readily applicable to other cell culture models or tissues.

To capture the various temporal stages of these virus replication cycles, proteins across all classes of herpesvirus gene expression and different virion components were targeted. Detection of canonical markers of infection progression was focused on for each virus, as was detection of viral proteins with diverse cellular functions and localizations. The assays were designed to monitor peptides generated by trypsin digestion given the widespread use and accessibility of this enzyme in experimental workflows. Additionally, it was found that the predicted lysine/arginine content of these viruses, as well as their predicted tryptic peptide content, was well suited to MS analysis. Moving forward, a set of signature peptides was manually curated for each virus by performing an iterative process of exploratory, data-dependent MS analyses of infected samples and experimental validation of peptide detection and reliability by PRM (FIG. 3B). To further advance the method to monitor proteins and peptides not identified in the exploratory analyses, existing literature and peptide databases were also queried for previously detected viral peptides and attempted to validate these via unscheduled PRM injections. The majority of the HSV-1, HCMV, and KSHV proteins were represented in these assays by 2-4 peptides ranging from 6-36 amino acids in length, with few additional viral proteins being captured by only one experimentally validated peptide. As a result, this allowed for the monitoring of peptides from viral proteins belonging to all temporal classes of viral genes for all three viruses, representing the IE, DE, E, LL, and L replication stages, as well as components of the virion (e.g. capsid, tegument, and envelope proteins) (FIG. 3C, Tables 1-3).

Overall, these assays measure the levels of proteins representing 50-80% of the reported proteomes for each virus. Of the three viruses discussed here, HSV-1 expresses the smallest number of proteins and replicates in the fastest amount of time. This HSV-1 PRM assay quantifies up to 60 viral proteins with 3-4 peptides being monitored for most targets. Comparatively, HCMV and KSHV express substantially more proteins, and these assays monitor up to 90 and up to 62 viral proteins, respectively. Moreover, greater than 50% of the proteins quantified by the assays represent targets without commercially available antibodies.

The assay monitors these viral peptides of interest using 6-minute retention time windows across a series of one (HSV-1 and KSHV) or two (HCMV) 60-minute injections using ˜1.5 μg of input sample (FIG. 3D). To reliably quantify protein abundance across different biological samples, the assay leverages internal reference standard peptides that help account for variability in input material due to natural variation in sample preparation and other factors. To serve this purpose several ubiquitously expressed cytoskeletal factors were chosen, including tubulin (TUBA1A), myosin 5A (MYOSA), and a myosin II heavy chain (MYH9), which exhibit stable expression levels throughout infection (FIG. 3E, Tables 1-3). After normalizing for differences in input sample, measurements were obtained that were reproducible and exhibited low mass errors for the relative protein abundances across the different infections. Coefficients of variation (CVs) averaged less than 30% across peptides corresponding to a given protein and across different biological replicates (FIG. 3F). Altogether, this process culminated in the establishment of virus-specific peptide libraries that proved effective at robustly detecting HSV-1, HCMV, and KSHV peptides during wild type infections. Given the robustness and accuracy of detection offered by targeted mass spectrometry, this assay was named TRUSTED (Targeted herpesviRUS proTEin Detection).

### Herpesvirus PRM Assay Captures the Signature Temporal Cascade of Viral Gene Expression

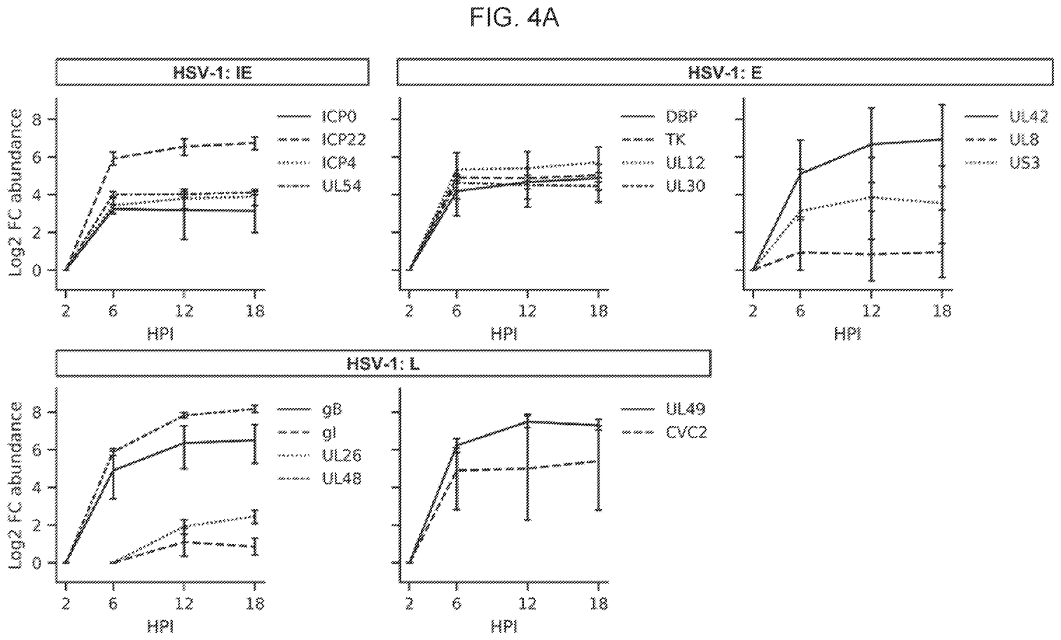

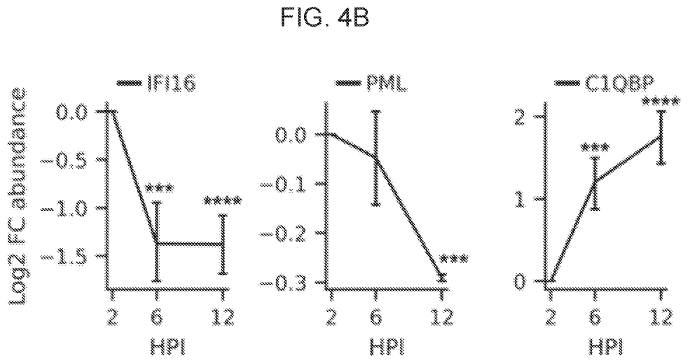

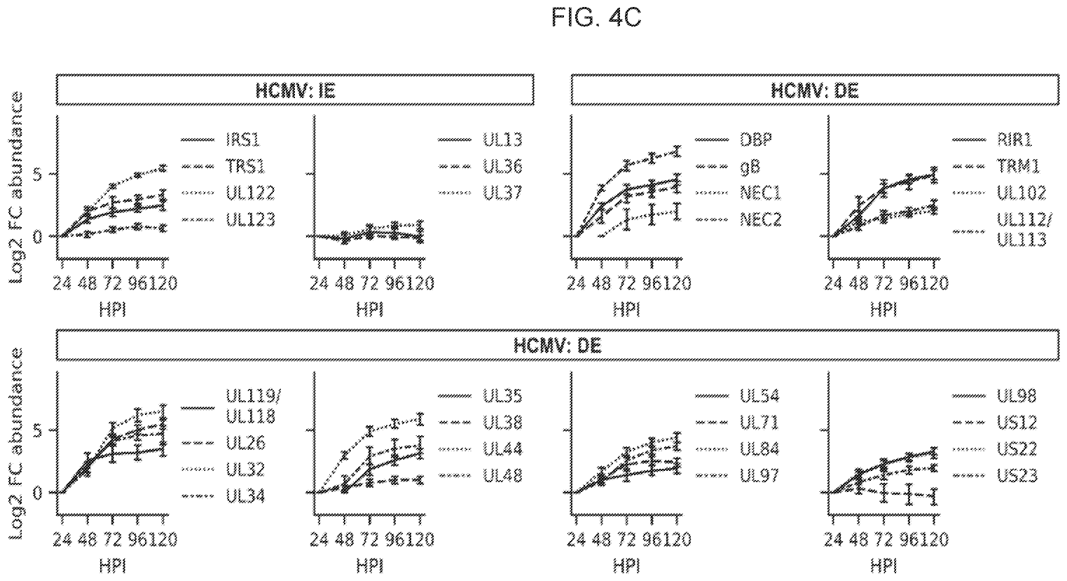

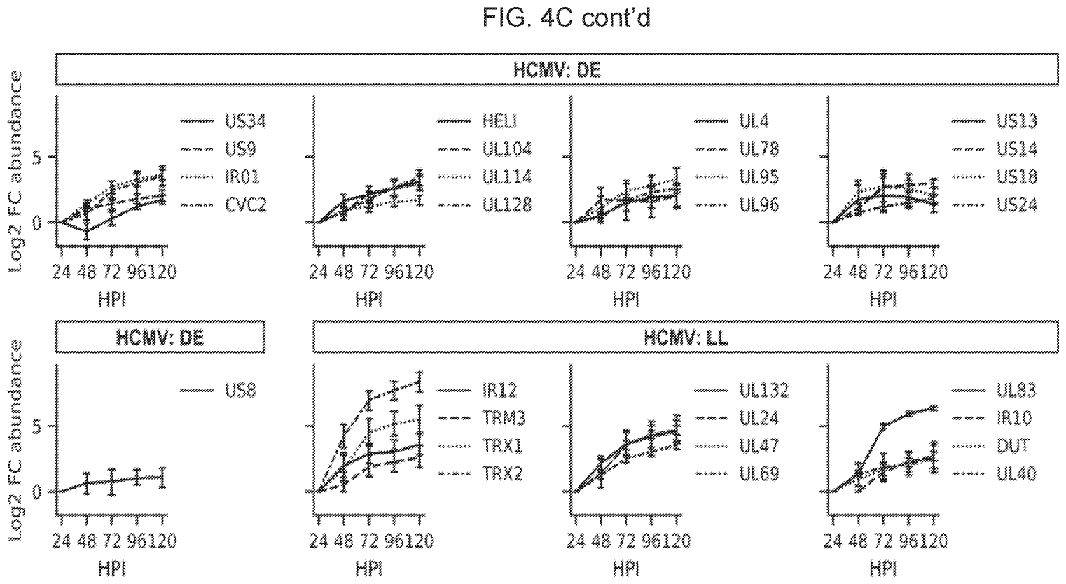

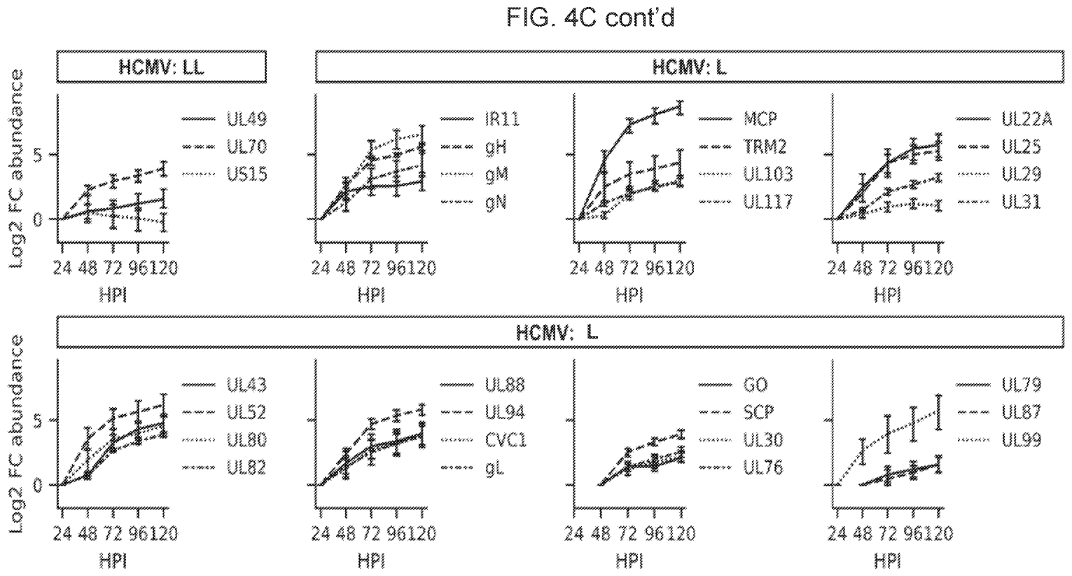

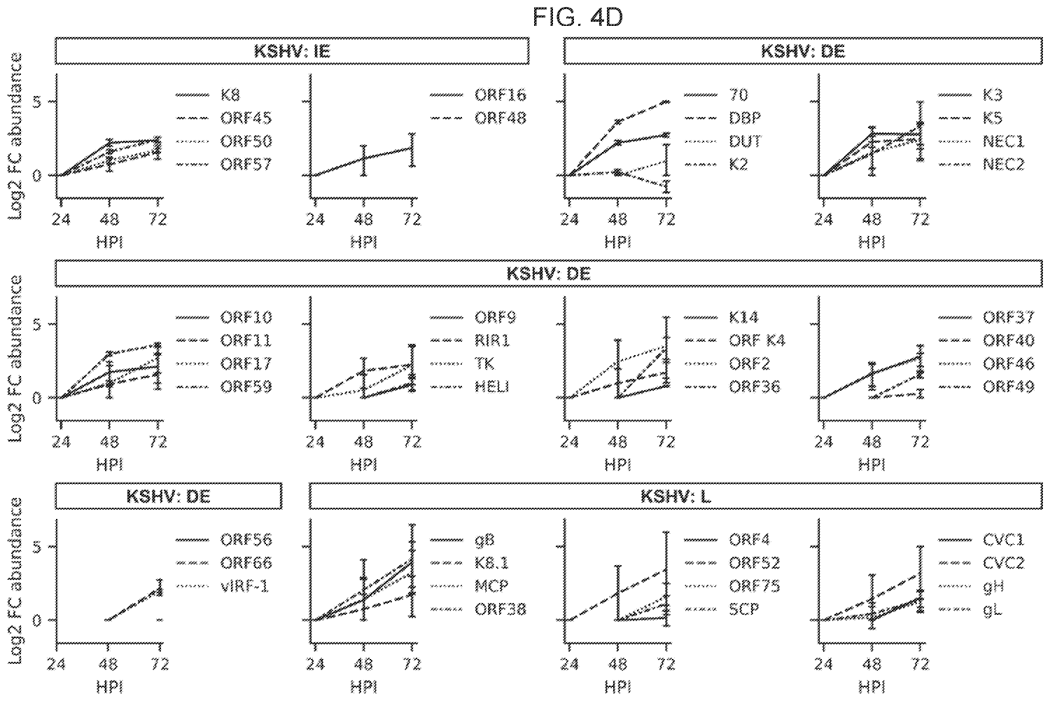

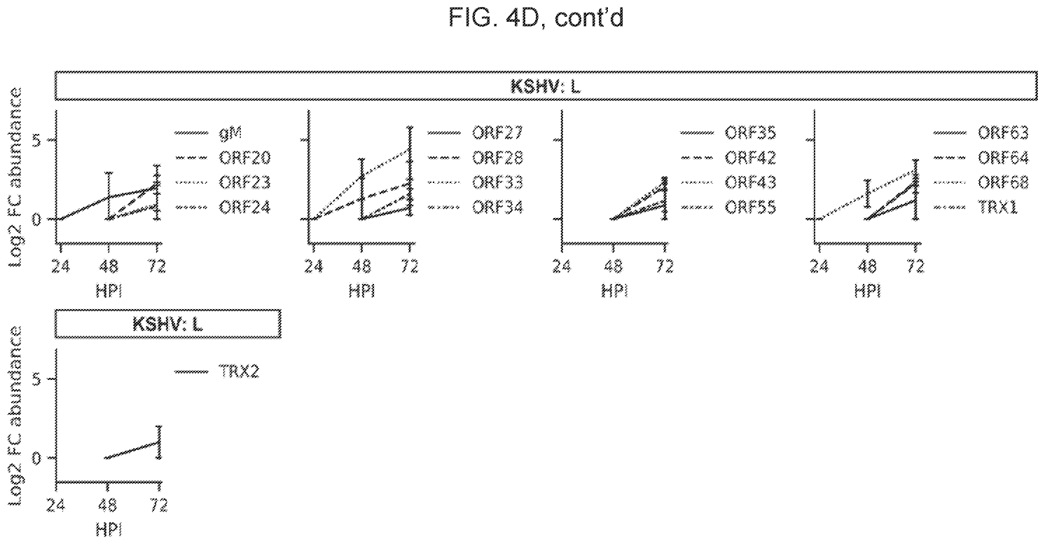

An essential aspect of herpesvirus replication is the temporal cascade of gene expression that ensues following viral entry into cells. Having demonstrated that the assays can accurately detect viral proteins, whether it can also capture the temporality of their abundances during the progression of infection was next assessed. For HSV-1, infected fibroblasts were harvested at 2, 6, 12, and 18 hours post-infection (HPI), while for HCMV cells at 24, 48, 72, 96, and 120 HPI were collected. For KSHV, the latent virus was reactivated in iSLK.219 cells and collected samples at 24, 48, and 72 hours post-reactivation (HPR). For each virus, these time points represent the specific stages of virus gene expression (immediate early through late), virion assembly, and egress. Measurements of protein levels at each time point demonstrated the sequential nature of viral protein levels, as expected from the well-established cascades of gene expression that are characteristic of herpesvirus infections (depicted as fold-change in FIG. 4).

For HSV-1 infection, viral protein levels increased throughout the course of infection, with an approximately 32-fold median increase observed at 18 HPI relative to the first time point of detection for each protein (FIG. 4A). To further confirm the adequate progression through infection, this PRM assay was also designed to include peptides from host factors known to be inhibited or repurposed by HSV-1 (FIG. 4B). Indeed, in accordance with previous studies (Boutell et al., J. Virol. 76, 841-850, 2002; Johnson et al., Virol. 87, 5005-5018, 2013; Liu et al., J. Virol. 89, 8982-8998, 2015; Orzalli et al., Proc. Natl. Acad. Sci. U.S.A 109, 2012), virus-induced degradation of the defense factors, interferon-inducible protein 16 (IFI16) and PML, and an increase in the levels of the pro-viral host protein C1QBP (p32) were observed.

During HCMV infection, viral protein levels increased up to 1000-fold, with a median increase of ˜10-fold by 120 HPI (FIG. 4C). It was also noted that some HCMV IE proteins (UL13, UL36, UL37, and UL123) exhibited less induction (<2-fold, on average), compared to most other HCMV proteins. This agrees with literature reports that many of these IE proteins are highly induced early upon infection, afterwards maintaining similar levels throughout the virus replication cycle (Jean Beltran et al., Cell Syst. 3, 361-373, 2016; Lu & Everett, J. Virol. 89, 3062-3075, 2015; McCormick et al., J. Virol. 77, 631-641, 2003).

The reactivation of KSHV led to milder temporal increases of ˜4-fold by 72 HPR (FIG. 4D). Like most HSV-1 and HCMV proteins, KSHV protein levels also generally increased following reactivation, with the exception of the DE protein K2, which was decreased by ˜40% by 72 HPR. This agrees with a previous study showing that K2 is robustly expressed in latently infected iSLK.219 cells, but its levels are decreased following reactivation (Park et al., Sci. Rep. 9, 1-13, 2019).

### Differing Levels of Infection (MOI) are Robustly Detected Via PRM

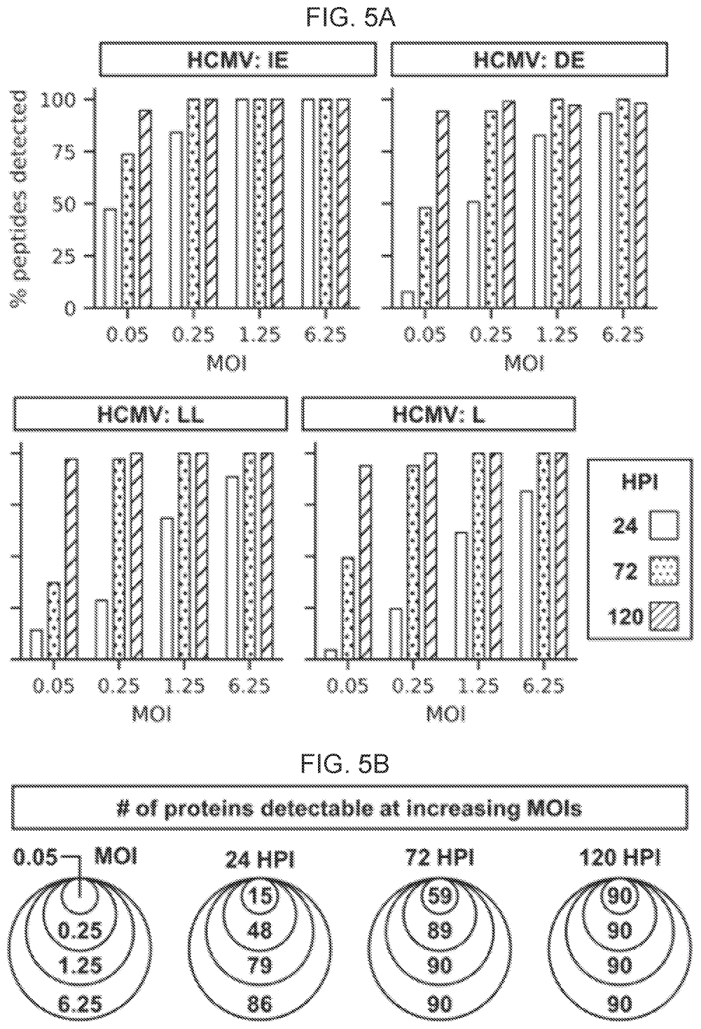

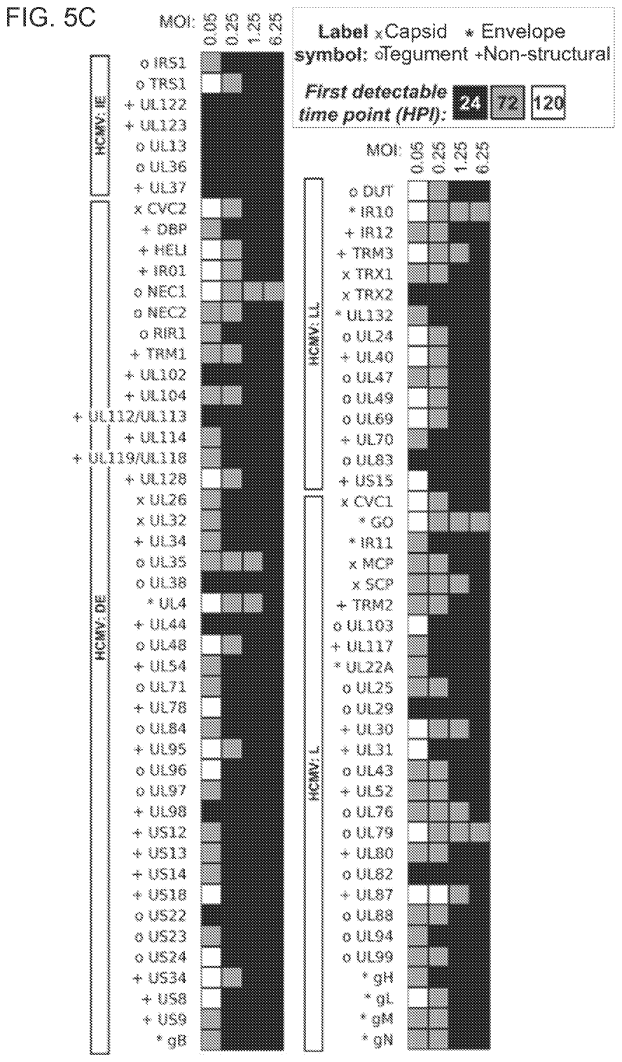

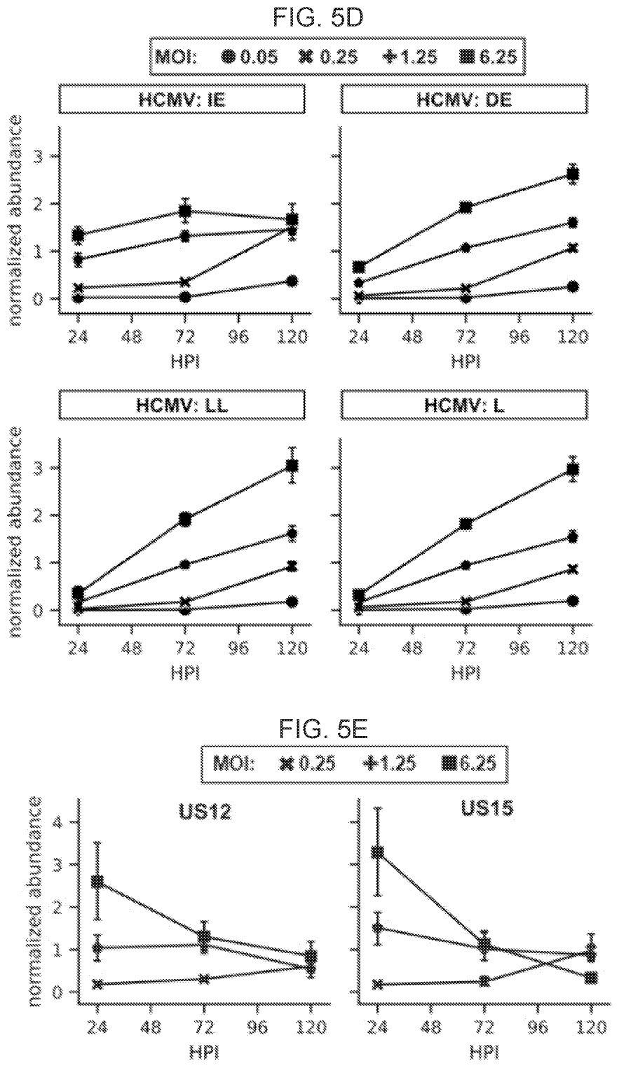

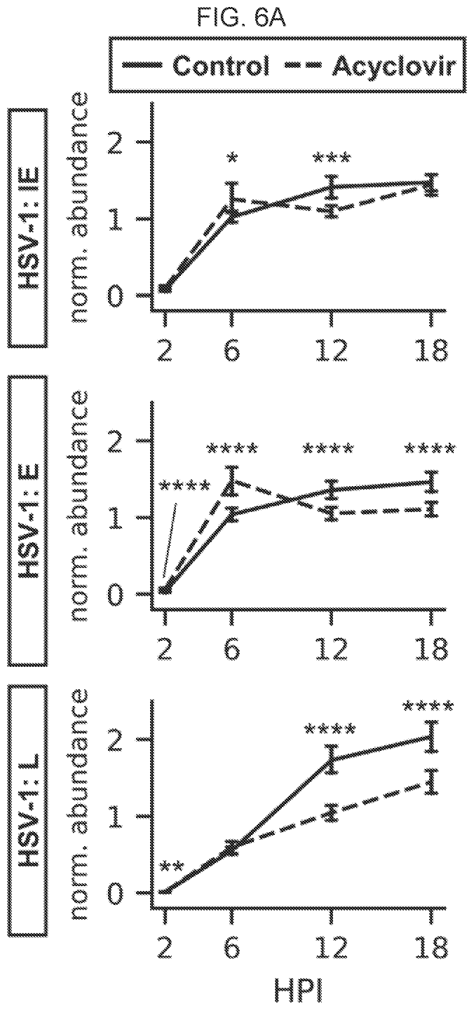

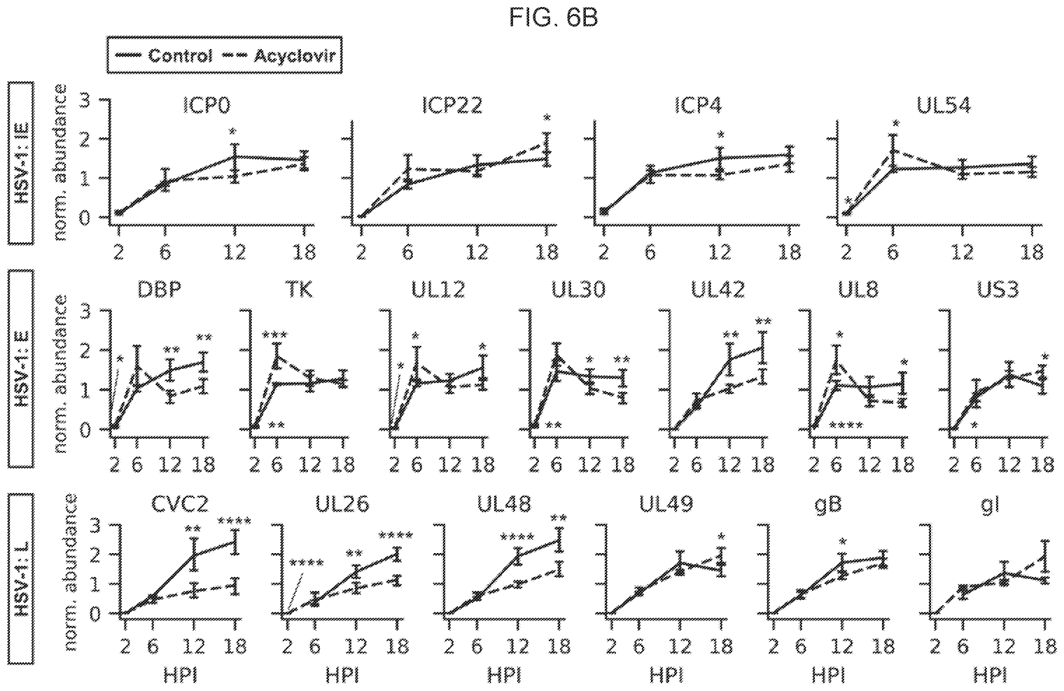

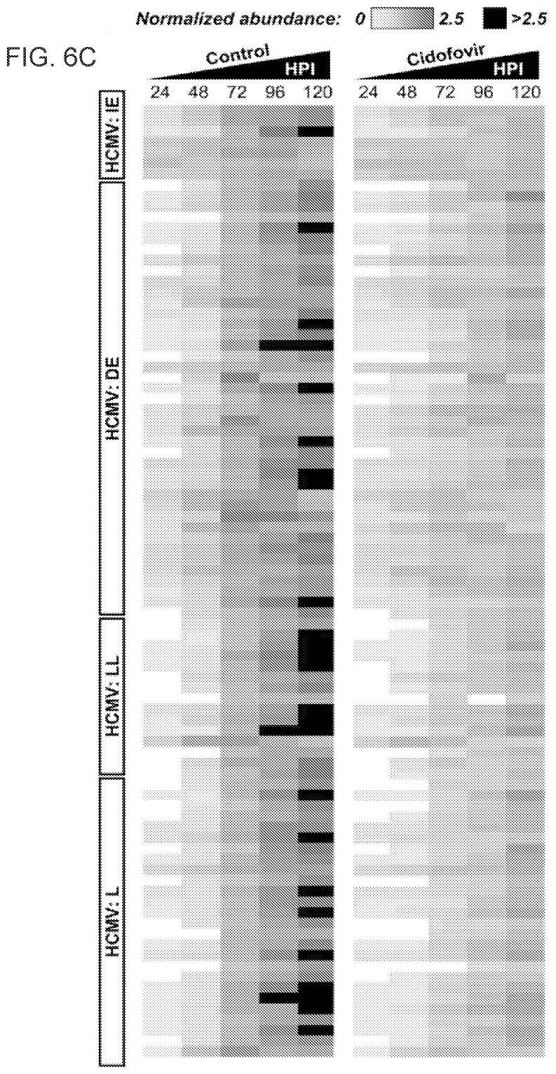

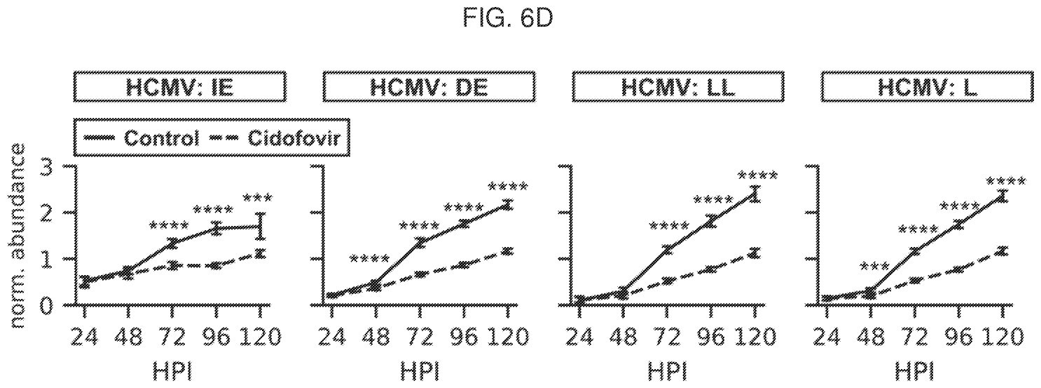

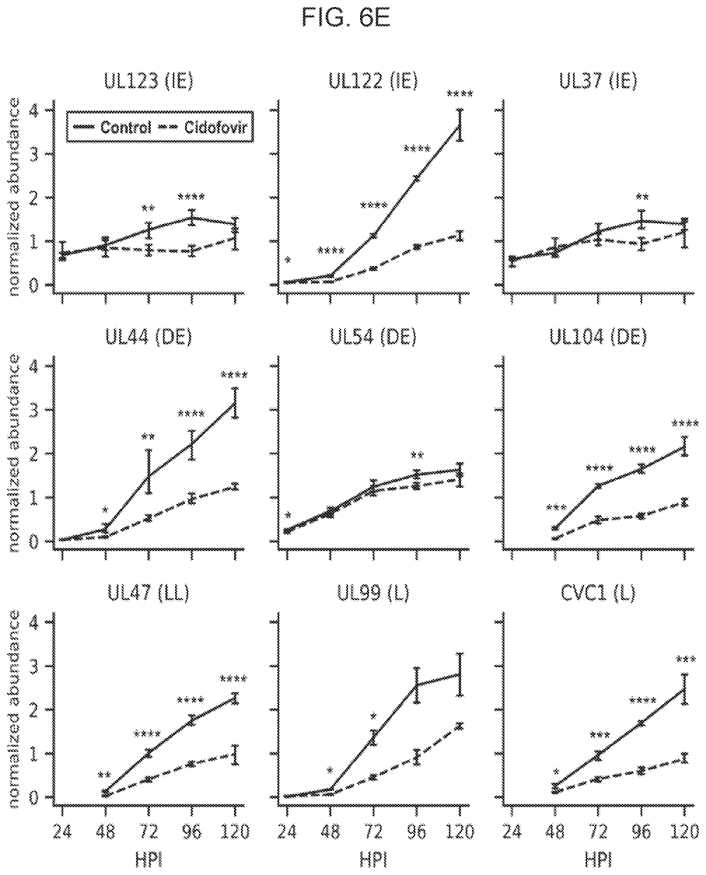

Having established that the TRUSTED assays reliably capture herpesvirus temporal gene expression, the performance of the assay in recognizing different infection levels, i.e. the number of incoming viral particles per cell (multiplicity of infection; MOI) was characterized. To this end, PRM was performed on cells that were subjected to increasing amounts of HCMV virus by infecting at MOIs of 0.05, 0.25, 1.25, and 6.25. Even at low MOIs (MOI=0.05 or 0.25), it was found that nearly all of the targeted peptides and proteins reached detectable levels by 120 HPI (FIGS. 5A-5C). At higher levels of infection (MOI=1.25 or 6.25) all were detectable even earlier, by 24-72 HPI. Among the proteins that were detectable at low MOIs early during infection were IE proteins, such as UL122, UL123, UL13, UL36, and UL37, as well as the most abundant HCMV viral tegument protein, UL83 (FIG. 6C) (Murphy et al., Proc. Natl. Acad. Sci. U.S.A 100, 14976-14981, 2003; Varnum et al., J. Virol. 78, 10960-10966, 2004). As expected, for most proteins, abundance increased with increasing MOI (FIG. 5D) and the extent of this increase was approximately linear with respect to the theoretical percent of cells infected at a given MOI. However, a subset of proteins within the US12 family did not conform to this pattern, including US12 (DE) and US15 (LL) (FIG. 5E). Proteins within the US12 family are known for their immunomodulatory capacity and have previously been shown to be targeted for lysosomal degradation (Fielding et al., Elife 6, 2017). As such, their decrease in abundance at high MOIs is perhaps unsurprising—yet, these results demonstrating that these proteins do not appear to be degraded at low MOIs suggest that there may be a previously unappreciated threshold of US12 family protein expression that must be reached before these proteins are targeted for degradation. Overall, these results demonstrate that these PRM assays are applicable to wide range of MOIs, with low MOIs being closer to physiological levels and high MOIs being commonly employed in research studies (e.g., for achieving synchronous infections).

### PRM Application to Investigations of Clinically Employed Herpesvirus Antiviral Drugs

To demonstrate the utility of the PRM assays for screening antiviral compounds, viral protein abundance dynamics upon treatment with canonical herpesvirus antiviral drugs was next monitored. Fibroblast cells were treated with acyclovir (ACV) or cidofovir (CDV), two compounds used in the clinic as treatments for HSV-1 and HCMV infections, respectively (Kimberlin & Whitley, In Human Herpesviruses: Biology, Therapy, and Immunoprophylaxis, (Cambridge University Press), pp. 1153-1174, 2007; Lurain & Chou, Clin. Microbiol. Rev. 23, 689-712, 2010). Both ACV and CDV hinder viral replication by acting as nucleoside (ACV) or nucleotide (CDV) analogues that selectively inhibit viral DNA polymerases (Biron, In Human Herpesviruses: Biology, Therapy, and Immunoprophylaxis, (Cambridge University Press), pp. 1219-1250, 2007). Both drugs target the same viral process, yet ACV is a more potent inhibitor of HSV-1 than HCMV and the converse is true for CDV (Kimberlin & Whitley, In Human Herpesviruses: Biology, Therapy, and Immunoprophylaxis, (Cambridge University Press), pp. 1153-1174, 2007; Lurain & Chou, Clin. Microbiol. Rev. 23, 689-712, 2010). Although their mechanism of action and impact on virus production are well-established, how these drugs broadly affect the landscape of viral protein abundances remains less understood, with the exception of a proteomics study performed for HSV-1 after ACV treatment (Bell et al., J. Proteome Res. 12, 1820-1829, 2013). Therefore, whether the PRM assay can provide context to viral protein regulation upon drug treatment during HSV-1 and HCMV infection was investigated. Given their mechanism of action, it was expected that following ACV or CDV treatment viral protein levels would be decreased after DNA replication is inhibited, which occurs around 6 HPI for HSV-1 and 24 HPI for HCMV. Indeed, upon treatment with 1 μM ACV (IC₅₀=2-3 μM in MRC5 cells (Bacon et al., J. Antimicrob. Chemother. 37:303-313, 1996; Brandi et al., Life Sci. 69:1285-1290, 2001; Leary et al., Antimicrob. Agents Chemother. 46:762-768, 2002)), a decrease was observed of ˜20% and ˜35% by and after 12 HPI in levels of E and L HSV-1 proteins, respectively (FIGS. 6A-6B). Among these, a significant reduction in the levels of many HSV-1 proteins known to be involved in viral DNA replication was noted, such as DBP, UL42, UL30, UL8, and UL12, as well as UL48, which is a major activator of viral gene expression (Cohan & Frappier, Virus Res. 298, 2021; Roizman & Campadelli-Fiume, In Human Herpesviruses: Biology, Therapy, and Immunoprophylaxis, (Cambridge University Press), pp. 70-92, 2007). Yet, little effect was observed for IE proteins. Considering that L gene expression is directly dependent on successful DNA replication (Honess & Roizman, J. Virol. 14, 8-19, 1974), it is perhaps anticipated that these proteins would show a more robust response to ACV treatment. It was also noted that among E and L proteins, only a subset were significantly downregulated upon ACV treatment, which is in agreement with a previous study showing that IE proteins are broadly unaffected and only a subset of E and L HSV-1 proteins are decreased by ACV treatment (Bell et al., J. Proteome Res. 12, 1820-1829, 2013).

In contrast to the varied response to ACV, upon treatment of HCMV-infected cells with 1 μM CDV (IC₅₀≈0.5 μM in MRC5 cells (Beadle et al., Antimicrob. Agents Chemother. 46, 2381-2386, 2002; Scott et al., Antimicrob. Agents Chemother. 51, 89-94, 2007)) substantial decreases were observed in HCMV protein levels across all temporal classes of gene expression (FIGS. 6C-6E). By 72 HPI more than 85% of the proteins monitored exhibited decreases of at least 35% compared to the PBS control. Moreover, among all 90 proteins, only a single protein, UL54, was decreased by less than 20% across all time points, further underscoring the global effects of CDV treatment on HCMV protein expression (FIG. 6E). Nevertheless, a phenotype that was conserved from these observations of ACV treatment was that IE genes were relatively less impacted by CDV treatment compared to other gene classes. In both cases, this likely reflects the relative independence of IE gene expression, as these proteins are characterized by their ability to be transcribed in the absence of de novo protein synthesis (Roizman & Zhou, Virology 479-480, 562-567, 2015). An exception to this observation, however, was that the abundance of the IE protein UL122 was decreased by ˜70% by 120 HPI. This observation may be explained by the fact that the UL122 locus produces at least two alternative protein isoforms that are expressed from alternative downstream promoters, and these isoforms are expressed with late kinetics and depend on successful viral genome replication (Puchtler & Stamminger, J. Virol. 65, 6301-6306, 1991; Stenberg et al., J. Virol. 63, 2699-2708, 1989). The peptides monitored by this PRM assay are within the C-terminal region of the UL122 protein, and thus common to both full-length UL122 and these shorter isoforms.

### Modulation of Antiviral Sirtuin Enzymatic Activity Differentially Regulates Viral Protein Levels During Herpesvirus Infections

In addition to those targeting DNA replication, a variety of other small molecules have been shown to impact herpesvirus production. These include compounds that target sirtuin proteins, which has previously been shown to exhibit antiviral activity against several viruses, including HSV-1 and HCMV (Koyuncu et al., MBio 5, 2014). Sirtuins are a diverse family of seven (SIRT1-7) NAD⁺-dependent deacetylases and deacylases that regulate a range of cellular processes including metabolism, the cell cycle, and gene expression (Choi & Mostoslaysky, Curr. Opin. Genet. Dev. 26, 24-32, 2014; Michan & Sinclair, Biochem. J. 404, 1-13, 2007). Accumulating evidence during infections with both DNA and RNA viruses suggests that sirtuins could serve as potential targets for therapeutic intervention (Budayeva et al., J. Virol. 90, 5-8, 2016). It was previously established that using EX-527 or CAY10602 compounds to inhibit or activate SIRT1 enzymatic activity results in increased or decreased HCMV titers, respectively (Koyuncu et al., MBio 5, 2014). Similarly, the broad-spectrum activator of sirtuins, trans-Resveratrol, decreased HCMV titers. The effects of these drugs on the HCMV viral proteome, however, have not been fully investigated, nor has their impact on HSV-1 or KSHV replication and viral protein levels been tested.

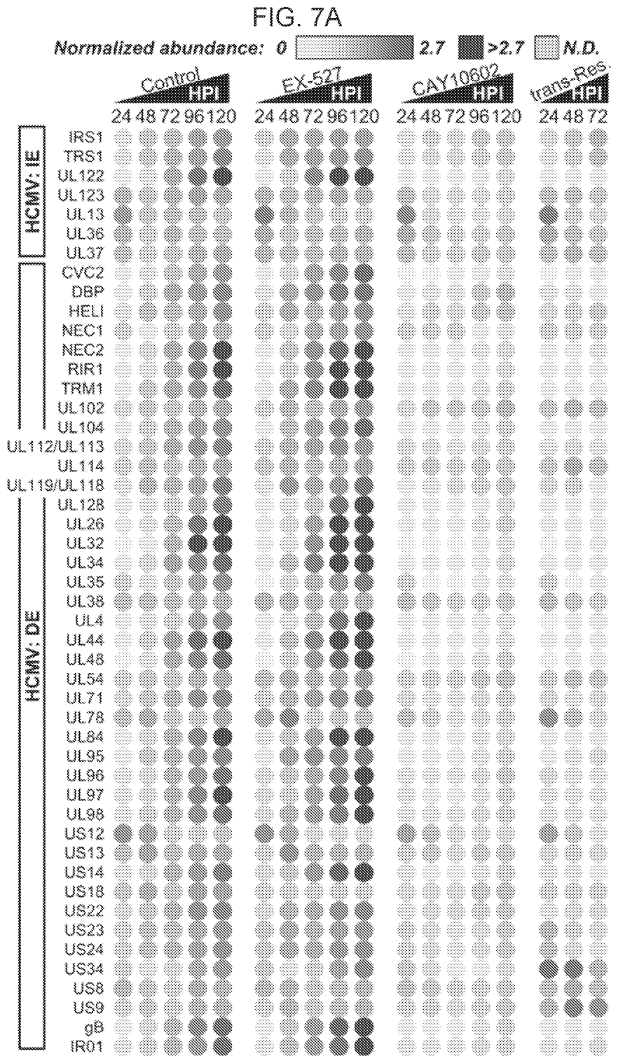

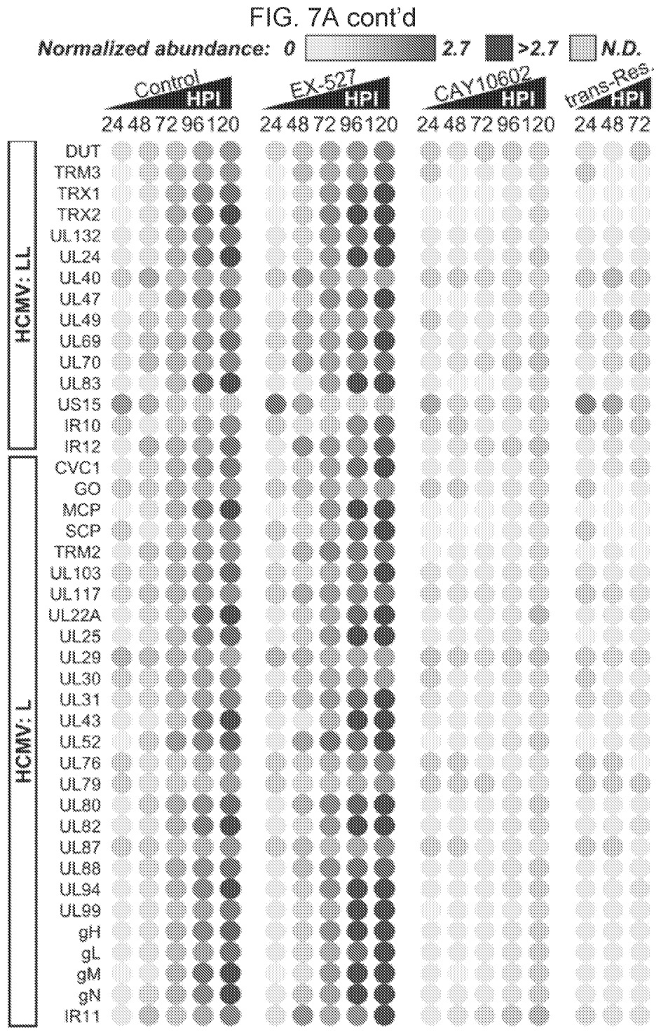

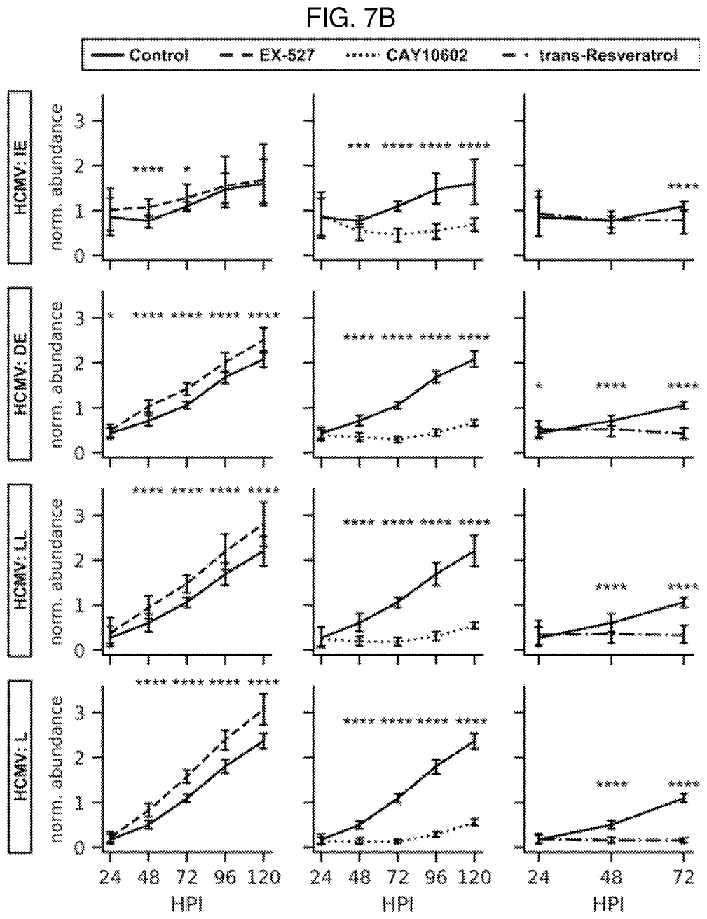

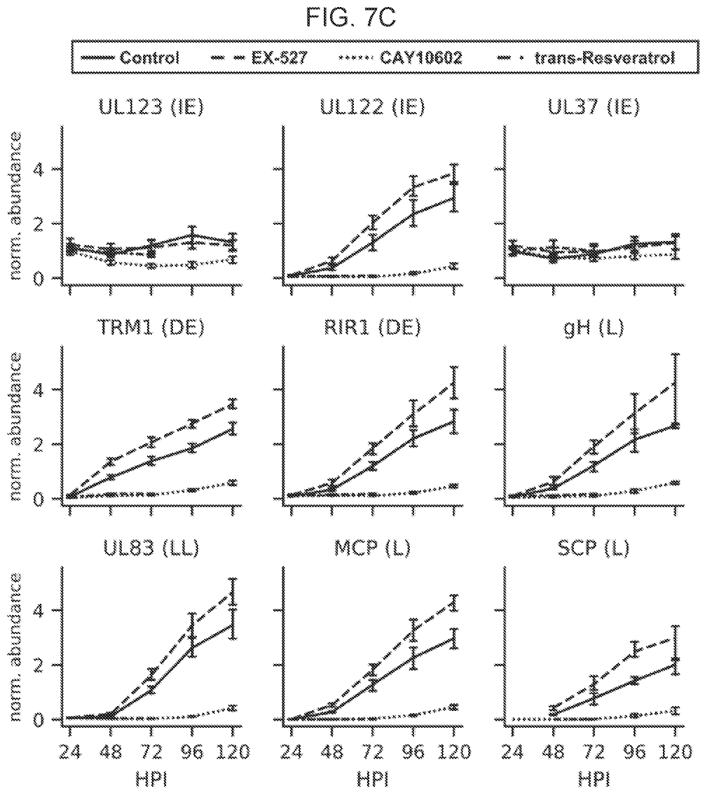

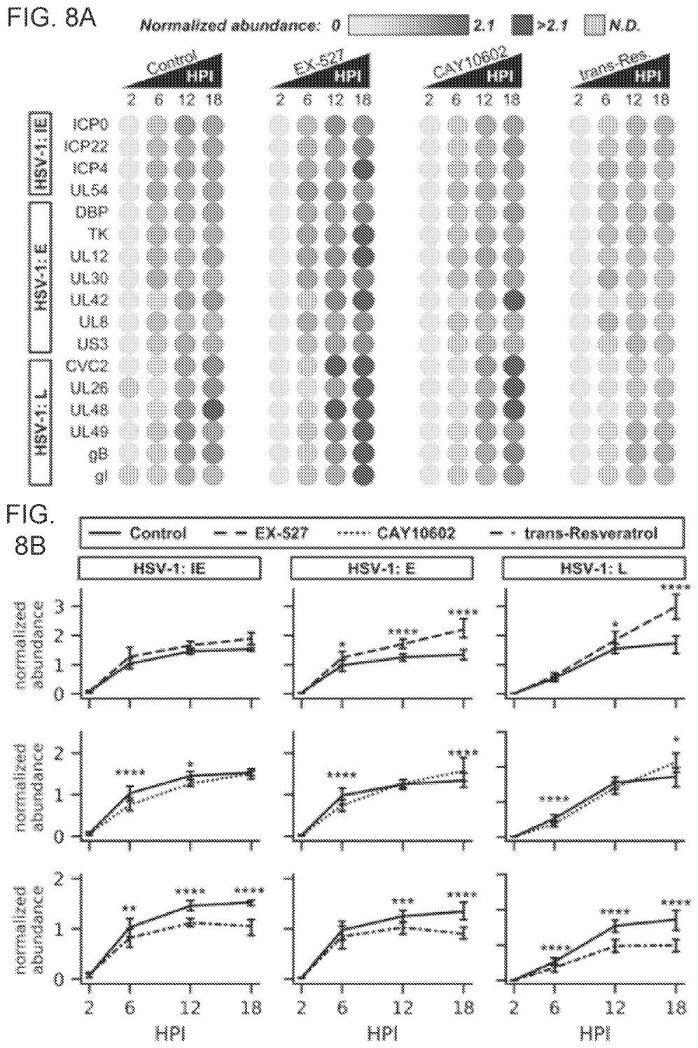

To characterize the effects of SIRT1 activation or inhibition on viral protein levels during HCMV infection, cells were treated with 10 μM EX-527, 12.5 μM CAY10602, or 50 μM trans-Resveratrol and performed the PRM assay. At these concentrations, an increase (EX-527) or decrease (CAY10602 and trans-Resveratrol) of ˜50% in HCMV titers (Koyuncu et al., MBio 5, 2014) had previously been observed. Of the small subset of proteins that had previously quantified been by western blot (UL123, UL26, and UL99) following CAY10602 and trans-Resveratrol treatment (Koyuncu et al., MBio 5, 2014), the PRM results were in agreement with previous observations; UL123 levels were unchanged at 24 HPI, UL26 levels were decreased at 48 HPI, and UL99 levels were robustly decreased by 72 HPI (FIG. 7A). However, these results also revealed that treatment with the sirtuin-activating compounds CAY10602 and trans-Resveratrol induces a global decrease of ˜70% by 120 HPI in viral protein levels (FIGS. 7A-7C). Decreased levels are already observed for IE proteins, in particular for UL122 and UL13. These effects become progressively compounded for DE, LL, and L proteins, an observation similar to the results following CDV treatment. A number of viral proteins (e.g., NEC1, UL76, UL79, UL87, and IR10) become undetectable at early HPIs, displaying delayed expression kinetics upon treatment with sirtuin activators. For most viral proteins, the sirtuin-modulatory effects became more pronounced as the infection progressed. In contrast, EX-527 treatment produced a moderate increase (˜20-40%) in viral protein levels, and these effects were primarily observed for DE, LL, and L proteins (FIGS. 7A-7C). Overall, these results match the previously reported changes in virus titers (Koyuncu et al., MBio 5, 2014), suggesting that alterations in protein levels contribute to the effect of sirtuin-modulatory compounds on virus production.

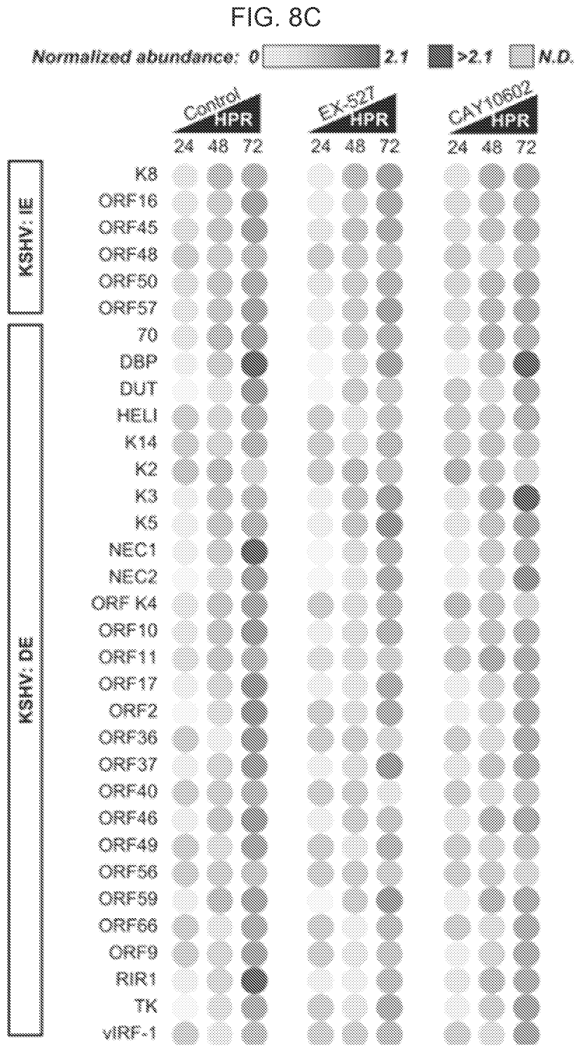

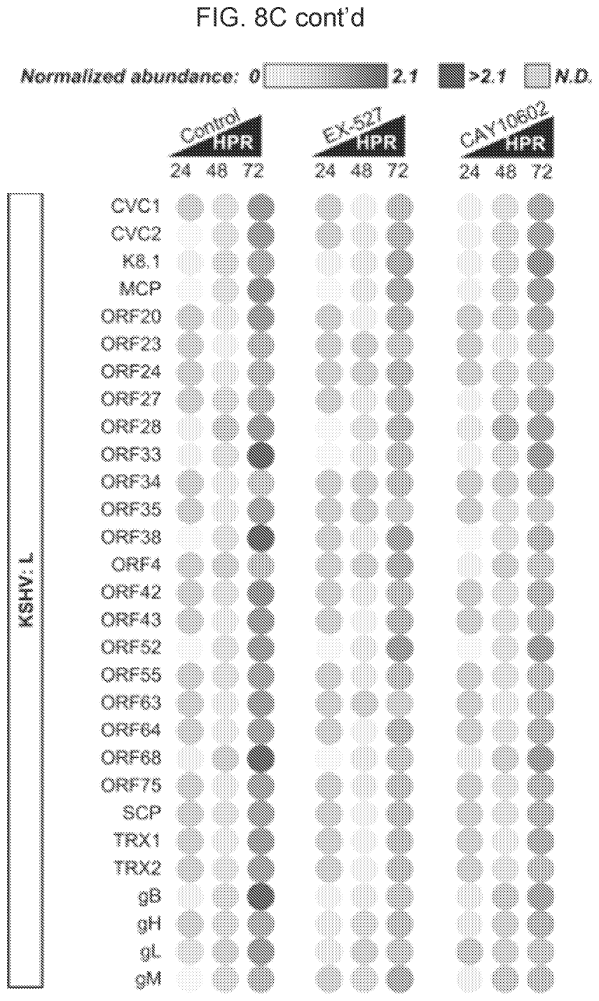

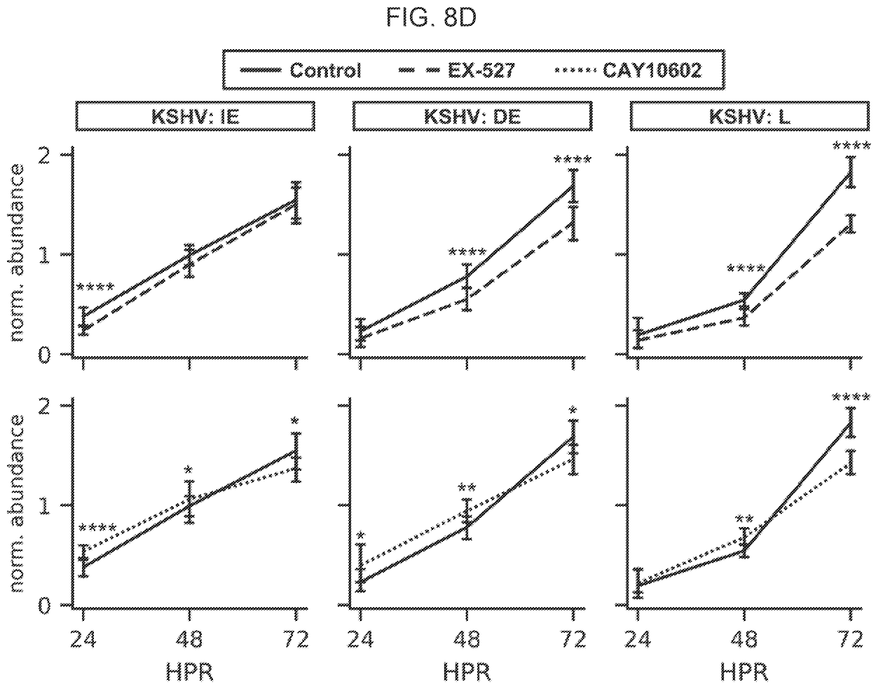

The next question asked was whether treatment with these compounds, at the same concentrations, would also impact viral protein levels in the context of HSV-1 infection and KSHV reactivation. Similar to EX-527 treatment during HCMV infection, an ˜60% increase in E and L HSV-1 protein levels was observed by late time points of infection (FIGS. 8A-8B). On the other hand, trans-Resveratrol decreased protein levels by ˜30-40% and CAY10602 treatment reduced HSV-1 protein levels only moderately at 6 HPI, with levels recovering later in infection. These findings following trans-Resveratrol treatment in fibroblasts agrees with another study that found that, upon a similar treatment, the levels of several monitored viral proteins were reduced in HSV-1-infected neurons (Leyton et al., Virus Res. 205, 63-72, 2015). Among the proteins that were detected, the viral transactivators ICP4 and UL48 (VP16) were strongly upregulated upon EX-527 treatment and downregulated by trans-Resveratrol treatment. Given their essential roles in stimulating viral IE gene expression (Fan et al., Front. Microbiol. 11, 1910, 2020; Roizman & Campadelli-Fiume, In Human Herpesviruses: Biology, Therapy, and Immunoprophylaxis, (Cambridge University Press), pp. 70-92, 2007), it is possible that regulation of ICP4 and UL48 levels could serve as a toggle for modulating HSV-1 gene expression at a more global level in a SIRT1-dependent manner.

Finally, for KSHV, the CAY10602 and EX-527 treatments led to contrasting effects compared to the HCMV and HSV-1 results (FIGS. 8C-8D). Although their changes were subtle (<10%), only seven viral proteins (ORF4, ORF24, ORF37, ORF52, ORF57, ORF59, and gM) displayed a pattern that included both increased and decreased abundances upon treatment with sirtuin inhibitor and activator, respectively. Overall, EX-527 treatment resulted in decreased protein levels at all reactivation time points tested, in particular for DE and L proteins. CAY10602 slightly increased protein levels at early time points (i.e., 24 and 48 HPR), while resulting in decreased proteins levels at the latest time point post-reactivation (i.e., 72 HPR). However, it is of note that most of these changes, although passing significance thresholds, were relatively mild in terms of the fold-change reached. The one exception is the overall decrease in virus protein abundances (by 20-30%) observed for both EX-527 and CAY10602 at 72 HPR.

Altogether, these results confirm and augment understanding of how sirtuin activity-modulating treatments impact protein expression throughout the course of HCMV, HSV-1 and KSHV infections. These results also demonstrate the ability of these assays to contextualize the effects of small molecule treatments, both at the individual and global viral protein levels.

### Conservation of TRUSTED Peptides Indicates Assay Utility Across Diverse Virus Strains

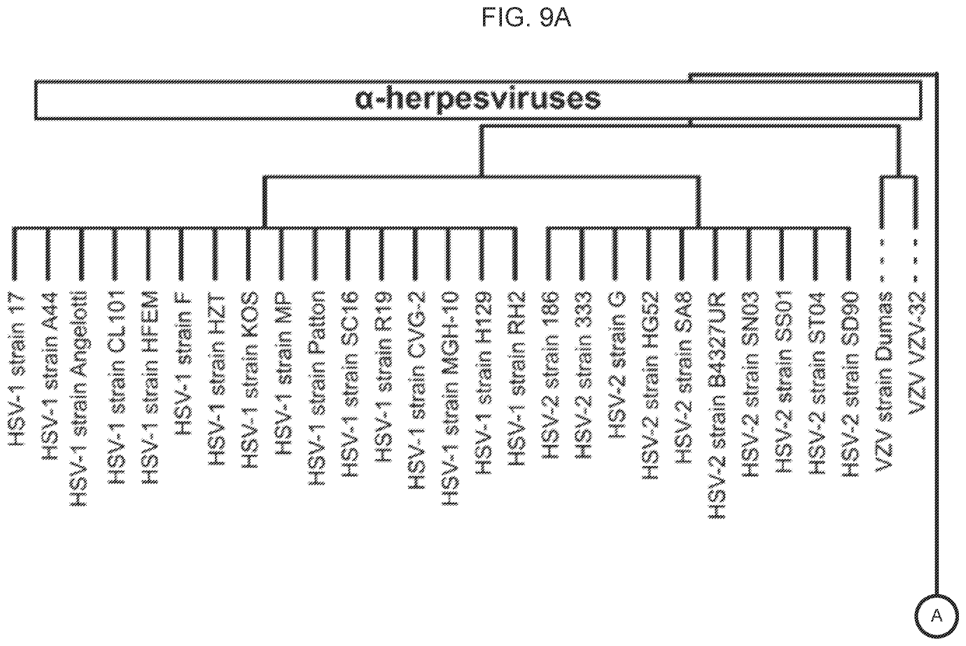

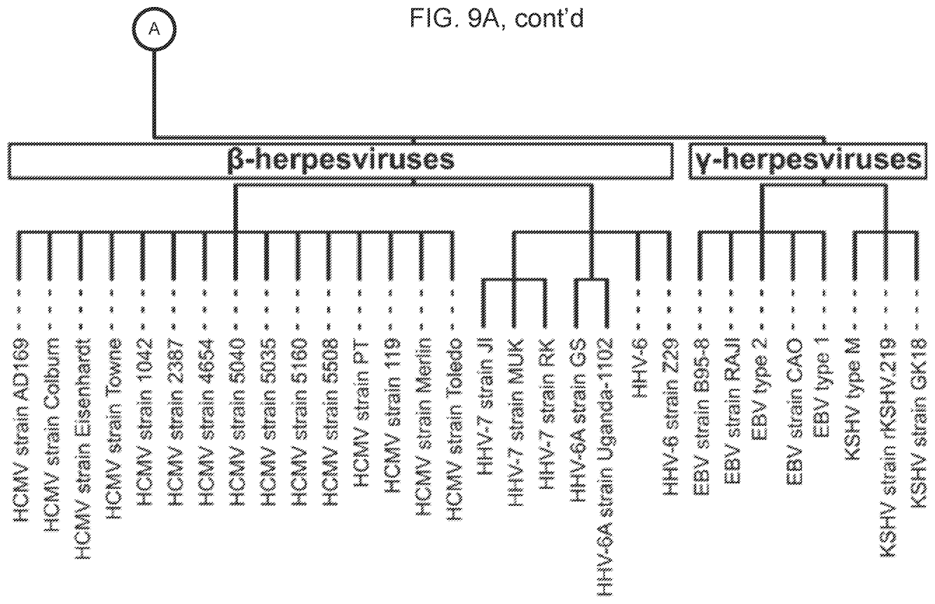

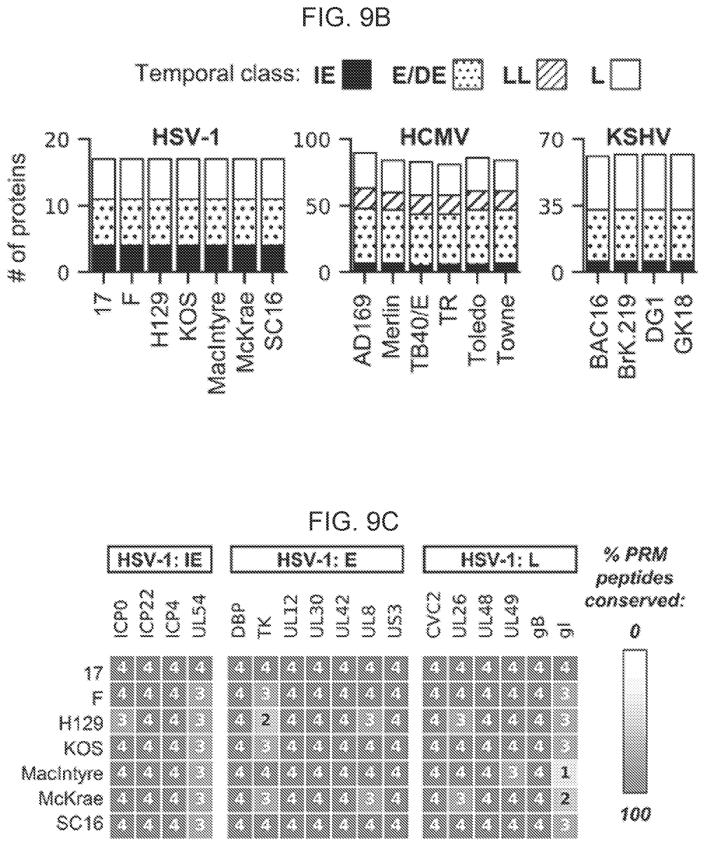

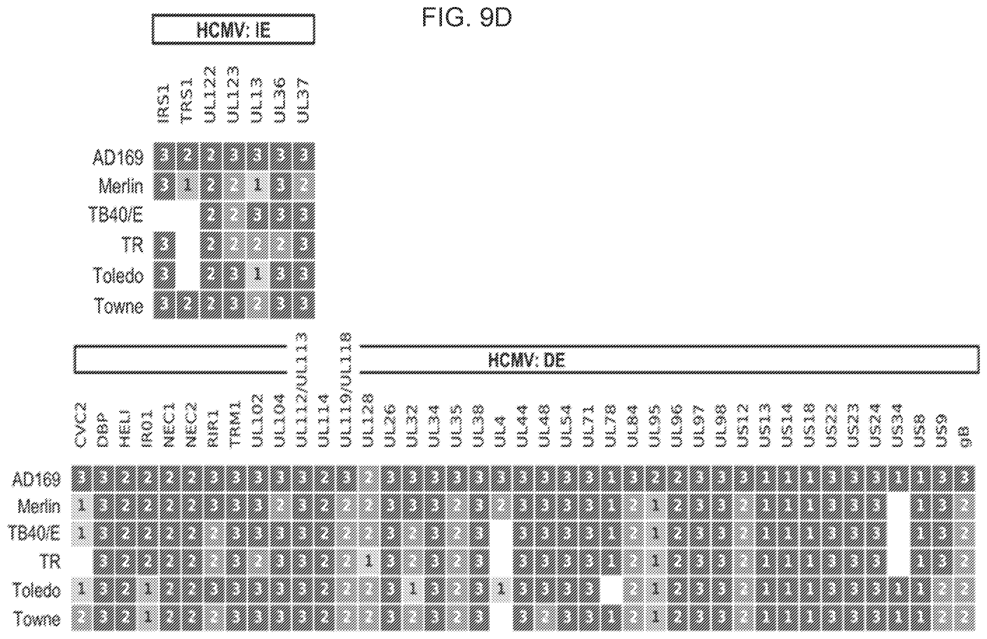

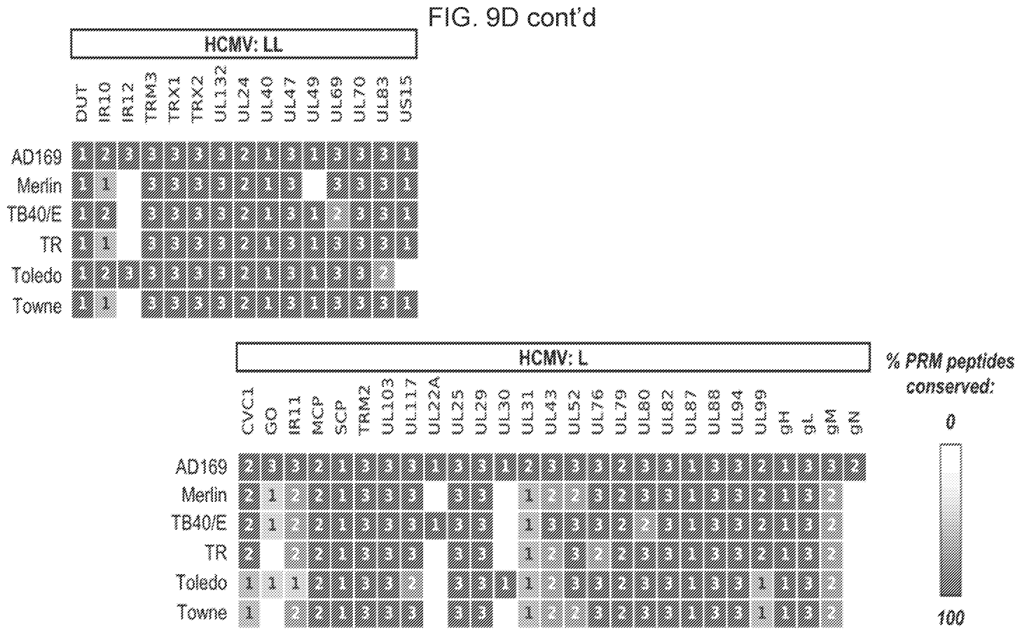

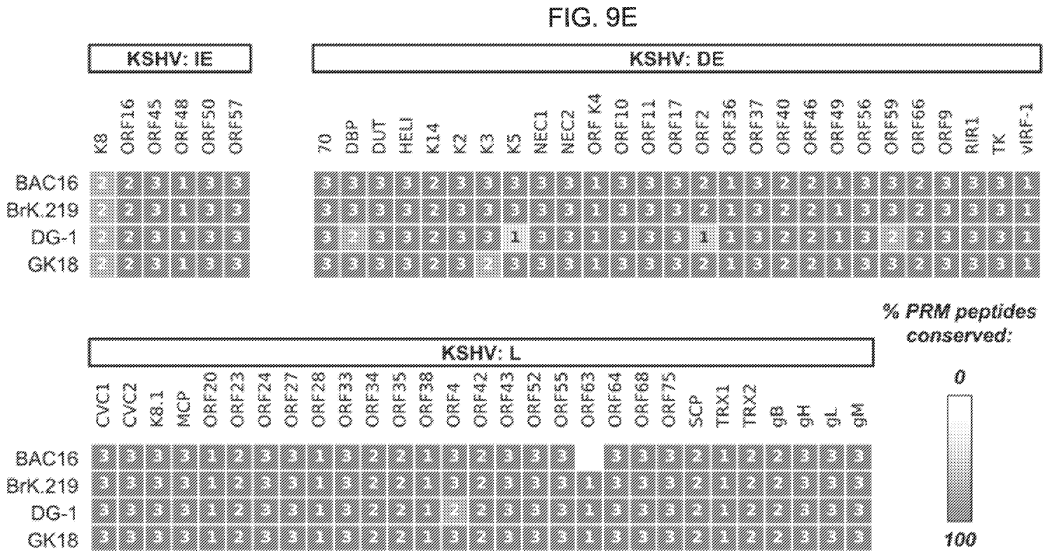

An important consideration when developing a detection assay is its broad applicability—in the current exemplar case, whether this PRM assay is suitable for detecting viral proteins upon infection with a range of HSV-1, HCMV, and KSHV strains. Several laboratory and clinical strains are implemented for the study of each of these viruses, and many have readily accessible complete genome sequences available in online databases (e.g., NCBI, Ensembl). To therefore address the applicability of the assay to different strains (FIG. 9A), a computational analysis was performed of potential peptide sequences represented by the genomes of different HSV-1, HCMV, and KSHV strains in the NCBI nucleotide database to determine the extent of conservation for peptides targeted by the PRM assay. As expected, the analysis demonstrated that ˜100% of the PRM peptides were conserved for HSV-1 strain 17 and HCMV strain AD169, the model strains upon which the PRM assays were developed (FIGS. 9B-9D). A direct comparison to the precise type of KSHV virus produced by the cell line used in this study (iSLK.219) was not possible given the lack of a fully sequenced genome in the NCBI database. However, nearly full conservation was observed when compared to BrK.219, a B-cell line latently infected with the same type of KSHV (rKSHV.219) that is also harbored by iSLK.219 cells (Kati et al., J. Virol. Methods 217, 79-86, 2015) (FIGS. 9B and 9E).

Next the peptide sequences targeted by the TRUSTED assay were compared to those predicted to be present in other HSV-1 strains: F, H129, KOS, MacIntyre, McKrae, and SC16. Among all of these strains near 100% conservation was observed for most proteins targeted by the assay, supporting its broader use for studies with a range of HSV-1 strains. The one exception was the glycoprotein gl (FIG. 9C). Although PRM peptides targeting this viral protein were available for all analyzed strains, most conservation was observed between 17, F, H129, KOS, and SC16 strains, where three-to-four of the optimized PRM peptides for gl were fully conserved. Alternatively, one-to-two peptides were available for the McIntyre and McKrae strains. This is in accordance with previous reports showing that gl exhibits relatively high levels of variation across different HSV-1 strains (Watson et al., Virology 433, 528-537, 2012).

Similarly, for both HCMV and KSHV >90% conservation was observed among the different strains assessed in this analysis. A comparison of laboratory/high-passage (AD169 and Towne) and clinical/low-passage (Toledo, TR, TB40/E, and Merlin) strains of HCMV demonstrated strong conservation across most proteins, with more than 85% of the proteins targeted by the PRM assay having at least one conserved peptide across all strains tested. Similar levels of conservation were observed for the different KSHV strains assessed, which included the laboratory strain BAC16, which was developed for KSHV recombinant virus production (Brulois et al., J. Virol. 86, 9708-9720, 2012), as well as two clinical strains GK18 and DG-1. An important limitation of this analysis, however, is that protein segments resulting from alternative splicing are not captured by this computationally predicted peptide sequences. For both HCMV and KSHV, it was observed that there was one protein for each virus with peptides targeted by the PRM assays that were not predicted to be conserved across any of the strains. In both cases, the proteins in question (UL128 for HCMV and K8 for KSHV) are known to be produced as the result of alternative splicing, and thus were not detected by this analysis. Despite this, overall, these results indicate that the PRM assay developed and described herein will be applicable across a range of virus strains and has the capacity to extend beyond cell culture experiments.

### Discussion

Here, TRUSTED, a targeted MS assay for detecting and quantifying proteins from three model viruses across herpesvirus subfamilies, is presented. The described assays for alpha-, beta-, and gamma-herpesviruses allow for a comprehensive overview of replication cycle progression, while simultaneously quantifying locus-specific changes covering much of the proteomes of these herpesviruses. By applying this technique, 1) the temporal characteristics of the herpesvirus gene expression cascade was captured, 2) a new perspective on canonical herpesvirus treatments has been provided, 3) its applicability to screening anti- and pro-viral compounds, as shown for the modulation of SIRT1 antiviral function, has been examined, and 4) its utility across different laboratory and clinical viral strains was proposed. Ultimately, this approach is broadly applicable to investigating the progression of herpesvirus replication in diverse model systems and in the context of a wide variety of perturbations including small-molecule treatment, antiviral screening, and genetic perturbations.

An important driver for the development of this assay was the lack of commercially available antibodies for a majority of the proteins expressed by these large viruses. By employing targeted MS, viral peptide levels were able to be directly measured in an antibody-independent manner. An equally important driver was the need for methods that provide high throughput detection of viral proteins. In comparison to standard antibody-based methods (e.g., western blot, ELISA), this assay also has the advantage of being highly parallelized, able to simultaneously measure a vast number of viral proteins. Although mRNA measurements also offer throughput, it is known that transcript levels do not always reflect the levels of functional protein products (Ruggles et al., Mol. Cell. Proteomics 16, 959-981, 2017; Vogel & Marcotte, Nat. Rev. Genet. 13, 227-232, 2012; Zhang et al., Nature 513, 382-387, 2014). The described HSV-1, HCMV and KSHV detection assays include peptides from viral proteins belonging to all temporal classes of viral genes, representing the IE, DE, E, LL, and L replication stages of these viruses. Therefore, an informed snapshot of the virus replication state is obtained at a previously unattainable level in 1-2 injections onto the instrument.

The provided herpesvirus detection assays benefit from other advantages characteristic for targeted MS, such as its affordability compared to purchasing equivalent number of antibodies or ELISA kits. Additionally, the detection parameters established for these herpesvirus proteins are readily exportable for use by other groups in a wide variety of model systems (e.g., different cell lines, tissues, animal models). In each of these contexts, it may be necessary to optimize the sample preparation procedure, for example by altering lysis conditions, but the overall parameters of the PRM assays are unlikely to need adjusting. With the exception of the rare scenario where one or more of the normalizing human proteins (e.g., TUBA1A, MYOSA, MHY9) are not expressed or their levels are substantially altered by infection, the peptides targeted in each assay should be readily detected for virus strains where these peptide sequences are conserved. Furthermore, experiments using low MOIs suggest the promise of these assays for detecting viral proteins in clinical samples, and future experiments would be needed to support their use in this context. The continuous increase in access to mass spectrometry instrumentation within academic, industry, and clinical settings further expands the ability to implement these targeted MS assays in a variety of biological and medical investigations.

Following the development of these assays, their performance was validated both in the context of canonical herpesvirus treatments and investigation of other potential antiviral compounds. In doing this, known, as well as previously unappreciated, aspects were uncovered of the effects of the canonical treatments, ACV and CDV, which act as inhibitors of virally encoded DNA polymerases (Biron, In Human Herpesviruses: Biology, Therapy, and Immunoprophylaxis, (Cambridge University Press), pp. 1219-1250, 2007). For both of these established drugs, a reduction in late gene expression was observed during HSV-1 and HCMV infections. However, in contrast to the decreased levels of IE and E proteins that were detected at early time points following CDV treatment during HCMV infection, these results indicate that the expression of IE and E HSV-1 genes increase at 6 HPI after ACV treatment. This has been observed previously (Furman & McGuirt, Antimicrob. Agents Chemother. 23, 332-334, 1983), and a possible explanation for this effect is that when DNA replication is inhibited by ACV, a greater fraction of viral genomes are available for IE and E gene transcription, since they are not actively being used to replicate new viral genomes. However, this increase in viral gene expression for ACV-treated cells relative to control cells could only occur at early time points of infection since the successful replication of viral genomes in control cells later during the infection cycle would ultimately overcome this effect. Alternatively, the increase in HSV-1 gene expression at early time points following ACV treatment could indicate a viral feedback response to the blockage in DNA synthesis, whereby increasing the production of DNA polymerase subunits and processing factors helps to overcome the blockage. Furthermore, this increase could be accomplished through a global increase in protein synthesis rates, as 6 HPI roughly coincides with the peak abundance of these particular IE and E transcripts (Harkness et al., J. Virol. 88, 6847-6861, 2014). Consistent with this model, an increase in total cellular protein synthesis rates was observed at the concentration of ACV used in the study (Furman & McGuirt, Antimicrob. Agents Chemother. 23, 332-334, 1983). Overall, these results not only capture the changes in viral protein abundances that are likely to underlie and result from the antiviral activity of these polymerase-inhibiting drugs, but also further underscore the complex regulation of viral protein levels.

Having assessed the performance of the TRUSTED assays for investigating clinically employed compounds, its applicability for characterizing putative anti- and pro-viral small molecule compounds was tested. As previously shown that the sirtuin family of NAD-deacetylases can restrict herpesvirus replication (Koyuncu et al., MBio 5, 2014), the assays were applied to determine the effects of modulating sirtuin activity on viral protein levels. Although siRNA knockdown or small-molecular modulation of SIRT1 has been shown to affect HCMV titers in a manner consistent with an antiviral role for SIRT1 (Koyuncu et al., MBio 5, 2014), it is not known how these effects are mediated or whether these changes in viral titer are also evident at the HCMV protein level. Here, this was indeed found to be the case, as treatment of HCMV-infected cells with the SIRT1 activators CAY10602 or trans-Resveratrol resulted in a global reduction in viral protein production by 48 HPI. Additionally, treatment with the SIRT1 inhibitor EX-527 was shown to increase HCMV protein levels, particularly toward the end of the virus replication cycle. Altogether, these results establish that SIRT1 enzymatic activity modulates HCMV protein expression—yet, whether these effects are mediated directly or indirectly remains to be investigated. Considering that one of the main targets of SIRT1 is histones, it is possible that SIRT1 enzymatic activity directly regulates viral protein expression by deacetylating histones on viral genomes (Cliffe & Knipe, J. Virol. 82, 12030-12038, 2008; Murphy et al., EMBO J. 21, 1112-1120, 2002; Zalckvar et al., Proc. Natl. Acad. Sci. U.S.A 110, 13126-13131, 2013). Alternatively, it remains to be seen whether SIRT1 can regulate the acetylation status of HCMV proteins, thereby impacting their levels and functions. It is also possible, however, that these effects are indirectly mediated SIRT1. For example, it is well established that SIRT1 deacetylates and inhibits the transcription factor NFκB (Kauppinen et al., Cell. Signal. 25, 1939-1948, 2013), which is essential for driving HCMV protein expression from the major immediate early promoter (MIEP) (Hancock & Nelson, Virol. 1, 2017). Consistent with this notion decreases in UL122 (IE2) and UL123 (IE1) levels were observed upon CAY10602 and trans-Resveratrol treatment, perhaps due to differential MIEP activity. Moreover, considering the robust and global reduction in HCMV protein levels observed following SIRT1 activation by CAY10602 or trans-Resveratrol, it follows that these effects could be driven by altering the levels of essential viral transcription factors like UL122 and UL123.

Ultimately, the impact of SIRT1 modulation on herpesvirus protein levels appears to be broad in nature, as an effect on viral protein levels during HSV-1 infection upon treatments with SIRT1 activators and inhibitors was also observed. Both in the case of HSV-1 and HCMV, it was found that modulating SIRT1 activity with small molecule compounds altered the levels of master viral transcriptional activators, such as ICP4 and UL48 (VP16) for HSV-1 and UL122 and UL123 for HCMV. However, the investigation of the effects of CAY10602 and EX-527 treatment on KSHV protein levels did not follow this pattern. For the KSHV infection model used in this study, reactivation is achieved, in part, by treating with sodium butyrate (NaB). NaB is a broad inhibitor of class I and II HDACs that promotes KSHV reactivation by strongly inhibiting HDAC-mediated silencing of the major lytic transactivator RTA (ORF50) (Lu et al., J Virol 77, 11425-11435, 2003). It has similarly been shown that SIRT1 regulates the reactivation of KSHV via a parallel mechanism (Li et al., J. Virol. 88, 6355-6367, 2014). Notably, the experiments demonstrating a role for SIRT1 in maintaining KSHV latency were performed in a reactivation model different than the one used in this study. As the established protocol for achieving robust KSHV reactivation in the iSLK.219 cell line uses relatively high levels of NaB (Hartenian et al., PLoS Patho. 16, e1008269, 2020), it is possible that the antiviral effects of SIRT1 on the RTA locus are negligible in this context. Therefore, considering the wealth of other SIRT1 targets, as well as the known pleiotropic effects of NaB, one would not necessarily expect the effects of modulating SIRT1 enzymatic activity in a NaB background to properly recapitulate its known antiviral role. Yet, despite the limitation of this reactivation workflow, in combination with the reported role for SIRT1 in regulating RTA, these results suggest that SIRT1 is poised to globally regulate herpesvirus protein levels, perhaps via the regulation of essential viral transcription factor levels.

In summary, this Example demonstrated the value of these TRUSTED assays for globally detecting and quantifying viral proteins from the three main Herpesviridae subfamilies with high accuracy and throughput. These targeted detection methods can offer information about virus biology, as well as provide the means to monitor the effects of small molecules or genetic perturbations in the context of infections. Given the promise for their broad applicability to a range of biological contexts and viral strains, these assays are believed to be of widespread utility. This assay enables development of additional targeted MS assays for the detection of diverse viral pathogens, as well as development of highly needed repositories of signature peptide for virus detection.

### Data and Code Availability

Skyline data analysis files and raw mass spectrometry data have been deposited to PanoramaWeb at online at panoramaweb.org/HerpesvirusPRM.url and are associated with the ProteomeXchange identifier PXD025879. The above data can be accessed with a reviewer account (email: panorama+reviewer29@proteinms.net, password: sUkAlhPS).

### Star Methods

### Cell Lines and Primary Cultures

MRC5 primary human fibroblasts (HFs) (ATCC CCL-171) were used as the model system for HSV-1 and HCMV infections and were cultured in complete growth medium (DMEM supplemented with 10% fetal bovine serum and 1% penicillin-streptomycin antibiotics) at 37° C. and 5% CO₂. iSLK.219 cells harboring latent KSHV (a gift from Dr. Britt Glaunsinger, University of California, Berkeley) were grown in complete growth medium supplemented with 500 μg/ml hygromycin (ThermoFisher Scientific, 10687010) at 37° C. and 5% CO₂. All cells were used for experiments within a maximum of 10 passages.

### Virus Strains and Infections

Wild type HSV-1 strain 17+ (a gift from Beate Sodeik, Hannover Medical School, Hannover, Germany) was propagated as previously described (Diner et al., 2015). Briefly, PO stocks were generated by electroporating pBAC-HSV-1 into U-2 OS cells. Working stocks were then generated from the PO stock by infecting U-2 OS cells at a low level (˜0.001 PFU/cell) and virus was collected ˜3 days later when cells exhibited 100% cytopathic effect. In a similar manner, wild type HCMV strain AD169 was produced from BAC electroporation into HFs and working stocks were propagated by infecting HFs at a low level. In both cases, cell-associated virus was released by sonication, combined with supernatant virus, then concentrated by ultracentrifugation (20,000 rpm, 2 hours, 4° C. with SW28 swinging bucket rotor [Beckman Coulter]) over a 10% ficoll (HSV-1) or 20% sorbitol (HCMV) cushion. Virus stock titers were determined by plaque assay for HSV-1 or tissue culture infectious dose (TCID₅₀) for HCMV and infections were performed at a multiplicity of infection (MOI) of 3. KSHV infections were performed by reactivating iSLK.219 cells with 1 mM sodium butyrate (Sigma-Aldrich, B5887) and 1 μg/ml doxycycline (Sigma, D9891), which resulted in 100% reactivation after 72 hours.

### Small Molecule Treatments and Sample Collection

Acyclovir (Cayman Chemical, 14160), cidofovir (Cayman Chemical, 13113), EX-527 (Cayman Chemical, 10009798), CAY10602 (Cayman Chemical, 10009796), and trans-Resveratrol (Cayman Chemical, 70675) were resuspended in DMSO (acyclovir, EX-527, CAY10602, trans-Resveratrol) or PBS (cidofovir) to generate 2000× stocks that were stored at −80° C. 12 hours prior to virus infection or reactivation, cells were treated with either the small molecule drug or DMSO/PBS control at an equivalent volume. Cell culture concentrations of each drug were as follows: acyclovir (1 μM), cidofovir (1 μM), EX-527 (10 μM), CAY10602 (12.5 μM), and trans-Resveratrol (50 μM). For infection cycles lasting longer than 24 hours, small molecule drugs were re-added to the cell culture medium every 24 hours. Upon collection, cells were rinsed with PBS, scraped into a microcentrifuge tube, pelleted by centrifugation, and rinsed again with PBS. After the addition of 2 μl of protease inhibitor cocktail (Sigma, P8340) sample pellets were snap frozen in liquid nitrogen and stored at −80° C. until ready for mass spectrometry analysis.

### Selection of Target Proteins and Peptides for Targeted Mass Spectrometry Analysis Via Parallel Reaction Monitoring

For all three viral infection models, initial data-dependent analysis runs using the same chromatography conditions as the targeted analyses were performed on the latest timepoint collected in order to identify as many viral proteins and peptides as possible. These identifications were compared to a FASTA file containing the complete viral proteomes of all three viruses plus the human proteome using Skyline (MacLean et al., Bioinformatics 26, 966-968, 2010). Up to four proteotypic peptides for each viral protein detected were selected. In cases where more than three unique peptides were available, peptides were prioritized for selection based first on originating from different regions of the protein and second based on eluting at different points in the chromatogram. Additional peptide selection for proteins not found via data-dependent analysis was performed by successively running unscheduled targeted runs for up to 30 peptides at a time. Peptides initially detected via targeted analysis were confirmed by both manual inspection and automated database search using Sequest HT and Proteome Discoverer™ 2.3. While not every viral protein was detected for each virus, proteins representing all of the temporal classes of viral protein expression are present in the final targeted method.

### Protein Sample Preparation for PRM Analysis

HCMV and KSHV samples: Frozen cell pellets were resuspended in lysis buffer (4% SDS, 50 mM Tris pH 7.5, 100 mM NaCl, 0.5 mM EDTA) and lysed by repeated steps of incubation at 95° C. for 3 min. followed by sonication in a cup-horn sonicator for 20 pulses. Protein concentration was determined by BCA assay and 50-100 μg of protein was then reduced and alkylated at 70° C. for 20 min. using 25 mM TCEP (Thermo Fisher #77720) and 50 mM 2-chloroacetamide (MP Biomedicals #ICN15495580). Protein was then extracted by methanol-chloroform precipitation, resuspended in 25 mM HEPES buffer (pH 8.2), and digested for 16 hours at 37° C. using a 1:50 ratio of trypsin to protein (w/w). The resulting peptides were then adjusted to 1% trifluoroacetic acid (TFA) and desalted using the StageTip method (Rappsilber et al., Nat. Protoc. 2(8):1896-1906, 2007) with C18 material (3M #2215). Finally, bound peptides were washed with 0.5% TFA, eluted with 70% acetonitrile (ACN) and 0.5% formic acid (FA), dried via SpeedVac™ (ThermoFisher), and resuspended in 1% FA and 1% ACN to a concentration of 0.75 μg/μl for peptide LC-MS/MS analysis.

HSV-1 samples: Due to a smaller amount of available starting sample and to demonstrate assay applicability to other peptide preparation methods, HSV-1 samples were prepared using S-Trap (Protifi, C02-micro-80) following the manufacturers protocol. Briefly, samples were resuspended in lysis buffer (9% SDS, 50 mM Tris pH 7.5, 100 mM NaCl, 0.5 mM EDTA) and lysed by repeated steps of incubation at 95° C. for 3 min. followed by sonication in a cup-horn sonicator for 20 pulses. Protein concentration was determined by BCA assay and 30 μg of protein was adjusted to a volume of 40 μl and reduced and alkylated at 70° C. for 20 min. using 25 mM TCEP and 50 mM 2-chloroacetamide. Samples were then acidified to a final concentration of 1.2% aqueous phosphoric acid, mixed with 165 μl of wash buffer solution (90% methanol, 100 mM triethanolamine bicarbonate [TEAB] pH 7.1), and loaded onto the S-trap column. Next, samples were washed 5× with 150 μl of wash buffer, and a 1 hour on-column digestion was performed at 47° C. using a 1:25 ratio of trypsin to protein (w/w) in 25 μl of 25 mM TEAB (pH 8). Digested peptides were then eluted with sequential addition of 40 μl of 25 mM TEAB (pH 8), 40 μl of 0.2% FA, and 70 μl of 50% ACN in 0.2% FA. Finally, pooled elutions were dried via SpeedVac and resuspended in 1% FA and 1% ACN to a concentration of 0.75 μg/μl for peptide LC-MS/MS analysis.

### Peptide LC-MS/MS Analysis

Samples prepared for parallel reaction monitoring (PRM) analysis were analyzed on a Q Exactive HF mass spectrometer (ThermoFisher Scientific) coupled to an EASYSpray ion source (ThermoFisher Scientific). Peptides were resolved for nLC-MS/MS analysis using a Dionex Ultimate 3000 nanoRSLC (ThermoFisher Scientific) equipped with a 25 cm EASYSpray C18 column (ThermoFisher Scientific, ES902). Peptides (1.5 μg) were separated by reverse phase chromatography with solvents A (0.1% formic acid) and B (90% acetonitrile, 0.1% formic acid) at a flow rate of 250 nL/min using a two-phase linear gradient of 2-22% solvent B for 45 min and 22-38% Solvent B for 15 min and were ionized at 1.7 kV. A single duty cycle consisted of an MS-SIM scan (400-2000 m/z range, 15,000 resolution, 15 ms max injection time (MIT), 3×10⁶ automatic gain control (AGC) target) followed by 30 PRM scans (30,000 resolution, 60 ms MIT, 1×10⁵ AGC target, 0.8 m/z isolation window, normalized collision energy (NCE) of 27, 125 m/z fixed first mass) and spectrum data were recorded in profile. Acquisition was controlled by a scheduled inclusion list using 6 min retention time windows. For HSV-1 and KSHV, all peptides were acquired in a single run. For HCMV, the peptide inclusion list was split in half and two injections per sample were made in order to obtain sufficient scans across the peak.

### PRM Data Processing and Analysis

Raw files containing PRM spectra were imported into Skyline and peak quality for all peptides monitored was assessed manually and compared to a reference spectral library. Peptides without convincing spectra or spectra with excessive interference were manually discarded. Following quality control, peptide abundance was calculated from the summed area under the curve (total peak area) for the top three most abundant transition ions per peptide and peptide quantification was exported as a csv file for programmatic analysis in Python. To normalize for differences in input sample, peptide abundances were scaled such that the values of global standard peptides were equivalent, on average, across all input files (e.g. conditions, replicates, injections, etc.). For example, if a single global standard peptide is considered, its summed peak area in a given file is divided by the mean summed peak area across all input files. For each input file, the average of these mean normalized values is then calculated across all global standard peptides that were monitored. Finally, the total peak area values for all peptides monitored by the assay are divided by the input file-specific scaling factor calculated via the above procedure. For data visualization and subsequent analysis, peptide values were then scaled to their mean across replicates, time points, and treatments (where applicable). In some cases, the log-2 fold change for all peptides was also calculated relative to either the first time point that a given peptide was detected (FIG. 4) or relative to a control treatment (FIGS. 6-8).

### Analysis of PRM Peptide Conservation Across Herpesvirus Species and Strains

Peptide conservation analysis was performed by downloading all herpesvirus-associated complete genomes from the NCBI nucleotide database. Potential peptide sequences were then generated for both strands in all reading frames and compared to each peptide targeted by the PRM assay to determine if a given peptide could be produced from a given genome. For virus strains with more than one reported, complete genome deposited in the database, peptides were considered to be conserved as long as they were computationally detected in at least one of these genomes.

### Programs, Software, and Statistics

Data processing and analyses were performed using Python 3.7 in conjunction with Pandas, NumPy, SciPy, Seaborn, and Matplotlib libraries. Significance was determined by two-tailed Student's t-test using the Python SciPy library unless otherwise stated. Where applicable: *p<0.05, ** p<0.01, *** p<0.001, and **** p<0.0001. Figures where constructed in Microsoft PowerPoint.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect, in this context, is an alteration of composition or method that results in a statistically significant change in detection or monitoring or measuring of protein level(s) associates with a herpes virus infection.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds and nucleic acid or amino acids sequences referenced herein that are available in a public database, the information in the database entry is incorporated herein by reference as of the date that the database identifier was first included in the text of an application in the priority chain.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

Table FIG. 6C, part 1 of 2: Numerical values corresponding heatmap in FIG. 6C.

| | | | treatment | | | | |
|---|---|---|---|---|---|---|---|
| | | | PBS | | | | |
| Protein | Protein | | timepoint | | | | |
| Gene | Acc'n | Tempor | 24 | 48 | 72 | 96 | 120 |
| IRS1 | P09715 | IE | 0.250785 | 0.756381 | 1.451441 | 1.694721 | 2.004621 |
| TRS1 | P09695 | IE | 0.140825 | 0.676574 | 1.620029 | 1.723719 | 2.095501 |
| UL122 | P19893 | IE | 0.068637 | 0.209524 | 1.124834 | 2.43485 | 3.650415 |
| UL123 | P13202 | IE | 0.681151 | 0.907494 | 1.262795 | 1.539325 | 1.386181 |
| UL13 | P16755 | IE | 0.631837 | 0.8794 | 1.604159 | 1.73045 | 1.131117 |
| UL36 | P16767 | IE | 0.918981 | 0.843071 | 1.080198 | 1.279079 | 1.009684 |
| UL37 | P16778 | IE | 0.592152 | 0.73698 | 1.219448 | 1.463375 | 1.390296 |
| CVC2 | P16726 | DE | | 0.258661 | 1.08198 | 1.299644 | 2.147224 |
| DBP | P17147 | DE | 0.052836 | 0.349198 | 1.213731 | 1.65021 | 2.421545 |
| HELI | P16736 | DE | 0.106083 | 0.572051 | 1.197691 | 1.778849 | 2.358703 |
| NEC1 | P16794 | DE | | | 0.720243 | 1.034519 | 1.569651 |
| NEC2 | P16791 | DE | 0.01666 | 0.225026 | 1.185219 | 2.090479 | 2.671696 |
| RIR1 | P16782 | DE | 0.049137 | 0.212205 | 1.222715 | 1.994853 | 2.493298 |
| TRM1 | P16724 | DE | | 0.2566 | 1.023039 | 1.600806 | 2.309575 |
| UL102 | P16827 | DE | 0.304174 | 0.846621 | 1.275958 | 1.424813 | 1.674066 |
| UL104 | P16735 | DE | | 0.295404 | 1.262875 | 1.650401 | 2.151989 |
| UL112/UL113 | P17151 | DE | 0.413674 | 0.504576 | 1.26149 | 1.587395 | 2.269971 |
| UL114 | P16769 | DE | 0.301202 | 0.753335 | 1.180486 | 1.59813 | 1.698169 |
| UL119/UL118 | P16739 | DE | 0.10991 | 0.870682 | 1.79184 | 1.722192 | 1.787611 |
| UL128 | P16837 | DE | 0.147551 | 0.221898 | 0.918828 | 1.577666 | 2.480548 |
| UL26 | P16762 | DE | 0.05108 | 0.207762 | 1.360138 | 1.944208 | 2.5785 |
| UL32 | P08318 | DE | 0.024197 | 0.078473 | 1.053011 | 1.971722 | 2.471693 |
| UL34 | P16812 | DE | 0.053016 | 0.287237 | 1.698087 | 2.581309 | 2.860096 |
| UL35 | P16766 | DE | | 0.108128 | 0.734779 | 1.502527 | 2.102323 |
| UL38 | P16779 | DE | 0.514737 | 0.744087 | 1.274677 | 1.528141 | 1.607453 |
| UL4 | P17146 | DE | | 0.544291 | 2.277638 | 0.978331 | 1.120444 |
| UL44 | P16790 | DE | 0.032732 | 0.273312 | 1.48212 | 2.215076 | 3.148486 |
| UL48 | P16785 | DE | | 0.163114 | 1.03521 | 1.661073 | 1.995695 |
| UL54 | P08546 | DE | 0.258098 | 0.687352 | 1.243172 | 1.520513 | 1.631654 |
| UL71 | P16823 | DE | 0.208212 | 0.63463 | 2.012666 | 1.605904 | 1.554933 |
| UL78 | P16751 | DE | 0.17223 | 1.147585 | 1.601803 | 1.706453 | 1.776326 |
| UL84 | P16727 | DE | 0.083629 | 0.333997 | 1.234403 | 2.136399 | 2.84944 |
| UL95 | P16801 | DE | | 0.313896 | 1.206258 | 1.705688 | 2.373253 |
| UL96 | P16787 | DE | 0.348955 | 0.52491 | 1.380551 | 2.184377 | 2.372318 |
| UL97 | P16788 | DE | 0.178576 | 0.353446 | 1.277154 | 2.27615 | 2.999907 |
| UL98 | P16789 | DE | 0.176646 | 0.596109 | 1.240219 | 1.934312 | 2.69132 |
| US12 | P09721 | DE | 0.586568 | 1.114854 | 1.63054 | 1.753413 | 0.990584 |
| US13 | P09720 | DE | 0.322888 | 1.099342 | 1.996525 | 1.661279 | 1.262476 |
| US14 | P09719 | DE | 0.221282 | 0.512589 | 2.33877 | 2.260259 | 1.998401 |
| US18 | P69334 | DE | 0.237715 | 1.196709 | 1.802059 | 1.409431 | 1.403785 |
| US22 | P09722 | DE | 0.161242 | 0.648367 | 1.385537 | 1.774462 | 2.428785 |
| US23 | P09701 | DE | 0.333897 | 0.751452 | 1.431044 | 2.043794 | 2.078385 |
| US24 | P09700 | DE | 0.447468 | 0.660882 | 1.430814 | 1.732604 | 2.296272 |
| US8 | P09730 | DE | 0.425692 | 0.981054 | 1.301518 | 1.332339 | 1.735514 |
| US9 | P09729 | DE | 0.276947 | 0.630381 | 1.258728 | 1.680793 | 1.988449 |
| gB | P06473 | DE | 0.067202 | 0.323045 | 1.371855 | 1.533585 | 1.879769 |
| sp\|P09710\|IR01_HCMVA | P09710 | DE | 0.166694 | 0.458022 | 1.583051 | 2.36612 | 2.899611 |
| DUT | P16824 | LL | | 0.426446 | 1.015092 | 1.607234 | 2.217037 |
| TRM3 | P16732 | LL | | 0.194632 | 1.042872 | 1.348334 | 1.685798 |
| TRX1 | P16783 | LL | | 0.105044 | 0.934396 | 1.699969 | 2.709225 |
| TRX2 | P16728 | LL | 0.009752 | 0.103028 | 1.017941 | 1.932234 | 3.225171 |
| UL132 | P69338 | LL | 0.053565 | 0.228255 | 1.465775 | 2.209159 | 2.693363 |
| UL24 | P16760 | LL | 0.065362 | 0.195111 | 1.156665 | 2.082414 | 2.653938 |
| UL40 | P16780 | LL | | 0.454859 | 1.227951 | 1.429929 | 2.019743 |
| UL47 | P16784 | LL | | 0.136439 | 1.009136 | 1.752108 | 2.265806 |
| UL49 | P16786 | LL | | | 0.907803 | 1.163751 | 1.30788 |
| UL69 | P16749 | LL | 0.127901 | 0.333797 | 1.146749 | 1.856518 | 2.622016 |
| UL70 | P17149 | LL | 0.081415 | 0.533152 | 1.306058 | 1.974665 | 2.738696 |
| UL83 | P06725 | LL | 0.032366 | 0.077552 | 1.264046 | 2.529601 | 2.893248 |
| US15 | P09718 | LL | 0.667066 | 1.513413 | 1.807614 | 1.313109 | 0.911894 |
| sp\|P16808\|IR10_HCMVA | P16808 | LL | | | 1.000214 | 1.548755 | 1.585485 |
| sp\|P16810\|IR12_HCMVA | P16810 | LL | | 0.42267 | 1.455845 | 1.495767 | 2.393318 |
| CVC1 | P16799 | L | | 0.25608 | 0.966758 | 1.701526 | 2.474723 |
| GO | P16750 | L | | 0.235202 | 1.13264 | 1.34771 | 2.068163 |
| MCP | P16729 | L | 0.007966 | 0.104896 | 1.096994 | 2.126841 | 3.451037 |
| SCP | Q7M6N6 | L | | 0.10908 | 0.84193 | 1.562422 | 2.21031 |
| TRM2 | P16792 | L | | 0.216727 | 1.131495 | 1.396104 | 1.901399 |
| UL103 | P16734 | L | 0.215361 | 0.271472 | 1.214228 | 2.021008 | 2.195014 |
| UL117 | P16770 | L | 0.222897 | 0.602105 | 1.297717 | 2.062811 | 2.903952 |
| UL22A | P16845 | L | | 0.146262 | 0.789563 | 1.946351 | 2.404015 |
| UL25 | P16761 | L | 0.027601 | 0.177959 | 1.227902 | 1.824382 | 2.137984 |
| UL29 | P16764 | L | 0.455785 | 0.733505 | 1.294604 | 1.6468 | 1.727337 |
| UL30 | P16765 | L | | 0.260338 | 0.811393 | 1.296451 | 2.099247 |
| UL31 | P16848 | L | 0.219951 | 0.302142 | 1.190181 | 1.931344 | 2.945038 |
| UL43 | P16781 | L | 0.072227 | 0.138191 | 1.019467 | 1.976613 | 2.372416 |
| UL52 | P16793 | L | 0.009554 | 0.273543 | 1.072243 | 1.839262 | 2.881923 |
| UL76 | P16725 | L | | 0.175862 | 0.925197 | 1.483273 | 2.073458 |
| UL79 | P16752 | L | | | 1.212119 | 1.353885 | 1.323589 |
| UL80 | P16753 | L | 0.009843 | 0.247173 | 1.222493 | 1.512581 | 2.233543 |
| UL82 | P06726 | L | 0.195721 | 0.300115 | 1.109869 | 2.061503 | 2.947351 |
| UL87 | P16730 | L | | | 0.800856 | 1.240519 | 1.854068 |
| UL88 | P16731 | L | | 0.200969 | 0.969586 | 1.573311 | 2.277961 |
| UL94 | P16800 | L | 0.028986 | 0.221683 | 1.345914 | 2.222315 | 3.391382 |
| UL99 | P13200 | L | 0.017936 | 0.179753 | 1.361859 | 2.557756 | 2.805152 |
| gH | P12824 | L | 0.043819 | 0.29768 | 1.391606 | 1.621845 | 2.88984 |
| gL | P16832 | L | 0.034155 | 0.252536 | 1.028485 | 1.573971 | 2.115138 |
| gM | P16733 | L | 0.056971 | 0.113649 | 1.185737 | 2.025884 | 2.634662 |
| gN | P16795 | L | | 0.224573 | 1.120667 | 1.644274 | 2.012461 |
| sp\|P16809\|IR11_HCMVA | P16809 | L | 0.063932 | 0.984503 | 1.916477 | 1.425699 | 1.4745 |

Table FIG. 6C, part 2 of 2: Numerical values corresponding heatmap in FIG. 6C.

| | | | treatment | | | | |
|---|---|---|---|---|---|---|---|
| | | | CDV | | | | |
| Protein | Protein | | timepoint | | | | |
| Gene | Acc'n | Tempor. | 24 | 48 | 72 | 96 | 120 |
| IRS1 | P09715 | IE | 0.199986 | 0.54076 | 1.017395 | 0.95363 | 1.003233 |
| TRS1 | P09695 | IE | 0.110313 | 0.402986 | 0.901374 | 0.789285 | 1.103319 |
| UL122 | P19893 | IE | 0.053644 | 0.075626 | 0.373843 | 0.8676 | 1.141027 |
| UL123 | P13202 | IE | 0.73244 | 0.851492 | 0.792374 | 0.773245 | 1.073504 |
| UL13 | P16755 | IE | 0.513069 | 0.781242 | 0.85747 | 0.796458 | 1.074796 |
| UL36 | P16767 | IE | 0.988532 | 0.93035 | 0.881822 | 0.833989 | 1.234293 |
| UL37 | P16778 | IE | 0.546603 | 0.865743 | 1.037712 | 0.936036 | 1.211654 |
| CVC2 | P16726 | DE | | | 0.229796 | 0.881199 | 0.958843 |
| DBP | P17147 | DE | 0.04283 | 0.238596 | 0.780163 | 1.215436 | 2.035455 |
| HELI | P16736 | DE | 0.102356 | 0.410569 | 0.713135 | 0.613421 | 1.12763 |
| NEC1 | P16794 | DE | | | 0.245633 | 0.664107 | 1.120549 |
| NEC2 | P16791 | DE | 0.015902 | 0.085671 | 0.691913 | 0.973118 | 1.797276 |
| RIR1 | P16782 | DE | 0.049612 | 0.106458 | 0.44297 | 0.847813 | 1.313438 |
| TRM1 | P16724 | DE | | 0.118782 | 0.544402 | 0.834651 | 1.312144 |
| UL102 | P16827 | DE | 0.291728 | 0.785271 | 1.111084 | 0.933199 | 1.353088 |
| UL104 | P16735 | DE | | 0.062375 | 0.485231 | 0.57286 | 0.893783 |
| UL112/UL113 | P17151 | DE | 0.334496 | 0.516317 | 0.713059 | 0.74387 | 0.879487 |
| UL114 | P16769 | DE | 0.30253 | 0.642947 | 1.007798 | 0.974251 | 1.541152 |
| UL119/UL118 | P16739 | DE | 0.075406 | 0.387858 | 0.917847 | 0.720627 | 0.867826 |
| UL128 | P16837 | DE | 0.147215 | 0.163049 | 0.315652 | 0.811144 | 1.192616 |
| UL26 | P16762 | DE | 0.049544 | 0.107348 | 0.497253 | 1.007169 | 1.563873 |
| UL32 | P08318 | DE | 0.016161 | 0.031134 | 0.230341 | 0.855086 | 1.478301 |
| UL34 | P16812 | DE | 0.053761 | 0.101766 | 0.575404 | 0.684934 | 0.944404 |
| UL35 | P16766 | DE | | | 0.334648 | 0.664653 | 1.132697 |
| UL38 | P16779 | DE | 0.500743 | 0.749414 | 0.934287 | 0.874868 | 1.271594 |
| UL4 | P17146 | DE | | 0.074474 | 0.081455 | 1.514433 | 0.634991 |
| UL44 | P16790 | DE | 0.032501 | 0.095565 | 0.519712 | 0.965386 | 1.23511 |
| UL48 | P16785 | DE | | 0.043328 | 0.466482 | 0.754106 | 1.099828 |
| UL54 | P08546 | DE | 0.215351 | 0.624271 | 1.148686 | 1.255569 | 1.415334 |
| UL71 | P16823 | DE | 0.297774 | 0.389946 | 0.767965 | 1.299094 | 1.228876 |
| UL78 | P16751 | DE | 0.183363 | 0.653348 | 1.047713 | 0.790342 | 0.920837 |
| UL84 | P16727 | DE | 0.082702 | 0.129705 | 0.394805 | 0.777919 | 1.217286 |
| UL95 | P16801 | DE | | 0.12153 | 0.333455 | 0.55959 | 0.727477 |
| UL96 | P16787 | DE | 0.338998 | 0.359515 | 0.511079 | 0.714223 | 1.265073 |
| UL97 | P16788 | DE | 0.168506 | 0.250163 | 0.525763 | 0.833774 | 1.136561 |
| UL98 | P16789 | DE | 0.178545 | 0.356072 | 0.674095 | 1.01228 | 1.140401 |
| US12 | P09721 | DE | 0.639747 | 0.719166 | 1.060776 | 0.668834 | 0.661029 |
| US13 | P09720 | DE | 0.217406 | 0.725446 | 1.061914 | 0.874107 | 0.778618 |
| US14 | P09719 | DE | 0.190274 | 0.284909 | 0.81128 | 0.684521 | 0.697716 |
| US18 | P69334 | DE | 0.316053 | 0.762455 | 1.076368 | 1.116389 | 0.679037 |
| US22 | P09722 | DE | 0.170113 | 0.400723 | 0.758502 | 1.104873 | 1.167395 |
| US23 | P09701 | DE | 0.343519 | 0.497448 | 0.711426 | 0.785243 | 1.023792 |
| US24 | P09700 | DE | 0.29728 | 0.475328 | 0.665269 | 0.783188 | 1.043264 |
| US8 | P09730 | DE | 0.331677 | 0.909091 | 1.064029 | 0.779572 | 1.139513 |
| US9 | P09729 | DE | 0.230799 | 0.611225 | 1.072598 | 0.907894 | 1.342186 |
| gB | P06473 | DE | 0.051334 | 0.189332 | 0.724313 | 0.995867 | 1.203063 |
| sp\|P09710\|IR01_HCMVA | P09710 | DE | 0.15142 | 0.216371 | 0.452832 | 0.496683 | 0.784905 |
| DUT | P16824 | LL | | 0.29049 | 0.62746 | 0.782325 | 1.033917 |
| TRM3 | P16732 | LL | | | 0.491309 | 0.702517 | 0.985491 |
| TRX1 | P16783 | LL | | 0.01277 | 0.336317 | 0.840918 | 1.114209 |
| TRX2 | P16728 | LL | 0.008754 | 0.019515 | 0.361876 | 0.84128 | 1.493121 |
| UL132 | P69338 | LL | 0.040479 | 0.113114 | 0.451618 | 0.688745 | 0.867732 |
| UL24 | P16760 | LL | | 0.107038 | 0.392259 | 0.708459 | 1.171435 |
| UL40 | P16780 | LL | | 0.390533 | 0.781149 | 0.746709 | 0.949128 |
| UL47 | P16784 | LL | | 0.030524 | 0.411373 | 0.760447 | 0.987849 |
| UL49 | P16786 | LL | | | 0.594833 | | 0.6463 |
| UL69 | P16749 | LL | 0.167573 | 0.255881 | 0.675926 | 0.90319 | 1.466744 |
| UL70 | P17149 | LL | 0.068242 | 0.314259 | 0.743004 | 0.832734 | 1.254916 |
| UL83 | P06725 | LL | 0.03389 | 0.037905 | 0.42756 | 1.026985 | 1.676848 |
| US15 | P09718 | LL | 0.587918 | 1.200814 | 0.889925 | 0.610799 | 0.497447 |
| sp\|P16808\|IR10_HCMVA | P16808 | LL | | | 0.274276 | 0.685864 | 0.905406 |
| sp\|P16810\|IR12_HCMVA | P16810 | LL | | 0.210264 | 0.687537 | 0.572596 | 0.762004 |
| CVC1 | P16799 | L | | 0.114247 | 0.407893 | 0.599964 | 0.877464 |
| GO | P16750 | L | | | 0.600957 | 0.567844 | 0.820979 |
| MCP | P16729 | L | 0.009303 | 0.014442 | 0.400636 | 0.875489 | 1.664133 |
| SCP | Q7M6N6 | L | | | 0.373261 | 0.67795 | 1.225047 |
| TRM2 | P16792 | L | | 0.061302 | 0.550456 | 0.834268 | 1.243658 |
| UL103 | P16734 | L | 0.236005 | 0.195378 | 0.564237 | 0.898964 | 1.157411 |
| UL117 | P16770 | L | 0.198288 | 0.387502 | 0.586738 | 0.585824 | 0.837033 |
| UL22A | P16845 | L | | 0.070247 | 0.419873 | 0.584165 | 1.639524 |
| UL25 | P16761 | L | 0.023156 | 0.064258 | 0.460618 | 0.844308 | 1.445088 |
| UL29 | P16764 | L | 0.435242 | 0.630488 | 0.854916 | 0.892122 | 1.329202 |
| UL30 | P16765 | L | | | 0.411482 | 0.544486 | 0.912512 |
| UL31 | P16848 | L | 0.263208 | 0.237948 | 0.422259 | 0.6245 | 1.093568 |
| UL43 | P16781 | L | 0.068983 | 0.09529 | 0.240246 | 0.777663 | 1.1093 |
| UL52 | P16793 | L | 0.013908 | 0.086712 | 0.465787 | 0.876604 | 1.492921 |
| UL76 | P16725 | L | | | 0.303513 | 0.420981 | 0.739568 |
| UL79 | P16752 | L | | | 0.471315 | 0.558584 | 0.8598 |
| UL80 | P16753 | L | 0.007269 | 0.040807 | 0.658131 | 1.01737 | 1.248889 |
| UL82 | P06726 | L | 0.21355 | 0.214759 | 0.494683 | 0.993159 | 1.469289 |
| UL87 | P16730 | L | | | | 0.318733 | 0.571648 |
| UL88 | P16731 | L | | 0.050213 | 0.428913 | 0.682707 | 0.808781 |
| UL94 | P16800 | L | 0.031925 | 0.092581 | 0.547424 | 0.852067 | 1.265724 |
| UL99 | P13200 | L | 0.017968 | 0.065129 | 0.455047 | 0.911138 | 1.628261 |
| gH | P12824 | L | 0.033031 | 0.130388 | 0.754226 | 1.267968 | 1.569596 |
| gL | P16832 | L | 0.032043 | 0.069084 | 0.507315 | 0.675982 | 1.175407 |
| gM | P16733 | L | 0.022403 | 0.031186 | 0.476981 | 0.996641 | 1.476228 |
| gN | P16795 | L | | 0.07597 | 0.514066 | 0.694728 | 1.395662 |
| sp\|P16809\|IR11_HCMVA | P16809 | L | 0.041091 | 0.502024 | 0.869003 | 0.696092 | 0.763362 |

Table FIG. 7A, part one of three: Numerical values corresponding heatmap in FIG. 7A.

| | | | treatment | | | | |
|---|---|---|---|---|---|---|---|
| | | | DMSO | | | | |
| Protein | Protein | | timepoint | | | | |
| Gene | Acc'n | Tempor. | 24 | 48 | 72 | 96 | 120 |
| IRS1 | P09715 | IE | 0.393104 | 0.879093 | 1.037837 | 1.489751 | 1.798934 |
| TRS1 | P09695 | IE | 0.289694 | 0.930518 | 1.175334 | 1.667247 | 2.06192 |
| UL122 | P19893 | IE | 0.0896 | 0.36789 | 1.311 | 2.35221 | 2.93474 |
| UL123 | P13202 | IE | 1.117581 | 0.869304 | 1.200658 | 1.576622 | 1.313666 |
| UL13 | P16755 | IE | 1.808719 | 0.867179 | 0.94627 | 0.972491 | 0.833624 |
| UL36 | P16767 | IE | 1.269471 | 0.756912 | 1.10423 | 0.969856 | 0.935186 |
| UL37 | P16778 | IE | 0.977619 | 0.710908 | 0.879852 | 1.258002 | 1.321659 |
| CVC2 | P16726 | DE | 0.033036 | 0.234398 | 0.843682 | 1.473524 | 1.742806 |
| DBP | P17147 | DE | 0.202937 | 0.725404 | 1.308883 | 1.802648 | 2.135764 |
| HELI | P16736 | DE | 0.375325 | 1.004965 | 0.970409 | 1.405546 | 1.842887 |
| NEC1 | P16794 | DE | | 0.199916 | 0.714128 | 1.415099 | 1.255646 |
| NEC2 | P16791 | DE | 0.047283 | 0.56915 | 1.416441 | 2.121626 | 2.94629 |
| RIR1 | P16782 | DE | 0.093042 | 0.331684 | 1.198452 | 2.209886 | 2.829697 |
| TRM1 | P16724 | DE | 0.071195 | 0.781348 | 1.377334 | 1.848543 | 2.558026 |
| UL102 | P16827 | DE | 0.506281 | 0.822891 | 0.764576 | 1.238179 | 1.430991 |
| UL104 | P16735 | DE | 0.079816 | 0.602142 | 1.160453 | 1.57307 | 1.94716 |
| UL112/UL113 | P17151 | DE | 0.490921 | 0.922144 | 1.447385 | 1.953647 | 2.151198 |
| UL114 | P16769 | DE | 0.687095 | 0.979833 | 0.824552 | 1.255677 | 1.361339 |
| UL119/UL118 | P16739 | DE | 0.279099 | 1.149815 | 1.104291 | 1.524356 | 2.198141 |
| UL128 | P16837 | DE | 0.136473 | 0.235374 | 0.610776 | 1.378586 | 2.301731 |
| UL26 | P16762 | DE | 0.09088 | 0.315592 | 1.083849 | 2.344631 | 3.355264 |
| UL32 | P08318 | DE | 0.021045 | 0.136302 | 1.176787 | 2.926972 | 3.259442 |
| UL34 | P16812 | DE | 0.131957 | 0.571032 | 1.532516 | 2.115785 | 2.611207 |
| UL35 | P16766 | DE | | 0.236243 | 0.856154 | 1.481481 | 1.945895 |
| UL38 | P16779 | DE | 1.024826 | 1.00979 | 1.031389 | 1.23675 | 1.211252 |
| UL4 | P17146 | DE | 0.050876 | 0.262743 | 0.665275 | 1.547969 | 1.940969 |
| UL44 | P16790 | DE | 0.09404 | 0.591005 | 1.585129 | 2.60947 | 2.75765 |
| UL48 | P16785 | DE | 0.023599 | 0.305841 | 1.395709 | 2.145967 | 2.608366 |
| UL54 | P08546 | DE | 0.728126 | 0.878315 | 0.8278 | 1.21377 | 1.404576 |
| UL71 | P16823 | DE | 0.543242 | 0.784585 | 1.155059 | 2.000245 | 2.086693 |
| UL78 | P16751 | DE | 0.978204 | 1.333013 | 0.774078 | 0.786681 | 1.413302 |
| UL84 | P16727 | DE | 0.154744 | 0.485524 | 1.196259 | 2.1062 | 2.969434 |
| UL95 | P16801 | DE | 0.133557 | 0.791183 | 1.273922 | 1.697682 | 2.092853 |
| UL96 | P16787 | DE | 0.395194 | 0.498143 | 1.105626 | 1.818972 | 2.420627 |
| UL97 | P16788 | DE | 0.323982 | 0.407571 | 1.106745 | 2.107496 | 2.743195 |
| UL98 | P16789 | DE | 0.343372 | 0.711319 | 1.204941 | 1.86116 | 2.486448 |
| US12 | P09721 | DE | 1.845409 | 1.611546 | 0.6772 | 0.709222 | 0.878613 |
| US13 | P09720 | DE | 0.952303 | 1.582174 | 1.073425 | 1.468487 | 1.311741 |
| US14 | P09719 | DE | 0.385377 | 0.526746 | 1.238294 | 1.713375 | 2.325983 |
| US18 | P69334 | DE | 0.913165 | 1.421354 | 0.897768 | 1.553597 | 1.663655 |
| US22 | P09722 | DE | 0.466248 | 0.889126 | 1.16792 | 1.961626 | 2.12487 |
| US23 | P09701 | DE | 0.707085 | 0.951967 | 1.043903 | 1.466673 | 1.83079 |
| US24 | P09700 | DE | 0.727117 | 1.032747 | 1.10738 | 1.502221 | 1.903099 |
| US34 | P09709 | DE | 0.64941 | 0.266953 | 0.560355 | 1.338793 | 1.752423 |
| US8 | P09730 | DE | 1.182054 | 0.910335 | 0.737573 | 1.31192 | 1.171373 |
| US9 | P09729 | DE | 0.613619 | 0.881808 | 0.738144 | 1.10986 | 1.178805 |
| gB | P06473 | DE | 0.143939 | 0.487999 | 1.176069 | 2.064371 | 2.610523 |
| IR01 | P09710 | DE | 0.301458 | 0.667166 | 1.138179 | 1.658432 | 2.429139 |
| DUT | P16824 | LL | 0.340306 | 0.70037 | 0.881236 | 1.309266 | 1.788989 |
| TRM3 | P16732 | LL | 0.005785 | 0.492149 | 1.080037 | 1.458589 | 1.800428 |
| TRX1 | P16783 | LL | 0.010183 | 0.233817 | 1.214315 | 2.083469 | 2.464961 |
| TRX2 | P16728 | LL | 0.004367 | 0.293278 | 1.359655 | 2.369769 | 3.039143 |
| UL132 | P69338 | LL | 0.105837 | 0.394871 | 1.148245 | 1.989573 | 2.449843 |
| UL24 | P16760 | LL | 0.125926 | 0.321343 | 1.126095 | 2.281626 | 2.925485 |
| UL40 | P16780 | LL | 0.629045 | 1.327183 | 1.060654 | 1.37884 | 1.494983 |
| UL47 | P16784 | LL | 0.010151 | 0.367838 | 1.322829 | 2.012307 | 2.474483 |
| UL49 | P16786 | LL | 0.129139 | 0.606789 | 0.873072 | 1.323194 | 1.633429 |
| UL69 | P16749 | LL | 0.279007 | 0.764161 | 1.269773 | 1.81729 | 2.431475 |
| UL70 | P17149 | LL | 0.267492 | 1.035599 | 1.102485 | 1.537487 | 2.080954 |
| UL83 | P06725 | LL | 0.045577 | 0.106815 | 1.074583 | 2.613546 | 3.459602 |
| US15 | P09718 | LL | 1.736876 | 1.136256 | 0.634881 | 0.703388 | 0.711988 |
| IR10 | P16808 | LL | | 0.100867 | 0.661253 | 1.105537 | 2.018289 |
| IR12 | P16810 | LL | 0.095529 | 1.214211 | 1.160935 | 1.395809 | 2.36916 |
| CVC1 | P16799 | L | 0.113008 | 0.52015 | 1.127732 | 1.738645 | 2.302152 |
| GO | P16750 | L | | 0.579512 | 1.151258 | 1.199106 | 1.809305 |
| MCP | P16729 | L | 0.004831 | 0.26752 | 1.273651 | 2.260747 | 2.96467 |
| SCP | Q7M6N6 | L | | 0.173111 | 0.773711 | 1.410959 | 2.005696 |
| TRM2 | P16792 | L | 0.046195 | 0.928792 | 1.460667 | 2.100253 | 2.25531 |
| UL103 | P16734 | L | 0.494035 | 0.480031 | 1.135776 | 1.946469 | 2.565965 |
| UL117 | P16770 | L | 0.429142 | 0.826665 | 1.220833 | 1.768765 | 1.872085 |
| UL22A | P16845 | L | 0.043747 | 0.430408 | 0.925235 | 2.382229 | 2.972968 |
| UL25 | P16761 | L | 0.035989 | 0.331394 | 1.053009 | 2.102861 | 2.66582 |
| UL29 | P16764 | L | 1.124493 | 0.981446 | 1.255175 | 1.517049 | 1.390968 |
| UL30 | P16765 | L | | 0.522517 | 1.145859 | 1.589368 | 1.953842 |
| UL31 | P16848 | L | 0.345362 | 0.572116 | 1.05512 | 1.579286 | 2.14508 |
| UL43 | P16781 | L | 0.103108 | 0.149933 | 0.772934 | 2.205217 | 3.10558 |
| UL52 | P16793 | L | 0.028902 | 0.702339 | 1.713372 | 2.124829 | 2.676017 |
| UL76 | P16725 | L | | 0.352753 | 0.753033 | 1.163988 | 1.574809 |
| UL79 | P16752 | L | | 0.398017 | 0.703113 | 0.912349 | 1.622292 |
| UL80 | P16753 | L | 0.048682 | 0.688282 | 1.35164 | 1.881246 | 2.543808 |
| UL82 | P06726 | L | 0.174561 | 0.287136 | 1.187686 | 2.276051 | 2.817713 |
| UL87 | P16730 | L | | 0.704093 | 0.768952 | 1.234206 | 1.820823 |
| UL88 | P16731 | L | 0.045945 | 0.559472 | 1.466787 | 1.883565 | 2.473639 |
| UL94 | P16800 | L | 0.064895 | 0.30074 | 1.187478 | 2.143727 | 2.755272 |
| UL99 | P13200 | L | 0.036289 | 0.289185 | 0.786987 | 1.578262 | 2.299944 |
| gH | P12824 | L | 0.070326 | 0.373848 | 1.219586 | 2.167575 | 2.675225 |
| gL | P16832 | L | 0.048344 | 0.370447 | 0.934334 | 1.631913 | 2.483173 |
| gM | P16733 | L | 0.019516 | 0.203496 | 1.135467 | 2.477414 | 3.039096 |
| gN | P16795 | L | 0.172056 | 0.558447 | 1.172849 | 2.127828 | 2.950601 |
| IR11 | P16809 | L | 0.170317 | 0.995013 | 0.820773 | 1.338677 | 2.149723 |

Table FIG. 7A, part 2 of 4: Numerical values corresponding heatmap in FIG. 7A.

| | | | treatment | | | | |
|---|---|---|---|---|---|---|---|
| | | | EX-527 | EX-527 | EX-527 | EX-527 | EX-527 |
| Protein | Protein | | timepoint | | | | |
| Gene | Acc'n | Tempor. | 24 | 48 | 72 | 96 | 120 |
| IRS1 | P09715 | IE | 0.591008 | 1.40896 | 1.45297 | 1.541626 | 1.656193 |
| TRS1 | P09695 | IE | 0.371537 | 1.435856 | 1.526488 | 1.764696 | 1.945819 |
| UL122 | P19893 | IE | 0.105395 | 0.63015 | 2.032761 | 3.33355 | 3.843622 |
| UL123 | P13202 | IE | 1.225628 | 1.057823 | 1.119377 | 1.31216 | 1.196422 |
| UL13 | P16755 | IE | 2.222267 | 1.115289 | 0.847079 | 0.757733 | 0.636139 |
| UL36 | P16767 | IE | 1.389102 | 0.885226 | 1.039287 | 0.974351 | 1.141714 |
| UL37 | P16778 | IE | 1.192159 | 0.956982 | 0.971239 | 1.136804 | 1.293862 |
| CVC2 | P16726 | DE | 0.047747 | 0.476888 | 1.581313 | 2.030158 | 2.650395 |
| DBP | P17147 | DE | 0.2975 | 1.313526 | 1.848136 | 2.320553 | 2.344675 |
| HELI | P16736 | DE | 0.594308 | 1.451514 | 1.217401 | 1.542745 | 1.963409 |
| NEC1 | P16794 | DE | | 0.466789 | 1.354085 | 1.524392 | 1.901636 |
| NEC2 | P16791 | DE | 0.069187 | 0.912545 | 1.890826 | 2.626156 | 3.323789 |
| RIR1 | P16782 | DE | 0.134451 | 0.57444 | 1.849171 | 3.098451 | 4.254559 |
| TRM1 | P16724 | DE | 0.135066 | 1.362245 | 2.072084 | 2.748651 | 3.47803 |
| UL102 | P16827 | DE | 0.733659 | 1.14895 | 0.965547 | 1.31481 | 1.703236 |
| UL104 | P16735 | DE | 0.0733 | 0.994871 | 1.634889 | 2.027069 | 2.632987 |
| UL112/UL113 | P17151 | DE | 0.608859 | 1.27523 | 1.652788 | 1.839454 | 1.752987 |
| UL114 | P16769 | DE | 0.830077 | 1.236007 | 0.981175 | 1.280067 | 1.561763 |
| UL119/UL118 | P16739 | DE | 0.402952 | 1.772877 | 1.333697 | 1.79256 | 2.466528 |
| UL128 | P16837 | DE | 0.123328 | 0.321942 | 0.930985 | 2.352975 | 3.323697 |
| UL26 | P16762 | DE | 0.116606 | 0.504816 | 1.482611 | 2.920353 | 4.14742 |
| UL32 | P08318 | DE | 0.030696 | 0.296457 | 1.867211 | 3.24201 | 3.991019 |
| UL34 | P16812 | DE | 0.220402 | 0.944509 | 2.267205 | 2.939025 | 3.253491 |
| UL35 | P16766 | DE | 0.014946 | 0.408894 | 1.180604 | 1.818817 | 2.282807 |
| UL38 | P16779 | DE | 1.285338 | 1.288563 | 1.137121 | 1.209506 | 1.30009 |
| UL4 | P17146 | DE | 0.066462 | 0.394409 | 1.091592 | 2.418042 | 3.577224 |
| UL44 | P16790 | DE | 0.114289 | 0.981904 | 2.041512 | 2.948001 | 3.235758 |
| UL48 | P16785 | DE | 0.029578 | 0.584717 | 1.786399 | 2.510836 | 3.146015 |
| UL54 | P08546 | DE | 0.99575 | 1.35587 | 1.105452 | 1.36112 | 1.68656 |
| UL71 | P16823 | DE | 0.753104 | 1.147403 | 1.660189 | 2.01117 | 2.29427 |
| UL78 | P16751 | DE | 1.348929 | 2.165044 | 0.723823 | 0.909449 | 1.081154 |
| UL84 | P16727 | DE | 0.241799 | 0.851559 | 1.673742 | 2.852737 | 3.937379 |
| UL95 | P16801 | DE | 0.209806 | 1.60345 | 1.943516 | 2.053504 | 2.186525 |
| UL96 | P16787 | DE | 0.522785 | 0.765395 | 1.376677 | 2.029292 | 2.905683 |
| UL97 | P16788 | DE | 0.416199 | 0.678445 | 1.499966 | 2.599692 | 3.557208 |
| UL98 | P16789 | DE | 0.426328 | 1.17023 | 1.698151 | 2.285939 | 2.735892 |
| US12 | P09721 | DE | 1.996846 | 1.435294 | 0.450758 | 0.418455 | 0.544134 |
| US13 | P09720 | DE | 0.585645 | 1.946569 | 1.1408 | 1.119837 | 1.262922 |
| US14 | P09719 | DE | 0.429486 | 0.776683 | 1.819842 | 2.676913 | 3.370413 |
| US18 | P69334 | DE | | | | | |
| US22 | P09722 | DE | 0.552733 | 1.298633 | 1.376625 | 1.85749 | 2.04501 |
| US23 | P09701 | DE | 0.908004 | 1.45506 | 1.204828 | 1.489183 | 1.808652 |
| US24 | P09700 | DE | 0.82496 | 1.445037 | 1.338863 | 1.689468 | 1.927756 |
| US34 | P09709 | DE | 0.777937 | 0.306615 | 0.625002 | 1.486055 | 2.076057 |
| US8 | P09730 | DE | 1.120794 | 1.147072 | 0.837019 | 1.053235 | 1.167256 |
| US9 | P09729 | DE | 0.725543 | 1.169757 | 0.777427 | 0.996418 | 1.115877 |
| gB | P06473 | DE | 0.174584 | 0.763851 | 1.749207 | 2.685096 | 3.300573 |
| IR01 | P09710 | DE | 0.264282 | 1.063209 | 1.623899 | 2.376679 | 2.882245 |
| DUT | P16824 | LL | 0.466081 | 0.892085 | 1.314353 | 1.795798 | 2.090512 |
| TRM3 | P16732 | LL | 0.018774 | 1.02261 | 1.588103 | 2.116128 | 2.654605 |
| TRX1 | P16783 | LL | 0.011353 | 0.499105 | 1.766105 | 2.613623 | 3.502978 |
| TRX2 | P16728 | LL | 0.006257 | 0.607848 | 2.026974 | 3.214779 | 4.315337 |
| UL132 | P69338 | LL | 0.133087 | 0.481624 | 1.752206 | 2.465814 | 2.837893 |
| UL24 | P16760 | LL | 0.174373 | 0.494823 | 1.69841 | 3.16897 | 4.18348 |
| UL40 | P16780 | LL | 0.742979 | 1.547923 | 1.310339 | 1.304801 | 1.300485 |
| UL47 | P16784 | LL | 0.019991 | 0.664088 | 1.8425 | 2.452856 | 2.88063 |
| UL49 | P16786 | LL | 0.304803 | 1.186641 | 1.150344 | 1.688097 | 2.22546 |
| UL69 | P16749 | LL | 0.418877 | 1.222353 | 1.612025 | 2.424229 | 3.274744 |
| UL70 | P17149 | LL | 0.374438 | 1.660989 | 1.540933 | 1.912518 | 2.340356 |
| UL83 | P06725 | LL | 0.063099 | 0.197338 | 1.617085 | 3.439916 | 4.663234 |
| US15 | P09718 | LL | 2.346331 | 1.4624 | 0.533046 | 0.516143 | 0.764521 |
| IR10 | P16808 | LL | | 0.271878 | 0.746938 | 1.991968 | 2.411238 |
| IR12 | P16810 | LL | 0.207656 | 1.93902 | 1.710055 | 1.771738 | 2.686219 |
| CVC1 | P16799 | L | 0.183779 | 0.727911 | 1.553613 | 2.453429 | 3.146933 |
| GO | P16750 | L | | 1.033524 | 1.460731 | 1.415713 | 1.549275 |
| MCP | P16729 | L | 0.005908 | 0.526119 | 1.815522 | 3.25922 | 4.298434 |
| SCP | Q7M6N6 | L | | 0.417337 | 1.29177 | 2.484876 | 2.983501 |
| TRM2 | P16792 | L | 0.106868 | 1.377131 | 2.616844 | 2.797888 | 2.914212 |
| UL103 | P16734 | L | 0.52735 | 0.565809 | 1.285224 | 2.322211 | 3.020124 |
| UL117 | P16770 | L | 0.679446 | 1.44872 | 1.692004 | 2.148533 | 2.302078 |
| UL22A | P16845 | L | 0.065004 | 0.628413 | 1.343917 | 2.572579 | 4.430641 |
| UL25 | P16761 | L | 0.057212 | 0.629548 | 1.727173 | 2.732889 | 4.208425 |
| UL29 | P16764 | L | 1.141514 | 1.20704 | 1.247057 | 1.398225 | 1.420313 |
| UL30 | P16765 | L | 0.134401 | 0.806592 | 1.440981 | 1.746032 | 1.615107 |
| UL31 | P16848 | L | 0.4765 | 1.032585 | 1.699196 | 2.532274 | 3.731496 |
| UL43 | P16781 | L | 0.099059 | 0.191361 | 1.150285 | 2.873035 | 4.456903 |
| UL52 | P16793 | L | 0.064504 | 1.489052 | 2.338652 | 2.489357 | 2.793375 |
| UL76 | P16725 | L | | 0.615674 | 1.295207 | 1.685269 | 2.070391 |
| UL79 | P16752 | L | | 0.532744 | 0.879508 | 1.266501 | 1.77947 |
| UL80 | P16753 | L | 0.065986 | 1.21443 | 2.042597 | 2.684956 | 2.976453 |
| UL82 | P06726 | L | 0.182723 | 0.501821 | 1.820873 | 3.011921 | 3.82947 |
| UL87 | P16730 | L | | 0.973663 | 0.966077 | 1.353789 | 1.964296 |
| UL88 | P16731 | L | 0.065985 | 0.988493 | 1.88573 | 2.357611 | 2.658042 |
| UL94 | P16800 | L | 0.110697 | 0.603771 | 1.910088 | 2.903675 | 3.756646 |
| UL99 | P13200 | L | 0.064869 | 0.476312 | 1.185196 | 2.77835 | 3.121477 |
| gH | P12824 | L | 0.102358 | 0.612662 | 1.920063 | 3.132675 | 4.259949 |
| gL | P16832 | L | 0.061456 | 0.54747 | 1.391021 | 2.425249 | 3.085682 |
| gM | P16733 | L | 0.02138 | 0.336849 | 1.720709 | 3.14536 | 4.05328 |
| gN | P16795 | L | 0.205163 | 0.833859 | 1.551963 | 2.766087 | 3.549374 |
| IR11 | P16809 | L | 0.30782 | 1.840514 | 1.308304 | 1.957091 | 2.909996 |

Table FIG. 7A, part 3 of 4: Numerical values corresponding heatmap in FIG. 7A.

| | | | treatment | | | | |
|---|---|---|---|---|---|---|---|
| | | | CAY10602 | | | | |
| Protein | Protein | | timepoint | | | | |
| Gene | Acc'n | Tempor. | 24 | 48 | 72 | 96 | 120 |
| IRS1 | P09715 | IE | 0.393856 | 0.581292 | 0.562487 | 0.782088 | 0.878562 |
| TRS1 | P09695 | IE | 0.264166 | 0.445964 | 0.528428 | 0.714633 | 0.793278 |
| UL122 | P19893 | IE | 0.059973 | 0.05121 | 0.054268 | 0.164207 | 0.433233 |
| UL123 | P13202 | IE | 0.983063 | 0.567181 | 0.445102 | 0.475776 | 0.679516 |
| UL13 | P16755 | IE | 2.054705 | 0.440516 | 0.360393 | 0.298632 | 0.426274 |
| UL36 | P16767 | IE | 1.409841 | 0.911679 | 0.601021 | 0.583624 | 0.811172 |
| UL37 | P16778 | IE | 1.032019 | 0.755659 | 0.719049 | 0.801743 | 0.871547 |
| CVC2 | P16726 | DE | 0.01787 | 0.027105 | 0.03983 | 0.171402 | 0.286394 |
| DBP | P17147 | DE | 0.175602 | 0.321512 | 0.391041 | 0.794094 | 1.236905 |
| HELI | P16736 | DE | 0.338898 | 0.633026 | 0.570933 | 0.801698 | 0.992441 |
| NEC1 | P16794 | DE | | | | 0.0869 | 0.244647 |
| NEC2 | P16791 | DE | 0.039686 | 0.070707 | 0.11001 | 0.288565 | 0.519505 |
| RIR1 | P16782 | DE | 0.093968 | 0.115661 | 0.109921 | 0.209787 | 0.45765 |
| TRM1 | P16724 | DE | 0.040874 | 0.1031 | 0.1317 | 0.313364 | 0.580927 |
| UL102 | P16827 | DE | 0.534341 | 0.78656 | 0.762628 | 0.90061 | 1.127833 |
| UL104 | P16735 | DE | 0.039031 | 0.073986 | 0.100476 | 0.306681 | 0.59692 |
| UL112/UL113 | P17151 | DE | 0.523554 | 0.423049 | 0.380833 | 0.511353 | 0.678676 |
| UL114 | P16769 | DE | 0.670445 | 0.770946 | 0.651681 | 0.788251 | 0.901107 |
| UL119/UL118 | P16739 | DE | 0.219773 | 0.340151 | 0.419498 | 0.669471 | 0.906369 |
| UL128 | P16837 | DE | 0.11131 | 0.067294 | 0.047277 | 0.193737 | 0.537015 |
| UL26 | P16762 | DE | 0.072733 | 0.080212 | 0.102274 | 0.282802 | 0.732739 |
| UL32 | P08318 | DE | 0.019523 | 0.02022 | 0.026785 | 0.116798 | 0.361664 |
| UL34 | P16812 | DE | 0.113716 | 0.079061 | 0.090479 | 0.223108 | 0.475984 |
| UL35 | P16766 | DE | | 0.007792 | 0.028712 | 0.125242 | 0.264786 |
| UL38 | P16779 | DE | 0.975481 | 0.813938 | 0.583926 | 0.667221 | 0.79167 |
| UL4 | P17146 | DE | 0.037361 | 0.071277 | 0.067649 | 0.212807 | 0.363478 |
| UL44 | P16790 | DE | 0.064739 | 0.089135 | 0.078867 | 0.176004 | 0.369143 |
| UL48 | P16785 | DE | 0.011748 | 0.034428 | 0.0801 | 0.280715 | 0.623851 |
| UL54 | P08546 | DE | 0.608876 | 0.717827 | 0.663928 | 0.907533 | 1.076395 |
| UL71 | P16823 | DE | 0.439031 | 0.345939 | 0.305392 | 0.475372 | 0.645052 |
| UL78 | P16751 | DE | 0.975463 | 0.713386 | 0.361614 | 0.423462 | 0.609856 |
| UL84 | P16727 | DE | 0.101209 | 0.075753 | 0.087852 | 0.232838 | 0.562785 |
| UL95 | P16801 | DE | 0.076253 | 0.143747 | 0.156969 | 0.311984 | 0.601709 |
| UL96 | P16787 | DE | 0.387012 | 0.258265 | 0.263185 | 0.411203 | 0.805568 |
| UL97 | P16788 | DE | 0.281246 | 0.172616 | 0.168695 | 0.303747 | 0.606391 |
| UL98 | P16789 | DE | 0.30052 | 0.379642 | 0.296671 | 0.467359 | 0.731457 |
| US12 | P09721 | DE | 1.67214 | 0.944636 | 0.42388 | 0.450544 | 0.468652 |
| US13 | P09720 | DE | 0.683816 | 0.912323 | 0.535105 | 0.889536 | 0.889014 |
| US14 | P09719 | DE | 0.395719 | 0.158868 | 0.155875 | 0.344664 | 0.665223 |
| US18 | P69334 | DE | 0.533463 | 0.536669 | 0.541546 | 0.928291 | 1.010493 |
| US22 | P09722 | DE | 0.396613 | 0.54202 | 0.481265 | 0.623066 | 0.77635 |
| US23 | P09701 | DE | 0.677624 | 0.428294 | 0.393282 | 0.530356 | 0.775833 |
| US24 | P09700 | DE | 0.619953 | 0.419988 | 0.311425 | 0.484959 | 0.818338 |
| US34 | P09709 | DE | 0.798142 | 0.411833 | 0.206614 | 0.212461 | 0.416762 |
| US8 | P09730 | DE | 1.056746 | 0.890473 | 0.579385 | 0.680108 | 0.785804 |
| US9 | P09729 | DE | 0.626989 | 0.611748 | 0.564904 | 0.7084 | 0.852919 |
| gB | P06473 | DE | 0.113671 | 0.096711 | 0.102184 | 0.284986 | 0.658201 |
| IR01 | P09710 | DE | 0.27398 | 0.315724 | 0.258781 | 0.457469 | 0.558401 |
| DUT | P16824 | LL | | 0.258241 | | | 0.544689 |
| TRM3 | P16732 | LL | | 0.016945 | 0.036583 | 0.163805 | 0.382985 |
| TRX1 | P16783 | LL | 0.0077 | 0.006112 | 0.020991 | 0.10701 | 0.339807 |
| TRX2 | P16728 | LL | 0.005245 | 0.006041 | 0.021927 | 0.142292 | 0.440109 |
| UL132 | P69338 | LL | 0.104428 | 0.096623 | 0.128671 | 0.229987 | 0.401115 |
| UL24 | P16760 | LL | 0.113689 | 0.115463 | 0.11656 | 0.236289 | 0.590661 |
| UL40 | P16780 | LL | 0.640295 | 0.629994 | 0.472709 | 0.538386 | 0.561222 |
| UL47 | P16784 | LL | 0.008892 | 0.020231 | 0.060079 | 0.233051 | 0.52868 |
| UL49 | P16786 | LL | | 0.187047 | 0.164335 | 0.265126 | 0.561868 |
| UL69 | P16749 | LL | 0.220265 | 0.161545 | 0.206227 | 0.350776 | 0.610094 |
| UL70 | P17149 | LL | 0.188217 | 0.393742 | 0.419399 | 0.610433 | 0.846022 |
| UL83 | P06725 | LL | 0.04505 | 0.020484 | 0.020481 | 0.100194 | 0.409142 |
| US15 | P09718 | LL | 1.317916 | 0.516807 | 0.432643 | 0.45761 | 0.618698 |
| IR10 | P16808 | LL | | | 0.035575 | 0.205656 | 0.44946 |
| IR12 | P16810 | LL | 0.068108 | 0.290824 | 0.449695 | 0.69096 | 0.796962 |
| CVC1 | P16799 | L | 0.061193 | 0.111536 | 0.096785 | 0.225234 | 0.517324 |
| GO | P16750 | L | | | 0.059887 | 0.244254 | 0.423632 |
| MCP | P16729 | L | 0.004968 | 0.006609 | 0.026238 | 0.148166 | 0.449652 |
| SCP | Q7M6N6 | L | 0.004248 | 0.004793 | 0.014999 | 0.124623 | 0.316896 |
| TRM2 | P16792 | L | 0.019806 | 0.056653 | 0.079497 | 0.189958 | 0.48826 |
| UL103 | P16734 | L | 0.357808 | 0.156695 | 0.092753 | 0.248852 | 0.520817 |
| UL117 | P16770 | L | 0.436212 | 0.382954 | 0.315065 | 0.395142 | 0.544234 |
| UL22A | P16845 | L | 0.043823 | 0.101509 | 0.214966 | 0.348455 | 0.946211 |
| UL25 | P16761 | L | 0.038885 | 0.062398 | 0.095999 | 0.235287 | 0.508109 |
| UL29 | P16764 | L | 0.833637 | 0.755564 | 0.505968 | 0.667421 | 0.814801 |
| UL30 | P16765 | L | | 0.153757 | 0.143097 | 0.235511 | 0.406439 |
| UL31 | P16848 | L | 0.327923 | 0.144514 | 0.144678 | 0.246161 | 0.527495 |
| UL43 | P16781 | L | 0.060882 | 0.049915 | 0.031757 | 0.053129 | 0.229447 |
| UL52 | P16793 | L | 0.015673 | 0.051106 | 0.119127 | 0.322407 | 0.616075 |
| UL76 | P16725 | L | | | 0.031921 | 0.151271 | 0.366014 |
| UL79 | P16752 | L | | | | 0.329911 | 0.399809 |
| UL80 | P16753 | L | 0.036298 | 0.035881 | 0.060522 | 0.267357 | 0.591225 |
| UL82 | P06726 | L | 0.184316 | 0.106086 | 0.104185 | 0.256563 | 0.556114 |
| UL87 | P16730 | L | | | 0.090288 | 0.264554 | 0.328096 |
| UL88 | P16731 | L | 0.038308 | 0.087535 | 0.16855 | 0.392623 | 0.633948 |
| UL94 | P16800 | L | 0.086256 | 0.074295 | 0.114538 | 0.277603 | 0.580289 |
| UL99 | P13200 | L | 0.029596 | 0.051654 | 0.055125 | 0.175331 | 0.474914 |
| gH | P12824 | L | 0.049246 | 0.063364 | 0.103196 | 0.283512 | 0.584909 |
| gL | P16832 | L | 0.037266 | 0.051915 | 0.082463 | 0.262318 | 0.576832 |
| gM | P16733 | L | 0.034853 | 0.024148 | 0.062423 | 0.265256 | 0.650841 |
| gN | P16795 | L | 0.156426 | 0.11178 | 0.112219 | 0.29996 | 0.64289 |
| IR11 | P16809 | L | 0.104426 | 0.370685 | 0.43444 | 0.907473 | 1.205348 |

TABLE
FIG. 7A, part 4 of 4: Numerical values
corresponding heatmap in FIG. 7A.

| | | | treatment | | |
|---|---|---|---|---|---|
| | | | Resveratrol | | |
| Protein | Protein | Tempo- | timepoint | | |
| Gene | Acc'n | rality | 24 | 48 | 72 |
| IRS1 | P09715 | IE | 0.529956 | 0.867139 | 1.155143 |
| TRS1 | P09695 | IE | 0.290693 | 0.712253 | 1.081477 |
| UL122 | P19893 | IE | 0.065837 | 0.071551 | 0.098803 |
| UL123 | P13202 | IE | 1.053418 | 0.949379 | 0.857325 |
| UL13 | P16755 | IE | 2.215506 | 0.664646 | 0.477702 |
| UL36 | P16767 | IE | 1.360005 | 1.044813 | 0.81251 |
| UL37 | P16778 | IE | 0.979705 | 1.143145 | 0.998048 |
| CVC2 | P16726 | DE | 0.024159 | 0.04895 | 0.05308 |
| DBP | P17147 | DE | 0.260316 | 0.317438 | 0.203067 |
| HELI | P16736 | DE | 0.523923 | 0.703469 | 0.762553 |
| NEC1 | P16794 | DE | | | |
| NEC2 | P16791 | DE | 0.050306 | 0.134738 | 0.231033 |
| RIR1 | P16782 | DE | 0.122928 | 0.146687 | 0.169564 |
| TRM1 | P16724 | DE | 0.049604 | 0.174351 | 0.173559 |
| UL102 | P16827 | DE | 0.888975 | 1.270298 | 1.099634 |
| UL104 | P16735 | DE | 0.07335 | 0.109096 | 0.110828 |
| UL112/UL113 | P17151 | DE | 0.360105 | 0.454537 | 0.391216 |
| UL114 | P16769 | DE | 0.800814 | 1.321974 | 1.097195 |
| UL119/UL118 | P16739 | DE | 0.559619 | 0.636653 | 0.22415 |
| UL128 | P16837 | DE | 0.080346 | 0.035958 | 0.037134 |
| UL26 | P16762 | DE | 0.087447 | 0.092237 | 0.087004 |
| UL32 | P08318 | DE | 0.02724 | 0.064069 | 0.098338 |
| UL34 | P16812 | DE | 0.109583 | 0.126462 | 0.194478 |
| UL35 | P16766 | DE | | 0.026531 | 0.066803 |
| UL38 | P16779 | DE | 0.969075 | 0.806915 | 0.657148 |
| UL4 | P17146 | DE | 0.027921 | 0.10705 | 0.18632 |
| UL44 | P16790 | DE | 0.059105 | 0.096567 | 0.10768 |
| UL48 | P16785 | DE | 0.021937 | 0.053935 | 0.076903 |
| UL54 | P08546 | DE | 0.629525 | 0.946427 | 0.892148 |
| UL71 | P16823 | DE | 0.525154 | 0.366053 | 0.420088 |
| UL78 | P16751 | DE | 1.895865 | 1.069589 | 0.437089 |
| UL84 | P16727 | DE | 0.140603 | 0.148911 | 0.180674 |
| UL95 | P16801 | DE | 0.067564 | 0.291435 | 0.48866 |
| UL96 | P16787 | DE | 0.47273 | 0.386106 | 0.280409 |
| UL97 | P16788 | DE | 0.365199 | 0.373237 | 0.288369 |
| UL98 | P16789 | DE | 0.297907 | 0.341223 | 0.261442 |
| US12 | P09721 | DE | 1.672542 | 0.896436 | 0.270936 |
| US13 | P09720 | DE | 0.547645 | 0.592286 | 0.506371 |
| US14 | P09719 | DE | 0.388327 | 0.230434 | 0.141255 |
| US18 | P69334 | DE | | | |
| US22 | P09722 | DE | 0.337908 | 0.537142 | 0.565354 |
| US23 | P09701 | DE | 1.208648 | 0.598219 | 0.5216 |
| US24 | P09700 | DE | 1.018754 | 0.485704 | 0.34223 |
| US34 | P09709 | DE | 2.474791 | 2.409233 | 1.230564 |
| US8 | P09730 | DE | 0.985558 | 1.342671 | 1.040625 |
| US9 | P09729 | DE | 1.11978 | 2.242142 | 1.96586 |
| gB | P06473 | DE | 0.203534 | 0.199137 | 0.207817 |
| IR01 | P09710 | DE | 0.239462 | 0.332489 | 0.449532 |
| DUT | P16824 | LL | 0.381506 | 0.226187 | |
| TRM3 | P16732 | LL | | 0.024259 | 0.027495 |
| TRX1 | P16783 | LL | 0.007402 | 0.012593 | 0.019945 |
| TRX2 | P16728 | LL | 0.006335 | 0.010032 | 0.019982 |
| UL132 | P69338 | LL | 0.151863 | 0.202743 | 0.215042 |
| UL24 | P16760 | LL | 0.115035 | 0.104562 | 0.107209 |
| UL40 | P16780 | LL | 0.681605 | 1.097644 | 0.685119 |
| UL47 | P16784 | LL | 0.013143 | 0.042567 | 0.088278 |
| UL49 | P16786 | LL | 0.380113 | 0.910012 | 1.208807 |
| UL69 | P16749 | LL | 0.259004 | 0.302363 | 0.375791 |
| UL70 | P17149 | LL | 0.215131 | 0.55462 | 0.682003 |
| UL83 | P06725 | LL | 0.048684 | 0.036714 | 0.038456 |
| US15 | P09718 | LL | 2.089457 | 1.334174 | 0.686865 |
| IR10 | P16808 | LL | | | |
| IR12 | P16810 | LL | 0.130465 | 0.261383 | 0.181978 |
| CVC1 | P16799 | L | 0.133548 | 0.333945 | 0.429535 |
| GO | P16750 | L | | 0.020748 | 0.038813 |
| MCP | P16729 | L | 0.005554 | 0.008603 | 0.011437 |
| SCP | Q7M6N6 | L | | 0.008886 | 0.004632 |
| TRM2 | P16792 | L | 0.012358 | 0.034579 | 0.02287 |
| UL103 | P16734 | L | 0.386143 | 0.148924 | 0.129388 |
| UL117 | P16770 | L | 0.74394 | 0.49095 | 0.303232 |
| UL22A | P16845 | L | 0.068781 | 0.218774 | 0.262339 |
| UL25 | P16761 | L | 0.021733 | 0.07603 | 0.126454 |
| UL29 | P16764 | L | 0.698949 | 0.564767 | 0.475613 |
| UL30 | P16765 | L | | 0.140005 | 0.13441 |
| UL31 | P16848 | L | 0.443437 | 0.321745 | 0.359492 |
| UL43 | P16781 | L | 0.062104 | 0.068869 | 0.072844 |
| UL52 | P16793 | L | 0.02488 | 0.097745 | 0.114328 |
| UL76 | P16725 | L | | | 0.026096 |
| UL79 | P16752 | L | | | |
| UL80 | P16753 | L | 0.036415 | 0.041819 | 0.054043 |
| UL82 | P06726 | L | 0.202418 | 0.219099 | 0.281263 |
| UL87 | P16730 | L | | | 0.038618 |
| UL88 | P16731 | L | 0.051737 | 0.094011 | 0.15546 |
| UL94 | P16800 | L | 0.102746 | 0.181131 | 0.240157 |
| UL99 | P13200 | L | 0.048054 | 0.098972 | 0.137407 |
| gH | P12824 | L | 0.086512 | 0.123344 | 0.171651 |
| gL | P16832 | L | 0.037563 | 0.058173 | 0.077717 |
| gM | P16733 | L | 0.040469 | 0.047109 | 0.069545 |
| gN | P16795 | L | 0.185358 | 0.084823 | 0.071192 |
| IR11 | P16809 | L | 0.409211 | 0.32185 | 0.253003 |

Table FIG. 8A, part 1 of 4: Numerical values corresponding heatmap in FIG. 8A.

| | | | treatment | | | |
|---|---|---|---|---|---|---|
| | | | DMSO | | | |
| Protein | Protein | | timepoint | | | |
| Gene | Acc'n | Temporality | 2 | 6 | 12 | 18 |
| ICP0 | P08393 | IE | 0.091601 | 0.831262 | 1.617589 | 1.556848 |
| ICP22 | P04485 | IE | 0.015224 | 0.877639 | 1.389603 | 1.548126 |
| ICP4 | P08392 | IE | 0.115776 | 1.116858 | 1.482325 | 1.570898 |
| UL54 | P10238 | IE | 0.081053 | 1.295314 | 1.341291 | 1.435079 |
| DBP | P04296 | E | 0.035458 | 0.956676 | 1.374226 | 1.572022 |
| TK | P03176 | E | 0.037772 | 1.117986 | 1.132964 | 1.25255 |
| UL12 | P04294 | E | 0.016795 | 1.117311 | 1.187313 | 1.505661 |
| UL30 | P04293 | E | 0.054658 | 1.347601 | 1.228628 | 1.204441 |
| UL42 | P10226 | E | 0.004685 | 0.471087 | 1.467427 | 1.717706 |
| UL8 | P10192 | E | 0.019022 | 1.00542 | 0.970042 | 1.04424 |
| US3 | P04413 | E | 0.012863 | 0.819327 | 1.406379 | 1.113226 |
| CVC2 | P10209 | L | 0.008226 | 0.473372 | 1.575599 | 1.951277 |
| UL26 | P10210 | L | | 0.327125 | 1.253707 | 1.799956 |
| UL48 | P06492 | L | 0.007555 | 0.438116 | 1.694589 | 2.178714 |
| UL49 | P10233 | L | 0.00999 | 0.724138 | 1.813934 | 1.542657 |
| gB | P10211 | L | 0.013898 | 0.608248 | 1.680034 | 1.844084 |
| gI | P06487 | L | | 0.587287 | 1.273557 | 1.038478 |

Table FIG. 8A, part 2 of 4: Numerical values corresponding heatmap in FIG. 8A.

| | | | treatment | | | |
|---|---|---|---|---|---|---|
| | | | EX527 | | | |
| Protein | Protein | | timepoint | | | |
| Gene | Acc'n | Temporality | 2 | 6 | 12 | 18 |
| ICP0 | P08393 | IE | 0.130747 | 0.933884 | 1.772462 | 1.714264 |
| ICP22 | P04485 | IE | 0.014161 | 1.072249 | 1.535313 | 1.978738 |
| ICP4 | P08392 | IE | 0.091539 | 1.324896 | 1.510708 | 2.197023 |
| UL54 | P10238 | IE | 0.048592 | 1.758885 | 1.814059 | 1.637077 |
| DBP | P04296 | E | 0.024651 | 1.405336 | 1.56787 | 2.002195 |
| TK | P03176 | E | 0.026538 | 1.44938 | 1.751106 | 2.225822 |
| UL12 | P04294 | E | 0.011959 | 1.502629 | 1.841384 | 2.271776 |
| UL30 | P04293 | E | 0.031238 | 1.509631 | 1.753595 | 2.062508 |
| UL42 | P10226 | E | 0.002331 | 0.597451 | 2.035243 | 3.214178 |
| UL8 | P10192 | E | 0.023466 | 1.251577 | 1.371002 | 1.783501 |
| US3 | P04413 | E | 0.01288 | 0.947623 | 1.573214 | 1.817608 |
| CVC2 | P10209 | L | 0.007823 | 0.543283 | 2.235611 | 3.555849 |
| UL26 | P10210 | L | 0.005455 | 0.354121 | 1.506767 | 2.826692 |
| UL48 | P06492 | L | 0.006829 | 0.561339 | 2.26784 | 3.714222 |
| UL49 | P10233 | L | 0.00849 | 0.802131 | 1.954359 | 2.145436 |
| gB | P10211 | L | 0.01179 | 0.622509 | 1.744297 | 2.559837 |
| gI | P06487 | L | 0.006707 | 0.76479 | 1.288333 | 3.078023 |

Table FIG. 8A, part 3 of 4: Numerical values corresponding heatmap in FIG. 8A.

| | | | treatment | | | |
|---|---|---|---|---|---|---|
| | | | CAY | CAY | CAY | CAY |
| Protein | Protein | | timepoint | | | |
| Gene | Acc'n | Temporality | 2 | 6 | 12 | 18 |
| ICP0 | P08393 | IE | 0.109689 | 0.559876 | 1.404967 | 1.467764 |
| ICP22 | P04485 | IE | 0.012207 | 0.691448 | 1.221374 | 1.666293 |
| ICP4 | P08392 | IE | 0.086075 | 0.801731 | 1.191422 | 1.57662 |
| UL54 | P10238 | IE | 0.051547 | 0.952677 | 1.257328 | 1.313714 |
| DBP | P04296 | E | 0.023522 | 0.758127 | 1.208481 | 1.69295 |
| TK | P03176 | E | 0.028755 | 0.863711 | 1.310912 | 1.303696 |
| UL12 | P04294 | E | 0.013418 | 0.896401 | 1.438763 | 1.546526 |
| UL30 | P04293 | E | 0.036594 | 0.980419 | 1.369071 | 1.587575 |
| UL42 | P10226 | E | 0.002416 | 0.351432 | 1.383397 | 2.445713 |
| UL8 | P10192 | E | 0.016257 | 0.791325 | 1.049431 | 1.213886 |
| US3 | P04413 | E | 0.011643 | 0.601446 | 1.132041 | 1.20266 |
| CVC2 | P10209 | L | 0.008425 | 0.340462 | 1.678938 | 2.616954 |
| UL26 | P10210 | L | 0.00159 | 0.222366 | 1.267067 | 2.207433 |
| UL48 | P06492 | L | 0.007094 | 0.297847 | 1.650076 | 2.515001 |
| UL49 | P10233 | L | 0.007747 | 0.461708 | 1.472174 | 1.715462 |
| gB | P10211 | L | 0.011622 | 0.382347 | 1.390461 | 1.982212 |
| gI | P06487 | L | 0.006169 | 0.556999 | 1.015615 | 1.740746 |

Table FIG. 8A, part 4 of 4: Numerical values corresponding heatmap in FIG. 8A.

| | | | treatment | | | |
|---|---|---|---|---|---|---|
| | | | Res | Res | Res | Res |
| Protein | Protein | | timepoint | | | |
| Gene | Acc'n | Temporality | 2 | 6 | 12 | 18 |
| ICP0 | P08393 | IE | 0.16223 | 0.533334 | 1.107601 | 1.159238 |
| ICP22 | P04485 | IE | 0.015004 | 0.742905 | 1.240564 | 1.213186 |
| ICP4 | P08392 | IE | 0.107283 | 1.06342 | 1.100592 | 1.049477 |
| UL54 | P10238 | IE | 0.0667 | 0.94154 | 1.026143 | 0.814809 |
| DBP | P04296 | E | 0.021206 | 0.912071 | 1.230058 | 1.251509 |
| TK | P03176 | E | 0.038321 | 0.970655 | 1.239877 | 0.980627 |
| UL12 | P04294 | E | 0.017633 | 0.741797 | 0.887778 | 0.742468 |
| UL30 | P04293 | E | 0.038758 | 1.353173 | 1.001291 | 0.792639 |
| UL42 | P10226 | E | 0.003041 | 0.286649 | 0.774309 | 0.783726 |
| UL8 | P10192 | E | 0.017105 | 1.056303 | 0.912267 | 0.825867 |
| US3 | P04413 | E | 0.016047 | 0.615979 | 1.156352 | 0.945653 |
| CVC2 | P10209 | L | 0.010031 | 0.259173 | 0.706668 | 0.600989 |
| UL26 | P10210 | L | 0.003453 | 0.198554 | 0.754941 | 0.889843 |
| UL48 | P06492 | L | 0.007183 | 0.181626 | 0.841061 | 0.927547 |
| UL49 | P10233 | L | 0.009894 | 0.428049 | 1.282968 | 1.225547 |
| gB | P10211 | L | 0.012795 | 0.509822 | 1.285611 | 1.305631 |
| gI | P06487 | L | 0.00718 | 0.570458 | 0.956029 | 1.002501 |

TABLE
FIG. 8C, part 1 of 3: Numerical values
corresponding heatmap in FIG. 8C.

| | | | treatment | | |
|---|---|---|---|---|---|
| | | | Mock | | |
| Protein | Protein | | timepoint | | |
| Gene | Acc'n | Tempor. | 24 | 48 | 72 |
| K8 | Q2HR82 | IE | 0.250872 | 1.161807 | 1.324475 |
| ORF16 | F5HGJ3 | IE | 0.311661 | 0.822163 | 1.70263 |
| ORF45 | F5HDE4 | IE | 0.520839 | 1.002488 | 1.812888 |
| ORF48 | Q2HR85 | IE | | | 1.147799 |
| ORF50 | F5HCV3 | IE | 0.485292 | 0.996571 | 1.574245 |
| ORF57 | Q2HR75 | IE | 0.324399 | 0.983233 | 1.720653 |
| 70 | P90463 | DE | 0.279352 | 1.284666 | 1.843208 |
| DBP | Q2HRD3 | DE | 0.075075 | 0.927189 | 2.373527 |
| DUT | Q2HR78 | DE | 0 | 0.277177 | 1.960806 |
| HELI | Q2HR89 | DE | | 0.631028 | 1.393923 |
| K14 | P0C788 | DE | | 0.761589 | 1.321663 |
| K2 | Q2HRC7 | DE | 1.066088 | 1.260581 | 0.644392 |
| K3 | P90495 | DE | 0.155329 | 1.075289 | 1.152274 |
| K5 | P90489 | DE | 0.211893 | 1.031406 | 1.428942 |
| NEC1 | F5H982 | DE | 0.161504 | 0.882364 | 2.19039 |
| NEC2 | F5HA27 | DE | 0.027672 | 0.494326 | 1.832928 |
| ORF K4 | Q98157 | DE | 0.340771 | 1.060912 | 1.679899 |
| ORF10 | Q2HRC9 | DE | 0.255515 | 1.052553 | 1.983122 |
| ORF11 | Q2HRC8 | DE | 0.560045 | 1.054019 | 1.319391 |
| ORF17 | Q2HRB6 | DE | 0.148286 | 0.694125 | 2.044174 |
| ORF2 | Q2HRC6 | DE | 0.050944 | 0.561067 | 2.014536 |
| ORF36 | F5HGH5 | DE | | 0.219931 | 1.974033 |
| ORF37 | Q2HR95 | DE | 0.216816 | 0.772489 | 1.886544 |
| ORF40 | Q2HR92 | DE | | 0.873853 | 1.382555 |
| ORF46 | F5HFA1 | DE | 0.233529 | 0.993466 | 1.975631 |
| ORF49 | Q2HR83 | DE | | 0.556444 | 1.721273 |
| ORF56 | F5HIN0 | DE | | | 1.106301 |
| ORF59 | F5HID2 | DE | 0.148183 | 1.162132 | 1.756203 |
| ORF66 | F5HG20 | DE | | 0.368537 | 1.519143 |
| ORF9 | Q2HRD0 | DE | | 0.769739 | 1.690263 |
| RIR1 | Q2HR67 | DE | 0.175293 | 0.773715 | 2.737441 |
| TK | F5HB62 | DE | 0.049232 | 0.456436 | 1.495669 |
| vIRF-1 | F5HF68 | DE | | 0.288905 | 1.136165 |
| CVC1 | F5HB39 | L | | 0.534377 | 1.933347 |
| CVC2 | Q2HRB3 | L | 0.031014 | 0.56842 | 1.950497 |
| K8.1 | F5HB98 | L | 0.155327 | 0.619327 | 1.48734 |
| MCP | Q2HRA7 | L | 0.039025 | 0.585137 | 2.015966 |
| ORF20 | Q2HRB2 | L | | 0.404988 | 1.893899 |
| ORF23 | F5HIM6 | L | | 0.132345 | 1.442602 |
| ORF24 | F5HFD2 | L | | 0.404033 | 1.336459 |
| ORF27 | F5HDY6 | L | | 0.631181 | 1.380048 |
| ORF28 | F5HI25 | L | 0.151046 | 0.986466 | 1.906399 |
| ORF33 | F5HEF2 | L | 0.06944 | 0.600258 | 3.017602 |
| ORF34 | Q2HR98 | L | | 0.396368 | 1.124734 |
| ORF35 | F5HCD4 | L | | 0.320572 | 1.789647 |
| ORF38 | F5HHY1 | L | 0.028723 | 0.546813 | 2.338524 |
| ORF4 | Q2HRD4 | L | | 0.847298 | 1.12695 |
| ORF42 | F5HAI6 | L | | 0.50304 | 2.051213 |
| ORF43 | F5HGK9 | L | | 0.395142 | 1.979972 |
| ORF52 | Q2HR80 | L | 0.012767 | 0.543412 | 1.890341 |
| ORF55 | F5H9W9 | L | | 0.599361 | 1.738063 |
| ORF63 | F5HEU7 | L | | 0.342482 | 1.872595 |
| ORF64 | Q2HR64 | L | | 0.359751 | 1.802176 |
| ORF68 | F5HF47 | L | 0.165706 | 0.823673 | 2.220449 |
| ORF75 | Q9QR70 | L | | 0.398109 | 1.824131 |
| SCP | Q2HR63 | L | | 0.520672 | 1.740542 |
| TRX1 | F5H8Y5 | L | | 0.419094 | 1.876895 |
| TRX2 | F5HGN8 | L | | 0.449372 | 1.704811 |
| gB | F5HB81 | L | 0.153946 | 0.673606 | 2.522058 |
| gH | F5HAK9 | L | 0.898514 | 0.700489 | 1.634211 |
| gL | F5HDB7 | L | 0.570127 | 0.789326 | 1.980407 |
| gM | F5HDD0 | L | 0.0522 | 0.693253 | 1.350728 |

TABLE
FIG. 8C, part 2 of 3: Numerical values
corresponding heatmap in FIG. 8C.

| | | | treatment | | |
|---|---|---|---|---|---|
| | | | EX | | |
| Protein | Protein | | timepoint | | |
| Gene | Acc'n | Tempor. | 24 | 48 | 72 |
| K8 | Q2HR82 | IE | 0.201633 | 1.14801 | 1.69662 |
| ORF16 | F5HGJ3 | IE | 0.234168 | 0.686419 | 1.622655 |
| ORF45 | F5HDE4 | IE | 0.264585 | 0.979881 | 1.541031 |
| ORF48 | Q2HR85 | IE | | | 1.065234 |
| ORF50 | F5HCV3 | IE | 0.313215 | 0.847011 | 1.365736 |
| ORF57 | Q2HR75 | IE | 0.171769 | 0.839816 | 1.721247 |
| 70 | P90463 | DE | 0.132778 | 0.819533 | 1.404251 |
| DBP | Q2HRD3 | DE | 0.03296 | 0.535677 | 1.722069 |
| DUT | Q2HR78 | DE | 0 | | 0.935547 |
| HELI | Q2HR89 | DE | | 0.326801 | 0.955653 |
| K14 | P0C788 | DE | | 0.578306 | 1.286936 |
| K2 | Q2HRC7 | DE | 0.833204 | 1.205792 | 1.082347 |
| K3 | P90495 | DE | 0.092396 | 1.003864 | 1.842465 |
| K5 | P90489 | DE | 0.152053 | 0.968268 | 2.061201 |
| NEC1 | F5H982 | DE | 0.054822 | 0.451124 | 1.399405 |
| NEC2 | F5HA27 | DE | 0.015266 | 0.244264 | 1.532691 |
| ORF K4 | Q98157 | DE | | 0.486906 | 1.191785 |
| ORF10 | Q2HRC9 | DE | 0.115198 | 0.580505 | 1.434879 |
| ORF11 | Q2HRC8 | DE | 0.45656 | 0.472621 | 0.779285 |
| ORF17 | Q2HRB6 | DE | 0.10648 | 0.404479 | 1.570592 |
| ORF2 | Q2HRC6 | DE | | 0.437974 | 1.637422 |
| ORF36 | F5HGH5 | DE | | | 0.621963 |
| ORF37 | Q2HR95 | DE | 0.092797 | 0.529641 | 1.993645 |
| ORF40 | Q2HR92 | DE | | | 0.249163 |
| ORF46 | F5HFA1 | DE | 0.126543 | 0.833771 | 1.053783 |
| ORF49 | Q2HR83 | DE | | 0.338501 | 1.413434 |
| ORF56 | F5HIN0 | DE | | | |
| ORF59 | F5HID2 | DE | 0.061268 | 0.795458 | 1.902301 |
| ORF66 | F5HG20 | DE | | 0.146811 | 1.215235 |
| ORF9 | Q2HRD0 | DE | | 0.4208 | 1.043832 |
| RIR1 | Q2HR67 | DE | 0.114925 | 0.172039 | 1.141737 |
| TK | F5HB62 | DE | | 0.392223 | 1.62304 |
| vIRF-1 | F5HF68 | DE | | | 1.269964 |
| CVC1 | F5HB39 | L | | 0.194165 | 1.207429 |
| CVC2 | Q2HRB3 | L | | 0.301861 | 1.371002 |
| K8.1 | F5HB98 | L | 0.223542 | 0.34868 | 1.653587 |
| MCP | Q2HRA7 | L | 0.021274 | 0.281983 | 1.614703 |
| ORF20 | Q2HRB2 | L | | 0.170939 | 1.391119 |
| ORF23 | F5HIM6 | L | | | 1.088129 |
| ORF24 | F5HFD2 | L | | | 1.416283 |
| ORF27 | F5HDY6 | L | | 0.381594 | 1.216247 |
| ORF28 | F5HI25 | L | 0.060266 | 0.553673 | 1.419655 |
| ORF33 | F5HEF2 | L | 0.031752 | 0.321573 | 1.262954 |
| ORF34 | Q2HR98 | L | | | 1.264619 |
| ORF35 | F5HCD4 | L | | | 0.92449 |
| ORF38 | F5HHY1 | L | | 0.426122 | 1.717284 |
| ORF4 | Q2HRD4 | L | | 0.803217 | 1.529984 |
| ORF42 | F5HAI6 | L | | 0.311363 | 1.238085 |
| ORF43 | F5HGK9 | L | | 0.181894 | 1.438427 |
| ORF52 | Q2HR80 | L | 0.012366 | 0.364312 | 1.937347 |
| ORF55 | F5H9W9 | L | | 0.305254 | 1.208399 |
| ORF63 | F5HEU7 | L | | | 0.845336 |
| ORF64 | Q2HR64 | L | | 0.205695 | 1.343859 |
| ORF68 | F5HF47 | L | 0.061954 | 0.314714 | 1.081687 |
| ORF75 | Q9QR70 | L | | 0.24762 | 1.15379 |
| SCP | Q2HR63 | L | | 0.206876 | 1.240679 |
| TRX1 | F5H8Y5 | L | | 0.11445 | 1.134226 |
| TRX2 | F5HGN8 | L | | 0.226945 | 1.17981 |
| gB | F5HB81 | L | 0.097263 | 0.264001 | 1.046872 |
| gH | F5HAK9 | L | 0.338935 | 0.662013 | 1.208988 |
| gL | F5HDB7 | L | 0.414566 | 0.8375 | 1.053092 |
| gM | F5HDD0 | L | | 0.762034 | 1.656842 |

TABLE
FIG. 8C, part 3 of 3: Numerical values
corresponding heatmap in FIG. 8C.

| | | | treatment | | |
|---|---|---|---|---|---|
| | | | CAY | | |
| Protein | Protein | | timepoint | | |
| Gene | Acc'n | Tempor. | 24 | 48 | 72 |
| K8 | Q2HR82 | IE | 0.388124 | 1.329386 | 1.499073 |
| ORF16 | F5HGJ3 | IE | 0.517516 | 1.053491 | 1.293577 |
| ORF45 | F5HDE4 | IE | 0.547881 | 1.041682 | 1.317436 |
| ORF48 | Q2HR85 | IE | | 0.508965 | 1.106384 |
| ORF50 | F5HCV3 | IE | 0.651305 | 1.244527 | 1.522098 |
| ORF57 | Q2HR75 | IE | 0.564089 | 1.18637 | 1.488422 |
| 70 | P90463 | DE | 0.451947 | 1.340581 | 1.443683 |
| DBP | Q2HRD3 | DE | 0.11166 | 1.101957 | 2.119885 |
| DUT | Q2HR78 | DE | | 0.516316 | 1.581764 |
| HELI | Q2HR89 | DE | | 0.949235 | 1.476599 |
| K14 | P0C788 | DE | | 1.065478 | 0.986027 |
| K2 | Q2HRC7 | DE | 1.41119 | 1.002016 | 0.677338 |
| K3 | P90495 | DE | 0.254321 | 1.3004 | 2.123664 |
| K5 | P90489 | DE | 0.333265 | 1.119622 | 1.69335 |
| NEC1 | F5H982 | DE | 0.170144 | 0.872546 | 1.432558 |
| NEC2 | F5HA27 | DE | 0.064413 | 0.712001 | 2.024777 |
| ORF K4 | Q98157 | DE | 1.155091 | 1.122112 | 0.63291 |
| ORF10 | Q2HRC9 | DE | 0.444376 | 1.064794 | 1.401755 |
| ORF11 | Q2HRC8 | DE | 1.08323 | 1.636782 | 1.51014 |
| ORF17 | Q2HRB6 | DE | 0.172963 | 0.745322 | 1.435201 |
| ORF2 | Q2HRC6 | DE | 0.089948 | 0.605622 | 1.49398 |
| ORF36 | F5HGH5 | DE | | 0.582354 | 1.601718 |
| ORF37 | Q2HR95 | DE | 0.361123 | 1.032245 | 1.632392 |
| ORF40 | Q2HR92 | DE | | 0.508197 | 1.007717 |
| ORF46 | F5HFA1 | DE | 0.357076 | 1.368402 | 1.653878 |
| ORF49 | Q2HR83 | DE | | 0.642917 | 0.996682 |
| ORF56 | F5HIN0 | DE | | | 0.681096 |
| ORF59 | F5HID2 | DE | 0.226801 | 1.311835 | 1.635818 |
| ORF66 | F5HG20 | DE | | 0.589573 | 1.52081 |
| ORF9 | Q2HRD0 | DE | 0.098675 | 0.760632 | 1.640208 |
| RIR1 | Q2HR67 | DE | 0.28733 | 1.143322 | 1.489698 |
| TK | F5HB62 | DE | 0.114656 | 0.807848 | 1.823661 |
| vIRF-1 | F5HF68 | DE | | 0.569482 | 1.735484 |
| CVC1 | F5HB39 | L | 0.119084 | 0.54238 | 1.505753 |
| CVC2 | Q2HRB3 | L | 0.100446 | 0.741668 | 1.689399 |
| K8.1 | F5HB98 | L | 0.16624 | 0.749003 | 2.095964 |
| MCP | Q2HRA7 | L | 0.068842 | 0.675883 | 1.783281 |
| ORF20 | Q2HRB2 | L | | 0.653535 | 1.48552 |
| ORF23 | F5HIM6 | L | | 0.356252 | 1.008057 |
| ORF24 | F5HFD2 | L | | 0.671061 | 0.980702 |
| ORF27 | F5HDY6 | L | 0.076732 | 0.767915 | 1.423831 |
| ORF28 | F5HI25 | L | 0.348909 | 1.404417 | 1.744692 |
| ORF33 | F5HEF2 | L | 0.144601 | 0.694402 | 1.851981 |
| ORF34 | Q2HR98 | L | | 0.903414 | 1.310864 |
| ORF35 | F5HCD4 | L | | 0.526872 | 0.806904 |
| ORF38 | F5HHY1 | L | 0.044738 | 0.586253 | 1.348275 |
| ORF4 | Q2HRD4 | L | 0.134492 | 0.849358 | 1.048987 |
| ORF42 | F5HAI6 | L | | 0.554666 | 1.341633 |
| ORF43 | F5HGK9 | L | | 0.44264 | 1.152873 |
| ORF52 | Q2HR80 | L | 0.017914 | 0.528212 | 1.884785 |
| ORF55 | F5H9W9 | L | | 0.555562 | 1.237576 |
| ORF63 | F5HEU7 | L | | 0.4013 | 1.209528 |
| ORF64 | Q2HR64 | L | | 0.445928 | 1.451584 |
| ORF68 | F5HF47 | L | 0.266188 | 0.892474 | 1.892521 |
| ORF75 | Q9QR70 | L | | 0.487687 | 1.318834 |
| SCP | Q2HR63 | L | | 0.529627 | 1.454643 |
| TRX1 | F5H8Y5 | L | | 0.425996 | 1.586564 |
| TRX2 | F5HGN8 | L | | 0.479186 | 1.161251 |
| gB | F5HB81 | L | 0.343295 | 0.995086 | 1.747287 |
| gH | F5HAK9 | L | 0.27268 | 0.903574 | 1.47578 |
| gL | F5HDB7 | L | 1.031252 | 0.793627 | 1.027628 |
| gM | F5HDD0 | L | 0.189537 | 1.115876 | 1.246972 |

Table FIG 9C: Numerical values corresponding heatmap in FIG. 9C.

| Protein | | HSV-1 strain* | | | | | | |
|---|---|---|---|---|---|---|---|---|
| Gene | Tempor. | 17 | F | H129 | KOS | MacIntyre | McKrae | Sc16 |
| CVC2 | L | 4/100 | 4/100 | 4/100 | 4/100 | 4/100 | 4/100 | 4/100 |
| DBP | E | 4/100 | 4/100 | 4/100 | 4/100 | 4/100 | 4/100 | 4/100 |
| ICP0 | IE | 4/100 | 4/100 | 3/75 | 4/100 | 4/100 | 4/100 | 4/100 |
| ICP22 | IE | 4/100 | 4/100 | 4/100 | 4/100 | 4/100 | 4/100 | 4/100 |
| ICP4 | IE | 4/100 | 4/100 | 4/100 | 4/100 | 4/100 | 4/100 | 4/100 |
| TK | E | 4/100 | 3/75 | 2/50 | 3/75 | 4/100 | 3/75 | 4/100 |
| UL12 | E | 4/100 | 4/100 | 4/100 | 4/100 | 4/100 | 4/100 | 4/100 |
| UL26 | L | 4/100 | 4/100 | 3/75 | 4/100 | 4/100 | 3/75 | 4/100 |
| UL30 | E | 4/100 | 4/100 | 4/100 | 4/100 | 4/100 | 4/100 | 4/100 |
| UL42 | E | 4/100 | 4/100 | 4/100 | 4/100 | 4/100 | 4/100 | 4/100 |
| UL48 | L | 4/100 | 4/100 | 4/100 | 4/100 | 4/100 | 4/100 | 4/100 |
| UL49 | L | 4/100 | 4/100 | 4/100 | 4/100 | 3/75 | 4/100 | 4/100 |
| UL54 | IE | 4/100 | 3/75 | 3/75 | 3/75 | 3/75 | 3/75 | 3/75 |
| UL8 | E | 4/100 | 4/100 | 3/75 | 4/100 | 4/100 | 3/75 | 4/100 |
| US3 | E | 4/100 | 4/100 | 4/100 | 4/100 | 4/100 | 4/100 | 4/100 |
| gB | L | 4/100 | 4/100 | 4/100 | 4/100 | 4/100 | 4/100 | 4/100 |
| gI | L | 4/100 | 3/75 | 3/75 | 3/75 | 1/25 | 2/50 | 3/75 |

*Number of Conserved Peptides/Percentage of Conserved Peptides

Table FIG. 9D: Numerical values corresponding heatmap in FIG. 9D.

| Protein | | HCMV strain* | | | | | |
|---|---|---|---|---|---|---|---|
| Gene | Tempor. | AD169 | Merlin | TB40 | TR | Toledo | Towne |
| CVC1 | L | 2/66.6 | 2/66.6 | 2/66.6 | 2/66.6 | 1/33.3 | 1/33.3 |
| CVC2 | DE | 3/100 | 1/33.3 | 1/33.3 | 0/0 | 2/66.6 | 2/66.6 |
| DBP | DE | 3/100 | 3/100 | 3/100 | 3/100 | 3/100 | 3/100 |
| DUT | LL | 1/33.3 | 1/33.3 | 1/33.3 | 1/33.3 | 1/33.3 | 1/33.3 |
| GO | L | 3/100 | 1/33.3 | 1/33.3 | 0/0 | 1/33.3 | 0/0 |
| HELI | DE | 2/66.6 | 2/66.6 | 2/66.6 | 2/66.6 | 2/66.6 | 2/66.6 |
| IR01 | DE | 2/66.6 | 2/66.6 | 2/66.6 | 2/66.6 | 1/33.3 | 1/33.3 |
| IR10 | LL | 2/66.6 | 1/33.3 | 2/66.6 | 1/33.3 | 2/66.6 | 1/33.3 |
| IR11 | L | 3/100 | 2/66.6 | 2/66.6 | 2/66.6 | 2/66.6 | 2/66.6 |
| IR12 | LL | 3/100 | 0/0 | 0/0 | 0/0 | 3/100 | 0/0 |
| IRS1 | IE | 3/100 | 3/100 | 0/0 | 3/100 | 3/100 | 3/100 |
| MCP | L | 2/66.6 | 2/66.6 | 2/66.6 | 2/66.6 | 2/66.6 | 2/66.6 |
| NEC1 | DE | 2/66.6 | 2/66.6 | 2/66.6 | 2/66.6 | 2/66.6 | 2/66.6 |
| NEC2 | DE | 2/66.6 | 2/66.6 | 2/66.6 | 2/66.6 | 2/66.6 | 2/66.6 |
| RIR1 | DE | 3/100 | 3/100 | 2/66.6 | 2/66.6 | 3/100 | 2/66.6 |
| SCP | L | 1/33.3 | 1/33.3 | 1/33.3 | 1/33.3 | 1/33.3 | 1/33.3 |
| TRM1 | DE | 3/100 | 3/100 | 3/100 | 3/100 | 3/100 | 3/100 |
| TRM2 | L | 3/100 | 3/100 | 3/100 | 3/100 | 3/100 | 3/100 |
| TRM3 | LL | 3/100 | 3/100 | 3/100 | 3/100 | 3/100 | 3/100 |
| TRS1 | IE | 2/66.6 | 1/33.3 | 0/0 | 0/0 | 0/0 | 2/66.6 |
| TRX1 | LL | 3/100 | 3/100 | 3/100 | 3/100 | 3/100 | 3/100 |
| TRX2 | LL | 3/100 | 3/100 | 3/100 | 3/100 | 3/100 | 3/100 |
| UL102 | DE | 3/100 | 3/100 | 3/100 | 2/66.6 | 3/100 | 3/100 |
| UL103 | L | 3/100 | 3/100 | 3/100 | 3/100 | 3/100 | 3/100 |
| UL104 | DE | 3/100 | 2/66.6 | 3/100 | 3/100 | 3/100 | 3/100 |
| UL112/UL113 | DE | 3/100 | 3/100 | 3/100 | 3/100 | 3/100 | |
| UL114 | DE | 2/66.6 | 2/66.6 | 2/66.6 | 2/66.6 | 2/66.6 | 2/66.6 |
| UL117 | L | 3/100 | 3/100 | 3/100 | 3/100 | 3/100 | 3/100 |
| UL119/UL118 | DE | 2/66.6 | 2/66.6 | 2/66.6 | 2/66.6 | 2/66.6 | |
| UL122 | IE | 2/66.6 | 2/66.6 | 2/66.6 | 2/66.6 | 2/66.6 | 2/66.6 |
| UL123 | IE | 3/100 | 2/66.6 | 2/66.6 | 2/66.6 | 3/100 | 3/100 |
| UL128 | DE | 2/66.6 | 2/66.6 | 2/66.6 | 1/33.3 | 2/66.6 | 2/66.6 |
| UL13 | IE | 3/100 | 1/33.3 | 3/100 | 2/66.6 | 2/66.6 | 2/66.6 |
| UL132 | LL | 3/100 | 3/100 | 3/100 | 3/100 | 3/100 | 3/100 |
| UL22A | L | 1/33.3 | 0/0 | 1/33.3 | 0/0 | 0/0 | 0/0 |
| UL24 | LL | 2/66.6 | 2/66.6 | 2/66.6 | 2/66.6 | 2/66.6 | 2/66.6 |
| UL25 | L | 3/100 | 3/100 | 3/100 | 3/100 | 3/100 | 3/100 |
| UL26 | DE | 3/100 | 3/100 | 3/100 | 3/100 | 3/100 | 3/100 |
| UL29 | L | 3/100 | 3/100 | 3/100 | 3/100 | 3/100 | 3/100 |
| UL30 | L | 1/33.3 | 0/0 | 0/0 | 0/0 | 1/33.3 | 0/0 |
| UL31 | L | 2/66.6 | 1/33.3 | 1/33.3 | 1/33.3 | 1/33.3 | 1/33.3 |
| UL32 | DE | 3/100 | 3/100 | 2/66.6 | 2/66.6 | 2/66.6 | 2/66.6 |
| UL34 | DE | 3/100 | 3/100 | 3/100 | 3/100 | 3/100 | 3/100 |
| UL35 | DE | 3/100 | 2/66.6 | 2/66.6 | 2/66.6 | 2/66.6 | 2/66.6 |
| UL36 | IE | 3/100 | 3/100 | 3/100 | 2/66.6 | 3/100 | 3/100 |
| UL37 | IE | 3/100 | 2/66.6 | 3/100 | 3/100 | 3/100 | 3/100 |
| UL38 | DE | 3/100 | 3/100 | 3/100 | 3/100 | 3/100 | 3/100 |
| UL4 | DE | 3/100 | 2/66.6 | 0/0 | 0/0 | 1/33.3 | 0/0 |
| UL40 | LL | 1/33.3 | 1/33.3 | 1/33.3 | 1/33.3 | 1/33.3 | 1/33.3 |
| UL43 | L | 3/100 | 2/66.6 | 3/100 | 2/66.6 | 2/66.6 | 2/66.6 |
| UL44 | DE | 3/100 | 3/100 | 3/100 | 3/100 | 3/100 | 3/100 |
| UL47 | LL | 3/100 | 3/100 | 3/100 | 3/100 | 3/100 | 3/100 |
| UL48 | DE | 3/100 | 3/100 | 3/100 | 3/100 | 3/100 | 2/66.6 |
| UL49 | LL | 1/33.3 | 0/0 | 1/33.3 | 1/33.3 | 1/33.3 | 1/33.3 |
| UL52 | L | 3/100 | 2/66.6 | 3/100 | 3/100 | 3/100 | 2/66.6 |
| UL54 | DE | 3/100 | 3/100 | 3/100 | 3/100 | 3/100 | 3/100 |
| UL69 | LL | 3/100 | 3/100 | 2/66.6 | 3/100 | 3/100 | 3/100 |
| UL70 | LL | 3/100 | 3/100 | 3/100 | 3/100 | 3/100 | 3/100 |
| UL71 | DE | 3/100 | 3/100 | 3/100 | 3/100 | 3/100 | 3/100 |
| UL76 | L | 3/100 | 3/100 | 3/100 | 2/66.6 | 3/100 | 3/100 |
| UL78 | DE | 1/33.3 | 1/33.3 | 1/33.3 | 1/33.3 | 1/33.3 | 1/33.3 |
| UL79 | L | 2/66.6 | 2/66.6 | 2/66.6 | 2/66.6 | 2/66.6 | 2/66.6 |
| UL80 | L | 3/100 | 3/100 | 2/66.6 | 3/100 | 3/100 | 3/100 |
| UL82 | L | 3/100 | 3/100 | 3/100 | 3/100 | 3/100 | 3/100 |
| UL83 | LL | 3/100 | 3/100 | 3/100 | 3/100 | 3/100 | 3/100 |
| UL84 | DE | 3/100 | 2/66.6 | 2/66.6 | 2/66.6 | 2/66.6 | 2/66.6 |
| UL87 | L | 1/33.3 | 1/33.3 | 1/33.3 | 1/33.3 | 1/33.3 | 1/33.3 |
| UL88 | L | 3/100 | 3/100 | 3/100 | 3/100 | 3/100 | 3/100 |
| UL94 | L | 3/100 | 3/100 | 3/100 | 3/100 | 3/100 | 3/100 |
| UL95 | DE | 2/66.6 | 1/33.3 | 1/33.3 | 1/33.3 | 1/33.3 | 1/33.3 |
| UL96 | DE | 2/66.6 | 2/66.6 | 2/66.6 | 2/66.6 | 2/66.6 | 2/66.6 |
| UL97 | DE | 3/100 | 3/100 | 3/100 | 3/100 | 3/100 | 3/100 |
| UL98 | DE | 3/100 | 3/100 | 3/100 | 3/100 | 3/100 | 3/100 |
| UL99 | L | 2/66.6 | 2/66.6 | 2/66.6 | 2/66.6 | 1/33.3 | 1/33.3 |
| US12 | DE | 3/100 | 2/66.6 | 2/66.6 | 2/66.6 | 2/66.6 | 2/66.6 |
| US13 | DE | 1/33.3 | 1/33.3 | 1/33.3 | 1/33.3 | 1/33.3 | 1/33.3 |
| US14 | DE | 1/33.3 | 1/33.3 | 1/33.3 | 1/33.3 | 1/33.3 | 1/33.3 |
| US15 | LL | 1/33.3 | 1/33.3 | 1/33.3 | 1/33.3 | 0/0 | 1/33.3 |
| US18 | DE | 1/33.3 | 1/33.3 | 1/33.3 | 1/33.3 | 1/33.3 | 1/33.3 |
| US22 | DE | 3/100 | 3/100 | 3/100 | 3/100 | 3/100 | 3/100 |
| US23 | DE | 3/100 | 3/100 | 3/100 | 3/100 | 3/100 | 3/100 |
| US24 | DE | 3/100 | 3/100 | 3/100 | 3/100 | 3/100 | 3/100 |
| US34 | DE | 1/33.3 | 0/0 | 0/0 | 0/0 | 1/33.3 | 1/33.3 |
| US8 | DE | 1/33.3 | 1/33.3 | 1/33.3 | 1/33.3 | 1/33.3 | 1/33.3 |
| US9 | DE | 3/100 | 3/100 | 3/100 | 3/100 | 2/66.6 | 2/66.6 |
| gB | DE | 3/100 | 2/66.6 | 2/66.6 | 2/66.6 | 2/66.6 | 2/66.6 |
| gH | L | 1/33.3 | 1/33.3 | 1/33.3 | 1/33.3 | 1/33.3 | 1/33.3 |
| gL | L | 3/100 | 3/100 | 3/100 | 3/100 | 3/100 | 3/100 |
| gM | L | 3/100 | 2/66.6 | 2/66.6 | 2/66.6 | 2/66.6 | 2/66.6 |
| gN | L | 2/66.6 | 0/0 | 0/0 | 0/0 | 0/0 | 0/0 |

*Number of Conserved Peptides/Percentage of Conserved Peptides

TABLE
FIG. 9E: Numerical values corresponding heatmap in FIG. 9E.

| Protein | | KSHV strain* | | | |
|---|---|---|---|---|---|
| Gene | Tempor. | BAC16 | BrK | DG1 | GK18 |
| 70 | DE | 3/100 | 3/100 | 3/100 | 3/100 |
| CVC1 | L | 3/100 | 3/100 | 3/100 | 3/100 |
| CVC2 | L | 3/100 | 3/100 | 3/100 | 3/100 |
| DBP | DE | 3/100 | 3/100 | 2/66.6 | 3/100 |
| DUT | DE | 3/100 | 3/100 | 3/100 | 3/100 |
| HELI | DE | 3/100 | 3/100 | 3/100 | 3/100 |
| K14 | DE | 2/66.6 | 2/66.6 | 2/66.6 | 2/66.6 |
| K2 | DE | 3/100 | 3/100 | 3/100 | 3/100 |
| K3 | DE | 3/100 | 3/100 | 3/100 | 2/66.6 |
| K5 | DE | 3/100 | 3/100 | 1/33.3 | 3/100 |
| K8 | IE | 2/66.6 | 2/66.6 | 2/66.6 | 2/66.6 |
| K8.1 | L | 3/100 | 3/100 | 3/100 | 3/100 |
| MCP | L | 3/100 | 3/100 | 3/100 | 3/100 |
| NEC1 | DE | 3/100 | 3/100 | 3/100 | 3/100 |
| NEC2 | DE | 3/100 | 3/100 | 3/100 | 3/100 |
| ORF K4 | DE | 1/33.3 | 1/33.3 | 1/33.3 | 1/33.3 |
| ORF10 | DE | 3/100 | 3/100 | 3/100 | 3/100 |
| ORF11 | DE | 3/100 | 3/100 | 3/100 | 3/100 |
| ORF16 | IE | 2/66.6 | 2/66.6 | 2/66.6 | 2/66.6 |
| ORF17 | DE | 3/100 | 3/100 | 3/100 | 3/100 |
| ORF2 | DE | 2/66.6 | 2/66.6 | 1/33.3 | 2/66.6 |
| ORF20 | L | 1/33.3 | 1/33.3 | 1/33.3 | 1/33.3 |
| ORF23 | L | 2/66.6 | 2/66.6 | 2/66.6 | 2/66.6 |
| ORF24 | L | 3/100 | 3/100 | 3/100 | 3/100 |
| ORF27 | L | 3/100 | 3/100 | 3/100 | 3/100 |
| ORF28 | L | 1/33.3 | 1/33.3 | 1/33.3 | 1/33.3 |
| ORF33 | L | 3/100 | 3/100 | 3/100 | 3/100 |
| ORF34 | L | 2/66.6 | 2/66.6 | 2/66.6 | 2/66.6 |
| ORF35 | L | 2/66.6 | 2/66.6 | 2/66.6 | 2/66.6 |
| ORF36 | DE | 1/33.3 | 1/33.3 | 1/33.3 | 1/33.3 |
| ORF37 | DE | 3/100 | 3/100 | 3/100 | 3/100 |
| ORF38 | L | 1/33.3 | 1/33.3 | 1/33.3 | 1/33.3 |
| ORF4 | L | 3/100 | 3/100 | 2/66.6 | 3/100 |
| ORF40 | DE | 2/66.6 | 2/66.6 | 2/66.6 | 2/66.6 |
| ORF42 | L | 2/66.6 | 2/66.6 | 2/66.6 | 2/66.6 |
| ORF43 | L | 3/100 | 3/100 | 3/100 | 3/100 |
| ORF45 | IE | 3/100 | 3/100 | 3/100 | 3/100 |
| ORF46 | DE | 2/66.6 | 2/66.6 | 2/66.6 | 2/66.6 |
| ORF48 | IE | 1/33.3 | 1/33.3 | 1/33.3 | 1/33.3 |
| ORF49 | DE | 1/33.3 | 1/33.3 | 1/33.3 | 1/33.3 |
| ORF50 | IE | 3/100 | 3/100 | 3/100 | 3/100 |
| ORF52 | L | 3/100 | 3/100 | 3/100 | 3/100 |
| ORF55 | L | 3/100 | 3/100 | 3/100 | 3/100 |
| ORF56 | DE | 3/100 | 3/100 | 3/100 | 3/100 |
| ORF57 | IE | 3/100 | 3/100 | 3/100 | 3/100 |
| ORF59 | DE | 3/100 | 3/100 | 2/66.6 | 3/100 |
| ORF63 | L | 0/0 | 1/33.3 | 1/33.3 | 1/33.3 |
| ORF64 | L | 3/100 | 3/100 | 3/100 | 3/100 |
| ORF66 | DE | 2/66.6 | 2/66.6 | 2/66.6 | 2/66.6 |
| ORF68 | L | 3/100 | 3/100 | 3/100 | 3/100 |
| ORF75 | L | 3/100 | 3/100 | 3/100 | 3/100 |
| ORF9 | DE | 3/100 | 3/100 | 3/100 | 3/100 |
| RIR1 | DE | 3/100 | 3/100 | 3/100 | 3/100 |
| SCP | L | 2/66.6 | 2/66.6 | 2/66.6 | 2/66.6 |
| TK | DE | 3/100 | 3/100 | 3/100 | 3/100 |
| TRX1 | L | 1/33.3 | 1/33.3 | 1/33.3 | 1/33.3 |
| TRX2 | L | 2/66.6 | 2/66.6 | 2/66.6 | 2/66.6 |
| gB | L | 2/66.6 | 2/66.6 | 2/66.6 | 2/66.6 |
| gH | L | 3/100 | 3/100 | 3/100 | 3/100 |
| gL | L | 3/100 | 3/100 | 3/100 | 3/100 |
| gM | L | 3/100 | 3/100 | 3/100 | 3/100 |
| vIRF-1 | DE | 1/33.3 | 1/33.3 | 1/33.3 | 1/33.3 |

*Number of Conserved Peptides/Percentage of Conserved Peptides

1. An assay, comprising:
obtaining a sample comprising:
a cell or tissue infected with a herpesvirus,
an extract from a cell or tissue infected with a herpesvirus, or
a protein preparation from a cell or tissue infected with a herpesvirus; and determining abundance level of a plurality of herpesvirus proteins in the sample using parallel reaction monitoring (PRM) to quantify signature peptide(s) corresponding to the herpesvirus proteins;
wherein the herpesvirus is HSV-1 and the signature peptides comprise a sequence selected from one of SEQ ID NOs: 1-21; or the herpesvirus is HCMV and the signature peptides comprise a sequence selected from one of SEQ ID NOs: 220-453; or the herpesvirus is KSHV and the signature peptides comprise a sequence selected from one of SEQ ID NOs: 454-606.

2. The assay of claim 1, wherein for at least one herpesvirus protein for which the abundance level is determined, at least two signature peptides are quantified.

3. The assay of claim 1, wherein determining the abundance level of the plurality of herpesvirus proteins using PRM comprises subjecting the sample to liquid chromatography coupled to tandem mass spectrometry (LC-MS/MS).

4. The assay of claim 1, wherein:
the plurality of herpesvirus proteins comprises at least one herpesvirus protein from each temporal class of viral replication for that herpesvirus; and/or
the cell or tissue infected with the herpesvirus is a human cell or human tissue.

5. (canceled)

6. The assay of claim 1, wherein the plurality of herpesvirus proteins constitutes approximately 30-70% of the predicted viral proteome, or 50-80% of the predicted viral proteome.

7. A time course assay, comprising:
repeating the assay of claim 1 a plurality of times, where for each repetition the sample is obtained at a different timepoint in a time course.

8. The time course assay of claim 7, where the different timepoints are:
different times post infection of the cell or tissue with the herpesvirus;
different times post exposure of the cell or tissue to a compound variable; or
different times post exposure of the cell or tissue to an environmental variable.

9. The time course assay of claim 8, wherein the different times after infection of the cell or tissue with the herpesvirus include at least one time from each state of a replication cycle of the herpesvirus.

10. (canceled)

11. An exposure or dosage course assay, comprising:
repeating the assay of claim 1 a plurality of times, where for each repetition the sample is obtained from a cell or tissue that has been exposed to a different compound or condition or a different dosage of a compound or a condition.

12. The exposure or dosage course assay of claim 11, wherein the different compounds comprise one or more of known antiviral compounds, proposed antiviral compounds, test compounds, small molecule drugs or drug candidates, or siRNAs or other biologically active non-coding RNAs.

13. The exposure or dosage course assay of claim 12, wherein the known antiviral compounds comprise one or more of acyclovir, ganciclovir, another nucleoside, penciclovir, famciclovir, valacyclovir, valganciclovir, cidofovir, another nucleotide phosphonate, fomivirsen, foscarnet, or honokiol.

14. (canceled)

15. The exposure or dosage course assay of claim 11, wherein the different exposures comprise one or more of genetic modification of the cell or tissue, genetic modification of the herpesvirus, environmental conditions, or cell or tissue growth or harvesting conditions.

16. (canceled)

17. A method for quantification of herpesvirus proteins from multiple temporal classes of viral replication, comprising:
subjecting a cell sample or cell extract from a cell infected with a herpesvirus to parallel reaction monitoring (PRM) to generate abundance data;
analyzing the abundance data to quantify signature peptide(s) corresponding to at least one herpesvirus protein from each of at least two temporal classes of viral replication; and
providing the quantified peptide(s) results from the analyzing to a database, a computer memory, a display, a printer, or another output device;
wherein the herpesvirus is HSV-1 and one or more of the signature peptides comprise a sequence selected from one of SEQ ID NOs: 1-219; or the herpesvirus is HCMV and one or more of the signature peptides comprise a sequence selected from one of SEQ ID NOs: 220-453; or the herpesvirus is KSHV and one or more of the signature peptides comprise a sequence selected from one of SEQ ID NOs: 454-606.

18. Use of the assay of claim 1, to:
screen a drug candidate as a modulator of viral infection;
analyze a stage of infection at which a test compound acts;
determine what functional family(s) of viral proteins are affected by a drug or drug candidate;
characterize viral and/or host responses to viral infection;
characterize viral and/or host responses to drug treatment; or
characterize viral and/or host responses to genetic manipulation of either the viral genome or the host genome.

19. A kit for use with the assay of claim 1, comprising:
parameters for performing the assay for a target herpesvirus;
a set of heavy isotope-labeled peptides for use as controls; and
a USB drive or other non-transitory computer readable medium containing software for assay analysis and/or standardized report generation.

20-24. (canceled)

25. A non-naturally occurring, labeled peptide having an amino acid sequence selected from SEQ ID NOs: 1-606.

26. The non-naturally occurring, labeled peptide of claim 25, wherein the label enables the peptide to be distinguished from an unlabeled peptide with the same amino acid sequence in liquid chromatography coupled to tandem mass spectrometry (LC-MS/MS) analysis.

27. A plurality of the non-naturally occurring, labeled peptides of claim 25, which plurality is specific for HSV-1, comprising:
at least one peptide, at least two peptides, or at least three peptides each of the 60 proteins listed in Table 1, the peptides comprising a sequence selected from SEQ ID NOs: 1-4, 5-8, 9-12, 13-17, 18-21, 22-27, 28-31, 32-37, 38-42, 43-46, 47-50, 51-54, 55-58, 59-62, 63-66, 67-70, 71-76, 77-79, 80-83, 84-87, 88-92, 93-96, 97, 98-101, 102-103, 104-107, 108-109, 110-115, 116-118, 119-123, 124-126, 127-130, 131, 132-136, 137-141, 142, 143-145, 146-150, 151-156, 157, 158, 159-160, 161-165, 166-171, 172, 173-178, 179, 180-184, 185-189, 190-191, 192-193, 194-195, 196-198, 199-203, 204, 205-207, 208-211, 212, 213-217, 218, or 219;
at least one peptide from at least one protein from each temporal stage of HSV-viral replication, where the peptides from the Intermediate Early (IE) temporal stage are selected from SEQ ID NOs: 13-27, 59-62, and 212; the peptides from the Early (E) temporal stage are selected from SEQ ID NOs: 1-4, 28-37, 43-50, 63-70, 80-83, 108-109, 124-130, 146-150, 192-198, 205-207, and 218-219; and the peptides from the Late (L) temporal stage are selected from SEQ ID NOs: 5-12, 38-42, 51-58, 71-79, 84-107, 110-123, 131-145, 151-191, 199-204, 208-211, and 213-217;
at least 17 peptides comprising sequences selected from SEQ ID NOs: 1-219;
more than 17 peptides each of which comprises a sequence selected from SEQ ID NOs: 1-219;
at least 30 peptides each of which comprises a sequence selected from SEQ ID NOs: 1-219;
at least 50 peptides each of which comprises a sequence selected from SEQ ID NOs: 1-219;
at least 60 peptides each of which comprises a sequence selected from SEQ ID NOs: 1-219;
219 peptides each of which has a sequence of one of SEQ ID NOs: 1-219;
wherein each peptide comprises a label that enables the labeled peptide to be distinguished from an unlabeled peptide with the same amino acid sequence in liquid chromatography coupled to tandem mass spectrometry (LC-MS/MS) analysis.

28. A plurality of the non-naturally occurring, labeled peptides of claim 25, which plurality is specific for HCMV, comprising:
at least one peptide, at least two peptides, or at least three peptides from each of the 90 proteins listed in Table 2, the peptides comprising a sequence selected from SEQ ID NOs: 220-225, 226-231, 232-234, 235-237, 238-239, 240-242, 243-244, 245-247, 248-250, 251-252, 253-254, 255-257, 258-260, 261-263, 264-266, 267-268, 269-271, 272-274, 275-277, 278-280, 281-283, 284-286, 287-289, 290-291, 292-294, 295-297, 298-300, 301, 302-303, 304-306, 307-309, 310-312, 313-314, 315-317, 318-320, 321-323, 324-326, 327-329, 330-332, 333-335, 336-338, 339-341, 342-344, 345-347, 348-350, 351-353, 354-356, 357-359, 360-362, 363-365, 366-368, 369-371, 372-374, 375-377, 378-380, 381-383, 384-386, 387-389, 390, 391-393, 394-397, 398-400, 401-402, 403-405, 406, 407-409, 410-412, 413-414, 415, 416-418, 419-420, 421-423, 424, 425-427, 428, 429, 430-432, 433-435, 436, 437-438, 439, 440-441, 442-443, 444-445, 446, 447, 448, 449, 450-452, or 453;
at least one peptide from at least one protein from each temporal stage of HCMV-viral replication, where the peptides from the Intermediate Early (IE) temporal stage are selected from SEQ ID NOs: 245-247, 267-268, 290-297, and 324-329; the peptides from the Late (L) temporal stage are selected from SEQ ID NOs: 226-231, 238-244, 248-250, 261-263, 278-280, 284-286, 301, 304-306, 310-314, 333-335, 345-347, 357-362, 369-374, 401-402, 407-412, 415, 424, 433-435, 437-439, and 444-445; and the peptides from the Late Late (LL) temporal stage are selected from SEQ ID NOs: 220-225, 264-266, 269-274, 298-300, 302-303, 339-341, 351-353, 363-365, 394-397, 406, 428-432, and 448;
at least 90 peptides each of which comprises a sequence selected from SEQ ID NOs: 220-453;
more than 90 peptides each of which comprises a sequence selected from SEQ ID NOs: 220-453;
at least 30 peptides each of which comprises a sequence selected from SEQ ID NOs: 220-453;
at least 50 peptides each of which comprises a sequence selected from SEQ ID NOs: 220-453;
at least 100 peptides each of which comprises a sequence selected from SEQ ID NOs: 220-453;
at least 150 peptides each of which comprises a sequence selected from SEQ ID NOs: 220-453;
at least 200 peptides each of which comprises a sequence selected from SEQ ID NOs: 220-453; or
233 peptides each of which has a sequence of one of SEQ ID NOs: 220-253;
wherein each peptide in the collection comprises a label that enables the labeled peptide to be distinguished from an unlabeled peptide with the same amino acid sequence in liquid chromatography coupled to tandem mass spectrometry (LC-MS/MS) analysis.

29. A plurality of the non-naturally occurring, labeled peptides of claim 25, which plurality is specific for KSHV, comprising:
at least one peptide, at least two peptides, or at least three peptides from each of the 62 proteins listed in Table 3, the peptides comprising a sequence selected from SEQ ID NOs: 454-456, 457-459, 460-462, 463-464, 465-467, 468-470, 471-473, 474-476, 477-479, 480-482, 483-485, 486-488, 489-491, 492-494, 495-497, 498, 499-501, 502-504, 505-507, 508-510, 511-513, 514-516, 517-519, 520-522, 523-525, 526-527, 528-530, 531-533, 534-536, 537-539, 540-542, 543-545, 546-548, 549-550, 551, 552-553, 554-555, 556, 557-558, 559-561, 562-564, 565, 566-568, 569-570, 571-572, 573, 574-576, 577-578, 579-580, 581-583, 584-585, 586, 587, 588-590, 591-593, 594, 595-597, 598-599, 600-602, 603, 604-605, or 606;
at least one peptide from at least one protein from each temporal stage of KSHV-viral replication, where the peptides from the Intermediate Early (IE) temporal stage are selected from SEQ ID NOs: 474-476, 502-507, 511-513, 552-553, and 586; the peptides from the Delayed Early (DE) temporal stage are selected from SEQ ID NOs: 454-462, 465-473, 483-497, 514-516, 520-525, 528-530, 546-551, 554-555, 573-578, 584-585, 587, 591-593, 598-599, and 606; and the peptides from the Late (L) temporal stage are selected from SEQ ID NOs: 463-464, 477-482, 498, 499-501, 508-510, 517-519, 526-527, 531-545, 556-572, 579-583, 588-590, 594-597, and 600-605;
at least 62 peptides each of which comprising a sequence selected from SEQ ID NOs: 454-606;
more than 62 peptides each of which comprises a sequence selected from SEQ ID NOs: 454-606;
at least 30 peptides each of which comprises a sequence selected from SEQ ID NOs: 454-606;
at least 50 peptides each of which comprises a sequence selected from SEQ ID NOs: 454-606;
at least 75 peptides each of which comprises a sequence selected from SEQ ID NOs: 454-606;
at least 100 peptides each of which comprises a sequence selected from SEQ ID NOs: 454-606;
at least 150 peptides each of which comprises a sequence selected from SEQ ID NOs: 454-606;
151 peptides each of which has a sequence of one of SEQ ID NOs: 454-606;
wherein each peptide comprises a label that enables the labeled peptide to be distinguished from an unlabeled peptide with the same amino acid sequence in liquid chromatography coupled to tandem mass spectrometry (LC-MS/MS) analysis.

30. (canceled)
